SEQUENCE LISTING (146 sequences)

SEQ ID NO: 1 - DNA, 4910 bp, synthetic construct
  source 1..4910: mol_type = other DNA; note = Synthetic - URA 3 gene disruption fragment
ctcaaaactatttaattagttaattgtataaactgtatgtcattataaacagggaaggttgacattgtctagcggcaatcattgtctcatttggttcattaactttggttctgttcttggaaacgggtaccaactctctcagagtgcttcaaaaatttttcagcacatttggttagacatgaactttctctgctggttaaggattcagaggtgaagtcttgaacacaatcgttgaaacatctgtccacaagagatgtgtatagcctcatgaaatcagccatttgcttttgttcaacgatcttttgaaattgttgttgttcttggtagttaagttgatccatcttggcttatgttgtgtgtatgttgtagttattcttagtatattcctgtcctgagtttagtgaaacataatatcgccttgaaatgaaaatgctgaaattcgtcgacatacaatttttcaaactttttttttttcttggtgcacggacatgtttttaaaggaagtactctataccagttattcttcaccctgcagggtacgtagcatgcactcgcaagctgtgccatcgcccaacggttaattataagaaatcaacatcagccaacaactattttcgtccccctcttttcagtggtaacgagcaattacattagtaagagactattttcttcagtgatttgtaattttttttcagtgatttgtaattctttctcgaaatatgcgggcttaacttatccggacattcactacatgcaaggaaaaacgagaaccgcggagatttcctcagtaagtaacaatgatgatctttttacgcttcatcatcactttccaaagttctaagctataagttcaagcctagatacgctgaaaaactcctgaccaacaatgtaaagaaaacaattacaattgtaaggttgaaaacatctaaaaatgaaatattttattgtacatgcacaccctgatagtcattctcttacttcatccctgaaagacgtggctgtacaagagttggaatcgcaaggtcatgaggttaaagttagtgatctttatgctcaaaagtggaaggcctcaatagaccgtgacgacttcgagcagcttttcgcaagaagagaggttaaaaataccccaagcttcttatgaagcgtatgccagaggagcattaacaaaagacgtaaatcaggaacaggaaaaacttatttgggcggactttgtcattttgtcgtttcctatatggtggtcttctatgccggctagtcgaccccctcgaccccctcgagcgatctcgagatttgctgcaacggcaacatcaatgtccacgtttacacacctacatttatatctatatttatatttatatttatttatttatgctacttagcttctatagttagttaatgcactcacgatattcaaaattgacacccttcaactactccctactattgtctactactgtctactactcctctttactatagctgctcccaataggctccaccaataggctctgtcaatacattttgcgccgccacctttcaggttgtgtcactcctgaaggaccatattgggtaatcgtgcaatttctggaagagagtgccgcgagaagtgaggcccccactgtaaatcctcgagggggcatggagtatggggcatgnaggatggaggatggggggggggggggaaaataggtagcgaaaggacccgctatcaccccacccggagaactcgttgccgggaagtcatatttcgacactccggggagtctataaaaggcgggttttgtcttttgccagttgatgttgctgagaggacttgtttgccgtttcttccgatttaacagtatagaatcaaccactgttaattatacacgttatactaacacaacaaaaacaaaaacaacgacaacaacaacaacaatgtttgctttctactttctcaccgcatgcaccactttgaagggtgttttcggagtttctccgagttacaatggtcttggtctcaccccacagatgggttgggacagctggaatacgtttgcctgcgatgtcagtgaacagctacttctagacactgctgatagaatttctgacttggggctaaaggatatgggttacaagtatgtcatcctagatgactgttggtctagcggcagggattccgacggtttcctcgttgcagacaagcacaaatttcccaacggtatgggccatgttgcagaccacctgcataataacagctttcttttcggtatgtattcgtctgctggtgagtacacctgtgctgggtaccctgggtctctggggcgtgaggaagaagatgctcaattctttgcaaataaccgcgttgactacttgaagtatgataattgttacaataaaggtcaatttggtacaccagacgtttcttaccaccgttacaaggccatgtcagatgctttgaataaaactggtaggcctattttctattctctatgtaactggggtcaggatttgacattttactggggctctggtatcgccaattcttggagaatgagcggagatattactgctgagttcacccgtccagatagcagatgtccctgtgacggtgacgaatatgattgcaagtacgccggtttccattgttctattatgaatattcttaacaaggcagctccaatggggcaaaatgcaggtgttggtggttggaacgatctggacaatctagaggtcggagtcggtaatttgactgacgatgaggaaaaggcccatttctctatgtgggcaatggtaaagtccccacttatcattggtgccgacgtgaatcacttaaaggcatcttcgtactcgatctacagtcaagcctctgtcatcgcaattaatcaagatccaaagggtattccagccacaagagtctggagatattatgtttcagacaccgatgaatatggacaaggtgaaattcaaatgtggagtggtccgcttgacaatggtgaccaagtggttgctttattgaatggaggaagcgtagcaagaccaatgaacacgaccttggaagagattttctttgacagcaatttgggttcaaaggaactgacatcgacttgggatatttacgacttatgggccaacagagttgacaactctacggcgtctgctatccttgaacagaataaggcagccaccggtattctctacaatgctacagagcagtcttataaagacggtttgtctaagaatgatacaagactgtttggccagaaaattggtagtctttctccaaatgctatacttaacacaactgttccagctcatggtatcgccttctataggttgagaccctcggcttaagctcaatgttgagcaaagcaggacgagaaaaaaaaaaataatgattgttaagaagttcatgaaaaaaaaaaggaaaaatactcaaatacttataacagagtgattaaataataaacggcagtataccctatcaggtattgagatagttttatttttgtaggtatataatctgaagcctttgaactattttctcgtatatatcatggagtatacattgcattagcaacattgcatactagtcactcgcaagctgtgccatcgcccaacggttaattataagaaatcaacatcagccaacaactattttcgtccccctcttttcagtggtaacgagcaattacattagtaagagactattttcttcagtgatttgtaattttttttcagtgatttgtaattctttctcgaaatatgcgggctwaamtaatccggacattcactacatgcaaggaaaaacgagaaccgcggagatttcctcagtaagtaacaatgatgatctttttacgcttcatcatcactttccaaagttctaagctataagttcaagcctagatacgctgaaaaactcctgaccaacaatgtaaagaaaacaattacaattgtaaggttgaaaacatctaaaaatgaaatattttattgtacatgcacaccctgatagtcattctcttacttcatccctgaaagacgtggctgtacaagagttggaatcgcaaggtcatgaggttaaagttagtgatctttatgctcaaaagtggaaggcctcaatagaccgtgacgacwwmaaaaaamaaamrmaagaagagaggttaaaaataccccaagcttcttatgaagcgtatgccagaggagcattaacaaaagacgtaaatcaggaacaggaaaaacttatttgggcggactttgtcattttgtcgtttcctatatggtggtcttctatgccggctagcggccgggcaacaaagcctcccagatttgatanattttcaatttgtgctttgaatcatgacttccacctgtttggtccgcaagaacacgtaaatgcgcaatttgtttctcccttctgcttaaaaaccatgcacctttaatattatctggaaagataaagaacagaattgttgcgtagaaacaagtagcagagccgtaaatgagaaaaatatacttccaagctggtaatttcccctttattagtccaatacagtgtccgaagaccccaccaagaataccagcaagggtgttgaaatataatgtagatcttagtggttgttctgatttcttccaccacattccgctaataatcataaaagacggtaatattccggcttcaaatacgccaagaaaaaacctcacggtaaccaaaccaccaaagctatgacatgcagccatgcacataagtaagccgccccaaatgaacaaacaaatagacacaaatttgccaattctaactcgtggcaacaaaaaaaaggatatgaactcacctaataaataaccgaaataaaaagtagaagcaactgtggaaaattgagaaccatgtaaatttgtgtcttctttcaatgtataaacagccgcaatacctaggg

SEQ ID NO: 2 - DNA, 3704 bp, synthetic construct
  source 1..3704: mol_type = other DNA; note = Synthetic - PDC gene disruption fragment
cccccagttgttgttgcaattaacaaatttgctaccgacaccgagaaggaaattgagaccattagagaagaagccatcaaggctggtgcatttgatgctgttgagtcagaccattggtcacaaggtggtaagggtgcaatcaagttagctgaggcaattgtacgtgctaccgaggaaagaccgttggaagaaagtcaacctcctaactatctttattcattagatggttcgttagaagatagactaagaacaattgccaccaagatgtatggagcaaaagatattgaactatctgagttggccaagaaacagattgaagagtatgagagtcaaggttttggcaagctgcctgtttgtattgcaaagacgcaatattctctctcccatgatccaacattgaaaggtgttccaaaggatttcatcttcccaatcagagaagttagaataagtgcaggagcaggatatttatatgcactagctgcaaagatcatgacaattccaggtctatcaacttatgccggatttatgaatgttgaagtcaacgaagatggtgagattgatggattgttttagtttttattataaaattatatattattcttaattacatatcacccttctatcagggaagggagaaacgaaaatagagagtgacctatccaagctcgggggtctaagttttaatggcccagggaatcattacttttttttctcaatccttgatggataaaagtattacatacgtacaggattgtgtattagtgtatttcgttatatgattaaacaaagtttatagattgtaaagtagacgtaaagtttagtaattcattttaatgttcattttacattcagatggcggccgcggatccagatcccccggggcgttgaagatctattctccagcaattaaatttgtgaagaataactggtatagagtacttcctttaaaaacatgtccgtgcaccaagaaaaaaaaaaagtttgaaaaattgtatgtcgacgaatttcagcattttcatttcaaggcgatattatgtttcactaaactcaggacaggaatatactaagaataactacaacatacacacaacataagccaagatggatcaacttaactaccaagaacaacaacaatttcaaaagatcgttgaacaaaagcaaatggctgatttcatgaggctatctgcagatacgcggaacaatcaatcgataatgatttgactgataaagaaaaccatacttttgtttatgtttattagttatcgctttgctacattaaaaattcacatactaaagcctttgttaaacaactttttctaaatcttaagattttactctatctagtttttttggttgtaggtgaacgtaaagtacctcatttatttttttttttttgcttgtgtaattcttttcatgcttatttaaactagtgtacatgtatcaaatctttgtgtaagaatcatttaaatctgtttaaataagcattccaaccagcttgttggtatcttttagcttgctctataggatctcttccttgaccgtacaaacctctaccaacaattatgatatccgttccagtctttacaacttcatcaacagttctatattgttgaccaagtgcatcacctttgtcatctaaaccaacccctggagtcataatgatccagtcaaaaccttcttctctaccgcccatatcgtgttgcgcaataaaaccaatgacaaactctttatcagatttagcaatttctactgttttttctgtatattcaccatatgctaaagaaccctttgatgataactcagcaagcattagcaaacctctaggttcactggttgtttcttgggctgcctccttcaagccagaaacaatacctgcacccgttacaccatgtgcattagtgatgtcagcccattcggcaatacggaagacaccagatttatattgatttttaacagtgttaccaatatcagcaaattttctatcttcaaaaatcataaaattatgtttcttggcaagctccttcaaaggcaacacagttccttcatacgtaaaatcagaaacaatatcgatgtgtgttttaactagacagatgtaaggaccaatagtgtccaaaatagagagaagcttttcagtttcagtaatatccaatgatgcacaaaggttagacttcttttcctccatgatggagaaaagtctcctagcaacaggggaagtgtgtgattctgatctttctttgtatgacgccatccttgacaaacaaactactttattaaagcgttgaagatctattctccagcaattaaatttgtgaagaataactggtatagagtacttcctttaaaaacatgtccgtgcaccaagaaaaaaaaaaagtttgaaaaattgtatgtcgacgaatttcagcattttcatttcaaggcgatattatgtttcactaaactcaggacaggaatatactaagaataactacaacatacacacaacataagccaagatggatcaacttaactaccaagaacaacaacaatttcaaaagatcgttgaacaaaagcaaatggctgatttcatgaggctatgaattcttttattataaaattatatattattcttaattacatatcacccttctatcagggaagggagaaacgaaaatagagagtgacctatccaagcttgggggtctaagttttaatggcccagggaatcattacttttttttctcaatccttgatggataaaagtattacatacgtacaggattgtgtattagtgtatttcgttatatgattaaacaaagtttatagattgtaaagtagacgtaaagtttagtaattcattttaatgttcattttacattcagatgttaattaaggcctcgagggatccgcggccgctatttttgtgttttgctgtgttttgttttattttgttttattgggaagaaaatatataataatagaatattatattaacaaataattaaagaagctcaactgttattagaataaatgggttctccgtgtcctttttatacgccttctccgaaaagaaaaaaaccatcgtatcatttgtagcccacgccacccggaaaaaccaccattgtcctcagcagtccgcaaaaatatggatgcgctcaatcaatttccctcccccgtcaatgccaaaaggataacgacacactattaagagcgcatcatttgtaaaagccgaggaagggggatacgctgaccgagacgtctcgcctcactctcggagctgagccgccctccttaagaaattcatgggaagaacacccttcgcggcttctgaacggctcgccctcgtccattggtcacctcacagtggcaactaataaggacattatagcaatagaaattaaaatggtgcacagaaatacaataggatcgaataggataggatacaataagatacggaatattagactatactgtgatacggtacgctacgatacgctacgatacgatacgatagaggataccacggatataacgtagtgttatttttcattattggggttttttttctgtttgaattttccacgtcaagagtatcccatctgacaggaaccgatggactcgtcacagtacctatcgcccgagttcaatccatggacgctgcgggtgaaggatcttcgcccgctgttggcaagccatgggatcagggcgtcgccaagggacgggcc

SEQ ID NO: 3 - DNA, 6392 bp, synthetic construct
  source 1..6392: mol_type = other DNA; note = Synthetic - PYC gene integration fragment
ctaagtagtggtgttggtgaactcaagatggactctttaggtaattatattcttgaatagttgtgtaaagcgaatatgcaaatagatttgttttataattatgcatctctttgaaagaggtttagaggcaaagttcttgcatacaatattgtgattgttttaatgtcattcttgattttcataaagagattaaaaaaaaaaaaaaaaaacttataaaattgagtagaaccatttatatataagacaaagattgtctgtattagtcctcaacacactaaaccttacatacttagggtaaatttgctaatagagtgatatgttcatgagaactccaacgacaacacaaccacctatttgcacaacaaacaccattgtcgcacgctgcgcgccctagaagtagaaagaaagggaaatgacattaagagaatcataccccgtgcccgtaacgccgaaaaaatcacaccccgtcccccacaccttaaaacctcaaccgcttaacaccgccacaccctttctctttataaacgccgtttgcattactcattcttcttataaaccgcaccccccaaaacgcggaatagcttcaaccccccaatcagatatgagtttcccgggaaacccgcttttcccgacagccccacaaggggttggtctataaaagaggacgttttccccgtcatcgagattgaagattcttacaggcccatttattcaaattggagttgattcttcttgtctttactttctttctctctttttcttccttttttaatattatcttttgtcaagcctggttccctaagttgaactctcttttcttgtgatcctcctatatagatacgccttgccaaatgcggccgcgagtccatcggttcctgtcagatgggatactcttgacgtggaaaattcaaacagaaaaaaaaccccaataatgaaaaataacactacgttatatccgtggtatcctctatcgtatcgtatcgtagcgtatcgtagcgtaccgtatcacagtatagtctaatattccgtatcttattgtatcctatcctattcgatcctattgtatttcagtgcaccattttaatttctattgctataatgtccttattagttgccactgtgaggtgaccaatggacgagggcgagccgttcagaagccgcgaagggtgttcttcccatgaatttcttaaggagggcggctcagctccgagagtgaggcgagacgtctcggtcagcgtatcccccttcctcggcttttacaaatgatgcgctcttaatagtgtgtcgttatccttttggcattgacgggggagggaaattgattgagcgcatccatatttttgcggactgctgaggacaatggtggtttttccgggtggcgtgggctacaaatgatacgatggtttttttcttttcggagaaggcgtataaaaaggacacggagaacccatttattctaaaaacagttgagcttctttaattattttttgatataatattctattattatatattttcttcccaataaaacaaaataaaacaaaacacagcaaaacacaaaaattctagataaaatgtcaactgtggaagatcactcctccctacataaattgagaaaggaatctgagattctttccaatgcaaacaaaatcttagtggctaatagaggtgaaattccaattagaattttcaggtcagcccatgaattgtcaatgcatactgtggcgatctattcccatgaagatcggttgtccatgcataggttgaaggccgacgaggcttatgcaatcggtaagactggtcaatattcgccagttcaagcttatctacaaattgacgaaattatcaaaatagcaaaggaacatgatgtttccatgatccatccaggttatggtttcttatctgaaaactccgaattcgcaaagaaggttgaagaatccggtatgatttgggttgggcctcctgctgaagttattgattctgttggtgacaaggtttctgcaagaaatttggcaattaaatgtgacgttcctgttgttcctggtaccgatggtccaattgaagacattgaacaggctaaacagtttgtggaacaatatggttatcctgtcattataaaggctgcatttggtggtggtggtagaggtatgagagttgttagagaaggtgatgatatagttgatgctttccaaagagcgtcatctgaagcaaagtctgcctttggtaatggtacttgttttattgaaagatttttggataagccaaaacatattgaggttcaattattggctgataattatggtaacacaatccatctctttgaaagagattgttctgttcaaagaagacatcaaaaggttgttgaaattgcacctgccaaaactttacctgttgaagttagaaatgctatattaaaggatgctgtaacgttagctaaaaccgctaactatagaaatgctggtactgcagaatttttagttgattcccaaaacagacattattttattgaaattaatccaagaattcaagttgaacatacaattactgaagaaatcacgggtgttgatattgttgccgctcaaattcaaattgctgcaggtgcatcattggaacaattgggtctattacaaaacaaaattacaactagaggttttgcaattcaatgtagaattacaaccgaggatcctgctaagaattttgccccagatacaggtaaaattgaggtttatagatctgcaggtggtaacggtgtcagattagatggtggtaatgggtttgccggtgctgttatatctcctcattatgactcgatgttggttaaatgttcaacatctggttctaactatgaaattgccagaagaaagatgattagagctttagttgaatttagaatcagaggtgtcaagaccaatattcctttcttattggcattgctaactcatccagttttcatttcgggtgattgttggacaacttttattgatgataccccttcgttattcgaaatggtttcttcaaagaatagagcccaaaaattattggcatatattggtgacttgtgtgtcaatggttcttcaattaaaggtcaaattggtttccctaaattgaacaaggaagcagaaatcccagatttgttggatccaaatgatgaggttattgatgtttctaaaccttctaccaatggtctaagaccgtatctattaaagtatggaccagatgcgttttccaaaaaagttcgtgaattcgatggttgtatgattatggataccacctggagagatgcacatcaatcattattggctacaagagttagaactattgatttactgagaattgctccaacgactagtcatgccttacaaaatgcatttgcattagaatgttggggtggcgcaacatttgatgttgcgatgaggttcctctatgaagatccttgggagagattaagacaacttagaaaggcagttccaaatattcctttccaaatgttattgagaggtgctaatggtgttgcttattcgtcattacctgataatgcaattgatcattttgttaagcaagcaaaggataatggtgttgatattttcagagtctttgatgctttgaacgatttggaacaattgaaggttggtgttgatgctgtcaagaaagccggaggtgttgttgaagctacagtttgttactcaggtgatatgttaattccaggtaaaaagtataacttggattattatttagagactgttggaaagattgtggaaatgggtacccatattttaggtattaaggatatggctggcacgttaaagccaaaggctgctaagttgttgattggctcgatcagatcaaaataccctgacttggttatccatgtccatacccatgactctgctggtaccggtatttcaacttatgttgcatgcgcattggcaggtgccgacattgtcgattgtgcaatcaattcgatgtctggtttaacctctcaaccttcaatgagtgcttttattgctgctttagatggtgatatcgaaactggtgttccagaacattttgcaagacaattagatgcatactgggcagaaatgagattgttatactcatgtttcgaagccgacttgaagggaccagacccagaagtttataaacatgaaattccaggtggacagttgactaacctaatcttccaagcccaacaagttggtttgggtgaacaatgggaagaaactaagaagaagtatgaagatgctaacatgttgttgggtgatattgtcaaggttaccccaacctccaaggttgttggtgatttagcccaatttatggtttctaataaattagaaaaagaagatgttgaaaaacttgctaatgaattagatttcccagattcagttcttgatttctttgaaggattaatgggtacaccatatggtggattcccagagcctttgagaacaaatgtcatttccggcaagagaagaaaattaaagggtagaccaggtttagaattagaacctttcaacctcgaggaaatcagagaaaatttggtttccagatttggtccaggtattactgaatgtgatgttgcatcttataacatgtatccaaaggtttacgagcaatatcgtaaggtggttgaaaaatatggtgatttatctgttttaccaacaaaagcatttttggctcctccaactattggtgaagaagttcatgtggaaattgagcaaggtaagactttgattattaagttattagccatttctgacttgtctaaatctcatggtacaagagaagtatactttgaattgaatggtgaaatgagaaaggttacaattgaagataaaacagctgcaattgagactgttacaagagcaaaggctgacggacacaatccaaatgaagttggtgcgccaatggctggtgtcgttgttgaagttagagtgaagcatggaacagaagttaagaagggtgatccattagccgttttgagtgcaatgaaaatggaaatggttatttctgctcctgttagtggtagggtcggtgaagtttttgtcaacgaaggcgattccgttgatatgggtgatttgcttgtgaaaattgccaaagatgaagcgccagcagcttaattaattctgtctttgattttcttatgttattcaaaacatctgccccaaaatctaacgattatatatattcctacgtataactgtatagctaattattgatttatttgtacataaaaaccacataaatgtaaaagcaagaaaaaaaataactaaggagaaggatcaatatctcatttataatgctcgccaaagcagcgtacgtgaattttaatcaagacatcaacaaatcttgcaacttggttatatcgcttcttcacccactcacccgcttttctacattgttgaacacaaatatatacaggggtatgtctcaaggtcaagtgcagtttcaacagagactacctcaaggtacctcttcagaaatgcagaacttcactcttgatcagattttctccgaattaaaggtttaaacatagcctcatgaaatcagccatttgcttttgttcaacgatcttttgaaattgttgttgttcttggtagttaagttgatccatcttggcttatgttgtgtgtatgttgtagttattcttagtatattcctgtcctgagtttagtgaaacataatatcgccttgaaatgaaaatgctgaaattcgtcgacatacaatttttcaaactttttttttttcttggtgcacggacatgtttttaaaggaagtactctataccagttattcttcacaaatttaattgctggagaatagatcttcaacgctttaataaagtagtttgtttgtcaaggatggcgtcatacaaagaaagatcagaatcacacacttcccctgttgctaggagacttttctccatcatggaggaaaagaagtctaacctttgtgcatcattggatattactgaaactgaaaagcttctctctattttggacactattggtccttacatctgtctagttaaaacacacatcgatattgtttctgattttacgtatgaaggaactgtgttgcctttgaaggagcttgccaagaaacataattttatgatttttgaagatagaaaatttgctgatattggtaacactgttaaaaatcaatataaatctggtgtcttccgtattgccgaatgggctgacatcactaatgcacatggtgtaacgggtgcaggtattgtttctggcttgaaggaggcagcccaagaaacaaccagtgaacctagaggtttgctaatgcttgctgagttatcatcaaagggttctttagcatatggtgaatatacagaaaaaacagtagaaattgctaaatctgataaagagtttgttgag

SEQ ID NO: 4 - DNA, 3543 bp, Issatchenkia orientalis
  source 1..3543: mol_type = other DNA
atgtcaactgtggaagatcactcctccctacataaattgagaaaggaatctgagattctttccaatgcaaacaaaatcttagtggctaatagaggtgaaattccaattagaattttcaggtcagcccatgaattgtcaatgcatactgtggcgatctattcccatgaagatcggttgtccatgcataggttgaaggccgacgaggcttatgcaatcggtaagactggtcaatattcgccagttcaagcttatctacaaattgacgaaattatcaaaatagcaaaggaacatgatgtttccatgatccatccaggttatggtttcttatctgaaaactccgaattcgcaaagaaggttgaagaatccggtatgatttgggttgggcctcctgctgaagttattgattctgttggtgacaaggtttctgcaagaaatttggcaattaaatgtgacgttcctgttgttcctggtaccgatggtccaattgaagacattgaacaggctaaacagtttgtggaacaatatggttatcctgtcattataaaggctgcatttggtggtggtggtagaggtatgagagttgttagagaaggtgatgatatagttgatgctttccaaagagcgtcatctgaagcaaagtctgcctttggtaatggtacttgttttattgaaagatttttggataagccaaaacatattgaggttcaattattggctgataattatggtaacacaatccatctctttgaaagagattgttctgttcaaagaagacatcaaaaggttgttgaaattgcacctgccaaaactttacctgttgaagttagaaatgctatattaaaggatgctgtaacgttagctaaaaccgctaactatagaaatgctggtactgcagaatttttagttgattcccaaaacagacattattttattgaaattaatccaagaattcaagttgaacatacaattactgaagaaatcacgggtgttgatattgttgccgctcaaattcaaattgctgcaggtgcatcattggaacaattgggtctattacaaaacaaaattacaactagaggttttgcaattcaatgtagaattacaaccgaggatcctgctaagaattttgccccagatacaggtaaaattgaggtttatagatctgcaggtggtaacggtgtcagattagatggtggtaatgggtttgccggtgctgttatatctcctcattatgactcgatgttggttaaatgttcaacatctggttctaactatgaaattgccagaagaaagatgattagagctttagttgaatttagaatcagaggtgtcaagaccaatattcctttcttattggcattgctaactcatccagttttcatttcgggtgattgttggacaacttttattgatgataccccttcgttattcgaaatggtttcttcaaagaatagagcccaaaaattattggcatatattggtgacttgtgtgtcaatggttcttcaattaaaggtcaaattggtttccctaaattgaacaaggaagcagaaatcccagatttgttggatccaaatgatgaggttattgatgtttctaaaccttctaccaatggtctaagaccgtatctattaaagtatggaccagatgcgttttccaaaaaagttcgtgaattcgatggttgtatgattatggataccacctggagagatgcacatcaatcattattggctacaagagttagaactattgatttactgagaattgctccaacgactagtcatgccttacaaaatgcatttgcattagaatgttggggtggcgcaacatttgatgttgcgatgaggttcctctatgaagatccttgggagagattaagacaacttagaaaggcagttccaaatattcctttccaaatgttattgagaggtgctaatggtgttgcttattcgtcattacctgataatgcaattgatcattttgttaagcaagcaaaggataatggtgttgatattttcagagtctttgatgctttgaacgatttggaacaattgaaggttggtgttgatgctgtcaagaaagccggaggtgttgttgaagctacagtttgttactcaggtgatatgttaattccaggtaaaaagtataacttggattattatttagagactgttggaaagattgtggaaatgggtacccatattttaggtattaaggatatggctggcacgttaaagccaaaggctgctaagttgttgattggctcgatcagatcaaaataccctgacttggttatccatgtccatacccatgactctgctggtaccggtatttcaacttatgttgcatgcgcattggcaggtgccgacattgtcgattgtgcaatcaattcgatgtctggtttaacctctcaaccttcaatgagtgcttttattgctgctttagatggtgatatcgaaactggtgttccagaacattttgcaagacaattagatgcatactgggcagaaatgagattgttatactcatgtttcgaagccgacttgaagggaccagacccagaagtttataaacatgaaattccaggtggacagttgactaacctaatcttccaagcccaacaagttggtttgggtgaacaatgggaagaaactaagaagaagtatgaagatgctaacatgttgttgggtgatattgtcaaggttaccccaacctccaaggttgttggtgatttagcccaatttatggtttctaataaattagaaaaagaagatgttgaaaaacttgctaatgaattagatttcccagattcagttcttgatttctttgaaggattaatgggtacaccatatggtggattcccagagcctttgagaacaaatgtcatttccggcaagagaagaaaattaaagggtagaccaggtttagaattagaacctttcaacctcgaggaaatcagagaaaatttggtttccagatttggtccaggtattactgaatgtgatgttgcatcttataacatgtatccaaaggtttacgagcaatatcgtaaggtggttgaaaaatatggtgatttatctgttttaccaacaaaagcatttttggctcctccaactattggtgaagaagttcatgtggaaattgagcaaggtaagactttgattattaagttattagccatttctgacttgtctaaatctcatggtacaagagaagtatactttgaattgaatggtgaaatgagaaaggttacaattgaagataaaacagctgcaattgagactgttacaagagcaaaggctgacggacacaatccaaatgaagttggtgcgccaatggctggtgtcgttgttgaagttagagtgaagcatggaacagaagttaagaagggtgatccattagccgttttgagtgcaatgaaaatggaaatggttatttctgctcctgttagtggtagggtcggtgaagtttttgtcaacgaaggcgattccgttgatatgggtgatttgcttgtgaaaattgccaaagatgaagcgccagcagcttaa

SEQ ID NO: 5 - DNA, 4886 bp, synthetic construct
  source 1..4886: mol_type = other DNA; note = Synthetic - MAE gene integration fragment
ctttgaaggagcttgccaagaaacataattttatgatttttgaagatagaaaatttgctgatattggtaacactgttaaaaatcaatataaatctggtgtcttccgtattgccgaatgggctgacatcactaatgcacatggtgtaacgggtgcaggtattgtttctggcttgaaggaggcagcccaagaaacaaccagtgaacctagaggtttgctaatgcttgctgagttatcatcaaagggttctttagcatatggtgaatatacagaaaaaacagtagaaattgctaaatctgataaagagtttgtcattggttttattgcgcaacacgatatgggcggtagagaagaaggttttgactggatcattatgactccaggggttggtttagatgacaaaggtgatgcacttggtcaacaatatagaactgttgatgaagttgtaaagactggaacggatatcataattgttggtagaggtttgtacggtcaaggaagagatcctatagagcaagctaaaagataccaacaagctggttggaatgcttatttaaacagatttaaatgattcttacacaaagatttgatacatgtacactagtttaaataagcatgaaaagaattacacaagcaaaaaaaaaaaaataaatgaggtactttacgttcacctacaaccaaaaaaactagatagagtaaaatcttaagatttagaaaaagttgtttaacaaaggctttagtatgtgaatttttaatgtagcaaagcgataactaataaacataaacaaaagtatggttttctttatcagtcaaatcattatcgattgattgttccgcgtatctgcagatagcctcatgaaatcagccatttgcttttgttcaacgatcttttgaaattgttgttgttcttggtagttaagttgatccatcttggcttatgttgtgtgtatgttgtagttattcttagtatattcctgtcctgagtttagtgaaacataatatcgccttgaaatgaaaatgctgaaattcgtcgacatacaatttttcaaactttttttttttcttggtgcacggacatgtttttaaaggaagtactctataccagttattcttcacaaatttaattgctggagaatagatcttcaacgcgtttaaacagcaatttgaggaaggaataggagaaggagaagcaatttctaggaaagagcaaggtgtgcaacagcatgctctgaatgatattttcagcaatagttcagttgaagaacctgttggcgtatctacatcacttcctacaaacaacaccacgaattgcgtccgtggtgacgcaactacgaatggcattgtcaatgccaatgccagtgcacatacacgtgcaagtcccaccggttccctgcccggctatggtagagacaagaaggacgataccggcatcgacatcaacagtttcaacagcaatgcgtttggcgtcgacgcgtcgatggggctgccgtatttggatttggacgggctagatttcgatatggatatggatatggatatggatatggagatgaatttgaatttagatttgggtcttgatttggggttggaattaaaaggggataacaatgagggttttcctgttgatttaaacaatggacgtgggaggtgattgatttaacctgatccaaaaggggtatgtctattttttagagtgtgtctttgtgtcaaattatggtagaatgtgtaaagtagtataaactttcctctcaaatgacgaggtttaaaacaccccccgggtgagccgagccgagaatggggcaattgttcaatgtgaaatagaagtatcgagtgagaaacttgggtgttggccagccaagggggaaggaaaatggcgcgaatgctcaggtgagattgttttggaattgggtgaagcgaggaaatgagcgacccggaggttgtgactttagtggcggaggaggaacgggaggaaaaggccaagagggaaagtgtatataagggggagcaatttgccaaccaggatagaattggatgagttataattctactgtatttattgtataatttatttctccttttatatcaaacacattacaaaacacacaaaacatacaaacatacacagctagcatgggtgaattgaaagagattttgaaacaaagatatcatgaattacttgattggaatgttaaggcaccacatgtccctttatcccagagattgaagcactttacttggtcatggtttgcttgtactatggcaaccggtggtgttggtttgatcattggttccttcccattcagattctacggtttgaacaccattggcaagattgtttacatcttacaaatctttttgttttctctttttggctcttgtatgttgtttcgtttcatcaagtatccatctaccattaaggactcttggaatcatcacttggaaaagttgtttatcgcaacttgtttgttatctatttccacattcatcgacatgttagctatctatgcttatccagataccggtgaatggatggtctgggtcattagaatcttatactacatctatgtcgctgtctctttcatctactgtgttatggcctttttcaccattttcaacaatcatgtttacactattgaaactgcttctccagcttggattttgccaatcttccctccaatgatctgtggtgtcattgctggtgctgttaactccacccaacctgctcaccaattgaaaaacatggtcattttcggtatcttgtttcaaggtttaggtttttgggtttaccttttacttttcgccgttaatgttttgagattcttcacagtcggtttagcaaagccacaagatagaccaggtatgtttatgttcgttggtccaccagctttctctggtttagcattgattaacattgcaagaggtgcaatgggctcaagaccttacattttcgttggtgcaaactcttccgaatacttaggttttgtctcaaccttcatggccattttcatctggggtttagccgcatggtgttattgcttagctatggtttccttccttgccggctttttcactagagcaccattgaaattcgcttgtggttggttcgctttcatctttccaaatgttggttttgttaactgtactatcgaaatcggcaagatgattgattctaaggcttttcaaatgtttggtcacatcattggtgttatcttgtgtattcaatggattttgttaatgtacttaatggttagagcattccttgttaatgacttgtgctatcctggtaaagacgaagatgcacacccaccaccaaagccaaacactggtgtcttaaacccaactttcccaccagagaaggctccagcatcattagagaaggttgatactcatgttacatcaacaggtggtgaatccgatcctccatcttccgaacatgaatccgtttaaggcgcgccatctaatagtttaatcacagcttatagtctactatagttttcttttttaaacattgttgtattttgtcccccccctctaattgatgatgattatcctataagaatccaataaaacgatggaaactaataccctctcctttgtcatgtggtctttagtatttcttgaacattggctctgatttctcgactttatagtcctattaaaatcgctgttagttctcgatcgttgtatctcgtttcttgtctctttggtggatgattttgcgtgcgaacatgtttttttccctttctctcaccatcatcgtgtagttcttgtcaccatcccccccaccccttccttctctcattgattctataagagcttatccacagaggtgcagtaacgaggtagtttaaccttcgagtggatcaaaatgtcacacaggcctgcggccgctaccataatgtatgcgttgagcctcttgcaccttctttattaggaaatcagttgaaaaatttccggattgtctttattattggcccatttttttttggtcacacctttatttttgtacacttctcgggcaaagcaaaaactatagtaccggataggcctttataaaactccagtgtgtatgattttagttggtgtgccatctacacgttctcttagtttctttatcatgtcacagaaagcaagcatgcaaacccttacaaaaaataacaacatacaaatgcctaaacaactggactataatgatggtgagtcagttacgaaaagagcaagtgggttaatacgatttcgtaagggacagtctgaggaagactacaattttcaaaaggagcagttctggtccacgggtcctttagtacagaatcacacatttgtgactgaatttgttgaaaagtttattgaaaacacaattagtgaagattattcaatcacagatagatcgaaaatagaacgtgaaacaatcatacacggattggagaagctgtattttcaaagggaatatgagcgatgtctaaaagatgttcaactattgaaggacaatatcgataagttcaatcctaatttggatcttaatgaaaagaatttataatgagctgaattatatttcttggatgtgcatcaaaaagatccatgagagtaacgaaaagaaactgggggaaatctaataatttacaatttcaatatacacttctatatcctttaatgtaatggctttataaataaacacgaacttctacagcaccgacgtttctttttcttaccagctcctcttcttcttcttcttcttcttcttcttcttcttcttcttcttcttcttcttcttcttcttcttctttcttaccatcattgccattttccttttttcttatttgctcttgatcctctgttttttcaatttggacaaactcatctaatacaccaacacttttagggcccccgc

SEQ ID NO: 6 - DNA, 1317 bp, Schizosaccharomyces pombe
  source 1..1317: mol_type = other DNA
atgggtgaattgaaagagattttgaaacaaagatatcatgaattacttgattggaatgttaaggcaccacatgtccctttatcccagagattgaagcactttacttggtcatggtttgcttgtactatggcaaccggtggtgttggtttgatcattggttccttcccattcagattctacggtttgaacaccattggcaagattgtttacatcttacaaatctttttgttttctctttttggctcttgtatgttgtttcgtttcatcaagtatccatctaccattaaggactcttggaatcatcacttggaaaagttgtttatcgcaacttgtttgttatctatttccacattcatcgacatgttagctatctatgcttatccagataccggtgaatggatggtctgggtcattagaatcttatactacatctatgtcgctgtctctttcatctactgtgttatggcctttttcaccattttcaacaatcatgtttacactattgaaactgcttctccagcttggattttgccaatcttccctccaatgatctgtggtgtcattgctggtgctgttaactccacccaacctgctcaccaattgaaaaacatggtcattttcggtatcttgtttcaaggtttaggtttttgggtttaccttttacttttcgccgttaatgttttgagattcttcacagtcggtttagcaaagccacaagatagaccaggtatgtttatgttcgttggtccaccagctttctctggtttagcattgattaacattgcaagaggtgcaatgggctcaagaccttacattttcgttggtgcaaactcttccgaatacttaggttttgtctcaaccttcatggccattttcatctggggtttagccgcatggtgttattgcttagctatggtttccttccttgccggctttttcactagagcaccattgaaattcgcttgtggttggttcgctttcatctttccaaatgttggttttgttaactgtactatcgaaatcggcaagatgattgattctaaggcttttcaaatgtttggtcacatcattggtgttatcttgtgtattcaatggattttgttaatgtacttaatggttagagcattccttgttaatgacttgtgctatcctggtaaagacgaagatgcacacccaccaccaaagccaaacactggtgtcttaaacccaactttcccaccagagaaggctccagcatcattagagaaggttgatactcatgttacatcaacaggtggtgaatccgatcctccatcttccgaacatgaatccgtttaa

SEQ ID NO: 7 - DNA, 6527 bp, synthetic construct
  source 1..6527: mol_type = other DNA; note = Synthetic - PYC gene inserter fragment
ctaaaagtgttggtgtattagatgagtttgtccaaattgaaaaaacagaggatcaagagcaaataagaaaaaaggaaaatggcaatgatggtaagaaagaagaagaagaagaagaagaagaagaagaagaagaagaagaagaagaagaagaagaagaagaagaggagctggtaagaaaaagaaacgtcggtgctgtagaagttcgtgtttatttataaagccattacattaaaggatatagaagtgtatattgaaattgtaaattattagatttcccccagtttcttttcgttactctcatggatctttttgatgcacatccaagaaatataattcagctcattataaattcttttcattaagatccaaattaggattgaacttatcgatattgtccttcaatagttgaacatcttttagacatcgctcatattccctttgaaaatacagcttctccaatccgtgtatgattgtttcacgttctattttcgatctatctgtgattgaataatcttcactaattgtgttttcaataaacttttcaacaaattcagtcacaaatgtgtgattctgtactaaaggacccgtggaccagaactgctccttttgaaaattgtagtcttcctcagactgtcccttacgaaatcgtattaacccacttgctcttttcgtaactgactcaccatcattatagtccagttgtttaggcatttgtatgttgttattttttgtaagggtttgcatgcttgctttctgtgacatgataaagaaactaagagaacgtgtagatggcacaccaactaaaatcatacacactggagttttataaaggcctatccggtactatagtttttgctttgcccgagaagtgtacaaaaataaaggtgtgaccaaaaaaaaatgggccaataataaagacaatccggaaatttttcaactgatttcctaataaagaaggtgcaagaggctcaacgcatacattatggtagcggccgcgagtccatcggttcctgtcagatgggatactcttgacgtggaaaattcaaacagaaaaaaaaccccaataatgaaaaataacactacgttatatccgtggtatcctctatcgtatcgtatcgtagcgtatcgtagcgtaccgtatcacagtatagtctaatattccgtatcttattgtatcctatcctattcgatcctattgtatttcagtgcaccattttaatttctattgctataatgtccttattagttgccactgtgaggtgaccaatggacgagggcgagccgttcagaagccgcgaagggtgttcttcccatgaatttcttaaggagggcggctcagctccgagagtgaggcgagacgtctcggtcagcgtatcccccttcctcggcttttacaaatgatgcgctcttaatagtgtgtcgttatccttttggcattgacgggggagggaaattgattgagcgcatccatatttttgcggactgctgaggacaatggtggtttttccgggtggcgtgggctacaaatgatacgatggtttttttcttttcggagaaggcgtataaaaaggacacggagaacccatttattctaaaaacagttgagcttctttaattattttttgatataatattctattattatatattttcttcccaataaaacaaaataaaacaaaacacagcaaaacacaaaaattctagataaaatgtcaactgtggaagatcactcctccctacataaattgagaaaggaatctgagattctttccaatgcaaacaaaatcttagtggctaatagaggtgaaattccaattagaattttcaggtcagcccatgaattgtcaatgcatactgtggcgatctattcccatgaagatcggttgtccatgcataggttgaaggccgacgaggcttatgcaatcggtaagactggtcaatattcgccagttcaagcttatctacaaattgacgaaattatcaaaatagcaaaggaacatgatgtttccatgatccatccaggttatggtttcttatctgaaaactccgaattcgcaaagaaggttgaagaatccggtatgatttgggttgggcctcctgctgaagttattgattctgttggtgacaaggtttctgcaagaaatttggcaattaaatgtgacgttcctgttgttcctggtaccgatggtccaattgaagacattgaacaggctaaacagtttgtggaacaatatggttatcctgtcattataaaggctgcatttggtggtggtggtagaggtatgagagttgttagagaaggtgatgatatagttgatgctttccaaagagcgtcatctgaagcaaagtctgcctttggtaatggtacttgttttattgaaagatttttggataagccaaaacatattgaggttcaattattggctgataattatggtaacacaatccatctctttgaaagagattgttctgttcaaagaagacatcaaaaggttgttgaaattgcacctgccaaaactttacctgttgaagttagaaatgctatattaaaggatgctgtaacgttagctaaaaccgctaactatagaaatgctggtactgcagaatttttagttgattcccaaaacagacattattttattgaaattaatccaagaattcaagttgaacatacaattactgaagaaatcacgggtgttgatattgttgccgctcaaattcaaattgctgcaggtgcatcattggaacaattgggtctattacaaaacaaaattacaactagaggttttgcaattcaatgtagaattacaaccgaggatcctgctaagaattttgccccagatacaggtaaaattgaggtttatagatctgcaggtggtaacggtgtcagattagatggtggtaatgggtttgccggtgctgttatatctcctcattatgactcgatgttggttaaatgttcaacatctggttctaactatgaaattgccagaagaaagatgattagagctttagttgaatttagaatcagaggtgtcaagaccaatattcctttcttattggcattgctaactcatccagttttcatttcgggtgattgttggacaacttttattgatgataccccttcgttattcgaaatggtttcttcaaagaatagagcccaaaaattattggcatatattggtgacttgtgtgtcaatggttcttcaattaaaggtcaaattggtttccctaaattgaacaaggaagcagaaatcccagatttgttggatccaaatgatgaggttattgatgtttctaaaccttctaccaatggtctaagaccgtatctattaaagtatggaccagatgcgttttccaaaaaagttcgtgaattcgatggttgtatgattatggataccacctggagagatgcacatcaatcattattggctacaagagttagaactattgatttactgagaattgctccaacgactagtcatgccttacaaaatgcatttgcattagaatgttggggtggcgcaacatttgatgttgcgatgaggttcctctatgaagatccttgggagagattaagacaacttagaaaggcagttccaaatattcctttccaaatgttattgagaggtgctaatggtgttgcttattcgtcattacctgataatgcaattgatcattttgttaagcaagcaaaggataatggtgttgatattttcagagtctttgatgctttgaacgatttggaacaattgaaggttggtgttgatgctgtcaagaaagccggaggtgttgttgaagctacagtttgttactcaggtgatatgttaattccaggtaaaaagtataacttggattattatttagagactgttggaaagattgtggaaatgggtacccatattttaggtattaaggatatggctggcacgttaaagccaaaggctgctaagttgttgattggctcgatcagatcaaaataccctgacttggttatccatgtccatacccatgactctgctggtaccggtatttcaacttatgttgcatgcgcattggcaggtgccgacattgtcgattgtgcaatcaattcgatgtctggtttaacctctcaaccttcaatgagtgcttttattgctgctttagatggtgatatcgaaactggtgttccagaacattttgcaagacaattagatgcatactgggcagaaatgagattgttatactcatgtttcgaagccgacttgaagggaccagacccagaagtttataaacatgaaattccaggtggacagttgactaacctaatcttccaagcccaacaagttggtttgggtgaacaatgggaagaaactaagaagaagtatgaagatgctaacatgttgttgggtgatattgtcaaggttaccccaacctccaaggttgttggtgatttagcccaatttatggtttctaataaattagaaaaagaagatgttgaaaaacttgctaatgaattagatttcccagattcagttcttgatttctttgaaggattaatgggtacaccatatggtggattcccagagcctttgagaacaaatgtcatttccggcaagagaagaaaattaaagggtagaccaggtttagaattagaacctttcaacctcgaggaaatcagagaaaatttggtttccagatttggtccaggtattactgaatgtgatgttgcatcttataacatgtatccaaaggtttacgagcaatatcgtaaggtggttgaaaaatatggtgatttatctgttttaccaacaaaagcatttttggctcctccaactattggtgaagaagttcatgtggaaattgagcaaggtaagactttgattattaagttattagccatttctgacttgtctaaatctcatggtacaagagaagtatactttgaattgaatggtgaaatgagaaaggttacaattgaagataaaacagctgcaattgagactgttacaagagcaaaggctgacggacacaatccaaatgaagttggtgcgccaatggctggtgtcgttgttgaagttagagtgaagcatggaacagaagttaagaagggtgatccattagccgttttgagtgcaatgaaaatggaaatggttatttctgctcctgttagtggtagggtcggtgaagtttttgtcaacgaaggcgattccgttgatatgggtgatttgcttgtgaaaattgccaaagatgaagcgccagcagcttaattaattctgtctttgattttcttatgttattcaaaacatctgccccaaaatctaacgattatatatattcctacgtataactgtatagctaattattgatttatttgtacataaaaaccacataaatgtaaaagcaagaaaaaaaataactaaggagaaggatcaatatctcatttataatgctcgccaaagcagcgtacgtgaattttaatcaagacatcaacaaatcttgcaacttggttatatcgcttcttcacccactcacccgcttttctacattgttgaacacaaatatatacaggggtatgtctcaaggtcaagtgcagtttcaacagagactacctcaaggtacctcttcagaaatgcagaacttcactcttgatcagattttctccgaattaaaggtttaaacatagcctcatgaaatcagccatttgcttttgttcaacgatcttttgaaattgttgttgttcttggtagttaagttgatccatcttggcttatgttgtgtgtatgttgtagttattcttagtatattcctgtcctgagtttagtgaaacataatatcgccttgaaatgaaaatgctgaaattcgtcgacatacaatttttcaaactttttttttttcttggtgcacggacatgtttttaaaggaagtactctataccagttattcttcacaaatttaattgctggagaatagatcttcaacgctttaataaagtagtttgtttgtcaaggatggcgtcatacaaagaaagatcagaatcacacacttcccctgttgctaggagacttttctccatcatggaggaaaagaagtctaacctttgtgcatcattggatattactgaaactgaaaagcttctctctattttggacactattggtccttacatctgtctagttaaaacacacatcgatattgtttctgattttacgtatgaaggaactgtgttgcctttgaaggagcttgccaagaaacataattttatgatttttgaagatagaaaatttgctgatattggtaacactgttaaaaatcaatataaatctggtgtcttccgtattgccgaatgggctgacatcactaatgcacatggtgtaacgggtgcaggtattgtttctggcttgaaggaggcagcccaagaaacaaccagtgaacctagaggtttgctaatgcttgctgagttatcatcaaagggttctttagcatatggtgaatatacagaaaaaacagtagaaattgctaaatctgataaagagtttgttgag

SEQ ID NO: 8 - DNA, 4760 bp, synthetic construct
  source 1..4760: mol_type = other DNA; note = Synthetic - MAE gene integration fragment
aattctttgaaggagcttgccaagaaacataattttatgatttttgaagatagaaaatttgctgatattggtaacactgttaaaaatcaatataaatctggtgtcttccgtattgccgaatgggctgacatcactaatgcacatggtgtaacgggtgcaggtattgtttctggcttgaaggaggcagcccaagaaacaaccagtgaacctagaggtttgctaatgcttgctgagttatcatcaaagggttctttagcatatggtgaatatacagaaaaaacagtagaaattgctaaatctgataaagagtttgtcattggttttattgcgcaacacgatatgggcggtagagaagaaggttttgactggatcattatgactccaggggttggtttagatgacaaaggtgatgcacttggtcaacaatatagaactgttgatgaagttgtaaagactggaacggatatcataattgttggtagaggtttgtacggtcaaggaagagatcctatagagcaagctaaaagataccaacaagctggttggaatgcttatttaaacagatttaaatgattcttacacaaagatttgatacatgtacactagtttaaataagcatgaaaagaattacacaagcaaaaaaaaaaaaataaatgaggtactttacgttcacctacaaccaaaaaaactagatagagtaaaatcttaagatttagaaaaagttgtttaacaaaggctttagtatgtgaatttttaatgtagcaaagcgataactaataaacataaacaaaagtatggttttctttatcagtcaaatcattatcgattgattgttccgcgtatctgcagatagcctcatgaaatcagccatttgcttttgttcaacgatcttttgaaattgttgttgttcttggtagttaagttgatccatcttggcttatgttgtgtgtatgttgtagttattcttagtatattcctgtcctgagtttagtgaaacataatatcgccttgaaatgaaaatgctgaaattcgtcgacatacaatttttcaaactttttttttttcttggtgcacggacatgtttttaaaggaagtactctataccagttattcttcacaaatttaattgctggagaatagatcttcaacgcgtttaaacagcaatttgaggaaggaataggagaaggagaagcaatttctaggaaagagcaaggtgtgcaacagcatgctctgaatgatattttcagcaatagttcagttgaagaacctgttggcgtatctacatcacttcctacaaacaacaccacgaattgcgtccgtggtgacgcaactacgaatggcattgtcaatgccaatgccagtgcacatacacgtgcaagtcccaccggttccctgcccggctatggtagagacaagaaggacgataccggcatcgacatcaacagtttcaacagcaatgcgtttggcgtcgacgcgtcgatggggctgccgtatttggatttggacgggctagatttcgatatggatatggatatggatatggatatggagatgaatttgaatttagatttgggtcttgatttggggttggaattaaaaggggataacaatgagggttttcctgttgatttaaacaatggacgtgggaggtgattgatttaacctgatccaaaaggggtatgtctattttttagagtgtgtctttgtgtcaaattatggtagaatgtgtaaagtagtataaactttcctctcaaatgacgaggtttaaaacaccccccgggtgagccgagccgagaatggggcaattgttcaatgtgaaatagaagtatcgagtgagaaacttgggtgttggccagccaagggggaaggaaaatggcgcgaatgctcaggtgagattgttttggaattgggtgaagcgaggaaatgagcgacccggaggttgtgactttagtggcggaggaggaacgggaggaaaaggccaagagggaaagtgtatataagggggagcaatttgccaaccaggatagaattggatgagttataattctactgtatttattgtataatttatttctccttttatatcaaacacattacaaaacacacaaaacatacaaacatacacagctagcatgggtgaattgaaagagattttgaaacaaagatatcatgaattacttgattggaatgttaaggcaccacatgtccctttatcccagagattgaagcactttacttggtcatggtttgcttgtactatggcaaccggtggtgttggtttgatcattggttccttcccattcagattctacggtttgaacaccattggcaagattgtttacatcttacaaatctttttgttttctctttttggctcttgtatgttgtttcgtttcatcaagtatccatctaccattaaggactcttggaatcatcacttggaaaagttgtttatcgcaacttgtttgttatctatttccacattcatcgacatgttagctatctatgcttatccagataccggtgaatggatggtctgggtcattagaatcttatactacatctatgtcgctgtctctttcatctactgtgttatggcctttttcaccattttcaacaatcatgtttacactattgaaactgcttctccagcttggattttgccaatcttccctccaatgatctgtggtgtcattgctggtgctgttaactccacccaacctgctcaccaattgaaaaacatggtcattttcggtatcttgtttcaaggtttaggtttttgggtttaccttttacttttcgccgttaatgttttgagattcttcacagtcggtttagcaaagccacaagatagaccaggtatgtttatgttcgttggtccaccagctttctctggtttagcattgattaacattgcaagaggtgcaatgggctcaagaccttacattttcgttggtgcaaactcttccgaatacttaggttttgtctcaaccttcatggccattttcatctggggtttagccgcatggtgttattgcttagctatggtttccttccttgccggctttttcactagagcaccattgaaattcgcttgtggttggttcgctttcatctttccaaatgttggttttgttaactgtactatcgaaatcggcaagatgattgattctaaggcttttcaaatgtttggtcacatcattggtgttatcttgtgtattcaatggattttgttaatgtacttaatggttagagcattccttgttaatgacttgtgctatcctggtaaagacgaagatgcacacccaccaccaaagccaaacactggtgtcttaaacccaactttcccaccagagaaggctccagcatcattagagaaggttgatactcatgttacatcaacaggtggtgaatccgatcctccatcttccgaacatgaatccgtttaaggcgcgccatctaatagtttaatcacagcttatagtctactatagttttcttttttaaacattgttgtattttgtcccccccctctaattgatgatgattatcctataagaatccaataaaacgatggaaactaataccctctcctttgtcatgtggtctttagtatttcttgaacattggctctgatttctcgactttatagtcctattaaaatcgctgttagttctcgatcgttgtatctcgtttcttgtctctttggtggatgattttgcgtgcgaacatgtttttttccctttctctcaccatcatcgtgtagttcttgtcaccatcccccccaccccttccttctctcattgattctataagagcttatccacagaggtgcagtaacgaggtagtttaaccttcgagtggatcaaaatgtcacacaggcctgcggccgcatttggcaaggcgtatctatataggaggatcacaagaaaagagagttcaacttagggaaccaggcttgacaaaagataatattaaaaaaggaagaaaaagagagaaagaaagtaaagacaagaagaatcaactccaatttgaataaatgggcctgtaagaatcttcaatctcgatgacggggaaaacgtcctcttttatagaccaaccccttgtggggctgtcgggaaaagcgggtttcccgggaaactcatatctgattggggggttgaagctattccgcgttttggggggtgcggtttataagaagaatgagtaatgcaaacggcgtttataaagagaaagggtgtggcggtgttaagcggttgaggttttaaggtgtgggggacggggtgtgattttttcggcgttacgggcacggggtatgattctcttaatgtcatttccctttctttctacttctagggcgcgcagcgtgcgacaatggtgtttgttgtgcaaataggtggttgtgttgtcgttggagttctcatgaacatatcactctattagcaaatttaccctaagtatgtaaggtttagtgtgttgaggactaatacagacaatctttgtcttatatataaatggttctactcaattttataagttttttttttttttttttaatctctttatgaaaatcaagaatgacattaaaacaatcacaatattgtatgcaagaactttgcctctaaacctctttcaaagagatgcataattataaaacaaatctatttgcatattcgctttacacaactattcaagaatataattacctaaagagtccatcttgagttcaccaacaccactacttagagctcggtacccgc

SEQ ID NO: 9 - DNA, 6874 bp, synthetic construct
  source 1..6874: mol_type = other DNA; note = Synthetic - FRD gene integration fragment
aaacctccgttatgtatgtttgtacccaaaaagaatgcgctatattagtttaatcttttataaacccggaattataaaaatacagttaggaataaagtaatagaaagatgaacaacgggcctaaaaagactaatgtgttgtggatcggaatgtttcgaatagagtattaaagttatgctttcttttctttttgaacatgcttggtattactttgatatgcaaaagatatcgacaaattgaaaatggttttgatgtctatagatgtggcatggtaaggttcatttcaatttagcaaatatcagacgagctcagcggccgcggatccctcgaggagtccatcggttcctgtcagatgggatactcttgacgtggaaaattcaaacagaaaaaaaaccccaataatgaaaaataacactacgttatatccgtggtatcctctatcgtatcgtatcgtagcgtatcgtagcgtaccgtatcacagtatagtctaatattccgtatcttattgtatcctatcctattcgatcctattgtatttcagtgcaccattttaatttctattgctataatgtccttattagttgccactgtgaggtgaccaatggacgagggcgagccgttcagaagccgcgaagggtgttcttcccatgaatttcttaaggagggcggctcagctccgagagtgaggcgagacgtctcggtcagcgtatcccccttcctcggcttttacaaatgatgcgctcttaatagtgtgtcgttatccttttggcattgacgggggagggaaattgattgagcgcatccatatttttgcggactgctgaggacaatggtggtttttccgggtggcgtgggctacaaatgatacgatggtttttttcttttcggagaaggcgtataaaaaggacacggagaacccatttattctaaaaacagttgagcttctttaattattttttgatataatattctattattatatattttcttcccaataaaacaaaataaaacaaaacacagcaaaacacaaaaattctagaatggctgatggcaaaacctctgcatcagttgttgctgttgatgctgaacgtgccgctaaggaaagagatgcagcagctagagctatgttgcaaggtggtggtgtctctcctgctggcaaggcacaattgttgaaaaagggtttggttcacactgttccatataccttaaaggttgtcgtcgcagatccaaaggaaatggagaaggcaactgctgacgcagaagaggttttacaagctgcatttcaagtcgtcgacacccttttgaacaactttaacgaaaactcagaagtttcaagagtcaataggttggcagttggtgaggaacatcaaatgtctgaaacattgaaacacgtcatggcctgttgtcaaaaggtttatcattcctccagaggtgtttttgacccagcagttggtccattagtccgtgaacttagagaagctgctcacaagggtaaaactgttccagccgaaagagttaatgatttgttatccaaatgtacccttaatgcatctttttcaattgatatgtccagaggtatgattgcaaggaagcatccagacgccatgttggatttgggtggtgtcaacaagggttatggtatcgactacattgttgaacacttaaactctttgggttatgatgatgtctttttcgaatggggtggtgatgttagagcatccggcaaaaaccagttatctcaaccttgggctgttggtattgttagaccacctgccttggccgacattagaactgttgtcccagaggacaaaagatcctttatccgtgtcgtcagattgaacaacgaagctattgctacctctggtgattatgagaatttggttgaaggtcctggttctaaggtttactcttccaccttcaatccaacttccaaaaacttgttggaacctaccgaagcaggtatggctcaagtttctgtcaagtgttgctcatgtatctacgctgatgctttagcaacagcagctttgttgaaaaacgatcctgctgccgttagaaggatcttagataactggagatatgtcagagatactgttactgactacaccacttacacaagggaaggtgaaagagttgctaagatgttggaaattgctaccgaagatgctgaaatgagagcaaagagaatcaagggctctttaccagcaagagttatcattgttggtggtggtttggccggttgttccgcagctatcgaagcagctaactgtggcgcccacgtcatcttgttagaaaaggaaccaaagttaggtggtaactctgcaaaggctacctccggtatcaacgcctggggtactagagcacaagcaaaacaaggtgtcatggacggcggcaagtttttcgaaagagatacccatagatccggcaagggtggtaattgcgatccatgccttgttaagactttgtccgttaagtcctctgatgcagttaagtggttatctgaattaggtgttccattgactgttttgtctcaattaggtggtgcttcaaggaaacgttgtcaccgtgcaccagataagtctgatggtacaccagtcccagttggtttcaccattatgaaaacccttgaaaaccacattgtcaacgatttgtccagacatgttacagttatgacaggtattaccgtcacagctttagaatctacatcaagagtcagacctgatggtgttttagtcaagcatgttactggtgttcacttgattcaggcatctggtcaatctatggttttgaatgcagacgctgttatcttagctactggtggtttctccaatgatcataccccaaactcccttttacaacaatacgccccacagttgtcatcttttccaacaaccaatggtgtctgggcaactggcgatggtgttaagatggcttccaagttgggtgtcgccttagttgatatggataaggtccaattacatcctaccggcttgttagacccaaaagatccatctaatagaaccaagtatcttggtccagaggccttaagaggttccggcggtgtcttgttaaacaaaaacggtgaaagatttgttaatgaattagacttaagatctgttgtctctcaagctatcatcgcacaagataatgagtacccaggctctggtggttccaagttcgcatactgtgttttgaacgaaactgcagcaaagttattcggcaaaaacttccttggtttctactggaatagattaggtcttttccaaaaggttgattccgttgctggtttagctaagttgattggttgtccagaagctaatgttgttgctacattgaagcaatatgaggagttatcttccaaaaagcttaatccttgtccattgactggcaagtctgtctttccttgtgttttaggcactcaaggtccatactatgttgccttggttaccccatccattcactacactatgggtggttgtttgatttccccatctgctgagatgcaaaccattgacaactctggtgttactcctgtcagacgtccaatcttaggcttattcggtgctggtgaagttactggcggtgtccatggtggtaacagattaggcggtaactctttgttagaatgtgttgttttcggcaagatcgctggtgacagagctgcaaccatcttgcaaaagaaaaacaccggcttatcaatgacagaatggtctactgtcgtcttaagagaagttagagaaggtggtgtctatggtgctggttccagagttttgaggtttaacatgcctggtgcattacagagaactggtttagctttaggtcaattcatcggtatcagaggtgattgggacggtcacagattgatcggttactattctccaatcactttacctgatgatgttggtgttattggtatcttagctagagcagacaagggtagattggcagaatggatttctgcattgcagccaggtgacgctgttgagatgaaggcctgcggtggtcttatcattgacagaagattcgctgaaagacatttctttttccgtggtcataagatcagaaagttggcccttatcggtggtggtactggtgttgcaccaatgttacaaatcgtcagagctgctgtcaaaaagccatttgtcgattcaattgagtccattcagttcatctatgctgcagaggatgtttccgagcttacatacagaaccttacttgaatcttacgaagaggaatatggttcagaaaagtttaagtgtcacttcgttttgaataacccaccagctcaatggactgacggtgttggtttcgttgatactgcattgttgagatccgcagttcaagcaccatcaaatgatttgcttgttgcaatttgtggtccaccaatcatgcaaagagcagttaagggtgcattgaaaggtttaggttacaatatgaatcttgttagaaccgttgacgaaactgaaccaccatcataattaattaacatctgaatgtaaaatgaacattaaaatgaattactaaactttacgtctactttacaatctataaactttgtttaatcatataacgaaatacactaatacacaatcctgtacgtatgtaatacttttatccatcaaggattgagaaaaaaaagtaatgattccctgggccattaaaacttagacccccaagcttggataggtcactctctattttcgtttctcccttccctgatagaagggtgatatgtaattaagaataatatataattttataataaaagaattcatagcctcatgaaatcagccatttgcttttgttcaacgatcttttgaaattgttgttgttcttggtagttaagttgatccatcttggcttatgttgtgtgtatgttgtagttattcttagtatattcctgtcctgagtttagtgaaacataatatcgccttgaaatgaaaatgctgaaattcgtcgacatacaatttttcaaactttttttttttcttggtgcacggacatgtttttaaaggaagtactctataccagttattcttcacaaatttaattgctggagaatagatcttcaacgctttaataaagtagtttgtttgtcaaggatggcgtcatacaaagaaagatcagaatcacacacttcccctgttgctaggagacttttctccatcatggaggaaaagaagtctaacctttgtgcatcattggatattactgaaactgaaaagcttctctctattttggacactattggtccttacatctgtctagttaaaacacacatcgatattgtttctgattttacgtatgaaggaactgtgttgcctttgaaggagcttgccaagaaacataattttatgatttttgaagatagaaaatttgctgatattggtaacactgttaaaaatcaatataaatctggtgtcttccgtattgccgaatgggctgacatcactaatgcacatggtgtaacgggtgcaggtattgtttctggcttgaaggaggcagcccaagaaacaaccagtgaacctagaggtttgctaatgcttgctgagttatcatcaaagggttctttagcatatggtgaatatacagaaaaaacagtagaaattgctaaatctgataaagagtttgtcattggttttattgcgcaacacgatatgggcggtagagaagaaggttttgactggatcattatgactccaggggttggtttagatgacaaaggtgatgcacttggtcaacaatatagaactgttgatgaagttgtaaagactggaacggatatcataattgttggtagaggtttgtacggtcaaggaagagatcctatagagcaagctaaaagataccaacaagctggttggaatgcttatttaaacagatttaaatgattcttacacaaagatttgatacatgtacactagtttaaataagcatgaaaagaattacacaagcaaaaaaaaaaaaataaatgaggtactttacgttcacctacaaccaaaaaaactagatagagtaaaatcttaagatttagaaaaagttgtttaacaaaggctttagtatgtgaatttttaatgtagcaaagcgataactaataaacataaacaaaagtatggttttctttatcagtcaaatcattatcgattgattgttccgcgtatctgcagatagcctcatgaaatcagccatttgcttttgttcaacgatcttttgaaattgttgttgttcttggtagttaagttgatccatcttggcttatgttgtgtgtatgttgtagttattcttagtatattcctgtcctgagtttagtgaaacataatatcgccttgaaatgaaaatgctgaaattcgtcgacatacaatttttcaaactttttttttttcttggtgcacggacatgtttttaaaggaagtactctataccagttattcttcacaaatttaattgctggagaatagatcttcaacgccccgggggatctggatccgcggccgctcatatgtttgaaggtattatcactgctgttgatttacgttcttgaaaactgcacggataatattcacaatactaacaataaagaagactcattgtggaaggtgactcaatcatgctagaaaagctggggaataaaggcacttttatagtagccacattttggttcaaaagaatataaaggaaaaaaaaatattttccagtgaaaaagaaaagactctttctccgagaagccgagtttctacgaggccttgttgagtcataggggacctctgtggttgactccggcttattacgtgaatcatcgggggagccgcaccgtttgtccgcgacaggagaaaacgcaaggagtcaaacattaaattggtaggcactaccgaggttt

SEQ ID NO: 10 - DNA, 3435 bp, Leishmania mexicana
  source 1..3435: mol_type = other DNA
atggctgatggcaaaacctctgcatcagttgttgctgttgatgctgaacgtgccgctaaggaaagagatgcagcagctagagctatgttgcaaggtggtggtgtctctcctgctggcaaggcacaattgttgaaaaagggtttggttcacactgttccatataccttaaaggttgtcgtcgcagatccaaaggaaatggagaaggcaactgctgacgcagaagaggttttacaagctgcatttcaagtcgtcgacacccttttgaacaactttaacgaaaactcagaagtttcaagagtcaataggttggcagttggtgaggaacatcaaatgtctgaaacattgaaacacgtcatggcctgttgtcaaaaggtttatcattcctccagaggtgtttttgacccagcagttggtccattagtccgtgaacttagagaagctgctcacaagggtaaaactgttccagccgaaagagttaatgatttgttatccaaatgtacccttaatgcatctttttcaattgatatgtccagaggtatgattgcaaggaagcatccagacgccatgttggatttgggtggtgtcaacaagggttatggtatcgactacattgttgaacacttaaactctttgggttatgatgatgtctttttcgaatggggtggtgatgttagagcatccggcaaaaaccagttatctcaaccttgggctgttggtattgttagaccacctgccttggccgacattagaactgttgtcccagaggacaaaagatcctttatccgtgtcgtcagattgaacaacgaagctattgctacctctggtgattatgagaatttggttgaaggtcctggttctaaggtttactcttccaccttcaatccaacttccaaaaacttgttggaacctaccgaagcaggtatggctcaagtttctgtcaagtgttgctcatgtatctacgctgatgctttagcaacagcagctttgttgaaaaacgatcctgctgccgttagaaggatcttagataactggagatatgtcagagatactgttactgactacaccacttacacaagggaaggtgaaagagttgctaagatgttggaaattgctaccgaagatgctgaaatgagagcaaagagaatcaagggctctttaccagcaagagttatcattgttggtggtggtttggccggttgttccgcagctatcgaagcagctaactgtggcgcccacgtcatcttgttagaaaaggaaccaaagttaggtggtaactctgcaaaggctacctccggtatcaacgcctggggtactagagcacaagcaaaacaaggtgtcatggacggcggcaagtttttcgaaagagatacccatagatccggcaagggtggtaattgcgatccatgccttgttaagactttgtccgttaagtcctctgatgcagttaagtggttatctgaattaggtgttccattgactgttttgtctcaattaggtggtgcttcaaggaaacgttgtcaccgtgcaccagataagtctgatggtacaccagtcccagttggtttcaccattatgaaaacccttgaaaaccacattgtcaacgatttgtccagacatgttacagttatgacaggtattaccgtcacagctttagaatctacatcaagagtcagacctgatggtgttttagtcaagcatgttactggtgttcacttgattcaggcatctggtcaatctatggttttgaatgcagacgctgttatcttagctactggtggtttctccaatgatcataccccaaactcccttttacaacaatacgccccacagttgtcatcttttccaacaaccaatggtgtctgggcaactggcgatggtgttaagatggcttccaagttgggtgtcgccttagttgatatggataaggtccaattacatcctaccggcttgttagacccaaaagatccatctaatagaaccaagtatcttggtccagaggccttaagaggttccggcggtgtcttgttaaacaaaaacggtgaaagatttgttaatgaattagacttaagatctgttgtctctcaagctatcatcgcacaagataatgagtacccaggctctggtggttccaagttcgcatactgtgttttgaacgaaactgcagcaaagttattcggcaaaaacttccttggtttctactggaatagattaggtcttttccaaaaggttgattccgttgctggtttagctaagttgattggttgtccagaagctaatgttgttgctacattgaagcaatatgaggagttatcttccaaaaagcttaatccttgtccattgactggcaagtctgtctttccttgtgttttaggcactcaaggtccatactatgttgccttggttaccccatccattcactacactatgggtggttgtttgatttccccatctgctgagatgcaaaccattgacaactctggtgttactcctgtcagacgtccaatcttaggcttattcggtgctggtgaagttactggcggtgtccatggtggtaacagattaggcggtaactctttgttagaatgtgttgttttcggcaagatcgctggtgacagagctgcaaccatcttgcaaaagaaaaacaccggcttatcaatgacagaatggtctactgtcgtcttaagagaagttagagaaggtggtgtctatggtgctggttccagagttttgaggtttaacatgcctggtgcattacagagaactggtttagctttaggtcaattcatcggtatcagaggtgattgggacggtcacagattgatcggttactattctccaatcactttacctgatgatgttggtgttattggtatcttagctagagcagacaagggtagattggcagaatggatttctgcattgcagccaggtgacgctgttgagatgaaggcctgcggtggtcttatcattgacagaagattcgctgaaagacatttctttttccgtggtcataagatcagaaagttggcccttatcggtggtggtactggtgttgcaccaatgttacaaatcgtcagagctgctgtcaaaaagccatttgtcgattcaattgagtccattcagttcatctatgctgcagaggatgtttccgagcttacatacagaaccttacttgaatcttacgaagaggaatatggttcagaaaagtttaagtgtcacttcgttttgaataacccaccagctcaatggactgacggtgttggtttcgttgatactgcattgttgagatccgcagttcaagcaccatcaaatgatttgcttgttgcaatttgtggtccaccaatcatgcaaagagcagttaagggtgcattgaaaggtttaggttacaatatgaatcttgttagaaccgttgacgaaactgaaccaccatcataa

SEQ ID NO: 11 - DNA, 6874 bp, synthetic construct
  source 1..6874: mol_type = other DNA; note = Synthetic - FRD gene integration fragment
aaacctcggtagtgcctaccaatttaatgtttgactccttgcgttttctcctgtcgcggacaaacggtgcggctcccccgatgattcacgtaataagccggagtcaaccacagaggtcccctatgactcaacaaggcctcgtagaaactcggcttctcggagaaagagtcttttctttttcactggaaaatatttttttttcctttatattcttttgaaccaaaatgtggctactataaaagtgcctttattccccagcttttctagcatgattgagtcaccttccacaatgagtcttctttattgttagtattgtgaatattatccgtgcagttttcaagaacgtaaatcaacagcagtgataataccttcaaacatatgagcggccgcggatccctcgaggagtccatcggttcctgtcagatgggatactcttgacgtggaaaattcaaacagaaaaaaaaccccaataatgaaaaataacactacgttatatccgtggtatcctctatcgtatcgtatcgtagcgtatcgtagcgtaccgtatcacagtatagtctaatattccgtatcttattgtatcctatcctattcgatcctattgtatttcagtgcaccattttaatttctattgctataatgtccttattagttgccactgtgaggtgaccaatggacgagggcgagccgttcagaagccgcgaagggtgttcttcccatgaatttcttaaggagggcggctcagctccgagagtgaggcgagacgtctcggtcagcgtatcccccttcctcggcttttacaaatgatgcgctcttaatagtgtgtcgttatccttttggcattgacgggggagggaaattgattgagcgcatccatatttttgcggactgctgaggacaatggtggtttttccgggtggcgtgggctacaaatgatacgatggtttttttcttttcggagaaggcgtataaaaaggacacggagaacccatttattctaaaaacagttgagcttctttaattattttttgatataatattctattattatatattttcttcccaataaaacaaaataaaacaaaacacagcaaaacacaaaaattctagaatggctgatggcaaaacctctgcatcagttgttgctgttgatgctgaacgtgccgctaaggaaagagatgcagcagctagagctatgttgcaaggtggtggtgtctctcctgctggcaaggcacaattgttgaaaaagggtttggttcacactgttccatataccttaaaggttgtcgtcgcagatccaaaggaaatggagaaggcaactgctgacgcagaagaggttttacaagctgcatttcaagtcgtcgacacccttttgaacaactttaacgaaaactcagaagtttcaagagtcaataggttggcagttggtgaggaacatcaaatgtctgaaacattgaaacacgtcatggcctgttgtcaaaaggtttatcattcctccagaggtgtttttgacccagcagttggtccattagtccgtgaacttagagaagctgctcacaagggtaaaactgttccagccgaaagagttaatgatttgttatccaaatgtacccttaatgcatctttttcaattgatatgtccagaggtatgattgcaaggaagcatccagacgccatgttggatttgggtggtgtcaacaagggttatggtatcgactacattgttgaacacttaaactctttgggttatgatgatgtctttttcgaatggggtggtgatgttagagcatccggcaaaaaccagttatctcaaccttgggctgttggtattgttagaccacctgccttggccgacattagaactgttgtcccagaggacaaaagatcctttatccgtgtcgtcagattgaacaacgaagctattgctacctctggtgattatgagaatttggttgaaggtcctggttctaaggtttactcttccaccttcaatccaacttccaaaaacttgttggaacctaccgaagcaggtatggctcaagtttctgtcaagtgttgctcatgtatctacgctgatgctttagcaacagcagctttgttgaaaaacgatcctgctgccgttagaaggatcttagataactggagatatgtcagagatactgttactgactacaccacttacacaagggaaggtgaaagagttgctaagatgttggaaattgctaccgaagatgctgaaatgagagcaaagagaatcaagggctctttaccagcaagagttatcattgttggtggtggtttggccggttgttccgcagctatcgaagcagctaactgtggcgcccacgtcatcttgttagaaaaggaaccaaagttaggtggtaactctgcaaaggctacctccggtatcaacgcctggggtactagagcacaagcaaaacaaggtgtcatggacggcggcaagtttttcgaaagagatacccatagatccggcaagggtggtaattgcgatccatgccttgttaagactttgtccgttaagtcctctgatgcagttaagtggttatctgaattaggtgttccattgactgttttgtctcaattaggtggtgcttcaaggaaacgttgtcaccgtgcaccagataagtctgatggtacaccagtcccagttggtttcaccattatgaaaacccttgaaaaccacattgtcaacgatttgtccagacatgttacagttatgacaggtattaccgtcacagctttagaatctacatcaagagtcagacctgatggtgttttagtcaagcatgttactggtgttcacttgattcaggcatctggtcaatctatggttttgaatgcagacgctgttatcttagctactggtggtttctccaatgatcataccccaaactcccttttacaacaatacgccccacagttgtcatcttttccaacaaccaatggtgtctgggcaactggcgatggtgttaagatggcttccaagttgggtgtcgccttagttgatatggataaggtccaattacatcctaccggcttgttagacccaaaagatccatctaatagaaccaagtatcttggtccagaggccttaagaggttccggcggtgtcttgttaaacaaaaacggtgaaagatttgttaatgaattagacttaagatctgttgtctctcaagctatcatcgcacaagataatgagtacccaggctctggtggttccaagttcgcatactgtgttttgaacgaaactgcagcaaagttattcggcaaaaacttccttggtttctactggaatagattaggtcttttccaaaaggttgattccgttgctggtttagctaagttgattggttgtccagaagctaatgttgttgctacattgaagcaatatgaggagttatcttccaaaaagcttaatccttgtccattgactggcaagtctgtctttccttgtgttttaggcactcaaggtccatactatgttgccttggttaccccatccattcactacactatgggtggttgtttgatttccccatctgctgagatgcaaaccattgacaactctggtgttactcctgtcagacgtccaatcttaggcttattcggtgctggtgaagttactggcggtgtccatggtggtaacagattaggcggtaactctttgttagaatgtgttgttttcggcaagatcgctggtgacagagctgcaaccatcttgcaaaagaaaaacaccggcttatcaatgacagaatggtctactgtcgtcttaagagaagttagagaaggtggtgtctatggtgctggttccagagttttgaggtttaacatgcctggtgcattacagagaactggtttagctttaggtcaattcatcggtatcagaggtgattgggacggtcacagattgatcggttactattctccaatcactttacctgatgatgttggtgttattggtatcttagctagagcagacaagggtagattggcagaatggatttctgcattgcagccaggtgacgctgttgagatgaaggcctgcggtggtcttatcattgacagaagattcgctgaaagacatttctttttccgtggtcataagatcagaaagttggcccttatcggtggtggtactggtgttgcaccaatgttacaaatcgtcagagctgctgtcaaaaagccatttgtcgattcaattgagtccattcagttcatctatgctgcagaggatgtttccgagcttacatacagaaccttacttgaatcttacgaagaggaatatggttcagaaaagtttaagtgtcacttcgttttgaataacccaccagctcaatggactgacggtgttggtttcgttgatactgcattgttgagatccgcagttcaagcaccatcaaatgatttgcttgttgcaatttgtggtccaccaatcatgcaaagagcagttaagggtgcattgaaaggtttaggttacaatatgaatcttgttagaaccgttgacgaaactgaaccaccatcataattaattaacatctgaatgtaaaatgaacattaaaatgaattactaaactttacgtctactttacaatctataaactttgtttaatcatataacgaaatacactaatacacaatcctgtacgtatgtaatacttttatccatcaaggattgagaaaaaaaagtaatgattccctgggccattaaaacttagacccccaagcttggataggtcactctctattttcgtttctcccttccctgatagaagggtgatatgtaattaagaataatatataattttataataaaagaattcatagcctcatgaaatcagccatttgcttttgttcaacgatcttttgaaattgttgttgttcttggtagttaagttgatccatcttggcttatgttgtgtgtatgttgtagttattcttagtatattcctgtcctgagtttagtgaaacataatatcgccttgaaatgaaaatgctgaaattcgtcgacatacaatttttcaaactttttttttttcttggtgcacggacatgtttttaaaggaagtactctataccagttattcttcacaaatttaattgctggagaatagatcttcaacgctttaataaagtagtttgtttgtcaaggatggcgtcatacaaagaaagatcagaatcacacacttcccctgttgctaggagacttttctccatcatggaggaaaagaagtctaacctttgtgcatcattggatattactgaaactgaaaagcttctctctattttggacactattggtccttacatctgtctagttaaaacacacatcgatattgtttctgattttacgtatgaaggaactgtgttgcctttgaaggagcttgccaagaaacataattttatgatttttgaagatagaaaatttgctgatattggtaacactgttaaaaatcaatataaatctggtgtcttccgtattgccgaatgggctgacatcactaatgcacatggtgtaacgggtgcaggtattgtttctggcttgaaggaggcagcccaagaaacaaccagtgaacctagaggtttgctaatgcttgctgagttatcatcaaagggttctttagcatatggtgaatatacagaaaaaacagtagaaattgctaaatctgataaagagtttgtcattggttttattgcgcaacacgatatgggcggtagagaagaaggttttgactggatcattatgactccaggggttggtttagatgacaaaggtgatgcacttggtcaacaatatagaactgttgatgaagttgtaaagactggaacggatatcataattgttggtagaggtttgtacggtcaaggaagagatcctatagagcaagctaaaagataccaacaagctggttggaatgcttatttaaacagatttaaatgattcttacacaaagatttgatacatgtacactagtttaaataagcatgaaaagaattacacaagcaaaaaaaaaaaaataaatgaggtactttacgttcacctacaaccaaaaaaactagatagagtaaaatcttaagatttagaaaaagttgtttaacaaaggctttagtatgtgaatttttaatgtagcaaagcgataactaataaacataaacaaaagtatggttttctttatcagtcaaatcattatcgattgattgttccgcgtatctgcagatagcctcatgaaatcagccatttgcttttgttcaacgatcttttgaaattgttgttgttcttggtagttaagttgatccatcttggcttatgttgtgtgtatgttgtagttattcttagtatattcctgtcctgagtttagtgaaacataatatcgccttgaaatgaaaatgctgaaattcgtcgacatacaatttttcaaactttttttttttcttggtgcacggacatgtttttaaaggaagtactctataccagttattcttcacaaatttaattgctggagaatagatcttcaacgccccgggggatctggatccgcggccgctgagctcgtctgatatttgctaaattgaaatgaaccttaccatgccacatctatagacatcaaaaccattttcaatttgtcgatatcttttgcatatcaaagtaataccaagcatgttcaaaaagaaaagaaagcataactttaatactctattcgaaacattccgatccacaacacattagtctttttaggcccgttgttcatctttctattactttattcctaactgtatttttataattccgggtttataaaagattaaactaatatagcgcattctttttgggtacaaacatacataacggaggttt

SEQ ID NO: 12 - DNA, 4127 bp, synthetic construct
  source 1..4127: mol_type = other DNA; note = Synthetic - MDH gene integration fragment
gttaacccgtttcgatgggattcccagaagtggatactatactgtctgcaatgcactacactctaaaaaagtattatacattaccatacattagcaaatcaccaatactctgcactgtttcagtgtgtgcacattgctacccaattgggaaatcgcagggaaaatgagacaccccctccattcgtattacgtaagacaatatcagggctgccgaattcggcagaaaagccgagccggccgagtcctcttgcacggagtgtgtccgaaaagggcagctctgcagtgggggagaggaggtcgcacgtctatgcggtgttggcatggcctgtgcgtgtacctgtcccctccctgggcatcccccactgcgcgccttctccattgggcgctgcgggcactccgcgccgttaatacaggaggggggggggaaagcttaagattagagcgggtacagtcagtgggtgtattgaccccatttctgtcagtataaaccccccgttgagccgccggtttggttgtttatggataaaatttttttttccccgcatggagaagattgagggggggaaggaatgggaaaaaggccagagccatctccacagcggaatccgaccgttaatggggtgaaacacccccaccaggtagagcaggaagaatggggaaacaaggtggagagatggtcattgttgggaatagtgggaaaatgagggggaagagaatgactataaaatgggaagggggtccaagttatccaagcagtccatttagagaagggagcggcccctattggtagttctttccccctctcaagctggcgtgaaatgcaaccttacggcgtctacgttactacaaggtccagaaagtgtaggtattgctactatttttattttttattggttctggagaaatgcagacagtcaatgaacacaactgtctcaatatgcatctatgcacatgcacacacacacacatcacaggtacccctacaaagagaggtctcttgataatgtttcattaccacgtggcatccccccccccccccccaataaacaagtggccgagttcccctgttgcagaggaggacaaaaaaaccgctggtgttggtaccattatgcagcaactagcacaacaaacaaccgacccagacatacaaatcaacaacacttcgccaaagacaccctttccagggaggatccactcccaacgtctctccataatgtctctgttggcccatgtctctgtcgttgacaccgtaaccacaccaaccaacccgtccattgtactgggatggtcgtccatagacacctctccaacggggaacacctcattcgtaaaccgccaaggttaccgttcctcctgactcgccccgttgttgatgctgcgcacctgtggttgcccaacatggttgtatatcgtgtaaccacaccaacacatgtgcagcacatgtgtttaaaagagtgtcatggaggtggatcatgatggaagtggactttaccacttgggaactgtctccactcccgggaagaaaagacccggcgtatcacgcggttgcctcaatggggcaatttggaaggagaaatatagggaaaatcacgtcgctctcggacggggaagagttccagactatgaggggggggggtggtatataaagacaggagatgtccacccccagagagaggaagaagttggaactttagaagagagagataactttccccagtgtccatcaatacacaaccaaacacaaactctatatttacacatataaccccctctctagaatggttaaagttacagtttgtggtgctgctggtggtattggtcaacccctttctttactcttgaagcaatcctctcacattactcacttatctctttatgatatcgttaatactcctggtgttgctgctgatcttagtcatatcgataccaaatccaaggtcactggtcatgtaggtgctgctcaacttgaagaagctatcaaggattctgatgttgtcgttattcccgctggtgtcccaagaaagccaggtatgacgcgtgatgatcttttcaagattaatgctggtattgtacgtgatttggctacagctgctgcaaagtacgctccaaaggccttcatgtgtatcatttctaacccagtcaactcgactgtcccaatcgttactgaagtattcaaacagcacaatgtttatgaccccaaaagaatctttggtgtaacaacacttgatattgttcgtgcatccacctttgtatccgaattgattggaggtgaacctaattcacttcgtgttcccgtcattggtggtcacagcggcgtaaccatcttacctttactctcacaggtccccggcattgaaaagttaaaccaagaacaaattgagaaggtaactcatcgtattcaatttggtggcgatgaagttgtcaaggccaaggatggtgctggttctgccactctttccatggcttatgctggtgctcgttttgctacaaacatcattgaggctgcttttgctggaaagaagggcattgttgaatgtacctatgttcaattggatgctgataaatctggtgcccaatctgtcaaggatttggttggtagtgaacttgaatatttctctgttcccgttgaattgggtcctagtggtgttgaaaagattttacccattggaaacgttaatgaatatgaaaagaagttgttgaacgaggcttctcctgaattaaaaaccaacattgataaaggttgtacttttgttactgaaggctcaaagttgtaattaattaatttattttactagtttatttttgctcctgagaataggattacaaacacttaaagtctttaattacaactatatataatattctgttggttttcttgaattggttcgctgcgattcatgcctcccattcaccaaaggtggagtgggaaataacggttttactgcggtaattagcagaggcaagaacaggatacactttttgatgataaatctgtattatagtcgagcctatttaggaaatcaaattttcttgtgtttacttttcaaataaataatgttcgaaaatttttactttactccttcatttaactataccagacgttatatcatcaacaccttctgaccatatacagctcaagatgtttaagagtctgttaaattttttcaatccatttcatggagtaccaggaggtgctacaaaaggaattcatagcctcatgaaatcagccatttgcttttgttcaacgatcttttgaaattgttgttgttcttggtagttaagttgatccatcttggcttatgttgtgtgtatgttgtagttattcttagtatattcctgtcctgagtttagtgaaacataatatcgccttgaaatgaaaatgctgaaattcgtcgacatacaatttttcaaactttttttttttcttggtgcacggacatgtttttaaaggaagtactctataccagttattcttcacaaatttaattgctggagaatagatcttcaacgctttaataaagtagtttgtttgtcaaggatggcgtcatacaaagaaagatcagaatcacacacttcccctgttgctaggagacttttctccatcatggaggaaaagaagtctaacctttgtgcatcattggatattactgaaactgaaaagcttctctctattttggacactattggtccttacatctgtctagttaaaacacacatcgatattgtttctgattttacgtatgaaggaactgtgttgcctttgaaggagcttgccaagaaacataattttatgatttttgaagatagaaaatttgctgatattggtaacactgttaaaaatcaatataaatctggtgtcttccgtattgccgaatgggctgacatcactaatgcacatggtgtaacgggtgcaggtattgtttctggcttgaaggaggcagcccaagaaacaaccagtgaacctagaggtttgctaatgcttgctgagttatcatcaaagggttctttagcatatggtgaatatacagaaaaaacagtagaaattgctaaatctgataaagagtttgtcattggttttattgcgcaacacgatatgggcggtagagaagaaggttttgactccgcgg

SEQ ID NO: 13 - DNA, 987 bp, Rhizopus delemar
  source 1..987: mol_type = other DNA
atggttaaagttacagtttgtggtgctgctggtggtattggtcaacccctttctttactcttgaagcaatcctctcacattactcacttatctctttatgatatcgttaatactcctggtgttgctgctgatcttagtcatatcgataccaaatccaaggtcactggtcatgtaggtgctgctcaacttgaagaagctatcaaggattctgatgttgtcgttattcccgctggtgtcccaagaaagccaggtatgacgcgtgatgatcttttcaagattaatgctggtattgtacgtgatttggctacagctgctgcaaagtacgctccaaaggccttcatgtgtatcatttctaacccagtcaactcgactgtcccaatcgttactgaagtattcaaacagcacaatgtttatgaccccaaaagaatctttggtgttacaacacttgatattgttcgtgcatccacctttgtatccgaattgattggaggtgaacctaattcacttcgtgttcccgtcattggtggtcacagcggcgtaaccatcttacctttactctcacaggtccccggcattgaaaagttaaaccaagaacaaattgagaaggtaactcatcgtattcaatttggtggcgatgaagttgtcaaggccaaggatggtgctggttctgccactctttccatggcttatgctggtgctcgttttgctacaaacatcattgaggctgcttttgctggaaagaagggcattgttgaatgtacctatgttcaattggatgctgataaatctggtgcccaatctgtcaaggatttggttggtagtgaacttgaatatttctctgttcccgttgaattgggtcctagtggtgttgaaaagattttacccattggaaacgttaatgaatatgaaaagaagttgttgaacgaggcttctcctgaattaaaaaccaacattgataaaggttgtacttttgttactgaaggctaa

SEQ ID NO: 14 - DNA, 4112 bp, synthetic construct
  source 1..4112: mol_type = other DNA; note = Synthetic - A. succinogenes FUM gene integration fragment
aattctttgaaggagcttgccaagaaacataattttatgatttttgaagatagaaaatttgctgatattggtaacactgttaaaaatcaatataaatctggtgtcttccgtattgccgaatgggctgacatcactaatgcacatggtgtaacgggtgcaggtattgtttctggcttgaaggaggcagcccaagaaacaaccagtgaacctagaggtttgctaatgcttgctgagttatcatcaaagggttctttagcatatggtgaatatacagaaaaaacagtagaaattgctaaatctgataaagagtttgtcattggttttattgcgcaacacgatatgggcggtagagaagaaggttttgactggatcattatgactccaggggttggtttagatgacaaaggtgatgcacttggtcaacaatatagaactgttgatgaagttgtaaagactggaacggatatcataattgttggtagaggtttgtacggtcaaggaagagatcctatagagcaagctaaaagataccaacaagctggttggaatgcttatttaaacagatttaaatgattcttacacaaagatttgatacatgtacactagtttaaataagcatgaaaagaattacacaagcaaaaaaaaaaaaataaatgaggtactttacgttcacctacaaccaaaaaaactagatagagtaaaatcttaagatttagaaaaagttgtttaacaaaggctttagtatgtgaatttttaatgtagcaaagcgataactaataaacataaacaaaagtatggttttctttatcagtcaaatcattatcgattgattgttccgcgtatctgcagatagcctcatgaaatcagccatttgcttttgttcaacgatcttttgaaattgttgttgttcttggtagttaagttgatccatcttggcttatgttgtgtgtatgttgtagttattcttagtatattcctgtcctgagtttagtgaaacataatatcgccttgaaatgaaaatgctgaaattcgtcgacatacaatttttcaaactttttttttttcttggtgcacggacatgtttttaaaggaagtactctataccagttattcttcacaaatttaattgctggagaatagatcttcaacgcgtttcctcgacatttgctgcaacggcaacatcaatgtccacgtttacacacctacatttatatctatatttatatttatatttatttatttatgctacttagcttctatagttagttaatgcactcacgatattcaaaattgacacccttcaactactccctactattgtctactactgtctactactcctctttactatagctgctcccaataggctccaccaataggctctgtcaatacattttgcgccgccacctttcaggttgtgtcactcctgaaggaccatattgggtaatcgtgcaatttctggaagagagtccgcgagaagtgaggcccccactgtaaatcctcgagggggcatggagtatggggcatggaggatggaggatggggggggggggggggaaaataggtagcgaaaggacccgctatcaccccacccggagaactcgttgccgggaagtcatatttcgacactccggggagtctataaaaggcgggttttgtcttttgccagttgatgttgctgagaggacttgtttgccgtttcttccgatttaacagtatagaatcaaccactgttaattatacacgttatactaacacaacaaaaacaaaaacaacgacaacaacaacaacatctagaatgatcattatgactttccgtattgagaaggatactatgggtgaagttcaagtcccagctgataagtattgggctgcccagaccgaaagatctagaaacaacttcaagattggtccagctgcttctatgccacatgaaatcattgaagcttttggttacttgaaaaaggcagctgcatacgctaacgctgacttgggtgttttgccagctgaaaagagagatttgattgcccaagcttgtgacgaaatcttagccagaaagcttgacgatcagttcccattggttatctggcaaacaggttctggtacccaatccaatatgaacttgaatgaggttatcgctaatagagcacatgttatcaatggtggcaagttgggtgaaaagtctatcattcaccctaatgacgatgtcaacaaatcccaatcttctaatgacacttatccaacagcaatgcatattgccacttacaaaaaggttgttgaagctaccatccctgcaattgaaagattacaaaagaccttagcagctaagtcagaagagtttaaggatgttgtcaaaatcggtaggactcatcttatggatgccaccccattaaccttgggtcaagagttctctggttatgctgcacaattgtccttcggtttagcagcaatcaaaaacaccttgcctcatttgagacaattagcattaggtggtactgcagtcggtactggtcttaacactccaaaaggttatgatgttaaagttgcagaatacattgccaagtttactggtttaccattcatcactgctgaaaacaagttcgaggccttagcaactcacgatgctattgtcgaaacccacggtgccttaaagcaggttgcaatgtcacttttcaagatcgcaaacgacattagattgttggcatcaggtccaagatctggcattggcgagatccttatccctgaaaacgaaccaggttcatccattatgccaggcaaggttaaccctactcaatgtgaagcaatgacaatggttgcagcacaagtcttaggtaatgatacaacaatctccttcgctggctctcaaggtcacttcgaattgaatgtctttaagccagttatggctgctaactttttgcaatctgctcaacttattgctgatgtttgcatttcctttgacgaacactgtgcttccggtatcaagcctaataccccacgtattcaacatttgttagaatcctccttaatgttagtcaccgcattgaacacccacattggttacgaaaatgcagctaagattgctaagaccgctcacaaaaacggtactacattaagagaagaggccattaacttaggtttagtttctgctgaagattttgataagtgggttagaccagaagatatggttggttccttgaagtaattaattaacatctgaatgtaaaatgaacattaaaatgaattactaaactttacgtctactttacaatctataaactttgtttaatcatataacgaaatacactaatacacaatcctgtacgtatgtaatacttttatccatcaaggattgagaaaaaaaagtaatgattccctgggccattaaaacttagacccccaagcttggataggtcactctctattttcgtttctcccttccctgatagaagggtgatatgtaattaagaataatatataattttataataaaagcggccgcaccaggggtttagtgaagtcaccaattaagattgttggtttgagtgagttgccaaagatctatgaattgatggagcaaggtaagattttaggcagatatgttgttgacacttcgaaatgatgggctgacttgggtgtactggtgtgacgtttttatgtgtatattgatatgcatgggggatgtatagtgatgaggagtagagtatataacgaaatgaaatgaaataatatgatatgataagataagatgagatcaatacgataatataagatgcgacatgaggagttcaatgtagcatactacacgatgctgcagtacaactctgatacgctagactatactatacaaaactgtagtacactatacgttagtgtagtatccagaaacaacactgctttatagtacaatacaactctataatactatagtatactatgccaaaccacgtaataccataatatgctccacgacatggtacaatgtgctatacttcatactattataccatatatactccgatatattattgatatactattttatactataataccataccacacaacactacattacaacgagcaaccttaccataaatgtcagttatggccgc

SEQ ID NO: 15 - DNA, 1404 bp, Actinobacillus succinogenes
  source 1..1404: mol_type = other DNA
atgatcattatgactttccgtattgagaaggatactatgggtgaagttcaagtcccagctgataagtattgggctgcccagaccgaaagatctagaaacaacttcaagattggtccagctgcttctatgccacatgaaatcattgaagcttttggttacttgaaaaaggcagctgcatacgctaacgctgacttgggtgttttgccagctgaaaagagagatttgattgcccaagcttgtgacgaaatcttagccagaaagcttgacgatcagttcccattggttatctggcaaacaggttctggtacccaatccaatatgaacttgaatgaggttatcgctaatagagcacatgttatcaatggtggcaagttgggtgaaaagtctatcattcaccctaatgacgatgtcaacaaatcccaatcttctaatgacacttatccaacagcaatgcatattgccacttacaaaaaggttgttgaagctaccatccctgcaattgaaagattacaaaagaccttagcagctaagtcagaagagtttaaggatgttgtcaaaatcggtaggactcatcttatggatgccaccccattaaccttgggtcaagagttctctggttatgctgcacaattgtccttcggtttagcagcaatcaaaaacaccttgcctcatttgagacaattagcattaggtggtactgcagtcggtactggtcttaacactccaaaaggttatgatgttaaagttgcagaatacattgccaagtttactggtttaccattcatcactgctgaaaacaagttcgaggccttagcaactcacgatgctattgtcgaaacccacggtgccttaaagcaggttgcaatgtcacttttcaagatcgcaaacgacattagattgttggcatcaggtccaagatctggcattggcgagatccttatccctgaaaacgaaccaggttcatccattatgccaggcaaggttaaccctactcaatgtgaagcaatgacaatggttgcagcacaagtcttaggtaatgatacaacaatctccttcgctggctctcaaggtcacttcgaattgaatgtctttaagccagttatggctgctaactttttgcaatctgctcaacttattgctgatgtttgcatttcctttgacgaacactgtgcttccggtatcaagcctaataccccacgtattcaacatttgttagaatcctccttaatgttagtcaccgcattgaacacccacattggttacgaaaatgcagctaagattgctaagaccgctcacaaaaacggtactacattaagagaagaggccattaacttaggtttagtttctgctgaagattttgataagtgggttagaccagaagatatggttggttccttgaagtaa

SEQ ID NO: 16 - DNA, 3983 bp, synthetic construct
  source 1..3983: mol_type = other DNA; note = Synthetic - R. delemar MDH gene integration fragment
gggccccataactgacatttatggtaaggttgctcgttgtaatgtagtgttgtgtggtatggtattatagtataaaatagtatatcaataatatatcggagtatatatggtataatagtatgaagtatagcacattgtaccatgtcgtggagcatattatggtattacgtggtttggcatagtatactatagtattatagagttgtattgtactataaagcagtgttgtttctggatactacactaacgtatagtgtactacagttttgtatagtatagtctagcgtatcagagttgtactgcagcatcgtgtagtatgctacattgaactcctcatgtcgcatcttatattatcgtattgatctcatcttatcttatcatatcatattatttcatttcatttcgttatatactctactcctcatcactatacatcccccatgcatatcaatatacacataaaaacgtcacaccagtacacccaagtcagcccatcatttcgaagtgtcaacaacatatctgcctaaaatcttaccttgctccatcaattcatagatctttggcaactcactcaaaccaacaatcttaattggtgacttcactaaacccctggtgcggccgcggatccctcgagattggtagttctttccccctctcaagctggcgtgaaatgcaaccttacggcgtctacgttactacaaggtccagaaagtgtaggtattgctactatttttattttttattggttctggagaaatgcagacagtcaatgaacacaactgtctcaatatgcatctatgcacatgcacacacacacacatcacaggtacccctacaaagagaggtctcttgataatgtttcattaccacgtggcatccccccccccccccccaataaacaagtggccgagttcccctgttgcagaggaggacaaaaaaaccgctggtgttggtaccattatgcagcaactagcacaacaaacaaccgacccagacatacaaatcaacaacacttcgccaaagacaccctttccagggaggatccactcccaacgtctctccataatgtctctgttggcccatgtctctgtcgttgacaccgtaaccacaccaaccaacccgtccattgtactgggatggtcgtccatagacacctctccaacggggaacacctcattcgtaaaccgccaaggttaccgttcctcctgactcgccccgttgttgatgctgcgcacctgtggttgcccaacatggttgtatatcgtgtaaccacaccaacacatgtgcagcacatgtgtttaaaagagtgtcatggaggtggatcatgatggaagtggactttaccacttgggaactgtctccactcccgggaagaaaagacccggcgtatcacgcggttgcctcaatggggcaatttggaaggagaaatatagggaaaatcacgtcgctctcggacggggaagagttccagactatgaggggggggggtggtatataaagacaggagatgtccacccccagagagaggaagaagttggaactttagaagagagagataactttccccagtgtccatcaatacacaaccaaacacaaactctatatttacacatataaccccctctctagataaaatggttaaagttacagtttgtggtgctgctggtggtattggtcaacccctttctttactcttgaagcaatcctctcacattactcacttatctctttatgatatcgttaatactcctggtgttgctgctgatcttagtcatatcgataccaaatccaaggtcactggtcatgtaggtgctgctcaacttgaagaagctatcaaggattctgatgttgtcgttattcccgctggtgtcccaagaaagccaggtatgacgcgtgatgatcttttcaagattaatgctggtattgtacgtgatttggctacagctgctgcaaagtacgctccaaaggccttcatgtgtatcatttctaacccagtcaactcgactgtcccaatcgttactgaagtattcaaacagcacaatgtttatgaccccaaaagaatctttggtgtaacaacacttgatattgttcgtgcatccacctttgtatccgaattgattggaggtgaacctaattcacttcgtgttcccgtcattggtggtcacagcggcgtaaccatcttacctttactctcacaggtccccggcattgaaaagttaaaccaagaacaaattgagaaggtaactcatcgtattcaatttggtggcgatgaagttgtcaaggccaaggatggtgctggttctgccactctttccatggcttatgctggtgctcgttttgctacaaacatcattgaggctgcttttgctggaaagaagggcattgttgaatgtacctatgttcaattggatgctgataaatctggtgcccaatctgtcaaggatttggttggtagtgaacttgaatatttctctgttcccgttgaattgggtcctagtggtgttgaaaagattttacccattggaaacgttaatgaatatgaaaagaagttgttgaacgaggcttctcctgaattaaaaaccaacattgataaaggttgtacttttgttactgaaggctcaaagttgtaattaattaatttattttactagtttatttttgctcctgagaataggattacaaacacttaaagtctttaattacaactatatataatattctgttggttttcttgaattggttcgctgcgattcatgcctcccattcaccaaaggtggagtgggaaataacggttttactgcggtaattagcagaggcaagaacaggatacactttttgatgataaatctgtattatagtcgagcctatttaggaaatcaaattttcttgtgtttacttttcaaataaataatgttcgaaaatttttactttactccttcatttaactataccagacgttatatcatcaacaccttctgaccatatacagctcaagatgtttaagagtctgttaaattttttcaatccatttcatggagtaccaggaggtgctacaaaaggaattcatagcctcatgaaatcagccatttgcttttgttcaacgatcttttgaaattgttgttgttcttggtagttaagttgatccatcttggcttatgttgtgtgtatgttgtagttattcttagtatattcctgtcctgagtttagtgaaacataatatcgccttgaaatgaaaatgctgaaattcgtcgacatacaatttttcaaactttttttttttcttggtgcacggacatgtttttaaaggaagtactctataccagttattcttcacaaatttaattgctggagaatagatcttcaacgctttaataaagtagtttgtttgtcaaggatggcgtcatacaaagaaagatcagaatcacacacttcccctgttgctaggagacttttctccatcatggaggaaaagaagtctaacctttgtgcatcattggatattactgaaactgaaaagcttctctctattttggacactattggtccttacatctgtctagttaaaacacacatcgatattgtttctgattttacgtatgaaggaactgtgttgcctttgaaggagcttgccaagaaacataattttatgatttttgaagatagaaaatttgctgatattggtaacactgttaaaaatcaatataaatctggtgtcttccgtattgccgaatgggctgacatcactaatgcacatggtgtaacgggtgcaggtattgtttctggcttgaaggaggcagcccaagaaacaaccagtgaacctagaggtttgctaatgcttgctgagttatcatcaaagggttctttagcatatggtgaatatacagaaaaaacagtagaaattgctaaatctgataaagagtttgtcattggttttattgcgcaacacgatatgggcggtagagaagaaggttttgactccgcgg

SEQ ID NO: 17 - DNA, 4060 bp, synthetic construct
  source 1..4060: mol_type = other DNA; note = Synthetic - A. succinogenes FUM gene integration fragment
aattctttgaaggagcttgccaagaaacataattttatgatttttgaagatagaaaatttgctgatattggtaacactgttaaaaatcaatataaatctggtgtcttccgtattgccgaatgggctgacatcactaatgcacatggtgtaacgggtgcaggtattgtttctggcttgaaggaggcagcccaagaaacaaccagtgaacctagaggtttgctaatgcttgctgagttatcatcaaagggttctttagcatatggtgaatatacagaaaaaacagtagaaattgctaaatctgataaagagtttgtcattggttttattgcgcaacacgatatgggcggtagagaagaaggttttgactggatcattatgactccaggggttggtttagatgacaaaggtgatgcacttggtcaacaatatagaactgttgatgaagttgtaaagactggaacggatatcataattgttggtagaggtttgtacggtcaaggaagagatcctatagagcaagctaaaagataccaacaagctggttggaatgcttatttaaacagatttaaatgattcttacacaaagatttgatacatgtacactagtttaaataagcatgaaaagaattacacaagcaaaaaaaaaaaaataaatgaggtactttacgttcacctacaaccaaaaaaactagatagagtaaaatcttaagatttagaaaaagttgtttaacaaaggctttagtatgtgaatttttaatgtagcaaagcgataactaataaacataaacaaaagtatggttttctttatcagtcaaatcattatcgattgattgttccgcgtatctgcagatagcctcatgaaatcagccatttgcttttgttcaacgatcttttgaaattgttgttgttcttggtagttaagttgatccatcttggcttatgttgtgtgtatgttgtagttattcttagtatattcctgtcctgagtttagtgaaacataatatcgccttgaaatgaaaatgctgaaattcgtcgacatacaatttttcaaactttttttttttcttggtgcacggacatgtttttaaaggaagtactctataccagttattcttcacaaatttaattgctggagaatagatcttcaacgcgtttcctcgacatttgctgcaacggcaacatcaatgtccacgtttacacacctacatttatatctatatttatatttatatttatttatttatgctacttagcttctatagttagttaatgcactcacgatattcaaaattgacacccttcaactactccctactattgtctactactgtctactactcctctttactatagctgctcccaataggctccaccaataggctctgtcaatacattttgcgccgccacctttcaggttgtgtcactcctgaaggaccatattgggtaatcgtgcaatttctggaagagagtccgcgagaagtgaggcccccactgtaaatcctcgagggggcatggagtatggggcatggaggatggaggatggggggggggggggggaaaataggtagcgaaaggacccgctatcaccccacccggagaactcgttgccgggaagtcatatttcgacactccggggagtctataaaaggcgggttttgtcttttgccagttgatgttgctgagaggacttgtttgccgtttcttccgatttaacagtatagaatcaaccactgttaattatacacgttatactaacacaacaaaaacaaaaacaacgacaacaacaacaacatctagataaaatgatcattatgactttccgtattgagaaggatactatgggtgaagttcaagtcccagctgataagtattgggctgcccagaccgaaagatctagaaacaacttcaagattggtccagctgcttctatgccacatgaaatcattgaagcttttggttacttgaaaaaggcagctgcatacgctaacgctgacttgggtgttttgccagctgaaaagagagatttgattgcccaagcttgtgacgaaatcttagccagaaagcttgacgatcagttcccattggttatctggcaaacaggttctggtacccaatccaatatgaacttgaatgaggttatcgctaatagagcacatgttatcaatggtggcaagttgggtgaaaagtctatcattcaccctaatgacgatgtcaacaaatcccaatcttctaatgacacttatccaacagcaatgcatattgccacttacaaaaaggttgttgaagctaccatccctgcaattgaaagattacaaaagaccttagcagctaagtcagaagagtttaaggatgttgtcaaaatcggtaggactcatcttatggatgccaccccattaaccttgggtcaagagttctctggttatgctgcacaattgtccttcggtttagcagcaatcaaaaacaccttgcctcatttgagacaattagcattaggtggtactgcagtcggtactggtcttaacactccaaaaggttatgatgttaaagttgcagaatacattgccaagtttactggtttaccattcatcactgctgaaaacaagttcgaggccttagcaactcacgatgctattgtcgaaacccacggtgccttaaagcaggttgcaatgtcacttttcaagatcgcaaacgacattagattgttggcatcaggtccaagatctggcattggcgagatccttatccctgaaaacgaaccaggttcatccattatgccaggcaaggttaaccctactcaatgtgaagcaatgacaatggttgcagcacaagtcttaggtaatgatacaacaatctccttcgctggctctcaaggtcacttcgaattgaatgtctttaagccagttatggctgctaactttttgcaatctgctcaacttattgctgatgtttgcatttcctttgacgaacactgtgcttccggtatcaagcctaataccccacgtattcaacatttgttagaatcctccttaatgttagtcaccgcattgaacacccacattggttacgaaaatgcagctaagattgctaagaccgctcacaaaaacggtactacattaagagaagaggccattaacttaggtttagtttctgctgaagattttgataagtgggttagaccagaagatatggttggttccttgaagtaattaattaacatctgaatgtaaaatgaacattaaaatgaattactaaactttacgtctactttacaatctataaactttgtttaatcatataacgaaatacactaatacacaatcctgtacgtatgtaatacttttatccatcaaggattgagaaaaaaaagtaatgattccctgggccattaaaacttagacccccaagcttggataggtcactctctattttcgtttctcccttccctgatagaagggtgatatgtaattaagaataatatataattttataataaaagcggccgcctcccttctctaaatggactgcttggataacttggacccccttcccattttatagtcattctcttccccctcattttcccactattcccaacaatgaccatctctccaccttgtttccccattcttcctgctctacctggtgggggtgtttcaccccattaacggtcggattccgctgtggagatggctctggcctttttcccattccttccccccctcaatcttctccatgcggggaaaaaaaaattttatccataaacaaccaaaccggcggctcaacggggggtttatactgacagaaatggggtcaatacacccactgactgtacccgctctaatcttaagctttcccccccccctcctgtattaacggcgcggagtgcccgcagcgcccaatggagaaggcgcgcagtgggggatgcccagggaggggacaggtacacgcacaggccatgccaacaccgcatagacgtgcgacctcctctcccccactgcagagctgcccttttcggacacactccgtgcaagaggactcggccggctcggcttttctgccg

SEQ ID NO: 18 - DNA, 3835 bp, synthetic construct
  source 1..3835: mol_type = other DNA; note = Synthetic - 5' integration fragment
aactactatgtacactgtataagtaaaaagacgatacccccctcccactctgggtgctacggtgtagatctctccgtaaacacaaaaaggcggctcagatgataattggggtccgggcgcaaccggaaggggggagagaggggagcgatggcttctcctccggggggctacgggagtttcctctttgggaaggataaagaggggatggattgatacaagattctgagaacctattacgatgatgttcagtggtattttgtcttttgttatttaaagggaggggactttcctcaataccttagttgtaaaattacgctattatctttaaccctttcttttgagcaataattaaaaagagcggccgcgagtccatcggttcctgtcagatgggatactcttgacgtggaaaattcaaacagaaaaaaaaccccaataatgaaaaataacactacgttatatccgtggtatcctctatcgtatcgtatcgtagcgtatcgtagcgtaccgtatcacagtatagtctaatattccgtatcttattgtatcctatcctattcgatcctattgtatttcagtgcaccattttaatttctattgctataatgtccttattagttgccactgtgaggtgaccaatggacgagggcgagccgttcagaagccgcgaagggtgttcttcccatgaatttcttaaggagggcggctcagctccgagagtgaggcgagacgtctcggtcagcgtatcccccttcctcggcttttacaaatgatgcgctcttaatagtgtgtcgttatccttttggcattgacgggggagggaaattgattgagcgcatccatatttttgcggactgctgaggacaatggtggtttttccgggtggcgtgggctacaaatgatacgatggtttttttcttttcggagaaggcgtataaaaaggacacggagaacccatttattctaaaaacagttgagcttctttaattattttttgatataatattctattattatatattttcttcccaataaaacaaaataaaacaaaacacagcaaaacacaaaaagctagcggcgcgccttctgtctttgattttcttatgttattcaaaacatctgccccaaaatctaacgattatatatattcctacgtataactgtatagctaattattgatttatttgtacataaaaaccacataaatgtaaaagcaagaaaaaaaataactaaggagaaggatcaatatctcatttataatgctcgccaaagcagcgtacgtgaattttaatcaagacatcaacaaatcttgcaacttggttatatcgcttcttcacccactcacccgcttttctacattgttgaacacaaatatatacaggggtatgtctcaaggtcaagtgcagtttcaacagagactacctcaaggtacctcttcagaaatgcagaacttcactcttgatcagattttctccgaattaaaggaggcctattggtagttctttccccctctcaagctggcgtgaaatgcaaccttacggcgtctacgttactacaaggtccagaaagtgtaggtattgctactatttttattttttattggttctggagaaatgcagacagtcaatgaacacaactgtctcaatatgcatctatgcacatgcacacacacacacatcacaggtacccctacaaagagaggtctcttgataatgtttcattaccacgtggcatccccccccccccccccaataaacaagtggccgagttcccctgttgcagaggaggacaaaaaaaccgctggtgttggtaccattatgcagcaactagcacaacaaacaaccgacccagacatacaaatcaacaacacttcgccaaagacaccctttccagggaggatccactcccaacgtctctccataatgtctctgttggcccatgtctctgtcgttgacaccgtaaccacaccaaccaacccgtccattgtactgggatggtcgtccatagacacctctccaacggggaacacctcattcgtaaaccgccaaggttaccgttcctcctgactcgccccgttgttgatgctgcgcacctgtggttgcccaacatggttgtatatcgtgtaaccacaccaacacatgtgcagcacatgtgtttaaaagagtgtcatggaggtggatcatgatggaagtggactttaccacttgggaactgtctccactcccgggaagaaaagacccggcgtatcacgcggttgcctcaatggggcaatttggaaggagaaatatagggaaaatcacgtcgctctcggacggggaagagttccagactatgaggggggggggtggtatataaagacaggagatgtccacccccagagagaggaagaagttggaactttagaagagagagataactttccccagtgtccatcaatacacaaccaaacacaaactctatatttacacatataaccccctctctagattaattaatttattttactagtttatttttgctcctgagaataggattacaaacacttaaagtctttaattacaactatatataatattctgttggttttcttgaattggttcgctgcgattcatgcctcccattcaccaaaggtggagtgggaaataacggttttactgcggtaattagcagaggcaagaacaggatacactttttgatgataaatctgtattatagtcgagcctatttaggaaatcaaattttcttgtgtttacttttcaaataaataatgttcgaaaatttttactttactccttcatttaactataccagacgttatatcatcaacaccttctgaccatatacagctcaagatgtttaagagtctgttaaattttttcaatccatttcatggagtaccaggaggtgctacaaaaggaattcatagcctcatgaaatcagccatttgcttttgttcaacgatcttttgaaattgttgttgttcttggtagttaagttgatccatcttggcttatgttgtgtgtatgttgtagttattcttagtatattcctgtcctgagtttagtgaaacataatatcgccttgaaatgaaaatgctgaaattcgtcgacatacaatttttcaaactttttttttttcttggtgcacggacatgtttttaaaggaagtactctataccagttattcttcacaaatttaattgctggagaatagatcttcaacgctttaataaagtagtttgtttgtcaaggatggcgtcatacaaagaaagatcagaatcacacacttcccctgttgctaggagacttttctccatcatggaggaaaagaagtctaacctttgtgcatcattggatattactgaaactgaaaagcttctctctattttggacactattggtccttacatctgtctagttaaaacacacatcgatattgtttctgattttacgtatgaaggaactgtgttgcctttgaaggagcttgccaagaaacataattttatgatttttgaagatagaaaatttgctgatattggtaacactgttaaaaatcaatataaatctggtgtcttccgtattgccgaatgggctgacatcactaatgcacatggtgtaacgggtgcaggtattgtttctggcttgaaggaggcagcccaagaaacaaccagtgaacctagaggtttgctaatgcttgctgagttatcatcaaagggttctttagcatatggtgaatatacagaaaaaacagtagaaattgctaaatctgataaagagtttgtcattggttttattgcgcaacacgatatgggcggtagagaagaaggttttgactccgc

SEQ ID NO: 19 - DNA, 3883 bp, synthetic construct
  source 1..3883: mol_type = other DNA; note = Synthetic - Integration fragment targeted to MAE gene
aattctttgaaggagcttgccaagaaacataattttatgatttttgaagatagaaaatttgctgatattggtaacactgttaaaaatcaatataaatctggtgtcttccgtattgccgaatgggctgacatcactaatgcacatggtgtaacgggtgcaggtattgtttctggcttgaaggaggcagcccaagaaacaaccagtgaacctagaggtttgctaatgcttgctgagttatcatcaaagggttctttagcatatggtgaatatacagaaaaaacagtagaaattgctaaatctgataaagagtttgtcattggttttattgcgcaacacgatatgggcggtagagaagaaggttttgactggatcattatgactccaggggttggtttagatgacaaaggtgatgcacttggtcaacaatatagaactgttgatgaagttgtaaagactggaacggatatcataattgttggtagaggtttgtacggtcaaggaagagatcctatagagcaagctaaaagataccaacaagctggttggaatgcttatttaaacagatttaaatgattcttacacaaagatttgatacatgtacactagtttaaataagcatgaaaagaattacacaagcaaaaaaaaaaaaataaatgaggtactttacgttcacctacaaccaaaaaaactagatagagtaaaatcttaagatttagaaaaagttgtttaacaaaggctttagtatgtgaatttttaatgtagcaaagcgataactaataaacataaacaaaagtatggttttctttatcagtcaaatcattatcgattgattgttccgcgtatctgcagatagcctcatgaaatcagccatttgcttttgttcaacgatcttttgaaattgttgttgttcttggtagttaagttgatccatcttggcttatgttgtgtgtatgttgtagttattcttagtatattcctgtcctgagtttagtgaaacataatatcgccttgaaatgaaaatgctgaaattcgtcgacatacaatttttcaaactttttttttttcttggtgcacggacatgtttttaaaggaagtactctataccagttattcttcacaaatttaattgctggagaatagatcttcaacgcgtttaaacagcaatttgaggaaggaataggagaaggagaagcaatttctaggaaagagcaaggtgtgcaacagcatgctctgaatgatattttcagcaatagttcagttgaagaacctgttggcgtatctacatcacttcctacaaacaacaccacgaattgcgtccgtggtgacgcaactacgaatggcattgtcaatgccaatgccagtgcacatacacgtgcaagtcccaccggttccctgcccggctatggtagagacaagaaggacgataccggcatcgacatcaacagtttcaacagcaatgcgtttggcgtcgacgcgtcgatggggctgccgtatttggatttggacgggctagatttcgatatggatatggatatggatatggatatggagatgaatttgaatttagatttgggtcttgatttggggttggaattaaaaggggataacaatgagggttttcctgttgatttaaacaatggacgtgggaggtgattgatttaacctgatccaaaaggggtatgtctattttttagagtgtgtctttgtgtcaaattatggtagaatgtgtaaagtagtataaactttcctctcaaatgacgaggtttaaaacaccccccgggtgagccgagccgagaatggggcaattgttcaatgtgaaatagaagtatcgagtgagaaacttgggtgttggccagccaagggggaaggaaaatggcgcgaatgctcaggtgagattgttttggaattgggtgaagcgaggaaatgagcgacccggaggttgtgactttagtggcggaggaggaacgggaggaaaaggccaagagggaaagtgtatataagggggagcaatttgccaaccaggatagaattggatgagttataattctactgtatttattgtataatttatttctccttttatatcaaacacattacaaaacacacaaaacatacaaacatacacagctagcaaaggcgcgccatctaatagtttaatcacagcttatagtctactatagttttcttttttaaacattgttgtattttgtcccccccctctaattgatgatgattatcctataagaatccaataaaacgatggaaactaataccctctcctttgtcatgtggtctttagtatttcttgaacattggctctgatttctcgactttatagtcctattaaaatcgctgttagttctcgatcgttgtatctcgtttcttgtctctttggtggatgattttgcgtgcgaacatgtttttttccctttctctcaccatcatcgtgtagttcttgtcaccatcccccccaccccttccttctctcattgattctataagagcttatccacagaggtgcagtaacgaggtagtttaaccttcgagtggatcaaaatgtcacacaggcctcgacatttgctgcaacggcaacatcaatgtccacgtttacacacctacatttatatctatatttatatttatatttatttatttatgctacttagcttctatagttagttaatgcactcacgatattcaaaattgacacccttcaactactccctactattgtctactactgtctactactcctctttactatagctgctcccaataggctccaccaataggctctgtcaatacattttgcgccgccacctttcaggttgtgtcactcctgaaggaccatattgggtaatcgtgcaatttctggaagagagtccgcgagaagtgaggcccccactgtaaatcctcgagggggcatggagtatggggcatggaggatggaggatggggggggggggggggaaaataggtagcgaaaggacccgctatcaccccacccggagaactcgttgccgggaagtcatatttcgacactccggggagtctataaaaggcgggttttgtcttttgccagttgatgttgctgagaggacttgtttgccgtttcttccgatttaacagtatagaatcaaccactgttaattatacacgttatactaacacaacaaaaacaaaaacaacgacaacaacaacaacatctagataattaattaacatctgaatgtaaaatgaacattaaaatgaattactaaactttacgtctactttacaatctataaactttgtttaatcatataacgaaatacactaatacacaatcctgtacgtatgtaatacttttatccatcaaggattgagaaaaaaaagtaatgattccctgggccattaaaacttagacccccaagcttggataggtcactctctattttcgtttctcccttccctgatagaagggtgatatgtaattaagaataatatataattttataataaaagcggccgcacacatacacattatcaaatgcatttattcctaatatcacactaaaacgtattatataattttaatctttatagacttcatagcaccaattggatttgctttctttcagaataccgcacttaatctcaatgtacgtaacgtaggcaaaatctgtcgataaggatctgtatgccgtaaacggaaactccaagcgcccagaaaacttacattatattcttgccagtttcatctcaccagccagtcacagtttaaaaggtttgattgcgtttcttgtttcgtcggattcagtgctaattggtaacgcactgtaccgccacaccaaagcaaaaatgcagaaacaaacaacaatgagtgtatgtttaccaactttgg

SEQ ID NO: 20 - DNA, 5722 bp, synthetic construct
  source 1..5722: mol_type = other DNA; note = Synthetic - E. coli SthA gene integration fragment
aactactatgtacactgtataagtaaaaagacgatacccccctcccactctgggtgctacggtgtagatctctccgtaaacacaaaaaggcggctcagatgataattggggtccgggcgcaaccggaaggggggagagaggggagcgatggcttctcctccggggggctacgggagtttcctctttgggaaggataaagaggggatggattgatacaagattctgagaacctattacgatgatgttcagtggtattttgtcttttgttatttaaagggaggggactttcctcaataccttagttgtaaaattacgctattatctttaaccctttcttttgagcaataattaaaaagagcggccgcgagtccatcggttcctgtcagatgggatactcttgacgtggaaaattcaaacagaaaaaaaaccccaataatgaaaaataacactacgttatatccgtggtatcctctatcgtatcgtatcgtagcgtatcgtagcgtaccgtatcacagtatagtctaatattccgtatcttattgtatcctatcctattcgatcctattgtatttcagtgcaccattttaatttctattgctataatgtccttattagttgccactgtgaggtgaccaatggacgagggcgagccgttcagaagccgcgaagggtgttcttcccatgaatttcttaaggagggcggctcagctccgagagtgaggcgagacgtctcggtcagcgtatcccccttcctcggcttttacaaatgatgcgctcttaatagtgtgtcgttatccttttggcattgacgggggagggaaattgattgagcgcatccatatttttgcggactgctgaggacaatggtggtttttccgggtggcgtgggctacaaatgatacgatggtttttttcttttcggagaaggcgtataaaaaggacacggagaacccatttattctaaaaacagttgagcttctttaattattttttgatataatattctattattatatattttcttcccaataaaacaaaataaaacaaaacacagcaaaacacaaaaagctagcctgaaagggaaccataatgggtaagatcgcaccacattagcgggctcgaagatggatcttgcgaatgggtgacaccagtcataaggcctcgttgtcccagcatacctcccgcgctatctaattgcttcgctctccattgttcttggtaaacatcactctggcttgatggtgtcatctatgcccgccaagcctatcggtctatggcccggagtttgctccgtcttccaattgcaatcgcacggaatccgggatagaaagaacgatacgcattcatacgattctcacgttattggttggtgaatcaaatgcacaacgaacccaatcgccctggactcagcgtctaggccccccgtatggccgacggggactcagagcgtcaatccacgttgaagtcgaggttttggcagttacagcccttgcaataaggtttttcggacagtctactttgtcggcgcgccttctgtctttgattttcttatgttattcaaaacatctgccccaaaatctaacgattatatatattcctacgtataactgtatagctaattattgatttatttgtacataaaaaccacataaatgtaaaagcaagaaaaaaaataactaaggagaaggatcaatatctcatttataatgctcgccaaagcagcgtacgtgaattttaatcaagacatcaacaaatcttgcaacttggttatatcgcttcttcacccactcacccgcttttctacattgttgaacacaaatatatacaggggtatgtctcaaggtcaagtgcagtttcaacagagactacctcaaggtacctcttcagaaatgcagaacttcactcttgatcagattttctccgaattaaaggaggcctattggtagttctttccccctctcaagctggcgtgaaatgcaaccttacggcgtctacgttactacaaggtccagaaagtgtaggtattgctactatttttattttttattggttctggagaaatgcagacagtcaatgaacacaactgtctcaatatgcatctatgcacatgcacacacacacacatcacaggtacccctacaaagagaggtctcttgataatgtttcattaccacgtggcatccccccccccccccccaataaacaagtggccgagttcccctgttgcagaggaggacaaaaaaaccgctggtgttggtaccattatgcagcaactagcacaacaaacaaccgacccagacatacaaatcaacaacacttcgccaaagacaccctttccagggaggatccactcccaacgtctctccataatgtctctgttggcccatgtctctgtcgttgacaccgtaaccacaccaaccaacccgtccattgtactgggatggtcgtccatagacacctctccaacggggaacacctcattcgtaaaccgccaaggttaccgttcctcctgactcgccccgttgttgatgctgcgcacctgtggttgcccaacatggttgtatatcgtgtaaccacaccaacacatgtgcagcacatgtgtttaaaagagtgtcatggaggtggatcatgatggaagtggactttaccacttgggaactgtctccactcccgggaagaaaagacccggcgtatcacgcggttgcctcaatggggcaatttggaaggagaaatatagggaaaatcacgtcgctctcggacggggaagagttccagactatgaggggggggggtggtatataaagacaggagatgtccacccccagagagaggaagaagttggaactttagaagagagagataactttccccagtgtccatcaatacacaaccaaacacaaactctatatttacacatataaccccctctctagaatgccacattcctacgattacgatgccatagtaataggttccggccccggcggcgaaggcgctgcaatgggcctggttaagcaaggtgcgcgcgtcgcagttatcgagcgttatcaaaatgttggcggcggttgcacccactggggcaccatcccgtcgaaagctctccgtcacgccgtcagccgcattatagaattcaatcaaaacccactttacagcgaccattcccgactgctccgctcttcttttgccgatatccttaaccatgccgataacgtgattaatcaacaaacgcgcatgcgtcagggattttacgaacgtaatcactgtgaaatattgcagggaaacgctcgctttgttgacgagcatacgttggcgctggattgcccggacggcagcgttgaaacactaaccgctgaaaaatttgttattgcctgcggctctcgtccatatcatccaacagatgttgatttcacccatccacgcatttacgacagcgactcaattctcagcatgcaccacgaaccgcgccatgtacttatctatggtgctggagtgatcggctgtgaatatgcgtcgatcttccgcggtatggatgtaaaagtggatctgatcaacacccgcgatcgcctgctggcatttctcgatcaagagatgtcagattctctctcctatcacttctggaacagtggcgtagtgattcgtcacaacgaagagtacgagaagatcgaaggctgtgacgatggtgtgatcatgcatctgaagtcgggtaaaaaactgaaagctgactgcctgctctatgccaacggtcgcaccggtaataccgattcgctggcgttacagaacattgggctagaaactgacagccgcggacagctgaaggtcaacagcatgtatcagaccgcacagccacacgtttacgcggtgggcgacgtgattggttatccgagcctggcgtcggcggcctatgaccaggggcgcattgccgcgcaggcgctggtaaaaggcgaagccaccgcacatctgattgaagatatccctaccggtatttacaccatcccggaaatcagctctgtgggcaaaaccgaacagcagctgaccgcaatgaaagtgccatatgaagtgggccgcgcccagtttaaacatctggcacgcgcacaaatcgtcggcatgaacgtgggcacgctgaaaattttgttccatcgggaaacaaaagagattctgggtattcactgctttggcgagcgcgctgccgaaattattcatatcggtcaggcgattatggaacagaaaggtggcggcaacactattgagtacttcgtcaacaccacctttaactacccgacgatggcggaagcctatcgggtagctgcgttaaacggtttaaaccgcctgttttaaaactttatcgaaatggccatccattcttgcgcggatttaattaatttattttactagtttatttttgctcctgagaataggattacaaacacttaaagtctttaattacaactatatataatattctgttggttttcttgaattggttcgctgcgattcatgcctcccattcaccaaaggtggagtgggaaataacggttttactgcggtaattagcagaggcaagaacaggatacactttttgatgataaatctgtattatagtcgagcctatttaggaaatcaaattttcttgtgtttacttttcaaataaataatgttcgaaaatttttactttactccttcatttaactataccagacgttatatcatcaacaccttctgaccatatacagctcaagatgtttaagagtctgttaaattttttcaatccatttcatggagtaccaggaggtgctacaaaaggaattcatagcctcatgaaatcagccatttgcttttgttcaacgatcttttgaaattgttgttgttcttggtagttaagttgatccatcttggcttatgttgtgtgtatgttgtagttattcttagtatattcctgtcctgagtttagtgaaacataatatcgccttgaaatgaaaatgctgaaattcgtcgacatacaatttttcaaactttttttttttcttggtgcacggacatgtttttaaaggaagtactctataccagttattcttcacaaatttaattgctggagaatagatcttcaacgctttaataaagtagtttgtttgtcaaggatggcgtcatacaaagaaagatcagaatcacacacttcccctgttgctaggagacttttctccatcatggaggaaaagaagtctaacctttgtgcatcattggatattactgaaactgaaaagcttctctctattttggacactattggtccttacatctgtctagttaaaacacacatcgatattgtttctgattttacgtatgaaggaactgtgttgcctttgaaggagcttgccaagaaacataattttatgatttttgaagatagaaaatttgctgatattggtaacactgttaaaaatcaatataaatctggtgtcttccgtattgccgaatgggctgacatcactaatgcacatggtgtaacgggtgcaggtattgtttctggcttgaaggaggcagcccaagaaacaaccagtgaacctagaggtttgctaatgcttgctgagttatcatcaaagggttctttagcatatggtgaatatacagaaaaaacagtagaaattgctaaatctgataaagagtttgtcattggttttattgcgcaacacgatatgggcggtagagaagaaggttttgactccgc

SEQ ID NO: 21 - DNA, 1401 bp, Escherichia coli
  source 1..1401: mol_type = other DNA
atgccacattcctacgattacgatgccatagtaataggttccggccccggcggcgaaggcgctgcaatgggcctggttaagcaaggtgcgcgcgtcgcagttatcgagcgttatcaaaatgttggcggcggttgcacccactggggcaccatcccgtcgaaagctctccgtcacgccgtcagccgcattatagaattcaatcaaaacccactttacagcgaccattcccgactgctccgctcttcttttgccgatatccttaaccatgccgataacgtgattaatcaacaaacgcgcatgcgtcagggattttacgaacgtaatcactgtgaaatattgcagggaaacgctcgctttgttgacgagcatacgttggcgctggattgcccggacggcagcgttgaaacactaaccgctgaaaaatttgttattgcctgcggctctcgtccatatcatccaacagatgttgatttcacccatccacgcatttacgacagcgactcaattctcagcatgcaccacgaaccgcgccatgtacttatctatggtgctggagtgatcggctgtgaatatgcgtcgatcttccgcggtatggatgtaaaagtggatctgatcaacacccgcgatcgcctgctggcatttctcgatcaagagatgtcagattctctctcctatcacttctggaacagtggcgtagtgattcgtcacaacgaagagtacgagaagatcgaaggctgtgacgatggtgtgatcatgcatctgaagtcgggtaaaaaactgaaagctgactgcctgctctatgccaacggtcgcaccggtaataccgattcgctggcgttacagaacattgggctagaaactgacagccgcggacagctgaaggtcaacagcatgtatcagaccgcacagccacacgtttacgcggtgggcgacgtgattggttatccgagcctggcgtcggcggcctatgaccaggggcgcattgccgcgcaggcgctggtaaaaggcgaagccaccgcacatctgattgaagatatccctaccggtatttacaccatcccggaaatcagctctgtgggcaaaaccgaacagcagctgaccgcaatgaaagtgccatatgaagtgggccgcgcccagtttaaacatctggcacgcgcacaaatcgtcggcatgaacgtgggcacgctgaaaattttgttccatcgggaaacaaaagagattctgggtattcactgctttggcgagcgcgctgccgaaattattcatatcggtcaggcgattatggaacagaaaggtggcggcaacactattgagtacttcgtcaacaccacctttaactacccgacgatggcggaagcctatcgggtagctgcgttaaacggtttaaaccgcctgttttaa

SEQ ID NO: 22 - DNA, 5335 bp, synthetic construct
  source 1..5335: mol_type = other DNA; note = Synthetic - RH E. coli integration fragment
aattctttgaaggagcttgccaagaaacataattttatgatttttgaagatagaaaatttgctgatattggtaacactgttaaaaatcaatataaatctggtgtcttccgtattgccgaatgggctgacatcactaatgcacatggtgtaacgggtgcaggtattgtttctggcttgaaggaggcagcccaagaaacaaccagtgaacctagaggtttgctaatgcttgctgagttatcatcaaagggttctttagcatatggtgaatatacagaaaaaacagtagaaattgctaaatctgataaagagtttgtcattggttttattgcgcaacacgatatgggcggtagagaagaaggttttgactggatcattatgactccaggggttggtttagatgacaaaggtgatgcacttggtcaacaatatagaactgttgatgaagttgtaaagactggaacggatatcataattgttggtagaggtttgtacggtcaaggaagagatcctatagagcaagctaaaagataccaacaagctggttggaatgcttatttaaacagatttaaatgattcttacacaaagatttgatacatgtacactagtttaaataagcatgaaaagaattacacaagcaaaaaaaaaaaaataaatgaggtactttacgttcacctacaaccaaaaaaactagatagagtaaaatcttaagatttagaaaaagttgtttaacaaaggctttagtatgtgaatttttaatgtagcaaagcgataactaataaacataaacaaaagtatggttttctttatcagtcaaatcattatcgattgattgttccgcgtatctgcagatagcctcatgaaatcagccatttgcttttgttcaacgatcttttgaaattgttgttgttcttggtagttaagttgatccatcttggcttatgttgtgtgtatgttgtagttattcttagtatattcctgtcctgagtttagtgaaacataatatcgccttgaaatgaaaatgctgaaattcgtcgacatacaatttttcaaactttttttttttcttggtgcacggacatgtttttaaaggaagtactctataccagttattcttcacaaatttaattgctggagaatagatcttcaacgcgtttaaacagcaatttgaggaaggaataggagaaggagaagcaatttctaggaaagagcaaggtgtgcaacagcatgctctgaatgatattttcagcaatagttcagttgaagaacctgttggcgtatctacatcacttcctacaaacaacaccacgaattgcgtccgtggtgacgcaactacgaatggcattgtcaatgccaatgccagtgcacatacacgtgcaagtcccaccggttccctgcccggctatggtagagacaagaaggacgataccggcatcgacatcaacagtttcaacagcaatgcgtttggcgtcgacgcgtcgatggggctgccgtatttggatttggacgggctagatttcgatatggatatggatatggatatggatatggagatgaatttgaatttagatttgggtcttgatttggggttggaattaaaaggggataacaatgagggttttcctgttgatttaaacaatggacgtgggaggtgattgatttaacctgatccaaaaggggtatgtctattttttagagtgtgtctttgtgtcaaattatggtagaatgtgtaaagtagtataaactttcctctcaaatgacgaggtttaaaacaccccccgggtgagccgagccgagaatggggcaattgttcaatgtgaaatagaagtatcgagtgagaaacttgggtgttggccagccaagggggaaggaaaatggcgcgaatgctcaggtgagattgttttggaattgggtgaagcgaggaaatgagcgacccggaggttgtgactttagtggcggaggaggaacgggaggaaaaggccaagagggaaagtgtatataagggggagcaatttgccaaccaggatagaattggatgagttataattctactgtatttattgtataatttatttctccttttatatcaaacacattacaaaacacacaaaacatacaaacatacacagctagcaaaggcgcgccatctaatagtttaatcacagcttatagtctactatagttttcttttttaaacattgttgtattttgtcccccccctctaattgatgatgattatcctataagaatccaataaaacgatggaaactaataccctctcctttgtcatgtggtctttagtatttcttgaacattggctctgatttctcgactttatagtcctattaaaatcgctgttagttctcgatcgttgtatctcgtttcttgtctctttggtggatgattttgcgtgcgaacatgtttttttccctttctctcaccatcatcgtgtagttcttgtcaccatcccccccaccccttccttctctcattgattctataagagcttatccacagaggtgcagtaacgaggtagtttaaccttcgagtggatcaaaatgtcacacaggcctcgacatttgctgcaacggcaacatcaatgtccacgtttacacacctacatttatatctatatttatatttatatttatttatttatgctacttagcttctatagttagttaatgcactcacgatattcaaaattgacacccttcaactactccctactattgtctactactgtctactactcctctttactatagctgctcccaataggctccaccaataggctctgtcaatacattttgcgccgccacctttcaggttgtgtcactcctgaaggaccatattgggtaatcgtgcaatttctggaagagagtccgcgagaagtgaggcccccactgtaaatcctcgagggggcatggagtatggggcatggaggatggaggatggggggggggggggggaaaataggtagcgaaaggacccgctatcaccccacccggagaactcgttgccgggaagtcatatttcgacactccggggagtctataaaaggcgggttttgtcttttgccagttgatgttgctgagaggacttgtttgccgtttcttccgatttaacagtatagaatcaaccactgttaattatacacgttatactaacacaacaaaaacaaaaacaacgacaacaacaacaacatctagaatgccacattcctacgattacgatgccatagtaataggttccggccccggcggcgaaggcgctgcaatgggcctggttaagcaaggtgcgcgcgtcgcagttatcgagcgttatcaaaatgttggcggcggttgcacccactggggcaccatcccgtcgaaagctctccgtcacgccgtcagccgcattatagaattcaatcaaaacccactttacagcgaccattcccgactgctccgctcttcttttgccgatatccttaaccatgccgataacgtgattaatcaacaaacgcgcatgcgtcagggattttacgaacgtaatcactgtgaaatattgcagggaaacgctcgctttgttgacgagcatacgttggcgctggattgcccggacggcagcgttgaaacactaaccgctgaaaaatttgttattgcctgcggctctcgtccatatcatccaacagatgttgatttcacccatccacgcatttacgacagcgactcaattctcagcatgcaccacgaaccgcgccatgtacttatctatggtgctggagtgatcggctgtgaatatgcgtcgatcttccgcggtatggatgtaaaagtggatctgatcaacacccgcgatcgcctgctggcatttctcgatcaagagatgtcagattctctctcctatcacttctggaacagtggcgtagtgattcgtcacaacgaagagtacgagaagatcgaaggctgtgacgatggtgtgatcatgcatctgaagtcgggtaaaaaactgaaagctgactgcctgctctatgccaacggtcgcaccggtaataccgattcgctggcgttacagaacattgggctagaaactgacagccgcggacagctgaaggtcaacagcatgtatcagaccgcacagccacacgtttacgcggtgggcgacgtgattggttatccgagcctggcgtcggcggcctatgaccaggggcgcattgccgcgcaggcgctggtaaaaggcgaagccaccgcacatctgattgaagatatccctaccggtatttacaccatcccggaaatcagctctgtgggcaaaaccgaacagcagctgaccgcaatgaaagtgccatatgaagtgggccgcgcccagtttaaacatctggcacgcgcacaaatcgtcggcatgaacgtgggcacgctgaaaattttgttccatcgggaaacaaaagagattctgggtattcactgctttggcgagcgcgctgccgaaattattcatatcggtcaggcgattatggaacagaaaggtggcggcaacactattgagtacttcgtcaacaccacctttaactacccgacgatggcggaagcctatcgggtagctgcgttaaacggtttaaaccgcctgttttaaaactttatcgaaatggccatccattcttgcgcggatttaattaacatctgaatgtaaaatgaacattaaaatgaattactaaactttacgtctactttacaatctataaactttgtttaatcatataacgaaatacactaatacacaatcctgtacgtatgtaatacttttatccatcaaggattgagaaaaaaaagtaatgattccctgggccattaaaacttagacccccaagcttggataggtcactctctattttcgtttctcccttccctgatagaagggtgatatgtaattaagaataatatataattttataataaaagcggccgcacacatacacattatcaaatgcatttattcctaatatcacactaaaacgtattatataattttaatctttatagacttcatagcaccaattggatttgctttctttcagaataccgcacttaatctcaatgtacgtaacgtaggcaaaatctgtcgataaggatctgtatgccgtaaacggaaactccaagcgcccagaaaacttacattatattcttgccagtttcatctcaccagccagtcacagtttaaaaggtttgattgcgtttcttgtttcgtcggattcagtgctaattggtaacgcactgtaccgccacaccaaagcaaaaatgcagaaacaaacaacaatgagtgtatgtttaccaactttggttttgaaagttaacccgc

SEQ ID NO: 23 - DNA, 5642 bp, synthetic construct
  source 1..5642: mol_type = other DNA; note = Synthetic - Codon-optimized E. coli Stha gene integration fragment
aaccccactctgggtgctacggtgtagatctctccgtaaacacaaaaaggcggctcagatgataattggggtccgggcgcaaccggaaggggggagagaggggagcgatggcttctcctccggggggctacgggagtttcctctttgggaaggataaagaggggatggattgatacaagattctgagaacctattacgatgatgttcagtggtattttgtcttttgttatttaaagggaggggactttcctcaataccttagttgtaaaattacgctattatctttaaccctttcttttgagcaataattaaaaagagcggccgcgagtccatcggttcctgtcagatgggatactcttgacgtggaaaattcaaacagaaaaaaaaccccaataatgaaaaataacactacgttatatccgtggtatcctctatcgtatcgtatcgtagcgtatcgtagcgtaccgtatcacagtatagtctaatattccgtatcttattgtatcctatcctattcgatcctattgtatttcagtgcaccattttaatttctattgctataatgtccttattagttgccactgtgaggtgaccaatggacgagggcgagccgttcagaagccgcgaagggtgttcttcccatgaatttcttaaggagggcggctcagctccgagagtgaggcgagacgtctcggtcagcgtatcccccttcctcggcttttacaaatgatgcgctcttaatagtgtgtcgttatccttttggcattgacgggggagggaaattgattgagcgcatccatatttttgcggactgctgaggacaatggtggtttttccgggtggcgtgggctacaaatgatacgatggtttttttcttttcggagaaggcgtataaaaaggacacggagaacccatttattctaaaaacagttgagcttctttaattattttttgatataatattctattattatatattttcttcccaataaaacaaaataaaacaaaacacagcaaaacacaaaaagctagcctgaaagggaaccataatgggtaagatcgcaccacattagcgggctcgaagatggatcttgcgaatgggtgacaccagtcataaggcctcgttgtcccagcatacctcccgcgctatctaattgcttcgctctccattgttcttggtaaacatcactctggcttgatggtgtcatctatgcccgccaagcctatcggtctatggcccggagtttgctccgtcttccaattgcaatcgcacggaatccgggatagaaagaacgatacgcattcatacgattctcacgttattggttggtgaatcaaatgcacaacgaacccaatcgccctggactcagcgtctaggccccccgtatggccgacggggactcagagcgtcaatccacgttgaagtcgaggttttggcagttacagcccttgcaataaggtttttcggacagtctactttgtcggcgcgccttctgtctttgattttcttatgttattcaaaacatctgccccaaaatctaacgattatatatattcctacgtataactgtatagctaattattgatttatttgtacataaaaaccacataaatgtaaaagcaagaaaaaaaataactaaggagaaggatcaatatctcatttataatgctcgccaaagcagcgtacgtgaattttaatcaagacatcaacaaatcttgcaacttggttatatcgcttcttcacccactcacccgcttttctacattgttgaacacaaatatatacaggggtatgtctcaaggtcaagtgcagtttcaacagagactacctcaaggtacctcttcagaaatgcagaacttcactcttgatcagattttctccgaattaaaggaggcctattggtagttctttccccctctcaagctggcgtgaaatgcaaccttacggcgtctacgttactacaaggtccagaaagtgtaggtattgctactatttttattttttattggttctggagaaatgcagacagtcaatgaacacaactgtctcaatatgcatctatgcacatgcacacacacacacatcacaggtacccctacaaagagaggtctcttgataatgtttcattaccacgtggcatccccccccccccccccaataaacaagtggccgagttcccctgttgcagaggaggacaaaaaaaccgctggtgttggtaccattatgcagcaactagcacaacaaacaaccgacccagacatacaaatcaacaacacttcgccaaagacaccctttccagggaggatccactcccaacgtctctccataatgtctctgttggcccatgtctctgtcgttgacaccgtaaccacaccaaccaacccgtccattgtactgggatggtcgtccatagacacctctccaacggggaacacctcattcgtaaaccgccaaggttaccgttcctcctgactcgccccgttgttgatgctgcgcacctgtggttgcccaacatggttgtatatcgtgtaaccacaccaacacatgtgcagcacatgtgtttaaaagagtgtcatggaggtggatcatgatggaagtggactttaccacttgggaactgtctccactcccgggaagaaaagacccggcgtatcacgcggttgcctcaatggggcaatttggaaggagaaatatagggaaaatcacgtcgctctcggacggggaagagttccagactatgaggggggggggtggtatataaagacaggagatgtccacccccagagagaggaagaagttggaactttagaagagagagataactttccccagtgtccatcaatacacaaccaaacacaaactctatatttacacatataaccccctctctagaatgccacattcctatgactacgatgccattgtcattggttccggtccaggtggtgaaggtgctgcaatgggcttagttaagcagggtgctagagttgctgtcatcgaaagatatcaaaatgttggtggtggttgtactcactggggtacaattccatctaaggcattgagacatgcagtttccagaattattgagtttaaccaaaaccctttatactctgatcattcaagattgttgagatcatcttttgctgatattttgaaccatgctgacaacgtcatcaaccaacaaactcgtatgcgtcaaggcttctatgagagaaatcattgtgagattttacaaggtaacgctagatttgtcgatgagcatactcttgcattagactgtccagacggttccgttgagactcttaccgctgaaaaattcgttattgcttgtggttccagaccataccacccaaccgatgtcgatttcactcaccctcgtatctacgattccgattctattttgtctatgcatcatgaaccaagacatgttttgatttatggtgctggtgttatcggttgtgaatatgcttctattttcagaggtatggatgttaaggttgacttgattaatacaagagacagattattagctttccttgatcaggaaatgtctgattccctttcctaccatttttggaactccggtgtcgtcatcagacacaacgaggaatatgaaaagattgaaggttgtgatgacggcgttattatgcaccttaagtctggtaaaaagttaaaagcagattgcttgttatatgcaaatggtagaaccggtaacacagactccttggctttacaaaacattggtttagaaaccgattcaagaggtcaattaaaggtcaattcaatgtatcaaactgcacaaccacacgtttacgcagttggtgacgttattggttacccttcattggcatctgccgcttacgatcaaggtagaatcgccgctcaagcacttgttaagggtgaagcaactgcacacttaatcgaagatatccctaccggtatctacactatcccagaaatctcttctgttggcaagactgaacaacaattaaccgcaatgaaggttccatacgaagtcggtcgtgcccagttcaagcatttggctagagcacaaattgttggtatgaatgttggtactttgaaaatcttgtttcacagagaaacaaaggaaatcttgggcattcactgtttcggcgaaagagctgcagagattattcacatcggtcaagccattatggaacaaaaaggcggtggtaataccattgaatatttcgttaataccaccttcaactacccaacaatggccgaagcatatagagtcgctgctttaaacggtttaaacagattgttttaattaatttattttactagtttatttttgctcctgagaataggattacaaacacttaaagtctttaattacaactatatataatattctgttggttttcttgaattggttcgctgcgattcatgcctcccattcaccaaaggtggagtgggaaataacggttttactgcggtaattagcagaggcaagaacaggatacactttttgatgataaatctgtattatagtcgagcctatttaggaaatcaaattttcttgtgtttacttttcaaataaataatgttcgaaaatttttactttactccttcatttaactataccagacgttatatcatcaacaccttctgaccatatacagctcaagatgtttaagagtctgttaaattttttcaatccatttcatggagtaccaggaggtgctacaaaaggaattcatagcctcatgaaatcagccatttgcttttgttcaacgatcttttgaaattgttgttgttcttggtagttaagttgatccatcttggcttatgttgtgtgtatgttgtagttattcttagtatattcctgtcctgagtttagtgaaacataatatcgccttgaaatgaaaatgctgaaattcgtcgacatacaatttttcaaactttttttttttcttggtgcacggacatgtttttaaaggaagtactctataccagttattcttcacaaatttaattgctggagaatagatcttcaacgctttaataaagtagtttgtttgtcaaggatggcgtcatacaaagaaagatcagaatcacacacttcccctgttgctaggagacttttctccatcatggaggaaaagaagtctaacctttgtgcatcattggatattactgaaactgaaaagcttctctctattttggacactattggtccttacatctgtctagttaaaacacacatcgatattgtttctgattttacgtatgaaggaactgtgttgcctttgaaggagcttgccaagaaacataattttatgatttttgaagatagaaaatttgctgatattggtaacactgttaaaaatcaatataaatctggtgtcttccgtattgccgaatgggctgacatcactaatgcacatggtgtaacgggtgcaggtattgtttctggcttgaaggaggcagcccaagaaacaaccagtgaacctagaggtttgctaatgcttgctgagttatcatcaaagggttctttagcatatggtgaatatacagaaaaaacagtagaaattgctaaatctgataaagagtttgtcattggttttattgcgcaacacgatatgggcggtagagaagaaggttttgactccgc

SEQ ID NO: 24 - DNA, 1401 bp, synthetic construct
  source 1..1401: mol_type = other DNA; note = Synthetic - Codon optimized E. coli SthA gene
atgccacattcctatgactacgatgccattgtcattggttccggtccaggtggtgaaggtgctgcaatgggcttagttaagcagggtgctagagttgctgtcatcgaaagatatcaaaatgttggtggtggttgtactcactggggtacaattccatctaaggcattgagacatgcagtttccagaattattgagtttaaccaaaaccctttatactctgatcattcaagattgttgagatcatcttttgctgatattttgaaccatgctgacaacgtcatcaaccaacaaactcgtatgcgtcaaggcttctatgagagaaatcattgtgagattttacaaggtaacgctagatttgtcgatgagcatactcttgcattagactgtccagacggttccgttgagactcttaccgctgaaaaattcgttattgcttgtggttccagaccataccacccaaccgatgtcgatttcactcaccctcgtatctacgattccgattctattttgtctatgcatcatgaaccaagacatgttttgatttatggtgctggtgttatcggttgtgaatatgcttctattttcagaggtatggatgttaaggttgacttgattaatacaagagacagattattagctttccttgatcaggaaatgtctgattccctttcctaccatttttggaactccggtgtcgtcatcagacacaacgaggaatatgaaaagattgaaggttgtgatgacggcgttattatgcaccttaagtctggtaaaaagttaaaagcagattgcttgttatatgcaaatggtagaaccggtaacacagactccttggctttacaaaacattggtttagaaaccgattcaagaggtcaattaaaggtcaattcaatgtatcaaactgcacaaccacacgtttacgcagttggtgacgttattggttacccttcattggcatctgccgcttacgatcaaggtagaatcgccgctcaagcacttgttaagggtgaagcaactgcacacttaatcgaagatatccctaccggtatctacactatcccagaaatctcttctgttggcaagactgaacaacaattaaccgcaatgaaggttccatacgaagtcggtcgtgcccagttcaagcatttggctagagcacaaattgttggtatgaatgttggtactttgaaaatcttgtttcacagagaaacaaaggaaatcttgggcattcactgtttcggcgaaagagctgcagagattattcacatcggtcaagccattatggaacaaaaaggcggtggtaataccattgaatatttcgttaataccaccttcaactacccaacaatggccgaagcatatagagtcgctgctttaaacggtttaaacagattgttttaa

SEQ ID NO: 25 - DNA, 5304 bp, synthetic construct
  source 1..5304: mol_type = other DNA; note = Synthetic - Codon-optimized E. coli SthA gene integration fragment
aattctttgaaggagcttgccaagaaacataattttatgatttttgaagatagaaaatttgctgatattggtaacactgttaaaaatcaatataaatctggtgtcttccgtattgccgaatgggctgacatcactaatgcacatggtgtaacgggtgcaggtattgtttctggcttgaaggaggcagcccaagaaacaaccagtgaacctagaggtttgctaatgcttgctgagttatcatcaaagggttctttagcatatggtgaatatacagaaaaaacagtagaaattgctaaatctgataaagagtttgtcattggttttattgcgcaacacgatatgggcggtagagaagaaggttttgactggatcattatgactccaggggttggtttagatgacaaaggtgatgcacttggtcaacaatatagaactgttgatgaagttgtaaagactggaacggatatcataattgttggtagaggtttgtacggtcaaggaagagatcctatagagcaagctaaaagataccaacaagctggttggaatgcttatttaaacagatttaaatgattcttacacaaagatttgatacatgtacactagtttaaataagcatgaaaagaattacacaagcaaaaaaaaaaaaataaatgaggtactttacgttcacctacaaccaaaaaaactagatagagtaaaatcttaagatttagaaaaagttgtttaacaaaggctttagtatgtgaatttttaatgtagcaaagcgataactaataaacataaacaaaagtatggttttctttatcagtcaaatcattatcgattgattgttccgcgtatctgcagatagcctcatgaaatcagccatttgcttttgttcaacgatcttttgaaattgttgttgttcttggtagttaagttgatccatcttggcttatgttgtgtgtatgttgtagttattcttagtatattcctgtcctgagtttagtgaaacataatatcgccttgaaatgaaaatgctgaaattcgtcgacatacaatttttcaaactttttttttttcttggtgcacggacatgtttttaaaggaagtactctataccagttattcttcacaaatttaattgctggagaatagatcttcaacgcgtttaaacagcaatttgaggaaggaataggagaaggagaagcaatttctaggaaagagcaaggtgtgcaacagcatgctctgaatgatattttcagcaatagttcagttgaagaacctgttggcgtatctacatcacttcctacaaacaacaccacgaattgcgtccgtggtgacgcaactacgaatggcattgtcaatgccaatgccagtgcacatacacgtgcaagtcccaccggttccctgcccggctatggtagagacaagaaggacgataccggcatcgacatcaacagtttcaacagcaatgcgtttggcgtcgacgcgtcgatggggctgccgtatttggatttggacgggctagatttcgatatggatatggatatggatatggatatggagatgaatttgaatttagatttgggtcttgatttggggttggaattaaaaggggataacaatgagggttttcctgttgatttaaacaatggacgtgggaggtgattgatttaacctgatccaaaaggggtatgtctattttttagagtgtgtctttgtgtcaaattatggtagaatgtgtaaagtagtataaactttcctctcaaatgacgaggtttaaaacaccccccgggtgagccgagccgagaatggggcaattgttcaatgtgaaatagaagtatcgagtgagaaacttgggtgttggccagccaagggggaaggaaaatggcgcgaatgctcaggtgagattgttttggaattgggtgaagcgaggaaatgagcgacccggaggttgtgactttagtggcggaggaggaacgggaggaaaaggccaagagggaaagtgtatataagggggagcaatttgccaaccaggatagaattggatgagttataattctactgtatttattgtataatttatttctccttttatatcaaacacattacaaaacacacaaaacatacaaacatacacagctagcaaaggcgcgccatctaatagtttaatcacagcttatagtctactatagttttcttttttaaacattgttgtattttgtcccccccctctaattgatgatgattatcctataagaatccaataaaacgatggaaactaataccctctcctttgtcatgtggtctttagtatttcttgaacattggctctgatttctcgactttatagtcctattaaaatcgctgttagttctcgatcgttgtatctcgtttcttgtctctttggtggatgattttgcgtgcgaacatgtttttttccctttctctcaccatcatcgtgtagttcttgtcaccatcccccccaccccttccttctctcattgattctataagagcttatccacagaggtgcagtaacgaggtagtttaaccttcgagtggatcaaaatgtcacacaggcctcgacatttgctgcaacggcaacatcaatgtccacgtttacacacctacatttatatctatatttatatttatatttatttatttatgctacttagcttctatagttagttaatgcactcacgatattcaaaattgacacccttcaactactccctactattgtctactactgtctactactcctctttactatagctgctcccaataggctccaccaataggctctgtcaatacattttgcgccgccacctttcaggttgtgtcactcctgaaggaccatattgggtaatcgtgcaatttctggaagagagtccgcgagaagtgaggcccccactgtaaatcctcgagggggcatggagtatggggcatggaggatggaggatggggggggggggggggaaaataggtagcgaaaggacccgctatcaccccacccggagaactcgttgccgggaagtcatatttcgacactccggggagtctataaaaggcgggttttgtcttttgccagttgatgttgctgagaggacttgtttgccgtttcttccgatttaacagtatagaatcaaccactgttaattatacacgttatactaacacaacaaaaacaaaaacaacgacaacaacaacaacatctagaatgccacattcctatgactacgatgccattgtcattggttccggtccaggtggtgaaggtgctgcaatgggcttagttaagcagggtgctagagttgctgtcatcgaaagatatcaaaatgttggtggtggttgtactcactggggtacaattccatctaaggcattgagacatgcagtttccagaattattgagtttaaccaaaaccctttatactctgatcattcaagattgttgagatcatcttttgctgatattttgaaccatgctgacaacgtcatcaaccaacaaactcgtatgcgtcaaggcttctatgagagaaatcattgtgagattttacaaggtaacgctagatttgtcgatgagcatactcttgcattagactgtccagacggttccgttgagactcttaccgctgaaaaattcgttattgcttgtggttccagaccataccacccaaccgatgtcgatttcactcaccctcgtatctacgattccgattctattttgtctatgcatcatgaaccaagacatgttttgatttatggtgctggtgttatcggttgtgaatatgcttctattttcagaggtatggatgttaaggttgacttgattaatacaagagacagattattagctttccttgatcaggaaatgtctgattccctttcctaccatttttggaactccggtgtcgtcatcagacacaacgaggaatatgaaaagattgaaggttgtgatgacggcgttattatgcaccttaagtctggtaaaaagttaaaagcagattgcttgttatatgcaaatggtagaaccggtaacacagactccttggctttacaaaacattggtttagaaaccgattcaagaggtcaattaaaggtcaattcaatgtatcaaactgcacaaccacacgtttacgcagttggtgacgttattggttacccttcattggcatctgccgcttacgatcaaggtagaatcgccgctcaagcacttgttaagggtgaagcaactgcacacttaatcgaagatatccctaccggtatctacactatcccagaaatctcttctgttggcaagactgaacaacaattaaccgcaatgaaggttccatacgaagtcggtcgtgcccagttcaagcatttggctagagcacaaattgttggtatgaatgttggtactttgaaaatcttgtttcacagagaaacaaaggaaatcttgggcattcactgtttcggcgaaagagctgcagagattattcacatcggtcaagccattatggaacaaaaaggcggtggtaataccattgaatatttcgttaataccaccttcaactacccaacaatggccgaagcatatagagtcgctgctttaaacggtttaaacagattgttttaattaacatctgaatgtaaaatgaacattaaaatgaattactaaactttacgtctactttacaatctataaactttgtttaatcatataacgaaatacactaatacacaatcctgtacgtatgtaatacttttatccatcaaggattgagaaaaaaaagtaatgattccctgggccattaaaacttagacccccaagcttggataggtcactctctattttcgtttctcccttccctgatagaagggtgatatgtaattaagaataatatataattttataataaaagcggccgccaagttagttagagctagagttaacacatacacattatcaaatgcatttattcctaatatcacactaaaacgtattatataattttaatctttatagacttcatagcaccaattggatttgctttctttcagaataccgcacttaatctcaatgtacgtaacgtaggcaaaatctgtcgataaggatctgtatgccgtaaacggaaactccaagcgcccagaaaacttacattatattcttgccagtttcatctcaccagccagtcacagtttaaaaggtttgattgcgtttcttgtttcgtcggattcagtgctaattggtaacgcactgtaccgccacaccaaagcaaaaatgcagaaacaaacaacaatgagtgtatgtttaccaactttggccgc

SEQ ID NO: 26 - DNA, 5676 bp, synthetic construct
  source 1..5676: mol_type = other DNA; note = Synthetic - A. vinelandii Stha gene integration fragment
aactactatgtacactgtataagtaaaaagacgatacccccctcccactctgggtgctacggtgtagatctctccgtaaacacaaaaaggcggctcagatgataattggggtccgggcgcaaccggaaggggggagagaggggagcgatggcttctcctccggggggctacgggagtttcctctttgggaaggataaagaggggatggattgatacaagattctgagaacctattacgatgatgttcagtggtattttgtcttttgttatttaaagggaggggactttcctcaataccttagttgtaaaattacgctattatctttaaccctttcttttgagcaataattaaaaagagcggccgcgagtccatcggttcctgtcagatgggatactcttgacgtggaaaattcaaacagaaaaaaaaccccaataatgaaaaataacactacgttatatccgtggtatcctctatcgtatcgtatcgtagcgtatcgtagcgtaccgtatcacagtatagtctaatattccgtatcttattgtatcctatcctattcgatcctattgtatttcagtgcaccattttaatttctattgctataatgtccttattagttgccactgtgaggtgaccaatggacgagggcgagccgttcagaagccgcgaagggtgttcttcccatgaatttcttaaggagggcggctcagctccgagagtgaggcgagacgtctcggtcagcgtatcccccttcctcggcttttacaaatgatgcgctcttaatagtgtgtcgttatccttttggcattgacgggggagggaaattgattgagcgcatccatatttttgcggactgctgaggacaatggtggtttttccgggtggcgtgggctacaaatgatacgatggtttttttcttttcggagaaggcgtataaaaaggacacggagaacccatttattctaaaaacagttgagcttctttaattattttttgatataatattctattattatatattttcttcccaataaaacaaaataaaacaaaacacagcaaaacacaaaaagctagcctgaaagggaaccataatgggtaagatcgcaccacattagcgggctcgaagatggatcttgcgaatgggtgacaccagtcataaggcctcgttgtcccagcatacctcccgcgctatctaattgcttcgctctccattgttcttggtaaacatcactctggcttgatggtgtcatctatgcccgccaagcctatcggtctatggcccggagtttgctccgtcttccaattgcaatcgcacggaatccgggatagaaagaacgatacgcattcatacgattctcacgttattggttggtgaatcaaatgcacaacgaacccaatcgccctggactcagcgtctaggccccccgtatggccgacggggactcagagcgtcaatccacgttgaagtcgaggttttggcagttacagcccttgcaataaggtttttcggacagtctactttgtcggcgcgccttctgtctttgattttcttatgttattcaaaacatctgccccaaaatctaacgattatatatattcctacgtataactgtatagctaattattgatttatttgtacataaaaaccacataaatgtaaaagcaagaaaaaaaataactaaggagaaggatcaatatctcatttataatgctcgccaaagcagcgtacgtgaattttaatcaagacatcaacaaatcttgcaacttggttatatcgcttcttcacccactcacccgcttttctacattgttgaacacaaatatatacaggggtatgtctcaaggtcaagtgcagtttcaacagagactacctcaaggtacctcttcagaaatgcagaacttcactcttgatcagattttctccgaattaaaggaggcctattggtagttctttccccctctcaagctggcgtgaaatgcaaccttacggcgtctacgttactacaaggtccagaaagtgtaggtattgctactatttttattttttattggttctggagaaatgcagacagtcaatgaacacaactgtctcaatatgcatctatgcacatgcacacacacacacatcacaggtacccctacaaagagaggtctcttgataatgtttcattaccacgtggcatccccccccccccccccaataaacaagtggccgagttcccctgttgcagaggaggacaaaaaaaccgctggtgttggtaccattatgcagcaactagcacaacaaacaaccgacccagacatacaaatcaacaacacttcgccaaagacaccctttccagggaggatccactcccaacgtctctccataatgtctctgttggcccatgtctctgtcgttgacaccgtaaccacaccaaccaacccgtccattgtactgggatggtcgtccatagacacctctccaacggggaacacctcattcgtaaaccgccaaggttaccgttcctcctgactcgccccgttgttgatgctgcgcacctgtggttgcccaacatggttgtatatcgtgtaaccacaccaacacatgtgcagcacatgtgtttaaaagagtgtcatggaggtggatcatgatggaagtggactttaccacttgggaactgtctccactcccgggaagaaaagacccggcgtatcacgcggttgcctcaatggggcaatttggaaggagaaatatagggaaaatcacgtcgctctcggacggggaagagttccagactatgaggggggggggtggtatataaagacaggagatgtccacccccagagagaggaagaagttggaactttagaagagagagataactttccccagtgtccatcaatacacaaccaaacacaaactctatatttacacatataaccccctctctagaatggcagtctataactatgatgttgttgtcattggtactggtccagctggtgaaggtgctgctatgaatgctgtcaaagctggcagaaaggttgctgtcgttgacgacagacctcaagtcggtggtaactgtactcatcttggtactatcccatccaaggcattaagacattcagttagacagatcatgcagtataacaacaacccattattcagacaaattggtgaacctagatggttttctttcgcagacgttcttaagtccgctgaacaagttatcgcaaagcaagtctcttcaagaaccggctattacgcaagaaatcgtattgatactttctttggcaccgcctcattctgtgatgaacatactatcgaagttgtccacttgaatggtatggttgaaaccttagttgctaagcaattcgttattgcaacaggttcaagaccatacagaccagctgacgtcgactttacccacccaagaatctacgattccgataccattctttccttgggtcatacaccaagacgtttgattatctacggtgccggtgtcattggctgtgagtacgcttcaattttctccggtttaggtgttttagttgatttgattgacaacagagatcagttgttgtcctttttggatgatgaaatttctgattctttgtcctatcacttaagaaataacaacgttttgattagacacaacgaagaatacgaaagagttgaaggtcttgataatggtgttatcttacacttaaagtctggtaaaaagattaaggcagatgcatttttgtggtctaacggtagaactggtaacactgataagttaggtttggaaaacattggtttgaaggctaatggcagaggtcaaattcaagttgatgagcattatcgtacagaagtctccaatatctacgcagccggtgacgtcatcggttggccatccttagcttcagcagcttatgatcaaggtagatctgctgctggttctattaccgagaatgactcttggcgtttcgttgatgatgttcctaccggtatctacaccatccctgaaatttcctctgttggtaaaaccgaaagagagttgacacaagcaaaagtcccatacgaggttggtaaagcctttttcaagggcatggctcgtgcacaaattgcagttgaaaaagccggtatgttaaagattctttttcatagagagactttagaaatcttgggtgtccactgcttcggttaccaagcatctgaaattgttcatattggtcaagcaattatgaaccaaaagggcgaagcaaatacattaaagtatttcatcaacactacattcaattatccaactatggctgaagcttatagagttgcagcctacgacggtttaaacagattgttttaattaatttattttactagtttatttttgctcctgagaataggattacaaacacttaaagtctttaattacaactatatataatattctgttggttttcttgaattggttcgctgcgattcatgcctcccattcaccaaaggtggagtgggaaataacggttttactgcggtaattagcagaggcaagaacaggatacactttttgatgataaatctgtattatagtcgagcctatttaggaaatcaaattttcttgtgtttacttttcaaataaataatgttcgaaaatttttactttactccttcatttaactataccagacgttatatcatcaacaccttctgaccatatacagctcaagatgtttaagagtctgttaaattttttcaatccatttcatggagtaccaggaggtgctacaaaaggaattcatagcctcatgaaatcagccatttgcttttgttcaacgatcttttgaaattgttgttgttcttggtagttaagttgatccatcttggcttatgttgtgtgtatgttgtagttattcttagtatattcctgtcctgagtttagtgaaacataatatcgccttgaaatgaaaatgctgaaattcgtcgacatacaatttttcaaactttttttttttcttggtgcacggacatgtttttaaaggaagtactctataccagttattcttcacaaatttaattgctggagaatagatcttcaacgctttaataaagtagtttgtttgtcaaggatggcgtcatacaaagaaagatcagaatcacacacttcccctgttgctaggagacttttctccatcatggaggaaaagaagtctaacctttgtgcatcattggatattactgaaactgaaaagcttctctctattttggacactattggtccttacatctgtctagttaaaacacacatcgatattgtttctgattttacgtatgaaggaactgtgttgcctttgaaggagcttgccaagaaacataattttatgatttttgaagatagaaaatttgctgatattggtaacactgttaaaaatcaatataaatctggtgtcttccgtattgccgaatgggctgacatcactaatgcacatggtgtaacgggtgcaggtattgtttctggcttgaaggaggcagcccaagaaacaaccagtgaacctagaggtttgctaatgcttgctgagttatcatcaaagggttctttagcatatggtgaatatacagaaaaaacagtagaaattgctaaatctgataaagagtttgtcattggttttattgcgcaacacgatatgggcggtagagaagaaggttttgactccgc

SEQ ID NO: 27 - DNA, 1395 bp, Azotobacter vinelandii
  source 1..1395: mol_type = other DNA
atggcagtctataactatgatgttgttgtcattggtactggtccagctggtgaaggtgctgctatgaatgctgtcaaagctggcagaaaggttgctgtcgttgacgacagacctcaagtcggtggtaactgtactcatcttggtactatcccatccaaggcattaagacattcagttagacagatcatgcagtataacaacaacccattattcagacaaattggtgaacctagatggttttctttcgcagacgttcttaagtccgctgaacaagttatcgcaaagcaagtctcttcaagaaccggctattacgcaagaaatcgtattgatactttctttggcaccgcctcattctgtgatgaacatactatcgaagttgtccacttgaatggtatggttgaaaccttagttgctaagcaattcgttattgcaacaggttcaagaccatacagaccagctgacgtcgactttacccacccaagaatctacgattccgataccattctttccttgggtcatacaccaagacgtttgattatctacggtgccggtgtcattggctgtgagtacgcttcaattttctccggtttaggtgttttagttgatttgattgacaacagagatcagttgttgtcctttttggatgatgaaatttctgattctttgtcctatcacttaagaaataacaacgttttgattagacacaacgaagaatacgaaagagttgaaggtcttgataatggtgttatcttacacttaaagtctggtaaaaagattaaggcagatgcatttttgtggtctaacggtagaactggtaacactgataagttaggtttggaaaacattggtttgaaggctaatggcagaggtcaaattcaagttgatgagcattatcgtacagaagtctccaatatctacgcagccggtgacgtcatcggttggccatccttagcttcagcagcttatgatcaaggtagatctgctgctggttctattaccgagaatgactcttggcgtttcgttgatgatgttcctaccggtatctacaccatccctgaaatttcctctgttggtaaaaccgaaagagagttgacacaagcaaaagtcccatacgaggttggtaaagcctttttcaagggcatggctcgtgcacaaattgcagttgaaaaagccggtatgttaaagattctttttcatagagagactttagaaatcttgggtgtccactgcttcggttaccaagcatctgaaattgttcatattggtcaagcaattatgaaccaaaagggcgaagcaaatacattaaagtatttcatcaacactacattcaattatccaactatggctgaagcttatagagttgcagcctacgacggtttaaacagattgttttaa

SEQ ID NO: 28 - DNA, 5298 bp, synthetic construct
  source 1..5298: mol_type = other DNA; note = Synthetic - A. vinelandii SthA gene integration fragment
aattctttgaaggagcttgccaagaaacataattttatgatttttgaagatagaaaatttgctgatattggtaacactgttaaaaatcaatataaatctggtgtcttccgtattgccgaatgggctgacatcactaatgcacatggtgtaacgggtgcaggtattgtttctggcttgaaggaggcagcccaagaaacaaccagtgaacctagaggtttgctaatgcttgctgagttatcatcaaagggttctttagcatatggtgaatatacagaaaaaacagtagaaattgctaaatctgataaagagtttgtcattggttttattgcgcaacacgatatgggcggtagagaagaaggttttgactggatcattatgactccaggggttggtttagatgacaaaggtgatgcacttggtcaacaatatagaactgttgatgaagttgtaaagactggaacggatatcataattgttggtagaggtttgtacggtcaaggaagagatcctatagagcaagctaaaagataccaacaagctggttggaatgcttatttaaacagatttaaatgattcttacacaaagatttgatacatgtacactagtttaaataagcatgaaaagaattacacaagcaaaaaaaaaaaaataaatgaggtactttacgttcacctacaaccaaaaaaactagatagagtaaaatcttaagatttagaaaaagttgtttaacaaaggctttagtatgtgaatttttaatgtagcaaagcgataactaataaacataaacaaaagtatggttttctttatcagtcaaatcattatcgattgattgttccgcgtatctgcagatagcctcatgaaatcagccatttgcttttgttcaacgatcttttgaaattgttgttgttcttggtagttaagttgatccatcttggcttatgttgtgtgtatgttgtagttattcttagtatattcctgtcctgagtttagtgaaacataatatcgccttgaaatgaaaatgctgaaattcgtcgacatacaatttttcaaactttttttttttcttggtgcacggacatgtttttaaaggaagtactctataccagttattcttcacaaatttaattgctggagaatagatcttcaacgcgtttaaacagcaatttgaggaaggaataggagaaggagaagcaatttctaggaaagagcaaggtgtgcaacagcatgctctgaatgatattttcagcaatagttcagttgaagaacctgttggcgtatctacatcacttcctacaaacaacaccacgaattgcgtccgtggtgacgcaactacgaatggcattgtcaatgccaatgccagtgcacatacacgtgcaagtcccaccggttccctgcccggctatggtagagacaagaaggacgataccggcatcgacatcaacagtttcaacagcaatgcgtttggcgtcgacgcgtcgatggggctgccgtatttggatttggacgggctagatttcgatatggatatggatatggatatggatatggagatgaatttgaatttagatttgggtcttgatttggggttggaattaaaaggggataacaatgagggttttcctgttgatttaaacaatggacgtgggaggtgattgatttaacctgatccaaaaggggtatgtctattttttagagtgtgtctttgtgtcaaattatggtagaatgtgtaaagtagtataaactttcctctcaaatgacgaggtttaaaacaccccccgggtgagccgagccgagaatggggcaattgttcaatgtgaaatagaagtatcgagtgagaaacttgggtgttggccagccaagggggaaggaaaatggcgcgaatgctcaggtgagattgttttggaattgggtgaagcgaggaaatgagcgacccggaggttgtgactttagtggcggaggaggaacgggaggaaaaggccaagagggaaagtgtatataagggggagcaatttgccaaccaggatagaattggatgagttataattctactgtatttattgtataatttatttctccttttatatcaaacacattacaaaacacacaaaacatacaaacatacacagctagcaaaggcgcgccatctaatagtttaatcacagcttatagtctactatagttttcttttttaaacattgttgtattttgtcccccccctctaattgatgatgattatcctataagaatccaataaaacgatggaaactaataccctctcctttgtcatgtggtctttagtatttcttgaacattggctctgatttctcgactttatagtcctattaaaatcgctgttagttctcgatcgttgtatctcgtttcttgtctctttggtggatgattttgcgtgcgaacatgtttttttccctttctctcaccatcatcgtgtagttcttgtcaccatcccccccaccccttccttctctcattgattctataagagcttatccacagaggtgcagtaacgaggtagtttaaccttcgagtggatcaaaatgtcacacaggcctcgacatttgctgcaacggcaacatcaatgtccacgtttacacacctacatttatatctatatttatatttatatttatttatttatgctacttagcttctatagttagttaatgcactcacgatattcaaaattgacacccttcaactactccctactattgtctactactgtctactactcctctttactatagctgctcccaataggctccaccaataggctctgtcaatacattttgcgccgccacctttcaggttgtgtcactcctgaaggaccatattgggtaatcgtgcaatttctggaagagagtccgcgagaagtgaggcccccactgtaaatcctcgagggggcatggagtatggggcatggaggatggaggatggggggggggggggggaaaataggtagcgaaaggacccgctatcaccccacccggagaactcgttgccgggaagtcatatttcgacactccggggagtctataaaaggcgggttttgtcttttgccagttgatgttgctgagaggacttgtttgccgtttcttccgatttaacagtatagaatcaaccactgttaattatacacgttatactaacacaacaaaaacaaaaacaacgacaacaacaacaacatctagaatggcagtctataactatgatgttgttgtcattggtactggtccagctggtgaaggtgctgctatgaatgctgtcaaagctggcagaaaggttgctgtcgttgacgacagacctcaagtcggtggtaactgtactcatcttggtactatcccatccaaggcattaagacattcagttagacagatcatgcagtataacaacaacccattattcagacaaattggtgaacctagatggttttctttcgcagacgttcttaagtccgctgaacaagttatcgcaaagcaagtctcttcaagaaccggctattacgcaagaaatcgtattgatactttctttggcaccgcctcattctgtgatgaacatactatcgaagttgtccacttgaatggtatggttgaaaccttagttgctaagcaattcgttattgcaacaggttcaagaccatacagaccagctgacgtcgactttacccacccaagaatctacgattccgataccattctttccttgggtcatacaccaagacgtttgattatctacggtgccggtgtcattggctgtgagtacgcttcaattttctccggtttaggtgttttagttgatttgattgacaacagagatcagttgttgtcctttttggatgatgaaatttctgattctttgtcctatcacttaagaaataacaacgttttgattagacacaacgaagaatacgaaagagttgaaggtcttgataatggtgttatcttacacttaaagtctggtaaaaagattaaggcagatgcatttttgtggtctaacggtagaactggtaacactgataagttaggtttggaaaacattggtttgaaggctaatggcagaggtcaaattcaagttgatgagcattatcgtacagaagtctccaatatctacgcagccggtgacgtcatcggttggccatccttagcttcagcagcttatgatcaaggtagatctgctgctggttctattaccgagaatgactcttggcgtttcgttgatgatgttcctaccggtatctacaccatccctgaaatttcctctgttggtaaaaccgaaagagagttgacacaagcaaaagtcccatacgaggttggtaaagcctttttcaagggcatggctcgtgcacaaattgcagttgaaaaagccggtatgttaaagattctttttcatagagagactttagaaatcttgggtgtccactgcttcggttaccaagcatctgaaattgttcatattggtcaagcaattatgaaccaaaagggcgaagcaaatacattaaagtatttcatcaacactacattcaattatccaactatggctgaagcttatagagttgcagcctacgacggtttaaacagattgttttaattaacatctgaatgtaaaatgaacattaaaatgaattactaaactttacgtctactttacaatctataaactttgtttaatcatataacgaaatacactaatacacaatcctgtacgtatgtaatacttttatccatcaaggattgagaaaaaaaagtaatgattccctgggccattaaaacttagacccccaagcttggataggtcactctctattttcgtttctcccttccctgatagaagggtgatatgtaattaagaataatatataattttataataaaagcggccgccaagttagttagagctagagttaacacatacacattatcaaatgcatttattcctaatatcacactaaaacgtattatataattttaatctttatagacttcatagcaccaattggatttgctttctttcagaataccgcacttaatctcaatgtacgtaacgtaggcaaaatctgtcgataaggatctgtatgccgtaaacggaaactccaagcgcccagaaaacttacattatattcttgccagtttcatctcaccagccagtcacagtttaaaaggtttgattgcgtttcttgtttcgtcggattcagtgctaattggtaacgcactgtaccgccacaccaaagcaaaaatgcagaaacaaacaacaatgagtgtatgtttaccaactttggccgc

SEQ ID NO: 29 - DNA, 6139 bp, synthetic construct
  source 1..6139: mol_type = other DNA; note = Synthetic - S. cerevisiae SthA gene integration fragment
aattctttgaaggagcttgccaagaaacataattttatgatttttgaagatagaaaatttgctgatattggtaacactgttaaaaatcaatataaatctggtgtcttccgtattgccgaatgggctgacatcactaatgcacatggtgtaacgggtgcaggtattgtttctggcttgaaggaggcagcccaagaaacaaccagtgaacctagaggtttgctaatgcttgctgagttatcatcaaagggttctttagcatatggtgaatatacagaaaaaacagtagaaattgctaaatctgataaagagtttgtcattggttttattgcgcaacacgatatgggcggtagagaagaaggttttgactggatcattatgactccaggggttggtttagatgacaaaggtgatgcacttggtcaacaatatagaactgttgatgaagttgtaaagactggaacggatatcataattgttggtagaggtttgtacggtcaaggaagagatcctatagagcaagctaaaagataccaacaagctggttggaatgcttatttaaacagatttaaatgattcttacacaaagatttgatacatgtacactagtttaaataagcatgaaaagaattacacaagcaaaaaaaaaaaaataaatgaggtactttacgttcacctacaaccaaaaaaactagatagagtaaaatcttaagatttagaaaaagttgtttaacaaaggctttagtatgtgaatttttaatgtagcaaagcgataactaataaacataaacaaaagtatggttttctttatcagtcaaatcattatcgattgattgttccgcgtatctgcagatagcctcatgaaatcagccatttgcttttgttcaacgatcttttgaaattgttgttgttcttggtagttaagttgatccatcttggcttatgttgtgtgtatgttgtagttattcttagtatattcctgtcctgagtttagtgaaacataatatcgccttgaaatgaaaatgctgaaattcgtcgacatacaatttttcaaactttttttttttcttggtgcacggacatgtttttaaaggaagtactctataccagttattcttcacaaatttaattgctggagaatagatcttcaacgcgtttaaacagcaatttgaggaaggaataggagaaggagaagcaatttctaggaaagagcaaggtgtgcaacagcatgctctgaatgatattttcagcaatagttcagttgaagaacctgttggcgtatctacatcacttcctacaaacaacaccacgaattgcgtccgtggtgacgcaactacgaatggcattgtcaatgccaatgccagtgcacatacacgtgcaagtcccaccggttccctgcccggctatggtagagacaagaaggacgataccggcatcgacatcaacagtttcaacagcaatgcgtttggcgtcgacgcgtcgatggggctgccgtatttggatttggacgggctagatttcgatatggatatggatatggatatggatatggagatgaatttgaatttagatttgggtcttgatttggggttggaattaaaaggggataacaatgagggttttcctgttgatttaaacaatggacgtgggaggtgattgatttaacctgatccaaaaggggtatgtctattttttagagtgtgtctttgtgtcaaattatggtagaatgtgtaaagtagtataaactttcctctcaaatgacgaggtttaaaacaccccccgggtgagccgagccgagaatggggcaattgttcaatgtgaaatagaagtatcgagtgagaaacttgggtgttggccagccaagggggaaggaaaatggcgcgaatgctcaggtgagattgttttggaattgggtgaagcgaggaaatgagcgacccggaggttgtgactttagtggcggaggaggaacgggaggaaaaggccaagagggaaagtgtatataagggggagcaatttgccaaccaggatagaattggatgagttataattctactgtatttattgtataatttatttctccttttatatcaaacacattacaaaacacacaaaacatacaaacatacacagctagcatggatggtcctaacttcgcccatcaaggcggtagatcccaaagaactaccgagttgtactcatgtgcacgttgccgtaagcttaagaagaaatgtggtaaacaaatcccaacttgtgcaaactgtgataagaacggtgcacattgttcttatcctggtagagctcctagacgtaccaagaaggagttggctgatgcaatgcttagaggtgaatatgttccagttaaacgtaacaagaaagtcggcaaatctcctttatctactaagtctatgccaaactcctcttctccattatccgctaacggtgcaatcactcctggtttttctccatatgaaaacgatgacgcccacaagatgaagcagttaaagccatctgatccaattaacttagtcatgggtgcatctccaaattcctccgagggcgtttcctctttgatttccgttttaacctcattgaacgacaattccaatccttcttctcacttgtcctctaacgaaaattccatgatcccttctcgttcccttccagcatccgttcaacaatcatctacaacttcttcctttggtggttataacaccccatcaccattgatttcctctcacgttccagccaatgcacaagcagtcccattacaaaacaacaacagaaacacctctaacggtgacaatggttctaacgttaatcatgacaacaataatggttccaccaacacaccacaattatccttgactccatacgctaataactctgctcctaacggcaaattcgattccgtccctgtcgatgcttcttccatcgagtttgagacaatgtcttgttgttttaagggtggcagaactacttcttgggttagagaagatggttcttttaagtctattgacagatcattattggacagattcatcgcagcttacttcaagcacaaccacagattgttcccaatgattgataagattgcattcttgaatgatgctgctactattaccgatttcgaaagattgtacgataacaagaactatccagattcttttgttttcaaggtttacatgattatggcaattggctgtactacattacaaagagctggcatggtttcccaagacgaagaatgtttgtctgaacatttggctttccttgctatgaagaagtttagatctgtcattatcttacaagatatcgaaactgttagatgcttgttgttattaggtatctactcattctttgaaccaaagggttcttcctcttggactatttcaggcattatcatgcgtcttactattggtttgggtttgaatagagaattgactgctaagaaattgaagtctatgtcagccttagaagcagaagcaagatatagagttttctggtctgcttattgttttgaaagattggtctgtacatccttgggtagaatttccggtattgacgacgaagatattactgttccattaccaagagcattgtatgtcgatgagagagatgatttggaaatgaccaagttaatgatctccttaagaaagatgggtggtagaatctacaagcaagtccattctgtttccgctggtagacaaaagttgaccatcgaacagaagcaggagattatctctggtttgagaaaagaacttgacgaaatctactccagagaatccgaaagaagaaagttaaagaagtctcaaatggaccaagtcgaaagagaaaacaattcaacaactaatgttatttcctttcactcatctgagatttggttagctatgagatactctcaattgcaaatcttgttgtatagaccatccgcccttatgccaaaaccacctattgattctttgtcaacccttggtgaattttgtttacaagcatggaaacacacttacacattgtacaagaaaagattgttaccattgaattggattaccttattcagaactttaactatttgtaacacaatcttatactgtttatgccaatggtccattgacttaattgaatctaagattgaaatccaacagtgtgttgaaatcttgcgtcattttggtgaaagatggattttcgccatgagatgtgctgatgttttccaaaacatttcaaataccattttagacatctccctttcccatggtaaagttccaaacatggatcaattaaccagagagttattcggtgcatctgactcctaccaagacatcttagacgaaaacaatgttgatgtttcttgggtcgataagttggtctaaggcgcgccatctaatagtttaatcacagcttatagtctactatagttttcttttttaaacattgttgtattttgtcccccccctctaattgatgatgattatcctataagaatccaataaaacgatggaaactaataccctctcctttgtcatgtggtctttagtatttcttgaacattggctctgatttctcgactttatagtcctattaaaatcgctgttagttctcgatcgttgtatctcgtttcttgtctctttggtggatgattttgcgtgcgaacatgtttttttccctttctctcaccatcatcgtgtagttcttgtcaccatcccccccaccccttccttctctcattgattctataagagcttatccacagaggtgcagtaacgaggtagtttaaccttcgagtggatcaaaatgtcacacaggcctcgacatttgctgcaacggcaacatcaatgtccacgtttacacacctacatttatatctatatttatatttatatttatttatttatgctacttagcttctatagttagttaatgcactcacgatattcaaaattgacacccttcaactactccctactattgtctactactgtctactactcctctttactatagctgctcccaataggctccaccaataggctctgtcaatacattttgcgccgccacctttcaggttgtgtcactcctgaaggaccatattgggtaatcgtgcaatttctggaagagagtccgcgagaagtgaggcccccactgtaaatcctcgagggggcatggagtatggggcatggaggatggaggatggggggggggggggggaaaataggtagcgaaaggacccgctatcaccccacccggagaactcgttgccgggaagtcatatttcgacactccggggagtctataaaaggcgggttttgtcttttgccagttgatgttgctgagaggacttgtttgccgtttcttccgatttaacagtatagaatcaaccactgttaattatacacgttatactaacacaacaaaaacaaaaacaacgacaacaacaacaacatctagataattaattaacatctgaatgtaaaatgaacattaaaatgaattactaaactttacgtctactttacaatctataaactttgtttaatcatataacgaaatacactaatacacaatcctgtacgtatgtaatacttttatccatcaaggattgagaaaaaaaagtaatgattccctgggccattaaaacttagacccccaagcttggataggtcactctctattttcgtttctcccttccctgatagaagggtgatatgtaattaagaataatatataattttataataaaagcggccgccaagttagttagagctagagttaacacatacacattatcaaatgcatttattcctaatatcacactaaaacgtattatataattttaatctttatagacttcatagcaccaattggatttgctttctttcagaataccgcacttaatctcaatgtacgtaacgtaggcaaaatctgtcgataaggatctgtatgccgtaaacggaaactccaagcgcccagaaaacttacattatattcttgccagtttcatctcaccagccagtcacagtttaaaaggtttgattgcgtttcttgtttcgtcggattcagtgctaattggtaacgcactgtaccgccacaccaaagcaaaaatgcagaaacaaacaacaatgagtgtatgtttaccaactttggccgc

SEQ ID NO: 30 - DNA, 2232 bp, Saccharomyces cerevisiae
  source 1..2232: mol_type = other DNA
atggatggtcctaacttcgcccatcaaggcggtagatcccaaagaactaccgagttgtactcatgtgcacgttgccgtaagcttaagaagaaatgtggtaaacaaatcccaacttgtgcaaactgtgataagaacggtgcacattgttcttatcctggtagagctcctagacgtaccaagaaggagttggctgatgcaatgcttagaggtgaatatgttccagttaaacgtaacaagaaagtcggcaaatctcctttatctactaagtctatgccaaactcctcttctccattatccgctaacggtgcaatcactcctggtttttctccatatgaaaacgatgacgcccacaagatgaagcagttaaagccatctgatccaattaacttagtcatgggtgcatctccaaattcctccgagggcgtttcctctttgatttccgttttaacctcattgaacgacaattccaatccttcttctcacttgtcctctaacgaaaattccatgatcccttctcgttcccttccagcatccgttcaacaatcatctacaacttcttcctttggtggttataacaccccatcaccattgatttcctctcacgttccagccaatgcacaagcagtcccattacaaaacaacaacagaaacacctctaacggtgacaatggttctaacgttaatcatgacaacaataatggttccaccaacacaccacaattatccttgactccatacgctaataactctgctcctaacggcaaattcgattccgtccctgtcgatgcttcttccatcgagtttgagacaatgtcttgttgttttaagggtggcagaactacttcttgggttagagaagatggttcttttaagtctattgacagatcattattggacagattcatcgcagcttacttcaagcacaaccacagattgttcccaatgattgataagattgcattcttgaatgatgctgctactattaccgatttcgaaagattgtacgataacaagaactatccagattcttttgttttcaaggtttacatgattatggcaattggctgtactacattacaaagagctggcatggtttcccaagacgaagaatgtttgtctgaacatttggctttccttgctatgaagaagtttagatctgtcattatcttacaagatatcgaaactgttagatgcttgttgttattaggtatctactcattctttgaaccaaagggttcttcctcttggactatttcaggcattatcatgcgtcttactattggtttgggtttgaatagagaattgactgctaagaaattgaagtctatgtcagccttagaagcagaagcaagatatagagttttctggtctgcttattgttttgaaagattggtctgtacatccttgggtagaatttccggtattgacgacgaagatattactgttccattaccaagagcattgtatgtcgatgagagagatgatttggaaatgaccaagttaatgatctccttaagaaagatgggtggtagaatctacaagcaagtccattctgtttccgctggtagacaaaagttgaccatcgaacagaagcaggagattatctctggtttgagaaaagaacttgacgaaatctactccagagaatccgaaagaagaaagttaaagaagtctcaaatggaccaagtcgaaagagaaaacaattcaacaactaatgttatttcctttcactcatctgagatttggttagctatgagatactctcaattgcaaatcttgttgtatagaccatccgcccttatgccaaaaccacctattgattctttgtcaacccttggtgaattttgtttacaagcatggaaacacacttacacattgtacaagaaaagattgttaccattgaattggattaccttattcagaactttaactatttgtaacacaatcttatactgtttatgccaatggtccattgacttaattgaatctaagattgaaatccaacagtgtgttgaaatcttgcgtcattttggtgaaagatggattttcgccatgagatgtgctgatgttttccaaaacatttcaaataccattttagacatctccctttcccatggtaaagttccaaacatggatcaattaaccagagagttattcggtgcatctgactcctaccaagacatcttagacgaaaacaatgttgatgtttcttgggtcgataagttggtctaa

SEQ ID NO: 31 - protein, 506 aa, Issatchenkia orientalis
MGVQFIENTIIVVFGASGDLAKKKTFPALFGLFREGQLSETTKIIGFARSKLSNDDLRNRIKPYLKLNKRTDAERQSLEKFLQILEYHQSNYDDSEGFEKLEKLINKYDDEANVKESHRLYYLALPPSVFTTVATMLKKHCHPGDSGIARLIVEKPFGHDLSSSRELQKSLAPLWNEDELFRIDHYLGKEMVKNLIPLRFSNTFLSSSWNNQFIDTIQITFKENFGTEGRGGYFDSIGIIRDVIQNHLLQVLTIVLMEKPADFNGESIRDEKVKVLKAIEQIDFNNVLVGQYDKSEDGSKPGYLDDDTVNPDSKAVTYAALVLNVANERWNNVPIILKAGKALNQSKVEIRIQFKPVENGIFKNSARNELVIRIQPNEAMYLKMNIKVPGVSNQVSISEMDLTYKNRYSSEFYIPEAYESLIKDALMDDHSNFVRDDELDISWALFTPLLEHIEGPDGPTPTKYPYGSRGPKEIDEFLRNHGYVKEPRENYQWPLTTPKELNSSKF

SEQ ID NO: 32 - DNA, 3435 bp, synthetic construct
  source 1..3435: mol_type = other DNA; note = Synthetic - Mutated L. mexicana FRD gene
atggctgatggcaaaacctctgcatcagttgttgctgttgatgctgaacgtgccgctaaggaaagagatgcagcagctagagctatgttgcaaggtggtggtgtctctcctgctggcaaggcacaattgttgaaaaagggtttggttcacactgttccatataccttaaaggttgtcgtcgcagatccaaaggaaatggagaaggcaactgctgacgcagaagaggttttacaagctgcatttcaagtcgtcgacacccttttgaacaactttaacgaaaactcagaagtttcaagagtcaataggttggcagttggtgaggaacatcaaatgtctgaaacattgaaacacgtcatggcctgttgtcaaaaggtttatcattcctccagaggtgtttttgacccagcagttggtccattagtccgtgaacttagagaagctgctcacaagggtaaaactgttccagccgaaagagttaatgatttgttatccaaatgtacccttaatgcatctttttcaattgatatgtccagaggtatgattgcaaggaagcatccagacgccatgttggatttgggtggtgtcaacaagggttatggtatcgactacattgttgaacacttaaactctttgggttatgatgatgtctttttcggacggggtggtgatgttagagcatccggcaaaaaccagttatctcaaccttgggctgttggtattgttagaccacctgccttggccgacattagaactgttgtcccagaggacaaaagatcctttatccgtgtcgtcagattgaacaacgaagctattgctacctctggtgattatgagaatttggttgaaggtcctggttctaaggtttactcttccaccttcaatccaacttccaaaaacttgttggaacctaccgaagcaggtatggctcaagtttctgtcaagtgttgctcatgtatctacgctgatgctttagcaacagcagctttgttgaaaaacgatcctgctgccgttagaaggatcttagataactggagatatgtcagagatactgttactgactacaccacttacacaagggaaggtgaaagagttgctaagatgttggaaattgctaccgaagatgctgaaatgagagcaaagagaatcaagggctctttaccagcaagagttatcattgttggtggtggtttggccggttgttccgcagctatcgaagcagctaactgtggcgcccacgtcatcttgttagaaaaggaaccaaagttaggtggtaactctgcaaaggctacctccggtatcaacgcctggggtactagagcacaagcaaaacaaggtgtcatggacggcggcaagtttttcgaaagagatacccatagatccggcaagggtggtaattgcgatccatgccttgttaagactttgtccgttaagtcctctgatgcagttaagtggttatctgaattaggtgttccattgactgttttgtctcaattaggtggtgcttcaaggaaacgttgtcaccgtgcaccagataagtctgatggtacaccagtcccagttggtttcaccattatgaaaacccttgaaaaccacattgtcaacgatttgtccagacatgttacagttatgacaggtattaccgtcacagctttagaatctacatcaagagtcagacctgatggtgttttagtcaagcatgttactggtgttcacttgattcaggcatctggtcaatctatggttttgaatgcagacgctgttatcttagctactggtggtttctccaatgatcataccccaaactcccttttacaacaatacgccccacagttgtcatcttttccaacaaccaatggtgtctgggcaactggcgatggtgttaagatggcttccaagttgggtgtcgccttagttgatatggataaggtccaattacatcctaccggcttgttagacccaaaagatccatctaatagaaccaagtatcttggtccagaggccttaagaggttccggcggtgtcttgttaaacaaaaacggtgaaagatttgttaatgaattagacttaagatctgttgtctctcaagctatcatcgcacaagataatgagtacccaggctctggtggttccaagttcgcatactgtgttttgaacgaaactgcagcaaagttattcggcaaaaacttccttggtttctactggaatagattaggtcttttccaaaaggttgattccgttgctggtttagctaagttgattggttgtccagaagctaatgttgttgctacattgaagcaatatgaggagttatcttccaaaaagcttaatccttgtccattgactggcaagtctgtctttccttgtgttttaggcactcaaggtccatactatgttgccttggttaccccatccattcactacactatgggtggttgtttgatttccccatctgctgagatgcaaaccattgacaactctggtgttactcctgtcagacgtccaatcttaggcttattcggtgctggtgaagttactggcggtgtccatggtggtaacagattaggcggtaactctttgttagaatgtgttgttttcggcaagatcgctggtgacagagctgcaaccatcttgcaaaagaaaaacaccggcttatcaatgacagaatggtctactgtcgtcttaagagaagttagagaaggtggtgtctatggtgctggttccagagttttgaggtttaacatgcctggtgcattacagagaactggtttagctttaggtcaattcatcggtatcagaggtgattgggacggtcacagattgatcggttactattctccaatcactttacctgatgatgttggtgttattggtatcttagctagagcagacaagggtagattggcagaatggatttctgcattgcagccaggtgacgctgttgagatgaaggcctgcggtggtcttatcattgacagaagattcgctgaaagacatttctttttccgtggtcataagatcagaaagttggcccttatcggtggtggtactggtgttgcaccaatgttacaaatcgtcagagctgctgtcaaaaagccatttgtcgattcaattgagtccattcagttcatctatgctgcagaggatgtttccgagcttacatacagaaccttacttgaatcttacgaagaggaatatggttcagaaaagtttaagtgtcacttcgttttgaataacccaccagctcaatggactgacggtgttggtttcgttgatactgcattgttgagatccgcagttcaagcaccatcaaatgatttgcttgttgcaatttgtggtccaccaatcatgcaaagagcagttaagggtgcattgaaaggtttaggttacaatatgaatcttgttagaaccgttgacgaaactgaaccaccatcataa

SEQ ID NO: 33 - DNA, 3444 bp, synthetic construct
  source 1..3444: mol_type = other DNA; note = Synthetic - Mutated L. mexicana FRD gene
atggctgatggcaaaacctctgcatcagttgttgctgttgatgctgaacgtgccgctaaggaaagagatgcagcagctagagctatgttgcaaggtggtggtgtctctcctgctggcaaggcacaattgttgaaaaagggtttggttcacactgttccatataccttaaaggttgtcgtcgcagatccaaaggaaatggagaaggcaactgctgacgcagaagaggttttacaagctgcatttcaagtcgtcgacacccttttgaacaactttaacgaaaactcagaagtttcaagagtcaataggttggcagttggtgaggaacatcaaatgtctgaaacattgaaacacgtcatggcctgttgtcaaaaggtttatcattcctccagaggtgtttttgacccagcagttggtccattagtccgtgaacttagagaagctgctcacaagggtaaaactgttccagccgaaagagttaatgatttgttatccaaatgtacccttaatgcatctttttcaattgatatgtccagaggtatgattgcaaggaagcatccagacgccatgttggatttgggtggtgtcaacaagggttatggtatcgactacattgttgaacacttaaactctttgggttatgatgatgtctttttcgaatggggtggtgatgttagagcatccggcaaaaaccagttatctcaaccttgggctgttggtattgttagaccacctgccttggccgacattagaactgttgtcccagaggacaaaagatcctttatccgtgtcgtcagattgaacaacgaagctattgctacctctggtgattatgagaatttggttgaaggtcctggttctaaggtttactcttccaccttcaatccaacttccaaaaacttgttggaacctaccgaagcaggtatggctcaagtttctgtcaagtgttgctcatgtatctacgctgatgctttagcaacagcagctttgttgaaaaacgatcctgctgccgttagaaggatcttagataactggagatatgtcagagatactgttactgactacaccacttacacaagggaaggtgaaagagttgctaagatgttggaaattgctaccgaagatgctgaaatgagagcaaagagaatcaagggctctttaccagcaagagttatcattgttggtggtggtttggccggttgttccgcagctatcgaagcagctaactgtggcgcccacgtcatcttgttaggtaaggaaccaaagttaggtggtaactctgcaaaggctacctccggtatcaacgcctggggtactagagcacaagcaaaacaaggtgtcatggacggcggcaagtttttcgaaagagatacccatagatccggcaagggtggtaattgcgatccatgccttgttaagactttgtccgttaagtcctctgatgcagttaagtggttatctgaattaggtgttccattgactgttttgtctcaattaggtggtgcttcaaggaaacgttgtcaccgtgcaccagataagtctgatggtacaccagtcccagttggtttcaccattatgaaaacccttgaaaaccacattgtcaacgatttgtccagacatgttacagttatgacaggtattaccgtcacagctttagaatctacatcaagagtcagacctgatggtgttttagtcaagcatgttactggtgttcacttgattcaggcatctggtcaatctatggttttgaatgcagacgctgttatcttagctactggtggtttctccaatgatcataccccaaactcccttttacaacaatacgccccacagttgtcatcttttccaacaaccaatggtgtctgggcaactggcgatggtgttaagatggcttccaagttgggtgtcgccttagttgatatggataaggtccaattacatcctaccggcttgttagacccaaaagatccatctaatagaaccaagtatcttggtccagaggccttaagaggttccggcggtgtcttgttaaacaaaaacggtgaaagatttgttaatgaattagacttaagatctgttgtctctcaagctatcatcgcacaagataatgagtacccaggctctggtggttccaagttcgcatactgtgttttgaacgaaactgcagcaaagttattcggcaaaaacttccttggtttctactggaatagattaggtcttttccaaaaggttgattccgttgctggtttagctaagttgattggttgtccagaagctaatgttgttgctacattgaagcaatatgaggagttatcttccaaaaagcttaatccttgtccattgactggcaagtctgtctttccttgtgttttaggcactcaaggtccatactatgttgccttggttaccccatccattcactacactatgggtggttgtttgatttccccatctgctgagatgcaaaccattgacaactctggtgttactcctgtcagacgtccaatcttaggcttattcggtgctggtgaagttactggcggtgtccatggtggtaacagattaggcggtaactctttgttagaatgtgttgttttcggcaagatcgctggtgacagagctgcaaccatcttgcaaaagaaaaacaccggcttatcaatgacagaatggtctactgtcgtcttaagagaagttagagaaggtggtgtctatggtgctggttccagagttttgaggtttaacatgcctggtgcattacagagaactggtttagctttaggtcaattcatcggtatcagaggtgattgggacggtcacagattgatcggttactattctccaatcactttacctgatgatgttggtgttattggtatcttagctagagcagacaagggtagattggcagaatggatttctgcattgcagccaggtgacgctgttgagatgaaggcctgcggtggtcttatcattgacagaagattcgctgaaagacatttctttttccgtggtcataagatcagaaagttggcccttatcggtggtggtactggtgttgcaccaatgttacaaatcgtcagagctgctgtcaaaaagccatttgtcgattcaattgagtccattcagttcatctatgctgcagaggatgtttccgagcttacatacagaaccttacttgaatcttacgaagaggaatatggttcagaaaagtttaagtgtcacttcgttttgaataacccaccagctcaatggactgacggtgttggtttcgttgatactgcattgttgagatccgcagttcaagcaccatcaaatgatttgcttgttgcaatttgtggtccaccaatcatgcaaagagcagttaagggtgcattgaaaggtttaggttacaatatgaatcttgttagaaccgttgacgaaactgaaccaccatcataattaattaac

SEQ ID NO: 34 - DNA, 3444 bp, synthetic construct
  source 1..3444: mol_type = other DNA; note = Synthetic - Mutated L. mexicana FRD gene
atggctgatggcaaaacctctgcatcagttgttgctgttgatgctgaacgtgccgctaaggaaagagatgcagcagctagagctatgttgcaaggtggtggtgtctctcctgctggcaaggcacaattgttgaaaaagggtttggttcacactgttccatataccttaaaggttgtcgtcgcagatccaaaggaaatggagaaggcaactgctgacgcagaagaggttttacaagctgcatttcaagtcgtcgacacccttttgaacaactttaacgaaaactcagaagtttcaagagtcaataggttggcagttggtgaggaacatcaaatgtctgaaacattgaaacacgtcatggcctgttgtcaaaaggtttatcattcctccagaggtgtttttgacccagcagttggtccattagtccgtgaacttagagaagctgctcacaagggtaaaactgttccagccgaaagagttaatgatttgttatccaaatgtacccttaatgcatctttttcaattgatatgtccagaggtatgattgcaaggaagcatccagacgccatgttggatttgggtggtgtcaacaagggttatggtatcgactacattgttgaacacttaaactctttgggttatgatgatgtctttttcgaatggggtggtgatgttagagcatccggcaaaaaccagttatctcaaccttgggctgttggtattgttagaccacctgccttggccgacattagaactgttgtcccagaggacaaaagatcctttatccgtgtcgtcagattgaacaacgaagctattgctacctctggtgattatgagaatttggttgaaggtcctggttctaaggtttactcttccaccttcaatccaacttccaaaaacttgttggaacctaccgaagcaggtatggctcaagtttctgtcaagtgttgctcatgtatctacgctgatgctttagcaacagcagctttgttgaaaaacgatcctgctgccgttagaaggatcttagataactggagatatgtcagagatactgttactgactacaccacttacacaagggaaggtgaaagagttgctaagatgttggaaattgctaccgaagatgctgaaatgagagcaaagagaatcaagggctctttaccagcaagagttatcattgttggtggtggtttggccggttgttccgcagctatcgaagcagctaactgtggcgcccacgtcatcttgttagaaaaggaaccaaagttaggtggtaactctgcaaaggctacctccggtatcaacgcctggggtactagagcacaagcaaaacaaggtgtcatggacggcggcaagtttttcgaaagagatacccatagatccggcaagggtggtaattgcgatccatgccttgttaagactttgtccgttaagtcctctgatgcagttaagtggttatctgaattaggtgttccattgactgttttgtctcaattaggtggtgcttcaaggaaacgttgtcaccgtgcaccagataagtctgatggtacaccagtcccagttggtttcaccattatgaaaacccttgaaaaccacattgtcaacgatttgtccagacatgttacagttatgacaggtattaccgtcacagctttagaatctacatcaagagtcagacctgatggtgttttagtcaagcatgttactggtgttcacttgattcaggcatctggtcaatctatggttttgaatgcagacgctgttatcttagctactggtggtttctccaatgatcataccccaaactcccttttacaacaatacgccccacagttgtcatcttttccaacaaccaatggtgtctgggcaactggcgatggtgttaagatggcttccaagttgggtgtcgccttagttgatatgggtaaggtccaattacatcctaccggcttgttagacccaaaagatccatctaatagaaccaagtatcttggtccagaggccttaagaggttccggcggtgtcttgttaaacaaaaacggtgaaagatttgttaatgaattagacttaagatctgttgtctctcaagctatcatcgcacaagataatgagtacccaggctctggtggttccaagttcgcatactgtgttttgaacgaaactgcagcaaagttattcggcaaaaacttccttggtttctactggaatagattaggtcttttccaaaaggttgattccgttgctggtttagctaagttgattggttgtccagaagctaatgttgttgctacattgaagcaatatgaggagttatcttccaaaaagcttaatccttgtccattgactggcaagtctgtctttccttgtgttttaggcactcaaggtccatactatgttgccttggttaccccatccattcactacactatgggtggttgtttgatttccccatctgctgagatgcaaaccattgacaactctggtgttactcctgtcagacgtccaatcttaggcttattcggtgctggtgaagttactggcggtgtccatggtggtaacagattaggcggtaactctttgttagaatgtgttgttttcggcaagatcgctggtgacagagctgcaaccatcttgcaaaagaaaaacaccggcttatcaatgacagaatggtctactgtcgtcttaagagaagttagagaaggtggtgtctatggtgctggttccagagttttgaggtttaacatgcctggtgcattacagagaactggtttagctttaggtcaattcatcggtatcagaggtgattgggacggtcacagattgatcggttactattctccaatcactttacctgatgatgttggtgttattggtatcttagctagagcagacaagggtagattggcagaatggatttctgcattgcagccaggtgacgctgttgagatgaaggcctgcggtggtcttatcattgacagaagattcgctgaaagacatttctttttccgtggtcataagatcagaaagttggcccttatcggtggtggtactggtgttgcaccaatgttacaaatcgtcagagctgctgtcaaaaagccatttgtcgattcaattgagtccattcagttcatctatgctgcagaggatgtttccgagcttacatacagaaccttacttgaatcttacgaagaggaatatggttcagaaaagtttaagtgtcacttcgttttgaataacccaccagctcaatggactgacggtgttggtttcgttgatactgcattgttgagatccgcagttcaagcaccatcaaatgatttgcttgttgcaatttgtggtccaccaatcatgcaaagagcagttaagggtgcattgaaaggtttaggttacaatatgaatcttgttagaaccgttgacgaaactgaaccaccatcataattaattaac

SEQ ID NO: 35 - DNA, 3435 bp, synthetic construct
  source 1..3435: mol_type = other DNA; note = Synthetic - Mutated L. mexicana FRD gene
atggctgatggcaaaacctctgcatcagttgttgctgttgatgctgaacgtgccgctaaggaaagagatgcagcagctagagctatgttgcaaggtggtggtgtctctcctgctggcaaggcacaattgttgaaaaagggtttggttcacactgttccatataccttaaaggttgtcgtcgcagatccaaaggaaatggagaaggcaactgctgacgcagaagaggttttacaagctgcatttcaagtcgtcgacacccttttgaacaactttaacgaaaactcagaagtttcaagagtcaataggttggcagttggtgaggaacatcaaatgtctgaaacattgaaacacgtcatggcctgttgtcaaaaggtttatcattcctccagaggtgtttttgacccagcagttggtccattagtccgtgaacttagagaagctgctcacaagggtaaaactgttccagccgaaagagttaatgatttgttatccaaatgtacccttaatgcatctttttcaattgatatgtccagaggtatgattgcaaggaagcatccagacgccatgttggatttgggtggtgtcaacaagggttatggtatcgactacattgttgaacacttaaactctttgggttatgatgatgtctttttcgaatggggtggtgatgttagagcatccggcaaaaaccagttatctcaaccttgggctgttggtattgttagaccacctgccttggccgacattagaactgttgtcccagaggacaaaagatcctttatccgtgtcgtcagattgaacaacgaagctattgctacctctggtgattatgagaatttggttgaaggtcctggttctaaggtttactcttccaccttcaatccaacttccaaaaacttgttggaacctaccgaagcaggtatggctcaagtttctgtcaagtgttgctcatgtatctacgctgatgctttagcaacagcagctttgttgaaaaacgatcctgctgccgttagaaggatcttagataactggagatatgtcagagatactgttactgactacaccacttacacaagggaaggtgaaagagttgctaagatgttggaaattgctaccgaagatgctgaaatgagagcaaagagaatcaagggctctttaccagcaagagttatcattgttggtggtggtttggccggttgttccgcagctatcgaagcagctaactgtggcgcccacgtcatcttgttagaaaaggaaccaaagttaggtggtaactctgcaaaggctacctccggtatcaacgcctggggtactagagcacaagcaaaacaaggtgtcatggacggcggcaagtttttcgaaagagatacccatagatccggcaagggtggtaattgcgatccatgccttgttaagactttgtccgttaagtcctctgatgcagttaagtggttatctgaattaggtgttccattgactgttttgtctcaattaggtggtgcttcaaggaaacgttgtcaccgtgcaccagataagtctgatggtacaccagtcccagttggtttcaccattatgaaaacccttgaaaaccacattgtcaacgatttgtccagacatgttacagttatgacaggtattaccgtcacagctttagaatctacatcaagagtcagacctgatggtgttttagtcaagcatgttactggtgttcacttgattcaggcatctggtcaatctatggttttgaatgcagacgctgttatcttagctactggtggtttctccaatgatcataccccaaactcccttttacaacaatacgccccacagttgtcatcttttccaacaaccaatggtgtctgggcaactggcgatggtgttaagatggcttccaagttgggtgtcgccttagttgatatggataaggtccaattacatcctaccggcttgttagacccaaaagatccatctaatagaaccaagtatcttggtccagaggccttaagaggttccggcggtgtcttgttaaacaaaaacggtgaaagatttgttaatgaattagacttaagatctgttgtctctcaagctatcatcgcacaagataatgagtacccaggctctggtggttccaagttcgcatactgtgttttgaacgaaactgcagcaaagttattcggcaaaaacttccttggtttctactggaatagattaggtcttttccaaaaggttgattccgttgctggtttagctaagttgattggttgtccagaagctaatgttgttgctacattgaagcaatatgaggagttatcttccaaaaagcttaatccttgtccattgactggcaagtctgtctttccttgtgttttaggcactcaaggtccatactatgttgccttggttaccccatccattcactacactatgggtggttgtttgatttccccatctgctgagatgcaaaccattgacaactctggtgttactcctgtcagacgtccaatcttaggcttattcggtgctggtgaagttactggcggtgtccatggtggtaacagattaggcggtaactctttgttaggacgtgttgttttcggcaagatcgctggtgacagagctgcaaccatcttgcaaaagaaaaacaccggcttatcaatgacagaatggtctactgtcgtcttaagagaagttagagaaggtggtgtctatggtgctggttccagagttttgaggtttaacatgcctggtgcattacagagaactggtttagctttaggtcaattcatcggtatcagaggtgattgggacggtcacagattgatcggttactattctccaatcactttacctgatgatgttggtgttattggtatcttagctagagcagacaagggtagattggcagaatggatttctgcattgcagccaggtgacgctgttgagatgaaggcctgcggtggtcttatcattgacagaagattcgctgaaagacatttctttttccgtggtcataagatcagaaagttggcccttatcggtggtggtactggtgttgcaccaatgttacaaatcgtcagagctgctgtcaaaaagccatttgtcgattcaattgagtccattcagttcatctatgctgcagaggatgtttccgagcttacatacagaaccttacttgaatcttacgaagaggaatatggttcagaaaagtttaagtgtcacttcgttttgaataacccaccagctcaatggactgacggtgttggtttcgttgatactgcattgttgagatccgcagttcaagcaccatcaaatgatttgcttgttgcaatttgtggtccaccaatcatgcaaagagcagttaagggtgcattgaaaggtttaggttacaatatgaatcttgttagaaccgttgacgaaactgaaccaccatcataa

SEQ ID NO: 36 - DNA, 3435 bp, synthetic construct
  source 1..3435: mol_type = other DNA; note = Synthetic - Mutated L. mexicana FRD gene
atggctgatggcaaaacctctgcatcagttgttgctgttgatgctgaacgtgccgctaaggaaagagatgcagcagctagagctatgttgcaaggtggtggtgtctctcctgctggcaaggcacaattgttgaaaaagggtttggttcacactgttccatataccttaaaggttgtcgtcgcagatccaaaggaaatggagaaggcaactgctgacgcagaagaggttttacaagctgcatttcaagtcgtcgacacccttttgaacaactttaacgaaaactcagaagtttcaagagtcaataggttggcagttggtgaggaacatcaaatgtctgaaacattgaaacacgtcatggcctgttgtcaaaaggtttatcattcctccagaggtgtttttgacccagcagttggtccattagtccgtgaacttagagaagctgctcacaagggtaaaactgttccagccgaaagagttaatgatttgttatccaaatgtacccttaatgcatctttttcaattgatatgtccagaggtatgattgcaaggaagcatccagacgccatgttggatttgggtggtgtcaacaagggttatggtatcgactacattgttgaacacttaaactctttgggttatgatgatgtctttttcgaatggggtggtgatgttagagcatccggcaaaaaccagttatctcaaccttgggctgttggtattgttagaccacctgccttggccgacattagaactgttgtcccagaggacaaaagatcctttatccgtgtcgtcagattgaacaacgaagctattgctacctctggtgattatgagaatttggttgaaggtcctggttctaaggtttactcttccaccttcaatccaacttccaaaaacttgttggaacctaccgaagcaggtatggctcaagtttctgtcaagtgttgctcatgtatctacgctgatgctttagcaacagcagctttgttgaaaaacgatcctgctgccgttagaaggatcttagataactggagatatgtcagagatactgttactgactacaccacttacacaagggaaggtgaaagagttgctaagatgttggaaattgctaccgaagatgctgaaatgagagcaaagagaatcaagggctctttaccagcaagagttatcattgttggtggtggtttggccggttgttccgcagctatcgaagcagctaactgtggcgcccacgtcatcttgttagaaaaggaaccaaagttaggtggtaactctgcaaaggctacctccggtatcaacgcctggggtactagagcacaagcaaaacaaggtgtcatggacggcggcaagtttttcgaaagagatacccatagatccggcaagggtggtaattgcgatccatgccttgttaagactttgtccgttaagtcctctgatgcagttaagtggttatctgaattaggtgttccattgactgttttgtctcaattaggtggtgcttcaaggaaacgttgtcaccgtgcaccagataagtctgatggtacaccagtcccagttggtttcaccattatgaaaacccttgaaaaccacattgtcaacgatttgtccagacatgttacagttatgacaggtattaccgtcacagctttagaatctacatcaagagtcagacctgatggtgttttagtcaagcatgttactggtgttcacttgattcaggcatctggtcaatctatggttttgaatgcagacgctgttatcttagctactggtggtttctccaatgatcataccccaaactcccttttacaacaatacgccccacagttgtcatcttttccaacaaccaatggtgtctgggcaactggcgatggtgttaagatggcttccaagttgggtgtcgccttagttgatatggataaggtccaattacatcctaccggcttgttagacccaaaagatccatctaatagaaccaagtatcttggtccagaggccttaagaggttccggcggtgtcttgttaaacaaaaacggtgaaagatttgttaatgaattagacttaagatctgttgtctctcaagctatcatcgcacaagataatgagtacccaggctctggtggttccaagttcgcatactgtgttttgaacgaaactgcagcaaagttattcggcaaaaacttccttggtttctactggaatagattaggtcttttccaaaaggttgattccgttgctggtttagctaagttgattggttgtccagaagctaatgttgttgctacattgaagcaatatgaggagttatcttccaaaaagcttaatccttgtccattgactggcaagtctgtctttccttgtgttttaggcactcaaggtccatactatgttgccttggttaccccatccattcactacactatgggtggttgtttgatttccccatctgctgagatgcaaaccattgacaactctggtgttactcctgtcagacgtccaatcttaggcttattcggtgctggtgaagttactggcggtgtccatggtggtaacagattaggcggtaactctttgttagaatgtgttgttttcggcaagatcgctggtgacagagctgcaaccatcttgcaaaagaaaaacaccggcttatcaatgacagaatggtctactgtcgtcttaagagaagttagagaaggtggtgtctatggtgctggttccagagttttgaggtttaacatgcctggtgcattacagagaactggtttagctttaggtcaattcatcggtatcagaggtgattgggacggtcacagattgatcggttactattctccaatcactttacctgatgatgttggtgttattggtatcttagctagagcagacaagggtagattggcagaatggatttctgcattgcagccaggtgacgctgttgagatgaaggcctgcggtggtcttatcattgacagaagattcgctgaaagacatttctttttccgtggtcataagatcagaaagttggcccttatcggtggtggtactggtgttgcaccaatgttacaaatcgtcagagctgctgtcaaaaagccatttgtcggtcgaattgagtccattcagttcatctatgctgcagaggatgtttccgagcttacatacagaaccttacttgaatcttacgaagaggaatatggttcagaaaagtttaagtgtcacttcgttttgaataacccaccagctcaatggactgacggtgttggtttcgttgatactgcattgttgagatccgcagttcaagcaccatcaaatgatttgcttgttgcaatttgtggtccaccaatcatgcaaagagcagttaagggtgcattgaaaggtttaggttacaatatgaatcttgttagaaccgttgacgaaactgaaccaccatcataa

SEQ ID NO: 37 - DNA, 3420 bp, synthetic construct
  source 1..3420: mol_type = other DNA; note = Synthetic - Mutated T.brucei FRD gene
atggttgatggtagatcttcagcttctattgttgcagttgatccagaaagagcagcaagagaaagagatgctgcagctagagctttgttacaagattctccattgcacactaccatgcaatatgctacctccggtttagaattgaccgtcccttatgcattgaaagttgttgcatctgccgacaccttcgatagagctaaggaagttgcagatgaagtccttagatgtgcctggcaattggctgatacagtccttaactcctttaacccaaactctgaagtctctcttgttggtagacttccagtcggtcagaagcatcaaatgtccgccccacttaagagagttatggcttgttgtcaaagagtttacaattcctctgctggttgtttcgacccatccaccgccccagttgcaaaggctttgcgtgaaatcgctttaggcaaggagagaaacaatgcctgtttggaggctttaacacaagcatgcactttgccaaactctttcgtcattgactttgaagcaggtactatctcacgtaaacatgaacatgcttcacttgacttaggtggtgtttcaaagggttacatcgttgactatgttattgataacattaacgcagctggtttccaaaatgtctttttcgattggggtggtgattgtagagcctccggtatgaatgctagaaataccccttgggttgttggtattactagaccaccatcattagatatgttaccaaacccaccaaaggaagcatcctatatctctgttatctcattggacaacgaagctttggcaacctccggtgattacgagaatttgatctacacagctgatgacaagcctttaacttgtacttacgattggaagggcaaggaacttatgaagccatctcaatcaaacattgcccaagtttcagttaagtgctattcagcaatgtacgctgacgctttagccaccgcttgtttcatcaaaagagatccagccaaggttagacaattgttagatggttggagatacgttagagatactgtcagagattacagagtttatgttagagaaaatgagagagtcgctaagatgtttgaaattgcaaccgaagatgctgaaatgagaaaaagacgtatctctaatactttgcctgcaagagtcatcgttgtcggtggcggtttagcaggtttatctgcagcaattgaagctgcaggctgcggtgcacaagtcgttttgatgggaaaggaagctaagttaggtggtaactctgcaaaggcaacctctggtatcaatggttggggtactagagcccaagcaaaggcttccattgttgacggtggcaagtatttcgaaagagatacttacaaatctggtattggtggtaataccgacccagctttagttaagactctttccatgaagtctgctgacgctattggttggttaacatcattaggtgttcctttaacagtcttatcacaattgggtggtcattccagaaagagaactcacagagcaccagacaaaaaggatggcaccccattacctattggttttaccattatgaaaaccttagaagatcacgtcagaggtaatctttctggtagaattactatcatggaaaactgttccgttacctctttactttctgaaactaaggaaagaccagatggtactaaacaaatcagagttaccggtgttgagttcactcaagcaggctctggcaaaactaccattttggccgacgcagtcatcttggccactggtggtttctctaacgacaagaccgcagactctttgttgagagaacatgcccctcacttagttaactttcctacaactaacggtccttgggcaactggtgacggtgttaagcttgctcaaagattaggtgcacaattggtcgacatggataaggttcaattgcatccaactggtttgattaacccaaaagatccagctaatccaacaaagtttttgggtccagaagctttaagaggttccggtggtgtcttgttaaacaaacagggtaaaagatttgttaacgaattagatttgcgttctgttgtttccaaggccattatggaacaaggtgctgaatacccaggctctggtggttctatgttcgcatattgtgtccttaatgcagctgcacaaaagttgtttggtgtctcttcccacgagttctactggaaaaagatgggtttgttcgttaaggctgatactatgagagatttggcagcattgattggttgtccagtcgagtctgttcaacaaactttagaggaatatgaaagattatctatttctcagagatcctgtccaatcactagaaaatctgtttacccatgtgttttgggcactaagggtccatactacgttgctttcgtcaccccatctattcactatacaatgggtggttgtttgatttccccatcagcagaaattcagatgaaaaacacctcctcccgtgctccattgtcccattccaaccctatcttgggtttgttcggtgctggtgaagttactggtggtgtccacggtggcaatagattaggtggtaactcattgttagaatgtgttgtctttggtagaattgctggtgatagagcttctaccattttgcagagaaagtcctccgcattatctttcaaggtctggactaccgttgttttgagagaagttagagaaggtggcgtctatggtgccggttcaagagttttgagattcaacttgcctggtgctttacaaagatccggtttgtccttgggtcaattcatcgcaatcagaggtgactgggatggtcaacaattgattggttactattccccaattacattgccagatgacttgggtatgattgacattttggctagatccgataaaggtactttaagagaatggatttctgctttagaaccaggcgacgctgttgagatgaaagcatgcggtggtttagtcatcgagagaagattgtcagataagcactttgtctttatgggtcacatcattaacaagttatgtttgatcgctggtggtacaggcgttgcacctatgttacaaatcattaaggcagcattcatgaaaccttttatcgataccttagaatctgtccatcttatctatgctgcagaagatgttaccgagttaacttatagagaagttttagaggagcgtagaagagagtctcgtggcaagttcaaaaagacctttgttttgaacagacctccaccactttggactgatggtgttggtttcatcgatagaggtatcttaactaatcatgtccaaccaccatccgataaccttttggttgcaatctgtggtccacctgtcatgcagcgtattgttaaggccaccttaaagactttgggttacaatatgaatcttgttagaacagttgacgaaacagaaccatccggttcctaa

SEQ ID NO: 38 - DNA, 3889 bp, synthetic construct
  source 1..3889: mol_type = other DNA; note = Synthetic - GPD gene deletion fragment
ccttcatttacgaaataaagtgccgcggttacgcagcacacaccagcaatcacgtgcagtgtctttttcttttttttttcttttttttcctctttttcttttgttttgtttcgtttcttttccgccagttcccgttttccatttccggaacaacaatgggactccactgttttctttccccccttccgttttcggctcgcagtctgtacatgcacgtttatccgacacctgtcttgtttggcgcgtaattaatacagtttctccggagtccaggtctcggacgggtaatttacacgtcatcattcatttctgtgtcaagagaggtagcgcaaaaagtagaaatggtgaaccacgggaatgacttgctggaaatcgacgccagagtccatttgaaaacctacctctacaagagaggaaacacactacagggtgtccctggtccgtaaaatggcgtaatatgatgacttccctctatagacgttgtatttccagctccaacatggttaaactattgctatggtgatggtattacagatagtaaaagaaggaagggggggtggcaatctcaccctaacagttactaagaacgtctacttcatctactgtcaatatacattggccacatgccgagaaattacgtcgacgccaaagaagggctcagccgaaaaaagaaatggaaaacttggccgaaaagggaaacaaacaaaaaggtgatgtaaaattagcggaaaggggaattggcaaattgagggagaaaaaaaaaaggcagaaaaggaggcggaaagtcagtacgttttgaaggcgtcattggttttcccttttgcagagtgtttcatttcttttgtttcatgacgtagtggcgtttcttttcctgcactttagaaatctatcttttccttatcaagtaacaagcggttggcaaaggtgtatataaatcaaggaattcccactttgaaccctttgaattttgatatcgtttattttaaatttatttgcggccgcggatccctcgaggccttaattaacatctgaatgtaaaatgaacattaaaatgaattactaaactttacgtctactttacaatctataaactttgtttaatcatataacgaaatacactaatacacaatcctgtacgtatgtaatacttttatccatcaaggattgagaaaaaaaagtaatgattccctgggccattaaaacttagacccccaagcttggataggtcactctctattttcgtttctcccttccctgatagaagggtgatatgtaattaagaataatatataattttataataaaagaattcatagcctcatgaaatcagccatttgcttttgttcaacgatcttttgaaattgttgttgttcttggtagttaagttgatccatcttggcttatgttgtgtgtatgttgtagttattcttagtatattcctgtcctgagtttagtgaaacataatatcgccttgaaatgaaaatgctgaaattcgtcgacatacaatttttcaaactttttttttttcttggtgcacggacatgtttttaaaggaagtactctataccagttattcttcacaaatttaattgctggagaatagatcttcaacgctttaataaagtagtttgtttgtcaaggatggcgtcatacaaagaaagatcagaatcacacacttcccctgttgctaggagacttttctccatcatggaggaaaagaagtctaacctttgtgcatcattggatattactgaaactgaaaagcttctctctattttggacactattggtccttacatctgtctagttaaaacacacatcgatattgtttctgattttacgtatgaaggaactgtgttgcctttgaaggagcttgccaagaaacataattttatgatttttgaagatagaaaatttgctgatattggtaacactgttaaaaatcaatataaatctggtgtcttccgtattgccgaatgggctgacatcactaatgcacatggtgtaacgggtgcaggtattgtttctggcttgaaggaggcagcccaagaaacaaccagtgaacctagaggtttgctaatgcttgctgagttatcatcaaagggttctttagcatatggtgaatatacagaaaaaacagtagaaattgctaaatctgataaagagtttgtcattggttttattgcgcaacacgatatgggcggtagagaagaaggttttgactggatcattatgactccaggggttggtttagatgacaaaggtgatgcacttggtcaacaatatagaactgttgatgaagttgtaaagactggaacggatatcataattgttggtagaggtttgtacggtcaaggaagagatcctatagagcaagctaaaagataccaacaagctggttggaatgcttatttaaacagatttaaatgattcttacacaaagatttgatacatgtacactagtttaaataagcatgaaaagaattacacaagcaaaaaaaaaaaaataaatgaggtactttacgttcacctacaaccaaaaaaactagatagagtaaaatcttaagatttagaaaaagttgtttaacaaaggctttagtatgtgaatttttaatgtagcaaagcgataactaataaacataaacaaaagtatggttttctttatcagtcaaatcattatcgattgattgttccgcgtatctgcagatagcctcatgaaatcagccatttgcttttgttcaacgatcttttgaaattgttgttgttcttggtagttaagttgatccatcttggcttatgttgtgtgtatgttgtagttattcttagtatattcctgtcctgagtttagtgaaacataatatcgccttgaaatgaaaatgctgaaattcgtcgacatacaatttttcaaactttttttttttcttggtgcacggacatgtttttaaaggaagtactctataccagttattcttcacaaatttaattgctggagaatagatcttcaacgccccgggggatctggatccgcggccgcaataacctcagggagaactttggcattgtactctccattgacgagtccgccaacccattcttgttaaacctaaccttgcattatcacattccctttgaccccctttagctgcatttccacttgtctacattaagattcattacacattctttttcgtatttctcttacctccctcccccctccatggatcttatatataaatcttttctataacaataatatctactagagttaaacaacaattccacttggcatggctgtctcagcaaatctgcttctacctactgcacgggtttgcatgtcattgtttctagcagggaatcgtccatgtacgttgtcctccatgatggtcttcccgctgccactttctttagtatcttaaatagagcagatcttacgtccactgtgcatccgtgcaccccgaaaatcgtatggttttccttgccacctctcacaattttgaatatgctcaacgcgaaagagaggggaagaggaatcgcattcgtagagtggctacattcaaccctgacaaaggaactagcgtttgtgcaggagagagtggtttgcatagatttcctttcctttgcaagcatattatatagagtagccaatacagtaacagctacagcacaaaaaagagaacgagaacgagaacgagaacaagaacaagaactagcactactgtcactgccagcatcaacattactaccattattccaacatgtttgcaactagaaatataaccattggtgtcagaacactcagaccaaccagtttcttgaaaacaaggtcttttctgcaacagaggctacaatcaacgctaaagaagagctatgaaccaaccaaatccgagct

SEQ ID NO: 39 - DNA, 3889 bp, synthetic construct
  source 1..3889: mol_type = other DNA; note = Synthetic - GPD gene deletion fragment
ccttcatttacgaaataaagtgccgcggttacgcagcacacaccagcaatcacgtgcagtgtctttttcttttttttttcttttttttcctctttttcttttgttttgtttcgtttcttttccgccagttcccgttttccatttccggaacaacaatgggactccactgttttctttccccccttccgttttcggctcgcagtctgtacatgcacgtttatccgacacctgtcttgtttggcgcgtaattaatacagtttctccggagtccaggtctcggacgggtaatttacacgtcatcattcatttctgtgtcaagagaggtagcgcaaaaagtagaaatggtgaaccacgggaatgacttgctggaaatcgacgccagagtccatttgaaaacctacctctacaagagaggaaacacactacagggtgtccctggtccgtaaaatggcgtaatatgatgacttccctctatagacgttgtatttccagctccaacatggttaaactattgctatggtgatggtattacagatagtaaaagaaggaagggggggtggcaatctcaccctaacagttactaagaacgtctacttcatctactgtcaatatacattggccacatgccgagaaattacgtcgacgccaaagaagggctcagccgaaaaaagaaatggaaaacttggccgaaaagggaaacaaacaaaaaggtgatgtaaaattagcggaaaggggaattggcaaattgagggagaaaaaaaaaaggcagaaaaggaggcggaaagtcagtacgttttgaaggcgtcattggttttcccttttgcagagtgtttcatttcttttgtttcatgacgtagtggcgtttcttttcctgcactttagaaatctatcttttccttatcaagtaacaagcggttggcaaaggtgtatataaatcaaggaattcccactttgaaccctttgaattttgatatcgtttattttaaatttatttgcggccgcggatccagatcccccggggcgttgaagatctattctccagcaattaaatttgtgaagaataactggtatagagtacttcctttaaaaacatgtccgtgcaccaagaaaaaaaaaaagtttgaaaaattgtatgtcgacgaatttcagcattttcatttcaaggcgatattatgtttcactaaactcaggacaggaatatactaagaataactacaacatacacacaacataagccaagatggatcaacttaactaccaagaacaacaacaatttcaaaagatcgttgaacaaaagcaaatggctgatttcatgaggctatctgcagatacgcggaacaatcaatcgataatgatttgactgataaagaaaaccatacttttgtttatgtttattagttatcgctttgctacattaaaaattcacatactaaagcctttgttaaacaactttttctaaatcttaagattttactctatctagtttttttggttgtaggtgaacgtaaagtacctcatttatttttttttttttgcttgtgtaattcttttcatgcttatttaaactagtgtacatgtatcaaatctttgtgtaagaatcatttaaatctgtttaaataagcattccaaccagcttgttggtatcttttagcttgctctataggatctcttccttgaccgtacaaacctctaccaacaattatgatatccgttccagtctttacaacttcatcaacagttctatattgttgaccaagtgcatcacctttgtcatctaaaccaacccctggagtcataatgatccagtcaaaaccttcttctctaccgcccatatcgtgttgcgcaataaaaccaatgacaaactctttatcagatttagcaatttctactgttttttctgtatattcaccatatgctaaagaaccctttgatgataactcagcaagcattagcaaacctctaggttcactggttgtttcttgggctgcctccttcaagccagaaacaatacctgcacccgttacaccatgtgcattagtgatgtcagcccattcggcaatacggaagacaccagatttatattgatttttaacagtgttaccaatatcagcaaattttctatcttcaaaaatcataaaattatgtttcttggcaagctccttcaaaggcaacacagttccttcatacgtaaaatcagaaacaatatcgatgtgtgttttaactagacagatgtaaggaccaatagtgtccaaaatagagagaagcttttcagtttcagtaatatccaatgatgcacaaaggttagacttcttttcctccatgatggagaaaagtctcctagcaacaggggaagtgtgtgattctgatctttctttgtatgacgccatccttgacaaacaaactactttattaaagcgttgaagatctattctccagcaattaaatttgtgaagaataactggtatagagtacttcctttaaaaacatgtccgtgcaccaagaaaaaaaaaaagtttgaaaaattgtatgtcgacgaatttcagcattttcatttcaaggcgatattatgtttcactaaactcaggacaggaatatactaagaataactacaacatacacacaacataagccaagatggatcaacttaactaccaagaacaacaacaatttcaaaagatcgttgaacaaaagcaaatggctgatttcatgaggctatgaattcttttattataaaattatatattattcttaattacatatcacccttctatcagggaagggagaaacgaaaatagagagtgacctatccaagcttgggggtctaagttttaatggcccagggaatcattacttttttttctcaatccttgatggataaaagtattacatacgtacaggattgtgtattagtgtatttcgttatatgattaaacaaagtttatagattgtaaagtagacgtaaagtttagtaattcattttaatgttcattttacattcagatgttaattaaggcctcgagggatccgcggccgcaataacctcagggagaactttggcattgtactctccattgacgagtccgccaacccattcttgttaaacctaaccttgcattatcacattccctttgaccccctttagctgcatttccacttgtctacattaagattcattacacattctttttcgtatttctcttacctccctcccccctccatggatcttatatataaatcttttctataacaataatatctactagagttaaacaacaattccacttggcatggctgtctcagcaaatctgcttctacctactgcacgggtttgcatgtcattgtttctagcagggaatcgtccatgtacgttgtcctccatgatggtcttcccgctgccactttctttagtatcttaaatagagcagatcttacgtccactgtgcatccgtgcaccccgaaaatcgtatggttttccttgccacctctcacaattttgaatatgctcaacgcgaaagagaggggaagaggaatcgcattcgtagagtggctacattcaaccctgacaaaggaactagcgtttgtgcaggagagagtggtttgcatagatttcctttcctttgcaagcatattatatagagtagccaatacagtaacagctacagcacaaaaaagagaacgagaacgagaacgagaacaagaacaagaactagcactactgtcactgccagcatcaacattactaccattattccaacatgtttgcaactagaaatataaccattggtgtcagaacactcagaccaaccagtttcttgaaaacaaggtcttttctgcaacagaggctacaatcaacgctaaagaagagctatgaaccaaccaaatccgagct

SEQ ID NO: 40 - DNA, 3175 bp, synthetic construct
  source 1..3175: mol_type = other DNA; note = Synthtic - PGI gene deletion construct
cttcgctcgccatctatatcttcaacgaacaacggaattacaaacatgggcagtagttcaaacaatctccagactctcaactctctctcgctatcgttgaaacatccacagttccaaggcctattctccccactggatgtccacagtccgtacgaacagaacgtttcttccccactggcccccaccgttccggctgttccgggaaccgcaccttcattcgagtcggacgatctctacaatgcaacggctgcccgcaaaagagactctctcaagatgaagagaagatagacgctacatcattgtctgtgcagtacctaatatatagtacttggtataaggtataataaagctataaaattataataatcttaataataataaccatattaatggaaggatgaggcccgatgtccttttttttgcctttctactatagtgcttacattgtgtataaattctcgcggccgcggatccctcgaggccttaattaacatctgaatgtaaaatgaacattaaaatgaattactaaactttacgtctactttacaatctataaactttgtttaatcatataacgaaatacactaatacacaatcctgtacgtatgtaatacttttatccatcaaggattgagaaaaaaaagtaatgattccctgggccattaaaacttagacccccaagcttggataggtcactctctattttcgtttctcccttccctgatagaagggtgatatgtaattaagaataatatataattttataataaaagaattcatagcctcatgaaatcagccatttgcttttgttcaacgatcttttgaaattgttgttgttcttggtagttaagttgatccatcttggcttatgttgtgtgtatgttgtagttattcttagtatattcctgtcctgagtttagtgaaacataatatcgccttgaaatgaaaatgctgaaattcgtcgacatacaatttttcaaactttttttttttcttggtgcacggacatgtttttaaaggaagtactctataccagttattcttcacaaatttaattgctggagaatagatcttcaacgctttaataaagtagtttgtttgtcaaggatggcgtcatacaaagaaagatcagaatcacacacttcccctgttgctaggagacttttctccatcatggaggaaaagaagtctaacctttgtgcatcattggatattactgaaactgaaaagcttctctctattttggacactattggtccttacatctgtctagttaaaacacacatcgatattgtttctgattttacgtatgaaggaactgtgttgcctttgaaggagcttgccaagaaacataattttatgatttttgaagatagaaaatttgctgatattggtaacactgttaaaaatcaatataaatctggtgtcttccgtattgccgaatgggctgacatcactaatgcacatggtgtaacgggtgcaggtattgtttctggcttgaaggaggcagcccaagaaacaaccagtgaacctagaggtttgctaatgcttgctgagttatcatcaaagggttctttagcatatggtgaatatacagaaaaaacagtagaaattgctaaatctgataaagagtttgtcattggttttattgcgcaacacgatatgggcggtagagaagaaggttttgactggatcattatgactccaggggttggtttagatgacaaaggtgatgcacttggtcaacaatatagaactgttgatgaagttgtaaagactggaacggatatcataattgttggtagaggtttgtacggtcaaggaagagatcctatagagcaagctaaaagataccaacaagctggttggaatgcttatttaaacagatttaaatgattcttacacaaagatttgatacatgtacactagtttaaataagcatgaaaagaattacacaagcaaaaaaaaaaaaataaatgaggtactttacgttcacctacaaccaaaaaaactagatagagtaaaatcttaagatttagaaaaagttgtttaacaaaggctttagtatgtgaatttttaatgtagcaaagcgataactaataaacataaacaaaagtatggttttctttatcagtcaaatcattatcgattgattgttccgcgtatctgcagatagcctcatgaaatcagccatttgcttttgttcaacgatcttttgaaattgttgttgttcttggtagttaagttgatccatcttggcttatgttgtgtgtatgttgtagttattcttagtatattcctgtcctgagtttagtgaaacataatatcgccttgaaatgaaaatgctgaaattcgtcgacatacaatttttcaaactttttttttttcttggtgcacggacatgtttttaaaggaagtactctataccagttattcttcacaaatttaattgctggagaatagatcttcaacgccccgggggatctggatccgcggccgcgttaacgaaagttccaaactttatttataatgtgtttatgtttgtattttaatcactctttatgacctatatatgaagcttttagcattatcgcagcaagtataaatggatgcatgtaaattccatagttcatatagtgcgatttggtgaatttttgaaatttttgctaatggataatatactctatatttttacactgtgtttactgatgcctcttccgaatttctttctttcaccactcaacccatgaaaggcaaggaacacatacatcatgattacaataatatagatatcggggtaacaataacagttcccagaagaaggaaacaaaaacgtacaggatctacaaatagtcaaagcactgggtggaagaaattgttatggctcaaacaaccttatgacgataactacacagattcgagcttcttatcacaactgaaacgaaattcaacggttgtaaagtactcgtatgtaaagctagtcaatgatttttccatcattgtattgcatctgtcgtccattatgtttgttgttgttgtattttatgggatctatcagttaaattggaacccgattaaaccaacagtgataagtacgatttgtacactcattggattcattttttatgttgtaacattgaagataataagaaataaagaattgattgaacgagct

SEQ ID NO: 41 - DNA, 3175 bp, synthetic construct
  source 1..3175: mol_type = other DNA; note = Synthetic - PGI gene deletion construct
cgttcaatcaattctttatttcttattatcttcaatgttacaacataaaaaatgaatccaatgagtgtacaaatcgtacttatcactgttggtttaatcgggttccaatttaactgatagatcccataaaatacaacaacaacaaacataatggacgacagatgcaatacaatgatggaaaaatcattgactagctttacatacgagtactttacaaccgttgaatttcgtttcagttgtgataagaagctcgaatctgtgtagttatcgtcataaggttgtttgagccataacaatttcttccacccagtgctttgactatttgtagatcctgtacgtttttgtttccttcttctgggaactgttattgttaccccgatatctatattattgtaatcatgatgtatgtgttccttgcctttcatgggttgagtggtgaaagaaagaaattcggaagaggcatcagtaaacacagtgtaaaaatatagagtatattatccattagcaaaaatttcaaaaattcaccaaatcgcactatatgaactatggaatttacatgcatccatttatacttgctgcgataatgctaaaagcttcatatataggtcataaagagtgattaaaatacaaacataaacacattataaataaagtttggaactttcgttaacgcggccgcggatccctcgaggccttaattaacatctgaatgtaaaatgaacattaaaatgaattactaaactttacgtctactttacaatctataaactttgtttaatcatataacgaaatacactaatacacaatcctgtacgtatgtaatacttttatccatcaaggattgagaaaaaaaagtaatgattccctgggccattaaaacttagacccccaagcttggataggtcactctctattttcgtttctcccttccctgatagaagggtgatatgtaattaagaataatatataattttataataaaagaattcatagcctcatgaaatcagccatttgcttttgttcaacgatcttttgaaattgttgttgttcttggtagttaagttgatccatcttggcttatgttgtgtgtatgttgtagttattcttagtatattcctgtcctgagtttagtgaaacataatatcgccttgaaatgaaaatgctgaaattcgtcgacatacaatttttcaaactttttttttttcttggtgcacggacatgtttttaaaggaagtactctataccagttattcttcacaaatttaattgctggagaatagatcttcaacgctttaataaagtagtttgtttgtcaaggatggcgtcatacaaagaaagatcagaatcacacacttcccctgttgctaggagacttttctccatcatggaggaaaagaagtctaacctttgtgcatcattggatattactgaaactgaaaagcttctctctattttggacactattggtccttacatctgtctagttaaaacacacatcgatattgtttctgattttacgtatgaaggaactgtgttgcctttgaaggagcttgccaagaaacataattttatgatttttgaagatagaaaatttgctgatattggtaacactgttaaaaatcaatataaatctggtgtcttccgtattgccgaatgggctgacatcactaatgcacatggtgtaacgggtgcaggtattgtttctggcttgaaggaggcagcccaagaaacaaccagtgaacctagaggtttgctaatgcttgctgagttatcatcaaagggttctttagcatatggtgaatatacagaaaaaacagtagaaattgctaaatctgataaagagtttgtcattggttttattgcgcaacacgatatgggcggtagagaagaaggttttgactggatcattatgactccaggggttggtttagatgacaaaggtgatgcacttggtcaacaatatagaactgttgatgaagttgtaaagactggaacggatatcataattgttggtagaggtttgtacggtcaaggaagagatcctatagagcaagctaaaagataccaacaagctggttggaatgcttatttaaacagatttaaatgattcttacacaaagatttgatacatgtacactagtttaaataagcatgaaaagaattacacaagcaaaaaaaaaaaaataaatgaggtactttacgttcacctacaaccaaaaaaactagatagagtaaaatcttaagatttagaaaaagttgtttaacaaaggctttagtatgtgaatttttaatgtagcaaagcgataactaataaacataaacaaaagtatggttttctttatcagtcaaatcattatcgattgattgttccgcgtatctgcagatagcctcatgaaatcagccatttgcttttgttcaacgatcttttgaaattgttgttgttcttggtagttaagttgatccatcttggcttatgttgtgtgtatgttgtagttattcttagtatattcctgtcctgagtttagtgaaacataatatcgccttgaaatgaaaatgctgaaattcgtcgacatacaatttttcaaactttttttttttcttggtgcacggacatgtttttaaaggaagtactctataccagttattcttcacaaatttaattgctggagaatagatcttcaacgccccgggggatctggatccgcggccgcgagaatttatacacaatgtaagcactatagtagaaaggcaaaaaaaaggacatcgggcctcatccttccattaatatggttattattattaagattattataattttatagctttattataccttataccaagtactatatattaggtactgcacagacaatgatgtagcgtctatcttctcttcatcttgagagagtctcttttgcgggcagccgttgcattgtagagatcgtccgactcgaatgaaggtgcggttcccggaacagccggaacggtgggggccagtggggaagaaacgttctgttcgtacggactgtggacatccagtggggagaataggccttggaactgtggatgtttcaacgatagcgagagagagttgagagtctggagattgtttgaactactgcccatgtttgtaattccgttgttcgttgaagatatagatggcgagcgaagggcc

SEQ ID NO: 42 - DNA, 3420 bp, Trypanosoma brucei
  source 1..3420: mol_type = other DNA
atggttgatggtagatcttcagcttctattgttgcagttgatccagaaagagcagcaagagaaagagatgctgcagctagagctttgttacaagattctccattgcacactaccatgcaatatgctacctccggtttagaattgaccgtcccttatgcattgaaagttgttgcatctgccgacaccttcgatagagctaaggaagttgcagatgaagtccttagatgtgcctggcaattggctgatacagtccttaactcctttaacccaaactctgaagtctctcttgttggtagacttccagtcggtcagaagcatcaaatgtccgccccacttaagagagttatggcttgttgtcaaagagtttacaattcctctgctggttgtttcgacccatccaccgccccagttgcaaaggctttgcgtgaaatcgctttaggcaaggagagaaacaatgcctgtttggaggctttaacacaagcatgcactttgccaaactctttcgtcattgactttgaagcaggtactatctcacgtaaacatgaacatgcttcacttgacttaggtggtgtttcaaagggttacatcgttgactatgttattgataacattaacgcagctggtttccaaaatgtctttttcgattggggtggtgattgtagagcctccggtatgaatgctagaaataccccttgggttgttggtattactagaccaccatcattagatatgttaccaaacccaccaaaggaagcatcctatatctctgttatctcattggacaacgaagctttggcaacctccggtgattacgagaatttgatctacacagctgatgacaagcctttaacttgtacttacgattggaagggcaaggaacttatgaagccatctcaatcaaacattgcccaagtttcagttaagtgctattcagcaatgtacgctgacgctttagccaccgcttgtttcatcaaaagagatccagccaaggttagacaattgttagatggttggagatacgttagagatactgtcagagattacagagtttatgttagagaaaatgagagagtcgctaagatgtttgaaattgcaaccgaagatgctgaaatgagaaaaagacgtatctctaatactttgcctgcaagagtcatcgttgtcggtggcggtttagcaggtttatctgcagcaattgaagctgcaggctgcggtgcacaagtcgttttgatggaaaaggaagctaagttaggtggtaactctgcaaaggcaacctctggtatcaatggttggggtactagagcccaagcaaaggcttccattgttgacggtggcaagtatttcgaaagagatacttacaaatctggtattggtggtaataccgacccagctttagttaagactctttccatgaagtctgctgacgctattggttggttaacatcattaggtgttcctttaacagtcttatcacaattgggtggtcattccagaaagagaactcacagagcaccagacaaaaaggatggcaccccattacctattggttttaccattatgaaaaccttagaagatcacgtcagaggtaatctttctggtagaattactatcatggaaaactgttccgttacctctttactttctgaaactaaggaaagaccagatggtactaaacaaatcagagttaccggtgttgagttcactcaagcaggctctggcaaaactaccattttggccgacgcagtcatcttggccactggtggtttctctaacgacaagaccgcagactctttgttgagagaacatgcccctcacttagttaactttcctacaactaacggtccttgggcaactggtgacggtgttaagcttgctcaaagattaggtgcacaattggtcgacatggataaggttcaattgcatccaactggtttgattaacccaaaagatccagctaatccaacaaagtttttgggtccagaagctttaagaggttccggtggtgtcttgttaaacaaacagggtaaaagatttgttaacgaattagatttgcgttctgttgtttccaaggccattatggaacaaggtgctgaatacccaggctctggtggttctatgttcgcatattgtgtccttaatgcagctgcacaaaagttgtttggtgtctcttcccacgagttctactggaaaaagatgggtttgttcgttaaggctgatactatgagagatttggcagcattgattggttgtccagtcgagtctgttcaacaaactttagaggaatatgaaagattatctatttctcagagatcctgtccaatcactagaaaatctgtttacccatgtgttttgggcactaagggtccatactacgttgctttcgtcaccccatctattcactatacaatgggtggttgtttgatttccccatcagcagaaattcagatgaaaaacacctcctcccgtgctccattgtcccattccaaccctatcttgggtttgttcggtgctggtgaagttactggtggtgtccacggtggcaatagattaggtggtaactcattgttagaatgtgttgtctttggtagaattgctggtgatagagcttctaccattttgcagagaaagtcctccgcattatctttcaaggtctggactaccgttgttttgagagaagttagagaaggtggcgtctatggtgccggttcaagagttttgagattcaacttgcctggtgctttacaaagatccggtttgtccttgggtcaattcatcgcaatcagaggtgactgggatggtcaacaattgattggttactattccccaattacattgccagatgacttgggtatgattgacattttggctagatccgataaaggtactttaagagaatggatttctgctttagaaccaggcgacgctgttgagatgaaagcatgcggtggtttagtcatcgagagaagattgtcagataagcactttgtctttatgggtcacatcattaacaagttatgtttgatcgctggtggtacaggcgttgcacctatgttacaaatcattaaggcagcattcatgaaaccttttatcgataccttagaatctgtccatcttatctatgctgcagaagatgttaccgagttaacttatagagaagttttagaggagcgtagaagagagtctcgtggcaagttcaaaaagacctttgttttgaacagacctccaccactttggactgatggtgttggtttcatcgatagaggtatcttaactaatcatgtccaaccaccatccgataaccttttggttgcaatctgtggtccacctgtcatgcagcgtattgttaaggccaccttaaagactttgggttacaatatgaatcttgttagaacagttgacgaaacagaaccatccggttcctaa

SEQ ID NO: 43 - DNA, 3420 bp, Trypanosoma cruzi
  source 1..3420: mol_type = other DNA
atggctgacggtagatcctctgcatctgttgttgcagttgatccagaaaaggctgcaagagaaagagatgaagcagctcgtgctttgttaagagactctccattacaaactcatcttcagtacatgactaatggtttagagttgactgtcccattcaccttaaaggttgtcgctgaagcagttgcattttccagagcaaaggaagttgctgacgaagttttgaggtcagcctggcatcttgcagacaccgtcttgaacaactttaaccctaactccgagatttctatgattggtagattaccagttggtcaaaaacatacaatgtccgctacattgaagtctgttatcacatgctgtcagcatgttttcaattcatccagaggtgtttttgatccagctactggtcctatcattgaagctttaagagctaaggttgctgagaaagcctctgtttctgatgaacagatggagaagttgtttcgtgtttgtaacttctcttcctcattcatcgttgatttggaaatgggtactattgccagaaaacacgaagatgcaagatttgacttaggtggtgtttccaagggttacatcgttgactacgttgttgaaagattgaacgctgctggtattgtcgatgtctacttcgaatggggtggtgactgtagagcttccggtactaacgcaagacgtaccccatggatggttggtatcattagacctccatctttagaacaattgagaaacccaccaaaagatccatcctacattagggttttaccacttaacgatgaagcactttgtacctctggtgactatgagaatttgaccgaaggctctaacaaaaagttgtatacatccattttcgattggaaaaagagatccttgttggaaccagttgaatcagaattggcccaagtttccattagatgttattctgccatgtatgcagacgcattagcaacagcttctcttatcaagagagatatcaaaaaggttagacaaatgttggaagattggagacacgtccgtaatagggttactaactatgttacctataccagacaaggtgaaagagtcgcacgtatgtttgaaattgctactgataacgctgagattaggaaaaagagaattgcaggctctttacctgctagggttattgttgtcggtggtggtttagctggtttgtctgcagcaattgaagcaactgcatgtggtgcccaagttatccttttagaaaaggaacctaaagttggtggtaattccgcaaaggctacatctggtatcaacggttggggtactagagcacaagctgaacaagatgtctacgactctggcaagtacttcgaaagagatacacacaaatctggtttaggtggttctaccgatccaggcttagttcgtactttatcagtcaagtctggtgacgctatttcatggttatcttctcttggtgttccattaactgtcttgtcacaattaggcggtcattccagaaaaaggactcacagagcccctgataaggcagatggtactccagttccaattggtttcaccattatgcaaaccttagaacagcatgttagaaccaagttagcagacagagttactatcatggagaataccaccgttacctccttgctttctaagtccagagttagacatgatggtgcaaagcaagttagagtctacggtgttgaagtcttacaagacgaaggtgtcgtttctcgtatcttggccgatgctgtcattttggcaacaggtggtttctccaatgacaaaaccccaaactccttattgcaagagttcgctccacaattgtcaggttttccaacaaccaacggtccatgggctactggcgatggtgttaagttagcaagagaacttggtgtcaagttggttgatatggataaggtccaacttcatccaactggtttgattgaccctaaggacccagcaaatccaaccaaatacttaggtccagaagcattgagaggttctggtggtgtcttgttaaacaaaaagggtgaaagatttgtcaatgagttggacttgcgttccgtcgtttcaaatgctatcattgaacaaggtgatgaatatccagatgccggtggttccaagttcgccttctgtgttttgaatgatgcagcagttaagttattcggtgtcaactcccacggtttctactggaagagacttggtttgtttgttaaggctgataccgttgaaaagttagccgcattgatcggttgcccagtcgaaaatgttagaaacacattaggtgattatgagcaattgtccaaggaaaacagacaatgtccaaagactagaaaagttgtctatccatgtgttgttggtccacaaggtccattctatgttgcttttgttaccccatctattcactataccatgggtggttgtttgatctcaccatctgctgagatgcaattggaagagaacactacctccccatttggtcacagaaggcctatcttcggtcttttcggtgccggtgaagttactggtggtgtccatggtggtaacagattaggtggcaactctttgttggagtgtgttgtttttggtagaatcgctggtgatagagctgcaaccattttgcaaaagaaaccagttccactttcctttaagacttggaccaccgtcattttgagagaggtccgtgaaggtggcatgtacggtactggttcaagagtcttaagattcaatttgccaggtgctttacaaagatctggtttgcaattgggtcaattcatcgctattagaggcgaatgggatggtcaacaattgattggctactattccccaatcactttgccagacgatttgggtgtcatcggcattttggctagatccgataagggtactttgaaggaatggatttctgctttggaacctggtgatgcagttgagatgaagggttgtggcggtttagttattgaaaggagattctctgaaagatacttgtacttttctggtcacgctttgaaaaagttatgccttattgctggtggtactggtgtcgcaccaatgttacaaatcattagagcagcattgaaaaagccattccttgagaatatcgaatcaattagacttatctatgctgctgaggacgtttctgagttgacatacagggaattgttagaacatcaccaaagagattctaagggcaagtttagatccatcttcgttttgaatagaccacctccaatttggactgatggtgttggctttatcgacaaaaagttgttatcttcatccgttcagccacctgctaaggatttgttagtcgccatttgtggtcctcctatcatgcaacgtgttgtcaagacttgtcttaagtcattaggttatgatatgcagttagtcagaacagttgatgaagtcgaaactcaaaactcctaa

SEQ ID NO: 44 - DNA, 3435 bp, Leishmania braziliensis
  source 1..3435: mol_type = other DNA
atggctgatggtaaaacctctgcttccgttgttgctgtcgacccagagcgtgcagcaaaggagagagatgcagcagcaagagcaatgttacaagacggtggtgtttctccagttggtaaagctcagttgttgaaaaagggtttggcatatgctgtcccttacacccttaagattgttgttgcagatcctaaagctatggaaaagaccaccgcagacgttgagaaggtccttcaaaccgcattccaagtcgttgacactttgttaaacaatttcaacgaaaactccgaggtttctcgtatcaacagaatgccagtcggtgaggaacaccaaatgtctgctgcattgaagagagttatgggttgctgtcagcgtgtttacaattcatctcgtggtgcttttgacccagctgttggtccattggtcagagaattgagggaagctgcaagagaaggcagaactttaccagcagaaaggattaacgctttgttatccaagtgtaccttgaatatctccttttccattgatttgaacagaggtactattgccagaaaacacgcagatgcaatgttggatttgggtggtgtcaataagggttatggtgttgattatgttgtcgaacatttgaacaatttgggttatgatgatgtctttttcgaatggggtggtgatgttagagcatctggcaaaaacccatcaaaccaacattgggttgttggtattgctagaccaccagcacttgctgatatcagaaccgttgttccacaagacaagcaatccttcatcagagttgtttgtcttaatgatgaagcaattgccacctctggtgattacgaaaatcttgtcgaaggtcctggttctaaggtttactcctctaccttcaacgcaacctctaagtccttattggaaccaaccgaaaccaatatcgcacaagtctctgttaagtgttactcatgcatgtatgcagacgcattggctaccgctgccttattgaaaaacaatccaactgctgttcgtagaatgttagataactggagatatgttcgtgatactgttaccgactatacaacctattccagagaaggtgaaagagttgcaaagatgtttgagattgcaaccgaagataaggaaatgagagctaagagaatttccggttccttgccagcaagagtcattatcgtcggtggtggtttagctggttgttctgcagctattgaagcagtcaactgtggtgctcaagtcattttgttagaaaaggaagccaagattggtggcaactccgcaaaggctacctctggtatcaacgcctggggtactagagcccaggctaaacaaggtgttatggatggtggcaagtttttcgagagagacacccatagatccggtaaaggtggtcactgtgatccttgtttggttaagacactttccgttaagtcatcagacgcagttaagtggttgtctgaattgggtgttccattaaccgtcttatcccaattaggtggtgcatccagaaagaggtgtcatagagccccagataagtctgatggtactcctgttccaattggttttacaatcatgaaaacattagaaaatcacatcattaacgatctttctcaccaagttactgttatgactggtatcaaggttactggtttggagtccacttctcacgctcgtccagatggtgttttagttaagcacgttactggtgttagattgattcaaggtgatggccaatccagagttttgaatgctgatgccgttatcttagcaactggtggtttctccaatgaccatactgctaactctttacttcaacaatacgctccacaactttcatcctttccaaccactaatggtgtttgggccactggtgacggtgtcaaggcagctagagaattaggtgttgagttggttgacatggataaggtccaattgcatccaacaggtttgttagatccaaaggacccatccaacaggactaagtacttgggtccagaagctttaaggggttcaggcggtgtcttgttaaacaaaaacggtgaacgtttcgtcaacgaacttgatttgagatctgtcgtttctcaagccattatcgaacaaaacaacgtttaccctggttctggtggttccaagtttgcttactgcgttttgaacgaagcagcagctaagttgttcggcaaaaactttttgggtttctattggcatagattaggtctttttgaaaaggttgaagatgttgctggtttagccaaattgatcggttgtccagaggaaaatgttaccgctacattgaaggaatacaaggaattgtcctccaaaaagcttcatgcctgtcctttaaccaacaaaaacgtctttccttgcactttaggtactgaaggcccttactatgttgctttcgtcacaccttcaattcactacacaatgggtggttgtttgatctccccttcagcagaaatgcagaccattgataacactggtgtcacaccagttcgtagaccaatcttgggcttattcggtgctggtgaagttactggtggtgtccatggtggtaacagattgggtggtaattccttattggaatgtgttgtctttggtagaattgctggtgatagagccgctaccattttgcaaaagaagaatgctggtttatcaatgactgagtggtctacagttgtcttaagagaagtcagagaaggcggtgtttacggtactggttctcgtgtccttagattcaatatgccaggtgccttacaaaagactggcttagcattgggtcaattcatcgcaatgagaggtgattgggatggtcaacagttattgggttactattctccaattacattaccagacgacattggtgttattggtatcttagctagagctgacaaaggtagattagctgaatggatttctgcattacaaccaggtgatgctgttgagatgaaggcatgtggcggtttgattatccatagaagattcgctgctagacacttgtttttccgttctcacaagattagaaagcttgctcttattggtggtggtactggtgttgcaccaatgttgcaaattgtcagggctgcagtcaaaaagccatttgttgactctattgagtctattcagttcatctatgcagctgaagatgtctccgaacttacttatagaactttgttggaatcatatgaaaaggaatacggttctggcaaattcaagtgtcatttcgtcttgaataacccaccatcacaatggaccgagggcgttggtttcgttgatactgctttgttgcgttctgccgttcaagcaccttctaacgacttgttagtcgctatttgtggcccaccaatcatgcaaagagcagtcaaatcagccttaaagggtttaggttacaatatgaatttggttagaacagttgatgaaccagaaccattgtcttaa

SEQ ID NO: 45 - DNA, 3537 bp, Saccharomyces cerevisiae
  source 1..3537: mol_type = other DNA
atgtcgcaaagaaaattcgccggcttgagagataacttcaatctcttgggtgaaaagaacaaaatattggtggctaatagaggagaaattccaatcagaatttttcgtaccgctcatgaactgtctatgcagacggtagctatatattctcatgaagatcgtctttcaacgcacaaacaaaaggctgacgaagcatacgtcataggtgaagtaggccaatatacccccgtcggcgcttatttggccattgacgaaatcatttccattgcccaaaaacaccaggtagatttcatccatccaggttatgggttcttgtctgaaaattcggaatttgccgacaaagtagtgaaggccggtatcacttggattggccctccagctgaagttattgactccgtgggtgataaggtctcagctagaaacctggcagcaaaagctaatgtgcccaccgttcctggtacaccaggtcctatagaaactgtagaggaagcacttgacttcgtcaatgaatacggctacccggtgatcattaaggccgcctttggtggtggtggtagaggtatgagagtcgttagagaaggtgacgacgtggcagatgcctttcaacgtgctacctccgaagcccgtactgccttcggtaatggtacctgctttgtggaaagattcttggacaagccaaagcatattgaagttcaattgttggccgataaccacggaaacgtggttcatcttttcgaaagagactgttccgtgcagagaagacaccaaaaggttgtcgaagtggccccagcaaagactttaccccgtgaagtccgtgacgccattttgacagatgcagttaaattggccaaagagtgtggctacagaaatgcgggtactgctgaattcttggttgataaccaaaatagacactatttcattgaaattaatccaagaatccaagtggaacataccatcacagaagaaattaccggtatagatattgtggcggctcagatccaaattgcggcaggtgcctctctaccccagctgggcctattccaggacaaaattacgactcgtggctttgccattcagtgccgtattaccacggaagaccctgctaagaacttccaaccagataccggtagaatagaagtgtaccgttctgcaggtggtaatggtgttagactggatggtggtaacgcctatgcaggaacaataatctcacctcattacgactcaatgctggtcaaatgctcatgctccggttccacctacgaaatcgttcgtagaaaaatgattcgtgcattaatcgagttcagaattagaggtgtcaagaccaacattcccttcctattgactcttttgaccaatccagtatttattgagggtacatactggacgacttttattgacgacaccccacaactgttccaaatggtttcatcacaaaacagagcccaaaaacttttacattacctcgccgacgtggcagtcaatggttcatctatcaagggtcaaattggcttgccaaaattaaaatcaaatccaagtgtcccccatttgcacgatgctcagggcaatgtcatcaacgttacaaagtctgcaccaccatccggatggaggcaagtgctactagaaaaggggccagctgaatttgccagacaagttagacagttcaatggtactttattgatggacaccacctggagagacgctcatcaatctctacttgcaacaagagtcagaacccacgatttggctacaatcgctccaacaaccgcacatgcccttgcaggtcgtttcgccttagaatgttggggtggtgccacattcgatgttgcaatgagatttttgcatgaggatccatgggaacgtttgagaaaattaagatctctggtgcctaatattccattccaaatgttattgcgtggtgccaatggtgtggcttattcttcattgcctgacaatgctattgaccatttcgtcaagcaagccaaggataatggtgttgatatatttagagtctttgatgccttaaatgacttggaacaattgaaggtcggtgtagatgctgtgaagaaggcaggtggtgttgtagaagccactgtttgtttctctggggatatgcttcagccaggcaagaaatacaatttggattactacttggaaattgctgaaaaaattgtccaaatgggcactcatatcctgggtatcaaagatatggcaggtaccatgaagccagcagctgccaaactactgattggatctttgagggctaagtaccctgatctcccaatacatgttcacactcacgattctgcaggtactgctgttgcatcaatgactgcgtgtgctctggcgggcgccgatgtcgttgatgttgccatcaactcaatgtctggtttaacttcacaaccatcaatcaatgctctgttggcttcattagaaggtaatattgacactggtattaacgttgagcatgtccgtgaactagatgcatattgggcagagatgagattgttatactcttgtttcgaggctgacttgaagggcccagatccagaagtttatcaacatgaaatcccaggtggtcaattgacaaacttgttgtttcaagcccaacaattgggtcttggagaacaatgggccgaaacaaaaagagcttacagagaagccaattatttattgggtgatattgtcaaagttaccccaacttcgaaggtcgttggtgatctggcacaatttatggtctccaataaattaacttccgatgatgtgagacgcctggctaattctttggatttccctgactctgttatggatttcttcgaaggcttaatcggccaaccatacggtgggttcccagaaccatttagatcagacgttttaaggaacaagagaagaaagttgacttgtcgtccaggcctggaactagagccatttgatctcgaaaaaattagagaagacttgcagaatagatttggtgatgttgatgagtgcgacgttgcttcttataacatgtacccaagagtttatgaagacttccaaaagatgagagaaacgtatggtgatttatctgtattgccaacaagaagctttttgtctccactagagactgacgaagaaattgaagttgtaatcgaacaaggtaaaacgctaattatcaagctacaggctgtgggtgatttgaacaaaaagaccggtgaaagagaagtttactttgatttgaatggtgaaatgagaaaaattcgtgttgctgacagatcacaaaaagtggaaactgttactaaatccaaagcagacatgcatgatccattacacattggtgcaccaatggcaggtgtcattgttgaagttaaagttcataaaggatcactaataaagaagggccaacctgtagccgtattaagcgccatgaaaatggaaatgattatatcttctccatccgatggacaagttaaagaagtgtttgtctctgatggtgaaaatgtggactcttctgatttattagttctattagaagaccaagttcctgttgaaactaaggcataa

SEQ ID NO: 46 - DNA, 3528 bp, Kluyveromyces marxianus
  source 1..3528: mol_type = other DNA
atgtctacccaaaacgatctggccgggttgcgtgataactcgaacctattaggtgaaaagaacaagattcttgttgccaaccgtggtgaaattccaattagaatctttagaacggctcatgaactttcgatgaagactgttgcgatctattcgcacgaggatagactatctatgcacagattgaaggcagacgaagcttacgttattggtgagccaggaaaatacactccagttggtgcgtatttggcgatcgatgagattatcaagattgctcaattgcacggagtgagcttcatccaccctggttatgggttcttatcggaaaactctgagtttgccaagaaggtggccgactctggtatcacgtgggttggtcctccagccgatgtgatcgatgctgttggtgacaaggtttctgccagaaacttggccgagagagcggatgttccagtggttccaggtacgcctggtccaatagagacagttgaagaagcagttgaatttgtggagaagtacggatacccagtcatcatcaaggctgccttcggtggtggtggtcgtggtatgagagttgttcgtgaaggtgatgatatcgccgatgctttccaaagagccaagtccgaagctgttactgctttcggtaacggtacttgtttcgttgaaagattcttggacaagccaaagcacatcgaagttcagttgttggctgatcactacggtaatgtcatccatctattcgaaagagactgttctgtgcaaagaagacatcaaaaggtcgttgaagtagcgccagccaagactttgccagagagcgtgcgtaatgcaatcttgactgacgctgtcaagttggctaaggaggcaggatacagaaatgctggtaccgctgaatttttggtcgacaaccaaaacagacactactttattgaaatcaacccaagaattcaagtcgaacataccatcaccgaagaaattaccggtatcgacattgtcgccgcacaaattcaaatcgcagcaggtgcttccttggaacaattgggactattgcaagatagaatcaccacccgtggtttcgctattcaatgtcgtatcactactgaagatccttccaagaacttccagccagatactggtcgtatcgatgtttaccgttccgctggtggtaacggtgtcagattggatggtggtaacgcattcgctggttcggtcatttcacctcattatgattccatgttggtcaaatgttcttgttccggttccacttacgaaatcgttcgtcgtaagatgttgcgtgccttgatcgaattcagaatcagaggtgtgaagacaaacattccattcttgctaacgttgttgactcatcctgtgttcaagtccggtgactactggactaccttcatcgatgacactccacaattgttcgaaatggtttcttctcaaaacagagcacaaaaactattgcactacttggccgatcttgccgttaacggttcatcgatcaagggtcaaattggtctaccaaagttaaagactcatcctactatcccacatttgcataaggccgatggctccattctagatgtgtctgccaagcctcctgccgggtggagagatgttctattgcaacacggcccagaagaatttgcaaagcaagttagaaagttcaagggtactttgctaatggacaccacctggagagatgctcatcaatctctattggccactagagtcagaacttacgatttggctgccatcgctccaactactgctcatgctttgagcggtgctttcgctttggaatgttggggtggtgccactttcgatgtctccatgagattcttgcacgaagatccatgggaacgtttgagaactttgagaaagttggttcctaacattccattccaaatgttgctacgtggtgccaacggtgttgcatactcttctctaccagataacgctatcgaccactttgtcaagcaagcaaaggataacggtgttgacattttcagagtcttcgatgctctaaacgatttggagcaattgactgtcggtgttgacgctgtcaagaaggctggtggtgttgtcgaagctaccatttgttactccggtgacatgctagcaccaggtaagaagtacaaccttgactactacttggacattgttgaacaagtggttaagagaggtacccatattcttggtatcaaggatatggcaggtactttgaagccatctgctgctaagctcttgatcggttctatcagaacaaagtaccctgacttgccaattcacgtccatacccatgactccgccggtaccggtgttgcttccatggctgcatgtgctttcgctggtgctgatgttgttgatgttgcaaccaactctatgtctggtatgacttctcaaccatctgtcaatgcactattggctgctcttgatggtgaaatcgactgtaatgtcaacgtcagctacatcagtcagctagatgcttactgggctgaaatgagactattgtactcatgtttcgaagccgacttgaagggtcctgatccagaagtttacgtccatgaaattccaggtggtcaattgaccaacttgctcttccaagcccaacaattgggtcttggtgagcaatgggctgaaaccaagagagcttaccgtgaagcaaacctgttgttgggtgatgttgttaaggtcactccaacatccaaggttgtcggtgatttggctcaattcatggtcactaacaagttgacctcggatgatgttaagagattagcttcatctttggatttcccagactccgtcatggacttctttgaaggtttaatcggtcaaccatacggtggtttcccagaacctctaagatctgatgttttgaagaacaagagaagaaagttgaccaagagaccaggtttggaattggctccattcgatttggaaggcattaaggaagatttgactaacagatttggtgacattgacgactgtgatgttgcttcttacaacatgtatccaaaggtctacgaagatttccgtaagatcagagaaaagtacggtgatctatctgttttgccaaccaagaacttcttgtctccaccttcaatcggtgaagaaatcgtcgttacaattgaacaaggtaagactttgatcattaagccacaagctattggtgatttgaacaaggagactggtatcagagaagtttacttcgaattgaacggtgaattgagaaaggtctctgttgctgacagatctcaaaaggttgaaacgatctccaagccaaaggctgacgcccacgatccattccaagttggttctccaatggcaggtgttgttgtcgaagtcaaggtacacaagggttctttgatctccaagggccaaccagtcgctgtcctaagtgccatgaagatggaaatggttatctcctccccatctgatggtcaagtcaaggaagtgcttgtcaaggatggtgaaaacgttgacgcttctgacttgctcgttgttttggaagaagctccagctaaagaataa

SEQ ID NO: 47 - DNA, 22 bp, synthetic construct
  source 1..22: mol_type = other DNA; note = Synthetic - Primer
gcaactgatgttcacgaatgcg

SEQ ID NO: 48 - DNA, 20 bp, synthetic construct
  source 1..20: mol_type = other DNA; note = Synthetic - Primer
ttgccgttgcagcaaatctc

SEQ ID NO: 49 - DNA, 2652 bp, Escherichia coli
  source 1..2652: mol_type = other DNA
atgaacgaacaatattccgcattgcgtagtaatgtcagtatgctcggcaaagtgctgggagaaaccatcaaggatgcgttgggagaacacattcttgaacgcgtagaaactatccgtaagttgtcgaaatcttcacgcgctggcaatgatgctaaccgccaggagttgctcaccaccttacaaaatttgtcgaacgacgagctgctgcccgttgcgcgtgcgtttagtcagttcctgaacctggccaacaccgccgagcaataccacagcatttcgccgaaaggcgaagctgccagcaacccggaagtgatcgcccgcaccctgcgtaaactgaaaaaccagccggaactgagcgaagacaccatcaaaaaagcagtggaatcgctgtcgctggaactggtcctcacggctcacccaaccgaaattacccgtcgtacactgatccacaaaatggtggaagtgaacgcctgtttaaaacagctcgataacaaagatatcgctgactacgaacacaaccagctgatgcgtcgcctgcgccagttgatcgcccagtcatggcataccgatgaaatccgtaagctgcgtccaagcccggtagatgaagccaaatggggctttgccgtagtggaaaacagcctgtggcaaggcgtaccaaattacctgcgcgaactgaacgaacaactggaagagaacctcggctacaaactgcccgtcgaatttgttccggtccgttttacttcgtggatgggcggcgaccgcgacggcaacccgaacgtcactgccgatatcacccgccacgtcctgctactcagccgctggaaagccaccgatttgttcctgaaagatattcaggtgctggtttctgaactgtcgatggttgaagcgacccctgaactgctggcgctggttggcgaagaaggtgccgcagaaccgtatcgctatctgatgaaaaacctgcgttctcgcctgatggcgacacaggcatggctggaagcgcgcctgaaaggcgaagaactgccaaaaccagaaggcctgctgacacaaaacgaagaactgtgggaaccgctctacgcttgctaccagtcacttcaggcgtgtggcatgggtattatcgccaacggcgatctgctcgacaccctgcgccgcgtgaaatgtttcggcgtaccgctggtccgtattgatatccgtcaggagagcacgcgtcataccgaagcgctgggcgagctgacccgctacctcggtatcggcgactacgaaagctggtcagaggccgacaaacaggcgttcctgatccgcgaactgaactccaaacgtccgcttctgccgcgcaactggcaaccaagcgccgaaacgcgcgaagtgctcgatacctgccaggtgattgccgaagcaccgcaaggctccattgccgcctacgtgatctcgatggcgaaaacgccgtccgacgtactggctgtccacctgctgctgaaagaagcgggtatcgggtttgcgatgccggttgctccgctgtttgaaaccctcgatgatctgaacaacgccaacgatgtcatgacccagctgctcaatattgactggtatcgtggcctgattcagggcaaacagatggtgatgattggctattccgactcagcaaaagatgcgggagtgatggcagcttcctgggcgcaatatcaggcacaggatgcattaatcaaaacctgcgaaaaagcgggtattgagctgacgttgttccacggtcgcggcggttccattggtcgcggcggcgcacctgctcatgcggcgctgctgtcacaaccgccaggaagcctgaaaggcggcctgcgcgtaaccgaacagggcgagatgatccgctttaaatatggtctgccagaaatcaccgtcagcagcctgtcgctttataccggggcgattctggaagccaacctgctgccaccgccggagccgaaagagagctggcgtcgcattatggatgaactgtcagtcatctcctgcgatgtctaccgcggctacgtacgtgaaaacaaagattttgtgccttacttccgctccgctacgccggaacaagaactgggcaaactgccgttgggttcacgtccggcgaaacgtcgcccaaccggcggcgtcgagtcactacgcgccattccgtggatcttcgcctggacgcaaaaccgtctgatgctccccgcctggctgggtgcaggtacggcgctgcaaaaagtggtcgaagacggcaaacagagcgagctggaggctatgtgccgcgattggccattcttctcgacgcgtctcggcatgctggagatggtcttcgccaaagcagacctgtggctggcggaatactatgaccaacgcctggtagacaaagcactgtggccgttaggtaaagagttacgcaacctgcaagaagaagacatcaaagtggtgctggcgattgccaacgattcccatctgatggccgatctgccgtggattgcagagtctattcagctacggaatatttacaccgacccgctgaacgtattgcaggccgagttgctgcaccgctcccgccaggcagaaaaagaaggccaggaaccggatcctcgcgtcgaacaagcgttaatggtcactattgccgggattgcggcaggtatgcgtaataccggctaa

SEQ ID NO: 50 - DNA, 2643 bp, Mannheimia succiniciproducens
  source 1..2643: mol_type = other DNA
atgacagaagaatatttaatgatgcgtaataacatcaatatgctggggcgctttttgggcgaaactattcaggaggcgcaaggtgacgatattctcgaactgattgaaaatatccgcgtactgtcccgcaattcccgtagcggcgatgacaaagcccgggcggcattattagacaccctttccactatttcggcggataatattattccggttgcccgcgctttcagccagtttctgaacctgacaaatgtggcggaacaatatcaaaccatgtctcgctcccatgaagataaggtttctgcggaacgttccactgctgcgctgttcgcccgcctgaaagaacaacatgtttctcaggaagaaatcattaaaaccgtacagaaactgttgattgaaatcgtccttaccgctcacccgacggaagttacccgccgttcattaatgcacaaacaggttgaaatcaacaaatgtctggctcagctggatcatacggatttaaccgccgaagaacaaaaaaatattgagtataaattacttcgtcttatcgccgaagcctggcataccaatgaaatccgtaccaatcggccgacacctctggaagaagccaaatggggttttgccgttatcgaaaacagtttatgggaaggtttgcccgcctttatccgcaaacttaacgatgccgccgtcgaacatttaaattatgctttgccggtagacctcacaccggtacgcttctcttcctggatgggcggtgaccgtgacggcaaccccttcgttaccgcaaaaattacccgggaagcgctgcaacttgcgcgctggaaagcggcggatttatttttaaccgatattcaggaactctgcgacgagttgtcaatgacacaatgcactgcggaattccgagaaaaatacggtgatcatttagaaccctatcgtgtagttgtgaaggatttacgcagcaaattaaaaaatacgctggattattacaacgatatacttgcgggtcgcattccgccgtttaaacaagatgaaatcatcagtgaagaccaacaactctggcaaccgctttatgactgttatcaatccctaaccgcctgcggtatgcgtattattgccaatggattattgctggataccttacgccgcgttcgttgtttcggcgtcacattactgcgtttagatatccgtcaggaaagcacccgccatagcgacgccatcggcgaaattacccgctacatcggtttaggcgattacagccaatggacagaagatgacaaacaagccttcctgatccgggaattaagttcccgtcgtccgctaattccccataactggacgccttcggaacacactcgggaaattttagacacctgtaaagtcattgcaaaacagccggaaggcgttatttcctgctatatcatttccatggcgcgcaccgcttccgatgttttggcggtgcatttattattgaaagaagcgggcatttcataccatctgccggtagttcctctatttgaaacattggacgacctggacgcttctaaagaagtgatgacgcaactgtttaacgtaggctggtatcgcggcgtaatcaaaaaccgccaaatgatcatgatcggctattccgatagcgccaaagatgcgggcatgatggcggcctcatgggcgcaataccgggcgcaggacgctttagtcaaactttgcgaacaaaccggcatcgaacttaccctcttccacggccgcggcggcaccgtaggacgtggcggtgcaccggctcacgccgcattattatcccaaccgccacgttctctgaaaaacggcttacgggtaaccgaacaaggggaaatgatccgcttcaaactgggattaccggctatcgccgcagaaagtctggatctctacgccagcgccattcttgaggccaacctcctgccgccgccggaaccgaaagccagctggtgccgggtaatggacgaacttgccgtcgcttcttgcgaaatctatcgcaatgtggtgcgcggcgataaagattttgtgccttacttccgcagcgccacaccggaacaggaactggcaaaactgcctttaggttcccgaccggcaaaacgcaatccgaacggcggcgttgaaagcctgcgtgccattccctggatcttcgcctggatgcaaaaccgcctgatgctgcccgcctggctcggtgccggcgcctcaatccgtcaggcgatggaaagcggcaaagcggcggtgattgaagaaatgtgcaaccattggccgtttttcaatacccgaatcggcatgcttgaaatggtattcagtaaaaccgatagctggctgtccgaatattacgaccagcgtttagtgaaaaaagagctttggtatttaggcgaatcgctgcgcaaacagttaagcgaagatatcgctaccgtgttacggctttccggcaaaggcgatcaattaatgtcggatttgccttgggtggcggaatctattgcactgcgtaacgtttacaccgacccgttaaacttattgcaagtggaattattgcgtcgtttgcgagcggatcccgaacatccgaatccggatatcgagcaagcgctgatgatcaccattaccggtatcgccgcgggtatgcgtaatacgggttag

SEQ ID NO: 51 - DNA, 21 bp, synthetic construct
  source 1..21: mol_type = other DNA; note = Synthetic - Primer
acggcagtataccctatcagg

SEQ ID NO: 52 - DNA, 20 bp, synthetic construct
  source 1..20: mol_type = other DNA; note = Synthetic - Primer
aatgatccatggtccgagag

SEQ ID NO: 53 - DNA, 22 bp, synthetic construct
  source 1..22: mol_type = other DNA; note = Synthetic - Primer
gaagagacgtacaagatccgcc

SEQ ID NO: 54 - DNA, 47 bp, synthetic construct
  source 1..47: mol_type = other DNA; note = Synthetic - Primer
ggataaaagtattacatacgtacaggattgtgtattagtgtatttcg

SEQ ID NO: 55 - DNA, 22 bp, synthetic construct
  source 1..22: mol_type = other DNA; note = Synthetic - Primer
taggaatggtgcatcatccaac

SEQ ID NO: 56 - DNA, 44 bp, synthetic construct
  source 1..44: mol_type = other DNA; note = Synthetic - Primer
ccaaccaaacacgcgtacaatgaacgaacaatattccgcattgc

SEQ ID NO: 57 - DNA, 23 bp, synthetic construct
  source 1..23: mol_type = other DNA; note = Synthetic - Primer
ggacacggagaacccatttattc

SEQ ID NO: 58 - DNA, 1029 bp, Issatchenkia orientalis
  source 1..1029: mol_type = other DNA
atgtccaatgttaaagtagctctactaggtgccgctggtggtatcggccaaccacttgctctattacttaagcttaatccaaacataacccatttggcactctatgacgttgtgcatgttcctggagtggctgccgacctacaccatatagacacagatgtagtgattacccaccatttgaaagatgaagacggtacggccttggcaaacgccctcaaggacgctacgtttgttattgtccccgccggtgttccgagaaagcccggcatgactagaggtgatttgttcacaattaatgccggtatatgtgccgaattggctaatgctattagtttgaacgctcctaatgcattcacccttgtcattaccaatccggtcaactcgaccgttcctatatttaaggaaatatttgctaaaaatgaagccttcaatccaaggagactgtttggtgtaactgctctagatcatgttagatcaaatacttttctctcggaattaattgacggtaaaaatccccaacattttgatgtcactgttgttggcggacactctggtaactcaattgtccccctattctcccttgttaaggctgccgaaaatttagacgatgaaattatagatgccttgattcatagagttcaatacggtggagatgaagttgtggaagcaaagagcggtgcgggctcggcaactctttcaatggcttatgccgctaacaagttcttcaatatattgcttaatggatacttgggtttgaagaagacaatgatttcaagttatgtctttttagacgattcaatcaacggcgtccctcaattaaaggaaaatttgtctaaacttttgaaaggttccgaggttgagttaccaagttatttggctgttccaatgacctatggtaaagaaggtattgaacaagtcttttacgattgggtgtttgaaatgtcaccaaaggaaaaggaaaacttcattacagcgattgaatacattgatcaaaatattgaaaaaggtctgaattttatggtacgttaa

SEQ ID NO: 59 - DNA, 1011 bp, Issatchenkia orientalis
  source 1..1011: mol_type = other DNA
atggtcaaggtgactattttaggcgctgccggtggaattggacaaccactctcattgttattgagacttaatccatggattgacgaattggccttgtttgatattgtcaatacccccggcgtgagttgtgatttgtcgcatattcctgcatcacaggttgttaatggctatgctccgaaatcgaaatcagatacagagacaatcaagactgccttgaaaggtgctgatattgttgttattcctgcaggaattccacgtaaacctggtatgacaagaaacgatctctttaaaatcaatgccggaatcgttaagagtttgattcatagtgcaggaaccacttgccctgatgcatttatttgtgtcatttcgaaccctgtcaactcgacagttccaattgccgttgaagaactaaagcgtttgaatgtttttaatccacataaagttttcggtattaccacattggacaatttcagattagaagaatttctgagtggagaacttggtggaattgtcaaaccaaatgatttatatggtgatgtagttgctataggtggccattcgggcgactctatagtaccgatcttgaattcgtggaatttgaatttcatcaatgatggagattcttataacaatttggtcaagagggtccagtttggaggcgatgaggttgtcaaggcaaaggacgggaaaggttcggctacattgtcaatggctacagctgcatacaggtttgtcaacaacctcttggacgccattgtcaataacaagaaagtcaaggaagtggcctttgtgaaaatcgaccaattgccaactacaagggttccttattttgttgttgatgaaactcagtattttagtctacccattattctcggtagacaggggattgagagggtcacgttcccagaatctctgacagagcaagaggtgagaatgacaaagcacgctgttgctaaagttaaagttgacgttaataaaggcttcaattttgtccatggcccaaaactgtaa

SEQ ID NO: 60 - DNA, 1005 bp, Issatchenkia orientalis
  source 1..1005: mol_type = other DNA
atgttctccagaatctctgctagacaattctcctcctctgctgcttccgcttacaaggtcaccgttttaggtgctgcaggtggtattggccaaccactatctcttttgatgaagttgaaccacaaggtcaccaacttatccttgtacgacttgagattgggtgctggtgttgccactgacttgtcccacattccaaccaactccgttgtcaagggctatggtccagaaaacaatggtttgaaggacgccttgaccggctccgatgttgttcttattccagctggtgttccaagaaaaccaggtatgactagagacgatctcttcaacaccaatgcatcgattgtcagagacttggcaaaggctgctgcagaccactgtccaaacgccgtcttgttgatcatttcaaaccctgtcaactcaactgtcccaattgttgctgaggttttgaaatcaaagggcgtctacaacccaaagaagttgtttggtgtcaccactttggacgttttgagatcctcgagattcttgagtgaagtcgtcaacaccgacccaaccaccgaaaccgtcactgttgttggtggccactctggtgtcaccattgttcctttaatctcccaaaccaaacacaaggacttgccaaaggaaacctacgaagcattggtccacagaatccaattcggtggtgatgaggttgtcaaggccaaggacggtgcaggttccgctaccttgtccatggcccaagccggtgcaagaatggcctcctccgtcttgaagggtttggctggtgaagttgacattgtcgaaccaacctttattgactctccattgttcaagtccgaaggtgtcgaattcttctcctccagagtcacccttggtccagaaggtgtccaagaagtccacccattgggcgtcttatctactgctgaagaagaaatggttgctactgctaaggaaaccttgaagaagaacatccaaaagggtgtcgactttgtcaaggctaacccataa

SEQ ID NO: 61 - DNA, 1071 bp, Zygosaccharomyces rouxii
  source 1..1071: mol_type = other DNA
atgcctcattctatcaacggtgatgttaaaatcgcagtattgggagctgcaggtggtattggacaatcactttcgctacttttgaagacccagttaactagagaattgccaaatcatcgtcatgctcagttagccctatacgacgtcaatgctgacgcagttcggggtgtcgcagccgacttatctcatattgatacaggtgttactgtaacaggatatgaaggtgataggatcggcgaagcgttagaaggtacggatatcgtcctgatccctgcaggtgttcctagaaaacctggtatgacaagagaagatctattggttgttaatgcaaagattgtcaagagtatagggtcatcgattgcgcagcattgcgatttaaacaaagtgttcattctactaatctcaaacccaataaattcccttgttccagtactcgttaaggaactggaatctaaatctcaaggcactcaagttgagagacgtgtgcttggtctcactaagttggattccgttagagcaagtgcatttttgcacgaggttacgattaaacatggtctaaaacctaaatctaatactcttgatgatgttccagtagttggtggtcattctggtgaaactattgtacctttattctcccaagcccctaatggtaaccgtttatcacaggacgccttggaagctcttgttcagcgtgtacaattcggaggcgatgaagtcgttagagctaaaaatggtgctggtagtgccactctgtgtatggcccatgccgcttatactgttgctgcatcttttattccacttatcactggtcaaaagcgttccatctctggtacattctatgttgccttaaaggatgctcaaggtcagcctatcaacagtagcgctaagcgtcttttgggctcaatcaacgatttaccatattttgcagtgccattggagattacttctcagggtgtggatgaattagataccagcgttttggaaagaatgaccaagtatgagagagaaagactcttagctccttgtctgggtaaattggaaggtggtatcagaaacggtttgagtttgtaa

SEQ ID NO: 62 - DNA, 1017 bp, Kluyveromyces marxianus
  source 1..1017: mol_type = other DNA
atgcttagagccctaactcgccgtcaattttcctccactgccttcaacccatacaaggtcaccgttctaggtgctggtggtggtattggtcaaccattgtccttgttgttgaagctaaaccacaaggtcactgacttgagactatacgacttgaagggtgccaagggtgtcgctgctgacttgtctcacatcccaaccaactctaccgttactggttacactccagaatccaaggactctcaagaagaattggctgctgctttgaaggacactgaggttgttttgatcccagctggtgtgccaagaaagccaggtatgacccgtgacgatttgttcgccatcaatgccggtattgtcagagatttggccacttccatcgccaagaacgctccaaacgccgccatcttggtcatctccaacccagtcaactctactgtcccaatcgtcgccgaggtcttgaagcaaaacggcgtctacaacccaaagaagttgttcggtgtcaccactttggacgttatccgtgcctccagattcatctccgaggttagaggtaccgacccaaccactgagcacgtgaccgtcgtcggtggtcactccggtatcaccatcttgccgctagtgtcccagaccaagcacaagtccgtcatcaagggcgaggaattggacaacttgatccacagaatccaattcggtggtgacgaagtcgtccaggcaaagaacggtgctggttctgccactttgtccatggcccaagccggtgcccgtttcgctaacagcgttctaagcggtttcgaaggtgaaagagacgtcattgagccaactttcgtcgactccccattgttcaaggacgaaggtatcgaattcttcgcttccccagtcactttgggcccagaaggtgtcgaaaagatccacggtttgggtgtcttgtccgacaaggaagaacaaatgttggccacttgtaaggaaaccttgaagaagaacatcgaaaagggtcaaaactttgtcaagcaaaactaa

SEQ ID NO: 63 - DNA, 1017 bp, Kluyveromyces marxianus
  source 1..1017: mol_type = other DNA
atggttagcgttgcagtattaggatcatccggaggcattggccaaccactctcactcttgttgaagctggaccctcgcgtgtccagcttgagattgtacgacttgaagatgtcccacgggatcgccaccgatttgtcgcacatggactccaactccatctgcgagggcttcaacaccgacgagatcgcgctcgcgctcaagggcgcccagatcgtcgtcatccccgcgggtgtcccaagaaagcccgggatgtcacgtgacgaccttttcaagatcaacgccaagatcatcaagtcgttggcgttgcaaatagccgagcacgcgcccgaggcgcgcgtcctcgtgatctcgaacccggtcaactccttggtgcccattgtgtacgagactttgaagagcgtcggcaagttcgagccgggtaaagtgatgggaattaccacattggacattatccgctcacacacgttcctggtggacgtcttgggccgcaaggcgtacagcgtcgagaagttgcgcagcgcggttactgtggtgggcggccactcgggcgagaccattgttccgattttcaccgaccagaagttctacaggcgtctcagagacagagagctctatgacgcgtacgtgcatagggtccaattcggcggagacgaggtcgtgaaggccaaggacggcagcggtagtgctactttgtctatggcctgggcgggttacagttttgtgaagcagttgctcaacagcttgcacctagaaacaggcgaagacgtgcatccgatcccaacgtttgtgtacttgccgggtttaccgggcgggaaggagctccagcagaagttgggcacctctgttgagttttttgccgcgcccgtgaagctttccaagggtattgtggttgaagttgagcacgactgggtcgacaagttgaacgatgccgagaagaagttgattgcaaagtgtcttccaatccttgacaagaacatcaagaagggtctcgccttttcgcagcagacaaagttgtga

SEQ ID NO: 64 - DNA, 1125 bp, Kluyveromyces marxianus
  source 1..1125: mol_type = other DNA
atgccagcagtatcatatgatgtccagcaacgggatatcctcaagatcgcagttctaggggcggcaggcggtattggccaatccttgtcgctcttgttgaagtcgaacgcttcttttttgttaccacgtgactcgtcaagacacataagcctagcgctatacgacgtgaacaaagatgccatcgtgggcacagcagcagacttgtcacacatagacacccctatcaccaccactccacactacccaaatgatgggaatggcggtatcgcacggtgcttgcaagatgcagacatggtcatcatcccagcaggtgtgcccagaaaacccggtatgtcacgtgatgacctaatcggtgtcaacgccaagatcatcaagtcgctaggaaacgacatcgcagagtactgtgacttgtctaaagtgcatgtattggttatttcgaacccagtgaactcgttggtcccactgatggtgtcgactttggcaaacagcccacacagtgcgaacacaaacatcgaggctagagtgtacgggatcacccatttggacctagtgagagcttccacctttgtgcaacagctaaactctttcaaatcaaataacgcacctgacattccggtcattggtggtcattccggagataccatcatccccgttttttccgtcttgaatcaccgcgcttctaactccggatacgctaatttgctagataatggcgttaggcaaaagttggtccacagagttcaatatggtggggacgaaatcgtccaagcaaagaacggtaacgggagcgcgacattatccatggcatacgcgggcttcaaaatcgcagcacaattcatcgaccttttggtcggaaatatccgcactatcgaaaatatttgcatgtatgttccgctcactaacaggtataataccgagatcgccccaggctctgacgaattaagatcaaagtacatcaacggaaccctttatttctcgattccactttccatcggaataaacggtatcgaaagagtccactacgagatcatggaacatctagacagctacgagcgtgagacgctactaccgatctgcttggaaactctaaagggtaatattgacaagggtctaagcttggtataa

SEQ ID NO: 65 - DNA, 22 bp, synthetic construct
  source 1..22: mol_type = other DNA; note = Synthetic - Primer
ggaggaatggaacagtgatgac

SEQ ID NO: 66 - DNA, 939 bp, Escherichia coli
  source 1..939: mol_type = other DNA
atgaaagtcgcagtcctcggcgctgctggcggtattggccaggcgcttgcactactgttaaaaacccaactgccttcaggttcagaactctctctgtatgatatcgctccagtgactcccggtgtggctgtcgatctgagccatatccctactgctgtgaaaatcaaaggtttttctggtgaagatgcgactccggcgctggaaggcgcagatgtcgttcttatctctgcaggcgtagcgcgtaaaccgggtatggatcgttccgacctgtttaacgttaacgccggcatcgtgaaaaacctggtacagcaagttgcgaaaacctgcccgaaagcgtgcattggtattatcactaacccggttaacaccacagttgcaattgctgctgaagtgctgaaaaaagccggtgtttatgacaaaaacaaactgttcggcgttaccacgctggatatcattcgttccaacacctttgttgcggaactgaaaggcaaacagccaggcgaagttgaagtgccggttattggcggtcactctggtgttaccattctgccgctgctgtcacaggttcctggcgttagttttaccgagcaggaagtggctgatctgaccaaacgcatccagaacgcgggtactgaagtggttgaagcgaaggccggtggcgggtctgcaaccctgtctatgggccaggcagctgcacgttttggtctgtctctggttcgtgcactgcagggcgaacaaggcgttgtcgaatgtgcctacgttgaaggcgacggtcagtacgcccgtttcttctctcaaccgctgctgctgggtaaaaacggcgtggaagagcgtaaatctatcggtaccctgagcgcatttgaacagaacgcgctggaaggtatgctggatacgctgaagaaagatatcgccctgggcgaagagttcgttaataagtaa

SEQ ID NO: 67 - DNA, 20 bp, synthetic construct
  source 1..20: mol_type = other DNA; note = Synthetic - Primer
cacagaggtgcagtaacgag

SEQ ID NO: 68 - DNA, 1014 bp, Rhizopus delemar
  source 1..1014: mol_type = other DNA
atgtttgccgcctctcgtgttttctctattgctgccaagcgttctttctctacttctgctgctaatctttccaaggttgccgttcttggcgctgctggtggtattggtcaacccttgtctttgttgttgaaggaaaaccctcacgtcacccacctttctctttatgatattgtcaacactcctggtgtcgctgccgatcttagccacatcaacaccaactccaaggtcactggccacacccctgaaaacgatggtttgaagactgctcttgaaggtgctcacgttgttgttattcctgctggcgttcctcgtaagcctggtatgacccgtgatgatttattcaacaccaatgcttccattgttcgtgaccttgctgaagctgctgccaagcactgtcccgacgctcatttccttatcatctccaaccctgtcaactccactgttcccatctttgccgaaaccttaaagaaggctggtgtcttcaaccctaagcgtttgtatggtgtcaccactcttgatgtcgtccgtgcctctcgcttcgttgccgaagtcaagaacttggaccccaacgatgtcaaggttaccgttgtcggtggtcactctggtgtgactattgtccctctcctctctcaaaccggtctcgaattcagcaaggaagaactcgatgccttgacccaccgtatccaattcggtggtgatgaagtcgttcaagccaagaatggtactggttctgtcactctctccatggcctttgccggtgctcgtttcgccaactctgtcttggaagccactgttggtggtaagaagggtgttgttgaaccctcctttgtcaagtctgatgtctttgccaaggatggtgttgaatatttctctaccaacattgaacttggtcctgaaggtgttgaaaagatcaacgaactcggtcaaatctctgactatgaaaaggaacttattgctaaggccgttcctgaattaaagaagaacattgccaagggtaacagctttgttcaataa

SEQ ID NO: 69 - DNA, 31 bp, synthetic construct
  source 1..31: mol_type = other DNA; note = Synthetic - Primer
caagagtatcccatctgacaggaaccgatgg

SEQ ID NO: 70 - DNA, 1458 bp, Issatchenkia orientalis
  source 1..1458: mol_type = other DNA
atgttagctgctagatcattaaaggcaagaatgtcaacaagagctttctcaactacctcaattgcaaaaagaatcgaaaaagatgcatttggtgacattgaagtcccaaatgagaaatattggggtgctcaaactcaaagatctttacaaaatttcaaaattggtggtaagagagaagttatgccagaaccaatcatcaaatcttttggtattttaaagaaggctactgctaagatcaatgctgagtctggtgctttagacccaaagttatctgaagccatccaacaagctgcaaccgaagtttatgaaggtaaactaatggaccatttcccattagttgtctttcaaaccggttctggtactcaatctaacatgaatgccaatgaagtcatctctaatagagcaattgaaatcttgggtggtgaattaggctctaaaactccagtccatcctaatgatcatgttaatatgtcccaatcttctaatgatactttccctactgtcatgcatattgcagcagttacagaagtttcatcccatttattaccagaattaactgcactaagagatgcattgcaaaagaaatccgatgaatttaagaatattatcaaaatcggtagaacccatttacaagatgcaactcctttaactttaggtcaagaattttctggttatgttcaacaatgtactaatggtatcaaaagaatcgaaattgctcttgaacatttgagatacttagctcaaggtggtactgccgttggtactggtcttaacaccaagaaaggttttgctgaaaaggttgcaaatgaagtcactaaattgactggtttacaattctataccgctccaaataaattcgaagcccttgcagctcacgatgctgttgttgaaatgtctggtgctttgaataccgttgcagtctcattattcaaaatcgctcaagatatcagatatttgggttccggcccaagatgtggttatggtgaattggctttaccagaaaatgaaccaggttcttccatcatgccgggtaaagttaacccaactcaaaacgaagctttgactatgctttgtacccaagtctttggtaaccactcttgtattacctttgcaggtgcttcaggtcaattcgaattgaatgtctttaagccagttatgatctccaacttgttatcttctattaggttattaggtgatggttgtaattcttttagaatccactgtgttgaaggtatcattgcaaataccgacaagattgataaattactacatgaatctctcatgttagttactgctttgaacccacacattggttacgataaggcttccaagattgcaaagaatgcacacaagaagggcttgacattgaaacaatctgcattggaattaggttacttgaccgaagaacaattcaatgaatgggttagaccagaaaacatgattggtccaaaggattaa

SEQ ID NO: 71 - DNA, 27 bp, synthetic construct
  source 1..27: mol_type = other DNA; note = Synthetic - Primer
catcactgttaaaggaatgggtaaatc

SEQ ID NO: 72 - DNA, 27 bp, synthetic construct
  source 1..27: mol_type = other DNA; note = Synthetic - Primer
gctggagaatagatcttcaacgccccg

SEQ ID NO: 73 - DNA, 30 bp, synthetic construct
  source 1..30: mol_type = other DNA; note = Synthetic - Primer
gagaacttatacgcaccagaacgccttttg

SEQ ID NO: 74 - DNA, 31 bp, synthetic construct; identical to SEQ ID NO: 69
  source 1..31: mol_type = other DNA; note = Synthetic - Primer

SEQ ID NO: 75 - DNA, 1413 bp, Saccharomyces cerevisiae
  source 1..1413: mol_type = other DNA
atgtctctctctcccgttgttgttattggaaccggtttggccgggctggctgctgccaacgaattggttaacaagtataacatccctgtaaccatcctcgaaaaggcttcctcgatcggtgggaactctatcaaggcctccagtggtattaacggtgcttgcaccgagactcaacgtcacttccacatcgaggactccccacgcttatttgaagatgacaccatcaagtctgctaaaggtaaaggtgtccaagagttaatggctaagttggccaatgattctcccctggctattgaatggttgaaaaacgaatttgatttgaaattggacctattggctcaattgggtggccactctgtggcaagaactcacagatcgtctgggaagttgcctccaggtttcgaaattgtttctgccttatctaacaatttgaagaaattagctgagactaaaccagagttagttaagattaacttagacagtaaagtcgtagacatccatgaaaaggatggctccatttctgctgtagtgtacgaggataagaatggcgaaaagcacatggtgagtgctaacgatgtcgttttttgttctggagggtttggcttttctaaggaaatgcttaaagaatatgcacccgaactggtgaacttgccaacgacaaacgggcaacaaacaactggtgatggtcaaaggcttctgcagaagttaggcgctgatctgattgacatggaccaaattcaagttcatccaactgggttcattgatccaaatgaccgtagctcaagctggaaattcttggctgccgaatccttaagaggtcttggtggtatcctattaaaccctattaccggtagaagatttgtcaacgaattgaccacaagagatgtagtcactgcagctattcaaaaggtttgtcctcaagaggataacagagcactattggttatgggcgaaaaaatgtacacagatttgaagaataatttagatttttacatgttcaagaaacttgtacagaaattgacattatctcaagttgtgtctgaatataatttaccaatcactgtcacccaattatgcgaggaattgcaaacatactcttcgttcactaccaaggctgatccgttgggacgtaccgttattctcaacgaatttggctctgacgttactccagaaaccgtggtttttattggtgaagtaacaccggttgtccatttcaccatgggtggtgctagaatcaatgtcaaggctcaagtcattggcaagaacgacgaaaggctactaaaaggcctgtacgcggccggtgaagtttctggcggtgttcatggcgccaataggttgggtggttcaagtttgttagaatgcgttgtctttgggagaactgcagctgaatctattgccaatgaccgcaagtaa

SEQ ID NO: 76 - DNA, 1413 bp, Saccharomyces mikatae
  source 1..1413: mol_type = other DNA
atgtcatcttctccagttgtcgttattggtacaggcttggcaggtttggcaactgctaatgagttagtcaataagtacaacattcctgttaccattttggaaaaggcatcctctatcggtggcaattccattaaggcatcttctggtatcaatggtgcatgtacagaaacccaacgtcattttcacattgaagatactcctagactttttgaagatgatactgttcaatccgccaagggcaaaggtgttcaagagttaatgggtaaacttgctaatgattctccacttgctattgaatggttaaagactgaattcgacttaaagttagaccttttggctcagttaggtggtcactctgttgctagaactcatagatcttccggtaaacttccaccaggtttcgaaatcgtttccgccttatccaataacttgaaaaagttggcagaaaccaagccagagttagttaagattaacttagactcaaaggtcgttgacatccacaaaaaggacggctctatttccgcaattgtctatgatgacaaaaacggtgaaagacataccttatccacttcaaatgttgttttctgctctggtggtttcggtttttctaaggaaatgttaaacgagtatgctccacaattggtcaacttgccaaccactaacggtcagcaaacaacaggtgacggccaaagattgttacaaaagcttggtgcagatttgattgatatggatcaaattcaagtccatcctactggtttcatcgacccaaacgatagaaactcctcttggaagtttttggctgctgaatctttaagaggtttgggtggtatcttattgaatccaattactggtcgtagatttgtcaacgaattgaccactagagatgtcgttactgaagcaatccagaagcactgtccacaagatgataacagagctttgttagttatgtccgaaaagatgtatacagatttgaaaaacaatttggacttctacatgttcaaaaagttagttcaaaagttatctttgtcccaagttgtttccgagtataagttaccaattactgtttcccaattgtgtcaggaattacaaacctactcatcttttacttcaaaagccgatcctcttggtagaaccgttgtcttaaacgaattcggtgctgacatcaccccagaaacaatggttttcatcggcgaagttaccccagtcgttcactttaccatgggtggtgctagaatcaatgttaaggctcaagttatcggcaaaaacgatgagcctttgttaaacggtttgtacgcagcaggtgaagtttctggtggtgtccatggtgccaatagattaggtggttcatctttgcttgaatgtgtcgtttttggtagaactgcagcagaatcaattgccaataaccacaagtaa

SEQ ID NO: 77 - DNA, 1413 bp, Kluyveromyces polysporus
  source 1..1413: mol_type = other DNA
atgtcaaccaaaaagccagtcgtcatcattggtactggtttagccggtttgtctgctggtaatcaattggtcaatatgcataaagttcctatcattatgttggacaaggcatcctccattggtggtaattctacaaaggcttcctctggtatcaacggtgcttctactattactcaacagcaacttaatgttaaagactctcctgacttattccttcaagatactgttaagtctgctaagggtagaggtattgagtcccttatgaaaaagttatcacaagactccaactctgctatccattggttgcaacaggattttgatttgaagttggatttgttagctcaattgggtggtcattccgttcctagaacacaccgttcctcaggcaagttacctccaggcttcgaaattgtccaagctttatctaacaagttaaaggctatttctgagtccgatccagaattcgttagaatcttacttaactccaaggttgttgatgtttccgttaacaatgagggcaaggtcgaatctattgactatgttgatgcagaaggtaaacatcacaaaatcgctactgataacgttgtcttttgttccggtggtttcggtcactcagcagaaatgttgaacaagtatgcaccagaattagctaacttgccaactactaacggtcaacaaaccactggcgatggtcagagaatcttggagaaattgggtgcagacttgattgatatgtcccaaattcaagttcacccaacaggtttcatcgatccagcaaacagagattctaagtggaagtttttggctgccgaagcattaagaggtttaggtggtatcttacttaatccatctaccggcaagagattcgttaatgagttaaccaccagagatttggtcacagaagctatccaatcacaatgtccaagagatgacaataaggcattccttgttatgtctgaaaaggtctatgagaattacaaaaacaacatggacttttacttattcaaaaagttagtttccaagatgaccattaaggaatttgtcgaaacttacaagttgccaatttctgccgacgccgttacccaagacttaatcgactattcagttgataagaccgataagtttggtagaccattggttatcaacgtttttgatgaaaagttgaccgaagattccgaaatctatgttggtgaagttacaccagttgtccatttcactatgggtggtgcaaagatcaatactgaatctcaagttatcaacaaaaacggtcaagttttggcaaagggtatctacgcagcaggtgaagtctccggtggtgttcacggttctaatagattaggtggttcatctttgttagaatgcgtcgtttacggtagatctgctgcagataacattgccaaaaacattgaataa

SEQ ID NO: 78 - DNA, 1464 bp, Kluyveromyces marxianus
  source 1..1464: mol_type = other DNA
atgttgcacagatacatccgtttgttctccttctgcgtcatcttgtacttagtctatttgttacttactaaggagtcaaacgtcatgtctaagcctgttgttgttattggttctggtttagcaggcttaacaacatcttcacaattagcaaagtttaacattccaatcgtccttttagaaaagacatcttccattggtggtaattccattaaggcatcttctggtatcaatggcgcaggcaccgaaactcaatctcgtttacacgttgaagatcacccagaattgtttgctgatgataccattaagtctgcaaaaggtaaaggtgttgtcgctttgatggaaaagttatctaaagactcctctgatgctatttcctggttacaaaacgacttcaagattcctttggataagttagctcaattaggcggtcattccgttcctagaacccatagatcatccggcaagcttccaccaggtttccaaattgtcgataccttgaaaaaggccttggagtcttatgactctaaagcagttaagatccaattgaattctaaggtcgttgatgttaagcttgattccaataacagagtttcatctgttgttttcgaagatcaagatggtactcacaccattgaaaccaacaacgtcgttttctgtactggtggtttcggtttcaacaaaaagttattggagaagtatgcaccacacttggtcgacttgccaactaccaacggtgagcaaaccttaggtgaaggtcaggtcttattggaaaaacttggtgctaagttgattgatatggaccaaattcaagttcatccaactggctttatcgatccagccaatccagattctaattggaagtttttggctgccgaggccttaagaggtttaggtggtgtcttgatcaatccacacactggtcagagatttgttaacgaattgacaactagagacatggtcaccgaagctatccagtctaagtccgaatccaagactgcttacttggttatgtccgagtccttatacgagaactacaagccaaacatggacttctatatgttcaaaaagcttgtttccaaaaagaccgttgctgaatttgctgaagatttgccagtttctgttgaccaacttattgcagaactttcaacttattccgacttgtctaaggatgatcatttgggtagaaagtttagagaaaacacttttggttcctcattatcatcagactcaaccattttcgttggcaagattactcctgttgttcacttcacaatgggtggtgcaaagattgatgaacaagctagagtcttgaatgcagaaggtaaaccattagctactggtatctacgccgctggtgaagtttctggtggtgtccatggtgctaatagattaggtggttcctctttgttagaatgtgttgtctttggtagacaagcagcaaaatccattagagcaaacttgtaa

SEQ ID NO: 79 - DNA, 29 bp, synthetic construct
  source 1..29: mol_type = other DNA; note = Synthetic - Primer
gaggaagttcaaagtatgaaagacgtcag

SEQ ID NO: 80 - DNA, 33 bp, synthetic construct
  source 1..33: mol_type = other DNA; note = Synthetic - Primer
gatcgggcccgtcttggaagacgcactagtctc

SEQ ID NO: 81 - DNA, 20 bp, synthetic construct
  source 1..20: mol_type = other DNA; note = Synthetic - Primer
tgacaggaaccgatggactc

SEQ ID NO: 82 - protein, 1144 aa, Leishmania mexicana
MADGKTSASVVAVDAERAAKERDAAARAMLQGGGVSPAGKAQLLKKGLVHTVPYTLKVVVADPKEMEKATADAEEVLQAAFQVVDTLLNNFNENSEVSRVNRLAVGEEHQMSETLKHVMACCQKVYHSSRGVFDPAVGPLVRELREAAHKGKTVPAERVNDLLSKCTLNASFSIDMSRGMIARKHPDAMLDLGGVNKGYGIDYIVEHLNSLGYDDVFFEWGGDVRASGKNQLSQPWAVGIVRPPALADIRTVVPEDKRSFIRVVRLNNEAIATSGDYENLVEGPGSKVYSSTFNPTSKNLLEPTEAGMAQVSVKCCSCIYADALATAALLKNDPAAVRRILDNWRYVRDTVTDYTTYTREGERVAKMLEIATEDAEMRAKRIKGSLPARVIIVGGGLAGCSAAIEAANCGAHVILLEKEPKLGGNSAKATSGINAWGTRAQAKQGVMDGGKFFERDTHRSGKGGNCDPCLVKTLSVKSSDAVKWLSELGVPLTVLSQLGGASRKRCHRAPDKSDGTPVPVGFTIMKTLENHIVNDLSRHVTVMTGITVTALESTSRVRPDGVLVKHVTGVHLIQASGQSMVLNADAVILATGGFSNDHTPNSLLQQYAPQLSSFPTTNGVWATGDGVKMASKLGVALVDMDKVQLHPTGLLDPKDPSNRTKYLGPEALRGSGGVLLNKNGERFVNELDLRSVVSQAIIAQDNEYPGSGGSKFAYCVLNETAAKLFGKNFLGFYWNRLGLFQKVDSVAGLAKLIGCPEANVVATLKQYEELSSKKLNPCPLTGKSVFPCVLGTQGPYYVALVTPSIHYTMGGCLISPSAEMQTIDNSGVTPVRRPILGLFGAGEVTGGVHGGNRLGGNSLLECVVFGKIAGDRAATILQKKNTGLSMTEWSTVVLREVREGGVYGAGSRVLRFNMPGALQRTGLALGQFIGIRGDWDGHRLIGYYSPITLPDDVGVIGILARADKGRLAEWISALQPGDAVEMKACGGLIIDRRFAERHFFFRGHKIRKLALIGGGTGVAPMLQIVRAAVKKPFVDSIESIQFIYAAEDVSELTYRTLLESYEEEYGSEKFKCHFVLNNPPAQWTDGVGFVDTALLRSAVQAPSNDLLVAICGPPIMQRAVKGALKGLGYNMNLVRTVDETEPPS

SEQ ID NO: 83 - protein, 743 aa, Saccharomyces cerevisiae
MDGPNFAHQGGRSQRTTELYSCARCRKLKKKCGKQIPTCANCDKNGAHCSYPGRAPRRTKKELADAMLRGEYVPVKRNKKVGKSPLSTKSMPNSSSPLSANGAITPGFSPYENDDAHKMKQLKPSDPINLVMGASPNSSEGVSSLISVLTSLNDNSNPSSHLSSNENSMIPSRSLPASVQQSSTTSSFGGYNTPSPLISSHVPANAQAVPLQNNNRNTSNGDNGSNVNHDNNNGSTNTPQLSLTPYANNSAPNGKFDSVPVDASSIEFETMSCCFKGGRTTSWVREDGSFKSIDRSLLDRFIAAYFKHNHRLFPMIDKIAFLNDAATITDFERLYDNKNYPDSFVFKVYMIMAIGCTTLQRAGMVSQDEECLSEHLAFLAMKKFRSVIILQDIETVRCLLLLGIYSFFEPKGSSSWTISGIIMRLTIGLGLNRELTAKKLKSMSALEAEARYRVFWSAYCFERLVCTSLGRISGIDDEDITVPLPRALYVDERDDLEMTKLMISLRKMGGRIYKQVHSVSAGRQKLTIEQKQEIISGLRKELDEIYSRESERRKLKKSQMDQVERENNSTTNVISFHSSEIWLAMRYSQLQILLYRPSALMPKPPIDSLSTLGEFCLQAWKHTYTLYKKRLLPLNWITLFRTLTICNTILYCLCQWSIDLIESKIEIQQCVEILRHFGERWIFAMRCADVFQNISNTILDISLSHGKVPNMDQLTRELFGASDSYQDILDENNVDVSWVDKLV

SEQ ID NO: 84 - DNA, 17 bp, synthetic construct
  source 1..17: mol_type = other DNA; note = Synthetic - Primer
caatccaaccgccaccg

SEQ ID NO: 85 - DNA, 47 bp, synthetic construct; identical to SEQ ID NO: 54
  source 1..47: mol_type = other DNA; note = Synthetic - Primer

SEQ ID NO: 86 - DNA, 21 bp, synthetic construct
  source 1..21: mol_type = other DNA; note = Synthetic - Primer
ctttcattaggttggttgaag

SEQ ID NO: 87 - DNA, 1521 bp, Issatchenkia orientalis
  source 1..1521: mol_type = other DNA
atgggtgtccagtttatcgaaaataccattatcgttgtctttggtgcgtctggagatttagccaagaagaagactttccccgccctgtttggactattcagggagggccagctctcagaaacaaccaaaatcattgggtttgctcgatcaaaactatcaaatgatgacttgaggaacagaataaagccgtacttgaaattgaacaagagaacagatgctgaaaggcagtctctggagaagtttctgcagattctcgagtatcaccagtcaaactacgacgacagtgaaggttttgaaaaattggagaagctaatcaataagtacgacgatgaggcaaacgtgaaagagtctcacaggttgtactatttggctttaccaccgtctgtctttacaaccgttgcaacaatgttgaaaaaacattgtcatccaggtgattctggtattgctaggctaattgtcgagaaaccctttggccatgacttgagctcgtcccgtgagctacaaaagtctttagctccactttggaatgaagatgaattgtttagaattgatcattatttgggcaaagaaatggttaagaatttaattcctttgaggttttcaaatacgtttttgagcagttcttggaacaatcaatttattgacaccatccaaatcacttttaaggagaactttggaactgaaggacgtggtggttactttgattccattggtataataagagatgttatccaaaatcatttgttacaagtcttgactattgttttgatggaaaaaccagcggattttaatggagaatctatcagagatgaaaaggttaaagtgttaaaggcaattgaacaaattgatttcaataatgtgttggtaggtcaatatgataaatctgaagatggtagtaaacctggttacttggatgatgataccgtcaatccagattctaaagctgtcacttatgctgccttagttttaaatgtggcaaacgaaagatggaataatgttccgatcattctaaaggcaggcaaggccttgaatcaatccaaggtggaaattagaatccagttcaaaccagtagaaaatggaatcttcaaaaactctgctaggaatgagttggttattaggatccaaccaaacgaggcaatgtatttgaaaatgaacatcaaagtacctggtgtttccaatcaagtgtcgatttcagaaatggatttgacttacaagaataggtattcctccgaattttacattccagaagcttatgaatctttgattaaagatgccttaatggatgatcattcaaattttgttagagacgatgaattggacatttcatgggctttgttcactccattactagaacatatcgaaggccccgatggtccaactccaaccaagtatccttacggttccagaggtccaaaggagattgacgaatttttgagaaaccatggttatgtaaaggaaccaagagaaaattaccaatggccattaactactcctaaagaattgaacagttcaaagttttaa

SEQ ID NO: 88 - protein, 477 aa, Issatchenkia orientalis
MGQNLILNAADHGFTVVAYNRTVSKVDHFLQNEAKGKSIIGAHSIEELCAKLKKPRRIMLLVKAGNPVDQFIEQLLPHLDEGDIIIDGGNSHFPDSNRRYEELKKKGILFVGSGVSGGEEGARYGPSLMPGGAKEAWPHIKDIFQSISAKADGEPCCDWVGDAGAGHYVKMVHNGIEYGDMQLICEAYDLMKRVGGLTDKEISDVFGEWNEGVLDSFLVEITRDILAFNDKDGTPLVEKILDTAGQKGTGKWTAINALDLGMPVTLIGEAVFARCLSALKPERERASEILNGPEVEQVSAEGRAQFIADLMQALYASKIISYAQGFMLIREAAKEYNWKLNFPSIALMWRGGCIIRSVFLAEITAAYRENPDLENLLFNKFFQDAIHKAQSGWRKTVALAVTQGIPTPAFSTALSFYDGYRSKKLPANLLQAQRDYFGAHTFQILPECADDEKKVGDYIHVNWTGKGGNVSASTYDA

SEQ ID NO: 89 - DNA, 1434 bp, Issatchenkia orientalis
  source 1..1434: mol_type = other DNA
atgggtcaaaacttgattcttaatgcagcagatcatggttttactgttgttgcatacaacagaactgtctctaaagttgaccatttccttcaaaatgaagcaaagggtaaatccattattggtgcacactccattgaagaattatgtgctaagttgaagaaaccaagaagaattatgttgttagtcaaggcaggtaatccagttgatcaattcattgaacaattgttacctcatttagatgaaggcgatatcattattgacggtggtaactctcacttccctgactccaacagaagatacgaggaattaaagaagaagggtattctctttgtcggttctggtgtttctggtggtgaagaaggtgcaagatatggtccttctttgatgcctggtggtgcaaaggaagcatggcctcatattaaggacatcttccaatctatctctgcaaaggccgatggtgagccatgttgtgattgggttggtgatgcaggtgcaggtcattacgttaagatggtccacaatggtatcgagtatggtgatatgcagttgatctgtgaagcttacgatttgatgaagagagttggtggtttaactgacaaggaaatatctgatgttttcggtgaatggaacgagggtgttctcgattctttcttagttgaaattaccagagatatcttagctttcaacgataaggatggtaccccattagttgaaaagatcttagatactgccggacagaagggtactggtaaatggactgcaataaatgctttagacttgggtatgccagtcactttaattggtgaagctgtttttgcgagatgtttatccgctttgaagccagaaagagagagagcttctgaaatcttaaacggtccggaagttgaacaagtttctgctgaaggtagagcacaatttattgcagatttgatgcaagctttatatgcatcaaagattatttcttacgcacaaggtttcatgttaatcagagaagcagcaaaggaatacaactggaaattaaacttcccttctattgcacttatgtggagaggtggttgtattatcaggtctgttttcttggctgaaattactgcagcttatagggaaaaccctgacttagagaacttactattcaacaagttcttccaagatgctattcataaggcacagtctggttggagaaagactgttgcattagctgttacccaaggtattccaactccagcattctctactgcattgtctttctacgatggttacagatccaagaagttaccagctaacttgttgcaagcacaaagagattacttcggtgctcacactttccaaattttacctgaatgtgcagatgacgaaaagaaggttggtgattacatccatgtcaactggactggtaagggtggtaatgtttctgctagtacttacgatgcttaa

SEQ ID NO: 90 - protein, 438 aa, Schizosaccharomyces pombe
MGELKEILKQRYHELLDWNVKAPHVPLSQRLKHFTWSWFACTMATGGVGLIIGSFPFRFYGLNTIGKIVYILQIFLFSLFGSCMLFRFIKYPSTIKDSWNHHLEKLFIATCLLSISTFIDMLAIYAYPDTGEWMVWVIRILYYIYVAVSFIYCVMAFFTIFNNHVYTIETASPAWILPIFPPMICGVIAGAVNSTQPAHQLKNMVIFGILFQGLGFWVYLLLFAVNVLRFFTVGLAKPQDRPGMFMFVGPPAFSGLALINIARGAMGSRPYIFVGANSSEYLGFVSTFMAIFIWGLAAWCYCLAMVSFLAGFFTRAPLKFACGWFAFIFPNVGFVNCTIEIGKMIDSKAFQMFGHIIGVILCIQWILLMYLMVRAFLVNDLCYPGKDEDAHPPPKPNTGVLNPTFPPEKAPASLEKVDTHVTSTGGESDPPSSEHESV

SEQ ID NO: 91 - protein, 398 aa, Aspergillus oryzae
MFNNEHHIPPGSSHSDIEMLTPPKFEDEKQLGPVGIRERLRHFTWAWYTLTMSGGGLAVLIISQPFGFRGLREIGIAVYILNLILFALVCSTMAIRFILHGNLLESLRHDREGLFFPTFWLSVATIICGLSRYFGEESNESFQLALEALFWIYCVCTLLVAIIQYSFVFSSHKYGLQTMMPSWILPAFPIMLSGTIASVIGEQQPARAALPIIGAGVTFQGLGFSISFMMYAHYIGRLMESGLPHSDHRPGMFICVGPPAFTALALVGMSKGLPEDFKLLHDAHALEDGRIIELLAISAGVFLWALSLWFFCIAIVAVIRSPPEAFHLNWWAMVFPNTGFTLATITLGKALNSNGVKGVGSAMSICIVCMYIFVFVNNVRAVIRKDIMYPGKDEDVSD

SEQ ID NO: 92 - DNA, 1317 bp, Schizosaccharomyces pombe; identical to SEQ ID NO: 6
  source 1..1317: mol_type = other DNA

SEQ ID NO: 93 - DNA, 1205 bp, Aspergillus oryzae
  source 1..1205: mol_type = other DNA
atgtttaacaatgagcaccatattcctcctggttcctctcactctgatatcgaaatgttaacaccaccaaagtttgaggatgaaaaacagttaggtccagtcggtattagagaaagattgagacatttcacttgggcttggtataccttaaccatgtccggtggtggtttggcagttttgattatctctcagccattcggttttagaggtttaagagaaattggtattgcagtttacattttgaacttaatcttattcgctttggtttgttctaccatggctattcgtttcatcttgcacggtaaccttttggaatcccttagacatgacagagaaggtttgtttttccctactttctggttgtctgttgctaccatcatttgtggtttgtcaagatactttggtgaggaatccaacgaatccttccaattggcattagaagccttgttctggatctattgcgtttgtaccttgttggttgcaatcattcaatactcttttgttttctcatcccacaagtacggtttacaaacaatgatgccatcttggattttgccagcctttcctatcatgttgtcaggcacaattgcatctgttatcggtgaacaacaaccagccagagctgcattaccaatcattggtgccggtgtcaccttccaaggtttaggtttttctatttccttcatgatgtatgctcattacattggcagacttatggaatccggtttacctcactccgaccatagaccaggcatgttcatctgtgttggcccaccagcctttactgctttggctttagtcggtatgtccaagggtttaccagaagatttcaagcttttacatgacgctcatgcattagaggatggtagaatcattgaattgttagcaatttcagcaggtgttttcctttgggcattatccctttggtttttctgtattgctattgtcgctgtcattagatctccaccagaagctttccacttgaactggtgggctatggttttcccaaatactggtttcaccttagctactatcactttgggtaaagctttgaactcaaatggtgtcaagggtgtcggttctgcaatgtccatttgtattgtctgcatgtacatctttgttttcgttaacaatgttagagctgttattcgtaaggatatcatgtatccaggcaaagatgaggatgtttctgattaacctgcagg

SEQ ID NO: 94 - protein, 1180 aa, Issatchenkia orientalis
MSTVEDHSSLHKLRKESEILSNANKILVANRGEIPIRIFRSAHELSMHTVAIYSHEDRLSMHRLKADEAYAIGKTGQYSPVQAYLQIDEIIKIAKEHDVSMIHPGYGFLSENSEFAKKVEESGMIWVGPPAEVIDSVGDKVSARNLAIKCDVPVVPGTDGPIEDIEQAKQFVEQYGYPVIIKAAFGGGGRGMRVVREGDDIVDAFQRASSEAKSAFGNGTCFIERFLDKPKHIEVQLLADNYGNTIHLFERDCSVQRRHQKVVEIAPAKTLPVEVRNAILKDAVTLAKTANYRNAGTAEFLVDSQNRHYFIEINPRIQVEHTITEEITGVDIVAAQIQIAAGASLEQLGLLQNKITTRGFAIQCRITTEDPAKNFAPDTGKIEVYRSAGGNGVRLDGGNGFAGAVISPHYDSMLVKCSTSGSNYEIARRKMIRALVEFRIRGVKTNIPFLLALLTHPVFISGDCWTTFIDDTPSLFEMVSSKNRAQKLLAYIGDLCVNGSSIKGQIGFPKLNKEAEIPDLLDPNDEVIDVSKPSTNGLRPYLLKYGPDAFSKKVREFDGCMIMDTTWRDAHQSLLATRVRTIDLLRIAPTTSHALQNAFALECWGGATFDVAMRFLYEDPWERLRQLRKAVPNIPFQMLLRGANGVAYSSLPDNAIDHFVKQAKDNGVDIFRVFDALNDLEQLKVGVDAVKKAGGVVEATVCYSGDMLIPGKKYNLDYYLETVGKIVEMGTHILGIKDMAGTLKPKAAKLLIGSIRSKYPDLVIHVHTHDSAGTGISTYVACALAGADIVDCAINSMSGLTSQPSMSAFIAALDGDIETGVPEHFARQLDAYWAEMRLLYSCFEADLKGPDPEVYKHEIPGGQLTNLIFQAQQVGLGEQWEETKKKYEDANMLLGDIVKVTPTSKVVGDLAQFMVSNKLEKEDVEKLANELDFPDSVLDFFEGLMGTPYGGFPEPLRTNVISGKRRKLKGRPGLELEPFNLEEIRENLVSRFGPGITECDVASYNMYPKVYEQYRKVVEKYGDLSVLPTKAFLAPPTIGEEVHVEIEQGKTLIIKLLAISDLSKSHGTREVYFELNGEMRKVTIEDKTAAIETVTRAKADGHNPNEVGAPMAGVVVEVRVKHGTEVKKGDPLAVLSAMKMEMVISAPVSGRVGEVFVNEGDSVDMGDLLVKIAKDEAPAA

SEQ ID NO: 95 - protein, 1178 aa, Saccharomyces cerevisiae
MSQRKFAGLRDNFNLLGEKNKILVANRGEIPIRIFRTAHELSMQTVAIYSHEDRLSTHKQKADEAYVIGEVGQYTPVGAYLAIDEIISIAQKHQVDFIHPGYGFLSENSEFADKVVKAGITWIGPPAEVIDSVGDKVSARNLAAKANVPTVPGTPGPIETVEEALDFVNEYGYPVIIKAAFGGGGRGMRVVREGDDVADAFQRATSEARTAFGNGTCFVERFLDKPKHIEVQLLADNHGNVVHLFERDCSVQRRHQKVVEVAPAKTLPREVRDAILTDAVKLAKECGYRNAGTAEFLVDNQNRHYFIEINPRIQVEHTITEEITGIDIVAAQIQIAAGASLPQLGLFQDKITTRGFAIQCRITTEDPAKNFQPDTGRIEVYRSAGGNGVRLDGGNAYAGTIISPHYDSMLVKCSCSGSTYEIVRRKMIRALIEFRIRGVKTNIPFLLTLLTNPVFIEGTYWTTFIDDTPQLFQMVSSQNRAQKLLHYLADVAVNGSSIKGQIGLPKLKSNPSVPHLHDAQGNVINVTKSAPPSGWRQVLLEKGPAEFARQVRQFNGTLLMDTTWRDAHQSLLATRVRTHDLATIAPTTAHALAGRFALECWGGATFDVAMRFLHEDPWERLRKLRSLVPNIPFQMLLRGANGVAYSSLPDNAIDHFVKQAKDNGVDIFRVFDALNDLEQLKVGVDAVKKAGGVVEATVCFSGDMLQPGKKYNLDYYLEIAEKIVQMGTHILGIKDMAGTMKPAAAKLLIGSLRAKYPDLPIHVHTHDSAGTAVASMTACALAGADVVDVAINSMSGLTSQPSINALLASLEGNIDTGINVEHVRELDAYWAEMRLLYSCFEADLKGPDPEVYQHEIPGGQLTNLLFQAQQLGLGEQWAETKRAYREANYLLGDIVKVTPTSKVVGDLAQFMVSNKLTSDDVRRLANSLDFPDSVMDFFEGLIGQPYGGFPEPFRSDVLRNKRRKLTCRPGLELEPFDLEKIREDLQNRFGDVDECDVASYNMYPRVYEDFQKMRETYGDLSVLPTRSFLSPLETDEEIEVVIEQGKTLIIKLQAVGDLNKKTGEREVYFDLNGEMRKIRVADRSQKVETVTKSKADMHDPLHIGAPMAGVIVEVKVHKGSLIKKGQPVAVLSAMKMEMIISSPSDGQVKEVFVSDGENVDSSDLLVLLEDQVPVETKA

SEQ ID NO: 96 - protein, 1175 aa, Kluyvermyces marxianus
MSTQNDLAGLRDNSNLLGEKNKILVANRGEIPIRIFRTAHELSMKTVAIYSHEDRLSMHRLKADEAYVIGEPGKYTPVGAYLAIDEIIKIAQLHGVSFIHPGYGFLSENSEFAKKVADSGITWVGPPADVIDAVGDKVSARNLAERADVPVVPGTPGPIETVEEAVEFVEKYGYPVIIKAAFGGGGRGMRVVREGDDIADAFQRAKSEAVTAFGNGTCFVERFLDKPKHIEVQLLADHYGNVIHLFERDCSVQRRHQKVVEVAPAKTLPESVRNAILTDAVKLAKEAGYRNAGTAEFLVDNQNRHYFIEINPRIQVEHTITEEITGIDIVAAQIQIAAGASLEQLGLLQDRITTRGFAIQCRITTEDPSKNFQPDTGRIDVYRSAGGNGVRLDGGNAFAGSVISPHYDSMLVKCSCSGSTYEIVRRKMLRALIEFRIRGVKTNIPFLLTLLTHPVFKSGDYWTTFIDDTPQLFEMVSSQNRAQKLLHYLADLAVNGSSIKGQIGLPKLKTHPTIPHLHKADGSILDVSAKPPAGWRDVLLQHGPEEFAKQVRKFKGTLLMDTTWRDAHQSLLATRVRTYDLAAIAPTTAHALSGAFALECWGGATFDVSMRFLHEDPWERLRTLRKLVPNIPFQMLLRGANGVAYSSLPDNAIDHFVKQAKDNGVDIFRVFDALNDLEQLTVGVDAVKKAGGVVEATICYSGDMLAPGKKYNLDYYLDIVEQVVKRGTHILGIKDMAGTLKPSAAKLLIGSIRTKYPDLPIHVHTHDSAGTGVASMAACAFAGADVVDVATNSMSGMTSQPSVNALLAALDGEIDCNVNVSYISQLDAYWAEMRLLYSCFEADLKGPDPEVYVHEIPGGQLTNLLFQAQQLGLGEQWAETKRAYREANLLLGDVVKVTPTSKVVGDLAQFMVTNKLTSDDVKRLASSLDFPDSVMDFFEGLIGQPYGGFPEPLRSDVLKNKRRKLTKRPGLELAPFDLEGIKEDLTNRFGDIDDCDVASYNMYPKVYEDFRKIREKYGDLSVLPTKNFLSPPSIGEEIVVTIEQGKTLIIKPQAIGDLNKETGIREVYFELNGELRKVSVADRSQKVETISKPKADAHDPFQVGSPMAGVVVEVKVHKGSLISKGQPVAVLSAMKMEMVISSPSDGQVKEVLVKDGENVDASDLLVVLEEAPAKE

SEQ ID NO: 97 - protein, 883 aa, Escherichia coli
MNEQYSALRSNVSMLGKVLGETIKDALGEHILERVETIRKLSKSSRAGNDANRQELLTTLQNLSNDELLPVARAFSQFLNLANTAEQYHSISPKGEAASNPEVIARTLRKLKNQPELSEDTIKKAVESLSLELVLTAHPTEITRRTLIHKMVEVNACLKQLDNKDIADYEHNQLMRRLRQLIAQSWHTDEIRKLRPSPVDEAKWGFAVVENSLWQGVPNYLRELNEQLEENLGYKLPVEFVPVRFTSWMGGDRDGNPNVTADITRHVLLLSRWKATDLFLKDIQVLVSELSMVEATPELLALVGEEGAAEPYRYLMKNLRSRLMATQAWLEARLKGEELPKPEGLLTQNEELWEPLYACYQSLQACGMGIIANGDLLDTLRRVKCFGVPLVRIDIRQESTRHTEALGELTRYLGIGDYESWSEADKQAFLIRELNSKRPLLPRNWQPSAETREVLDTCQVIAEAPQGSIAAYVISMAKTPSDVLAVHLLLKEAGIGFAMPVAPLFETLDDLNNANDVMTQLLNIDWYRGLIQGKQMVMIGYSDSAKDAGVMAASWAQYQAQDALIKTCEKAGIELTLFHGRGGSIGRGGAPAHAALLSQPPGSLKGGLRVTEQGEMIRFKYGLPEITVSSLSLYTGAILEANLLPPPEPKESWRRIMDELSVISCDVYRGYVRENKDFVPYFRSATPEQELGKLPLGSRPAKRRPTGGVESLRAIPWIFAWTQNRLMLPAWLGAGTALQKVVEDGKQSELEAMCRDWPFFSTRLGMLEMVFAKADLWLAEYYDQRLVDKALWPLGKELRNLQEEDIKVVLAIANDSHLMADLPWIAESIQLRNIYTDPLNVLQAELLHRSRQAEKEGQEPDPRVEQALMVTIAGIAAGMRNTG

SEQ ID NO: 98 - protein, 342 aa, Issatchenkia orientalis
MSNVKVALLGAAGGIGQPLALLLKLNPNITHLALYDVVHVPGVAADLHHIDTDVVITHHLKDEDGTALANALKDATFVIVPAGVPRKPGMTRGDLFTINAGICAELANAISLNAPNAFTLVITNPVNSTVPIFKEIFAKNEAFNPRRLFGVTALDHVRSNTFLSELIDGKNPQHFDVTVVGGHSGNSIVPLFSLVKAAENLDDEIIDALIHRVQYGGDEVVEAKSGAGSATLSMAYAANKFFNILLNGYWGLKKTMISSYVFLDDSINGVPQLKENLSKLLKGSEVELPSYLAVPMTYGKEGIEQVFYDWVFEMSPKEKENFITAIEYIDQNIEKGLNFMVR

SEQ ID NO: 99 - protein, 333 aa, Issatchenkia orientalis
MVKVTILGAAGGIGQPLSLLLRLNPWIDELALFDIVNTPGVSCDLSHIPASQVVNGYAPKSKSDTETIKTALKGADIVVIPAGIPRKPGMTRNDLFKINAGIVKSLIHSAGTTCPDAFICVISNPVNSTVPIAVEELKRLNVFNPHKVFGITTLDNFRLEEFLSGELGGIVKPNDLYGDVVAIGGHSGDSIVPILNSWNLNFINDGDSYNNLVKRVQFGGDEVVKAKDGKGSATLSMATAAYRFVNNLLDAIVNNKKVKEVAFVKIDQLPTTRVPYFVVDETQYFSLPIILGRQGIERVTFPESLTEQEVRMTKHAVAKVKVDVNKGFNFVHG

SEQ ID NO: 100 - protein, 334 aa, Issatchenkia orientalis
MFSRISARQFSSSAASAYKVTVLGAAGGIGQPLSLLMKLNHKVTNLSLYDLRLGAGVATDLSHIPTNSVVKGYGPENNGLKDALTGSDVVLIPAGVPRKPGMTRDDLFNTNASIVRDLAKAAADHCPNAVLLIISNPVNSTVPIVAEVLKSKGVYNPKKLFGVTTLDVLRSSRFLSEVVNTDPTTETVTVVGGHSGVTIVPLISQTKHKDLPKETYEALVHRIQFGGDEVVKAKDGAGSATLSMAQAGARMASSVLKGLAGEVDIVEPTFIDSPLFKSEGVEFFSSRVTLGPEGVQEVHPLGVLSTAEEEMVATAKETLKKNIQKGVDFVKANP

SEQ ID NO: 101 - protein, 356 aa, Zygosaccharomyces rouxii
MPHSINGDVKIAVLGAAGGIGQSLSLLLKTQLTRELPNHRHAQLALYDVNADAVRGVAADLSHIDTGVTVTGYEGDRIGEALEGTDIVLIPAGVPRKPGMTREDLLVVNAKIVKSIGSSIAQHCDLNKVFILLISNPINSLVPVLVKELESKSQGTQVERRVLGLTKLDSVRASAFLHEVTIKHGLKPKSNTLDDVPVVGGHSGETIVPLFSQAPNGNRLSQDALEALVQRVQFGGDEVVRAKNGAGSATLCMAHAAYTVAASFIPLITGQKRSISGTFYVALKDAQGQPINSSAKRLLGSINDLPYFAVPLEITSQGVDELDTSVLERMTKYERERLLAPCLGKLEGGIRNGLSL

SEQ ID NO: 102 - protein, 338 aa, Kluyveromyces marxianus
MLRALTRRQFSSTAFNPYKVTVLGAGGGIGQPLSLLLKLNHKVTDLRLYDLKGAKGVAADLSHIPTNSTVTGYTPESKDSQEELAAALKDTEVVLIPAGVPRKPGMTRDDLFAINAGIVRDLATSIAKNAPNAAILVISNPVNSTVPIVAEVLKQNGVYNPKKLFGVTTLDVIRASRFISEVRGTDPTTEHVTVVGGHSGITILPLVSQTKHKSVIKGEELDNLIHRIQFGGDEVVQAKNGAGSATLSMAQAGARFANSVLSGFEGERDVIEPTFVDSPLFKDEGIEFFASPVTLGPEGVEKIHGLGVLSDKEEQMLATCKETLKKNIEKGQNFVKQN

SEQ ID NO: 103 - protein, 335 aa, Kluyveromyces marxianus
MVSVAVLGSSGGIGQPLSLLLKLDPRVSSLRLYDLKMSHGIATDLSHMDSNSICEGFNTDEIALALKGAQIVVIPAGVPRKPGMSRDDLFKINAKIIKSLALQIAEHAPEARVLVISNPVNSLVPIVYETLKSVGKFEPGKVMGITTLDIIRSHTFLVDVLGRKAYSVEKLRSAVTVVGGHSGETIVPIFTDQKFYRRLRDRELYDAYVHRVQFGGDEVVKAKDGSGSATLSMAWAGYSFVKQLLNSLHLETGEDVHPIPTFVYLPGLPGGKELQQKLGTSVEFFAAPVKLSKGIVVEVEHDWVDKLNDAEKKLIAKCLPILDKNIKKGLAFSQQ

SEQ ID NO: 104 - protein, 374 aa, Kluyveromyces marxianus
MPAVSYDVQQRDILKIAVLGAAGGIGQSLSLLLKSNASFLLPRDSSRHISLALYDVNKDAIVGTAADLSHIDTPITTTPHYPNDGNGGIARCLQDADMVIIPAGVPRKPGMSRDDLIGVNAKIIKSLGNDIAEYCDLSKVHVLVISNPVNSLVPLMVSTLANSPHSANTNIEARVYGITHLDLVRASTFVQQLNSFKSNNAPDIPVIGGHSGDTIIPVFSVLNHRASNSGYANLLDNGVRQKLVHRVQYGGDEIVQAKNGNGSATLSMAYAGFKIAAQFIDLLVGNIRTIENICMYVPLTNRYNTEIAPGSDELRSKYINGTLYFSIPLSIGINGIERVHYEIMEHLDSYERETLLPICLETLKGNIDKGLSLV

SEQ ID NO: 105 - protein, 312 aa, Escherichia coli
MKVAVLGAAGGIGQALALLLKTQLPSGSELSLYDIAPVTPGVAVDLSHIPTAVKIKGFSGEDATPALEGADVVLISAGVARKPGMDRSDLFNVNAGIVKNLVQQVAKTCPKACIGIITNPVNTTVAIAAEVLKKAGVYDKNKLFGVTTLDIIRSNTFVAELKGKQPGEVEVPVIGGHSGVTILPLLSQVPGVSFTEQEVADLTKRIQNAGTEVVEAKAGGGSATLSMGQAAARFGLSLVRALQGEQGVVECAYVEGDGQYARFFSQPLLLGKNGVEERKSIGTLSAFEQNALEGMLDTLKKDIALGEEFVNK

SEQ ID NO: 106 - protein, 337 aa, Rhizopus oryzae
MFAASRVFSIAAKRSFSTSAANLSKVAVLGAAGGIGQPLSLLLKENPHVTHLSLYDIVNTPGVAADLSHINTNSKVTGHTPENDGLKTALEGAHVVVIPAGVPRKPGMTRDDLFNTNASIVRDLAEAAAKHCPDAHFLIISNPVNSTVPIFAETLKKAGVFNPKRLYGVTTLDVVRASRFVAEVKNLDPNDVKVTVVGGHSGVTIVPLLSQTGLEFSKEELDALTHRIQFGGDEVVQAKNGTGSVTLSMAFAGARFANSVLEATVGGKKGVVEPSFVKSDVFAKDGVEYFSTNIELGPEGVEKINELGQISDYEKELIAKAVPELKKNIAKGNSFVQ

SEQ ID NO: 107 - protein, 485 aa, Issatchenkia orientalis
MLAARSLKARMSTRAFSTTSIAKRIEKDAFGDIEVPNEKYWGAQTQRSLQNFKIGGKREVMPEPIIKSFGILKKATAKINAESGALDPKLSEAIQQAATEVYEGKLMDHFPLVVFQTGSGTQSNMNANEVISNRAIEILGGELGSKTPVHPNDHVNMSQSSNDTFPTVMHIAAVTEVSSHLLPELTALRDALQKKSDEFKNIIKIGRTHLQDATPLTLGQEFSGYVQQCTNGIKRIEIALEHLRYLAQGGTAVGTGLNTKKGFAEKVANEVTKLTGLQFYTAPNKFEALAAHDAVVEMSGALNTVAVSLFKIAQDIRYLGSGPRCGYGELALPENEPGSSIMPGKVNPTQNEALTMLCTQVFGNHSCITFAGASGQFELNVFKPVMISNLLSSIRLLGDGCNSFRIHCVEGIIANTDKIDKLLHESLMLVTALNPHIGYDKASKIAKNAHKKGLTLKQSALELGYLTEEQFNEWVRPENMIGPKD

SEQ ID NO: 108 - protein, 470 aa, Saccharomyces cerevisiae
MSLSPVVVIGTGLAGLAAANELVNKYNIPVTILEKASSIGGNSIKASSGINGACTETQRHFHIEDSPRLFEDDTIKSAKGKGVQELMAKLANDSPLAIEWLKNEFDLKLDLLAQLGGHSVARTHRSSGKLPPGFEIVSALSNNLKKLAETKPELVKINLDSKVVDIHEKDGSISAVVYEDKNGEKHMVSANDVVFCSGGFGFSKEMLKEYAPELVNLPTTNGQQTTGDGQRLLQKLGADLIDMDQIQVHPTGFIDPNDRSSSWKFLAAESLRGLGGILLNPITGRRFVNELTTRDVVTAAIQKVCPQEDNRALLVMGEKMYTDLKNNLDFYMFKKLVQKLTLSQVVSEYNLPITVAQLCEELQTYSSFTTKADPLGRTVILNEFGSDVTPETVVFIGEVTPVVHFTMGGARINVKAQVIGKNDERLLKGLYAAGEVSGGVHGANRLGGSSLLECVVFGRTAAESIANDRK

SEQ ID NO: 109 - protein, 470 aa, Saccharomyces mikatae
MSSSPVVVIGTGLAGLATANELVNKYNIPVTILEKASSIGGNSIKASSGINGACTETQRHFHIEDTPRLFEDDTVQSAKGKGVQELMGKLANDSPLAIEWLKTEFDLKLDLLAQLGGHSVARTHRSSGKLPPGFEIVSALSNNLKKLAETKPELVKINLDSKVVDIHKKDGSISAIVYDDKNGERHTLSTSNVVFCSGGFGFSKEMLNEYAPQLVNLPTTNGQQTTGDGQRLLQKLGADLIDMDQIQVHPTGFIDPNDRNSSWKFLAAESLRGLGGILLNPITGRRFVNELTTRDVVTEAIQKHCPQDDNRALLVMSEKMYTDLKNNLDFYMFKKLVQKLSLSQVVSEYKLPITVSQLCQELQTYSSFTSKADPLGRTVVLNEFGADITPETMVFIGEVTPVVHFTMGGARINVKAQVIGKNDEPLLNGLYAAGEVSGGVHGANRLGGSSLLECVVFGRTAAESIANNHK

SEQ ID NO: 110 - protein, 470 aa, Kluyveromyces polyspora
MSTKKPVVIIGTGLAGLSAGNQLVNMHKVPIIMLDKASSIGGNSTKASSGINGASTITQQQLNVKDSPDLFLQDTVKSAKGRGIESLMKKLSQDSNSAIHWLQQDFDLKLDLLAQLGGHSVPRTHRSSGKLPPGFEIVQALSNKLKAISESDPEFVRILLNSKVVDVSVNNEGKVESIDYVDAEGKHHKIATDNVVFCSGGFGHSAEMLNKYAPELANLPTTNGQQTTGDGQRILEKLGADLIDMSQIQVHPTGFIDPANRDSKWKFLAAEALRGLGGILLNPSTGKRFVNELTTRDLVTEAIQSQCPRDDNKAFLVMSEKVYENYKNNMDFYLFKKLVSKMTIKEFVETYKLPISADAVTQDLIDYSVDKTDKFGRPLVINVFDEKLTEDSEIYVGEVTPVVHFTMGGAKINTESQVINKNGQVLAKGIYAAGEVSGGVHGSNRLGGSSLLECVVYGRSAADNIAKNIE

SEQ ID NO: 111 - protein, 487 aa, Kluyveromyces marxianus
MLHRYIRLFSFCVILYLVYLLLTKESNVMSKPVVVIGSGLAGLTTSSQLAKFNIPIVLLEKTSSIGGNSIKASSGINGAGTETQSRLHVEDHPELFADDTIKSAKGKGVVALMEKLSKDSSDAISWLQNDFKIPLDKLAQLGGHSVPRTHRSSGKLPPGFQIVDTLKKALESYDSKAVKIQLNSKVVDVKLDSNNRVSSVVFEDQDGTHTIETNNVVFCTGGFGFNKKLLEKYAPHLVDLPTTNGEQTLGEGQVLLEKLGAKLIDMDQIQVHPTGFIDPANPDSNWKFLAAEALRGLGGVLINPHTGQRFVNELTTRDMVTEAIQSKSESKTAYLVMSESLYENYKPNMDFYMFKKLVSKKTVAEFAEDLPVSVDQLIAELSTYSDLSKDDHLGRKFRENTFGSSLSSDSTIFVGKITPVVHFTMGGAKIDEQARVLNAEGKPLATGIYAAGEVSGGVHGANRLGGSSLLECVVFGRQAAKSIRANL

SEQ ID NO: 112 - protein, 1139 aa, Trypanosoma brucei
MVDGRSSASIVAVDPERAARERDAAARALLQDSPLHTTMQYATSGLELTVPYALKVVASADTFDRAKEVADEVLRCAWQLADTVLNSFNPNSEVSLVGRLPVGQKHQMSAPLKRVMACCQRVYNSSAGCFDPSTAPVAKALREIALGKERNNACLEALTQACTLPNSFVIDFEAGTISRKHEHASLDLGGVSKGYIVDYVIDNINAAGFQNVFFDWGGDCRASGMNARNTPWVVGITRPPSLDMLPNPPKEASYISVISLDNEALATSGDYENLIYTADDKPLTCTYDWKGKELMKPSQSNIAQVSVKCYSAMYADALATACFIKRDPAKVRQLLDGWRYVRDTVRDYRVYVRENERVAKMFEIATEDAEMRKRRISNTLPARVIVVGGGLAGLSAAIEAAGCGAQVVLMEKEAKLGGNSAKATSGINGWGTRAQAKASIVDGGKYFERDTYKSGIGGNTDPALVKTLSMKSADAIGWLTSLGVPLTVLSQLGGHSRKRTHRAPDKKDGTPLPIGFTIMKTLEDHVRGNLSGRITIMENCSVTSLLSETKERPDGTKQIRVTGVEFTQAGSGKTTILADAVILATGGFSNDKTADSLLREHAPHLVNFPTTNGPWATGDGVKLAQRLGAQLVDMDKVQLHPTGLINPKDPANPTKFLGPEALRGSGGVLLNKQGKRFVNELDLRSVVSKAIMEQGAEYPGSGGSMFAYCVLNAAAQKLFGVSSHEFYWKKMGLFVKADTMRDLAALIGCPVESVQQTLEEYERLSISQRSCPITRKSVYPCVLGTKGPYYVAFVTPSIHYTMGGCLISPSAEIQMKNTSSRAPLSHSNPILGLFGAGEVTGGVHGGNRLGGNSLLECVVFGRIAGDRASTILQRKSSALSFKVWTTVVLREVREGGVYGAGSRVLRFNLPGALQRSGLSLGQFIAIRGDWDGQQLIGYYSPITLPDDLGMIDILARSDKGTLREWISALEPGDAVEMKACGGLVIERRLSDKHFVFMGHIINKLCLIAGGTGVAPMLQIIKAAFMKPFIDTLESVHLIYAAEDVTELTYREVLEERRRESRGKFKKTFVLNRPPPLWTDGVGFIDRGILTNHVQPPSDNLLVAICGPPVMQRIVKATLKTLGYNMNLVRTVDETEPSGS

SEQ ID NO: 113 - protein, 1139 aa, Trypanosoma cruzi
MADGRSSASVVAVDPEKAARERDEAARALLRDSPLQTHLQYMTNGLELTVPFTLKVVAEAVAFSRAKEVADEVLRSAWHLADTVLNNFNPNSEISMIGRLPVGQKHTMSATLKSVITCCQHVFNSSRGVFDPATGPIIEALRAKVAEKASVSDEQMEKLFRVCNFSSSFIVDLEMGTIARKHEDARFDLGGVSKGYIVDYVVERLNAAGIVDVYFEWGGDCRASGTNARRTPWMVGIIRPPSLEQLRNPPKDPSYIRVLPLNDEALCTSGDYENLTEGSNKKLYTSIFDWKKRSLLEPVESELAQVSIRCYSAMYADALATASLIKRDIKKVRQMLEDWRHVRNRVTNYVTYTRQGERVARMFEIATDNAEIRKKRIAGSLPARVIVVGGGLAGLSAAIEATACGAQVILLEKEPKVGGNSAKATSGINGWGTRAQAEQDVYDSGKYFERDTHKSGLGGSTDPGLVRTLSVKSGDAISWLSSLGVPLTVLSQLGGHSRKRTHRAPDKADGTPVPIGFTIMQTLEQHVRTKLADRVTIMENTTVTSLLSKSRVRHDGAKQVRVYGVEVLQDEGVVSRILADAVILATGGFSNDKTPNSLLQEFAPQLSGFPTTNGPWATGDGVKLARELGVKLVDMDKVQLHPTGLIDPKDPANPTKYLGPEALRGSGGVLLNKKGERFVNELDLRSVVSNAIIEQGDEYPDAGGSKFAFCVLNDAAVKLFGVNSHGFYWKRLGLFVKADTVEKLAALIGCPVENVRNTLGDYEQLSKENRQCPKTRKVVYPCVVGPQGPFYVAFVTPSIHYTMGGCLISPSAEMQLEENTTSPFGHRRPIFGLFGAGEVTGGVHGGNRLGGNSLLECVVFGRIAGDRAATILQKKPVPLSFKTWTTVILREVREGGMYGTGSRVLRFNLPGALQRSGLQLGQFIAIRGEWDGQQLIGYYSPITLPDDLGVIGILARSDKGTLKEWISALEPGDAVEMKGCGGLVIERRFSERYLYFSGHALKKLCLIAGGTGVAPMLQIIRAALKKPFLENIESIRLIYAAEDVSELTYRELLEHHQRDSKGKFRSIFVLNRPPPIWTDGVGFIDKKLLSSSVQPPAKDLLVAICGPPIMQRVVKTCLKSLGYDMQLVRTVDEVETQNS

SEQ ID NO: 114 - protein, 1144 aa, Leishmania braziliensis
MADGKTSASVVAVDPERAAKERDAAARAMLQDGGVSPVGKAQLLKKGLAYAVPYTLKIVVADPKAMEKTTADVEKVLQTAFQVVDTLLNNFNENSEVSRINRMPVGEEHQMSAALKRVMGCCQRVYNSSRGAFDPAVGPLVRELREAAREGRTLPAERINALLSKCTLNISFSIDLNRGTIARKHADAMLDLGGVNKGYGVDYVVEHLNNLGYDDVFFEWGGDVRASGKNPSNQHWVVGIARPPALADIRTVVPQDKQSFIRVVCLNDEAIATSGDYENLVEGPGSKVYSSTFNATSKSLLEPTETNIAQVSVKCYSCMYADALATAALLKNNPTAVRRMLDNWRYVRDTVTDYTTYSREGERVAKMFEIATEDKEMRAKRISGSLPARVIIVGGGLAGCSAAIEAVNCGAQVILLEKEAKIGGNSAKATSGINAWGTRAQAKQGVMDGGKFFERDTHRSGKGGHCDPCLVKTLSVKSSDAVKWLSELGVPLTVLSQLGGASRKRCHRAPDKSDGTPVPIGFTIMKTLENHIINDLSHQVTVMTGIKVTGLESTSHARPDGVLVKHVTGVRLIQGDGQSRVLNADAVILATGGFSNDHTANSLLQQYAPQLSSFPTTNGVWATGDGVKAARELGVELVDMDKVQLHPTGLLDPKDPSNRTKYLGPEALRGSGGVLLNKNGERFVNELDLRSVVSQAIIEQNNVYPGSGGSKFAYCVLNEAAAKLFGKNFLGFYWHRLGLFEKVEDVAGLAKLIGCPEENVTATLKEYKELSSKKLHACPLTNKNVFPCTLGTEGPYYVAFVTPSIHYTMGGCLISPSAEMQTIDNTGVTPVRRPILGLFGAGEVTGGVHGGNRLGGNSLLECVVFGRIAGDRAATILQKKNAGLSMTEWSTVVLREVREGGVYGTGSRVLRFNMPGALQKTGLALGQFIAMRGDWDGQQLLGYYSPITLPDDIGVIGILARADKGRLAEWISALQPGDAVEMKACGGLIIHRRFAARHLFFRSHKIRKLALIGGGTGVAPMLQIVRAAVKKPFVDSIESIQFIYAAEDVSELTYRTLLESYEKEYGSGKFKCHFVLNNPPSQWTEGVGFVDTALLRSAVQAPSNDLLVAICGPPIMQRAVKSALKGLGYNMNLVRTVDEPEPLS

SEQ ID NO: 115 - protein, 880 aa, Mannheimia succiniciproducens
MTEEYLMMRNNINMLGRFLGETIQEAQGDDILELIENIRVLSRNSRSGDDKARAALLDTLSTISADNIIPVARAFSQFLNLTNVAEQYQTMSRSHEDKVSAERSTAALFARLKEQHVSQEEIIKTVQKLLIEIVLTAHPTEVTRRSLMHKQVEINKCLAQLDHTDLTAEEQKNIEYKLLRLIAEAWHTNEIRTNRPTPLEEAKWGFAVIENSLWEGLPAFIRKLNDAAVEHLNYALPVDLTPVRFSSWMGGDRDGNPFVTAKITREALQLARWKAADLFLTDIQELCDELSMTQCTAEFREKYGDHLEPYRVVVKDLRSKLKNTLDYYNDILAGRIPPFKQDEIISEDQQLWQPLYDCYQSLTACGMRIIANGLLLDTLRRVRCFGVTLLRLDIRQESTRHSDAIGEITRYIGLGDYSQWTEDDKQAFLIRELSSRRPLIPHNWTPSEHTREILDTCKVIAKQPEGVISCYIISMARTASDVLAVHLLLKEAGISYHLPVVPLFETLDDLDASKEVMTQLFNVGWYRGVIKNRQMIMIGYSDSAKDAGMMAASWAQYRAQDALVKLCEQTGIELTLFHGRGGTVGRGGAPAHAALLSQPPRSLKNGLRVTEQGEMIRFKLGLPAIAAESLDLYASAILEANLLPPPEPKASWCRVMDELAVASCEIYRNVVRGDKDFVPYFRSATPEQELAKLPLGSRPAKRNPNGGVESLRAIPWIFAWMQNRLMLPAWLGAGASIRQAMESGKAAVIEEMCNHWPFFNTRIGMLEMVFSKTDSWLSEYYDQRLVKKELWYLGESLRKQLSEDIATVLRLSGKGDQLMSDLPWVAESIALRNVYTDPLNLLQVELLRRLRADPEHPNPDIEQALMITITGIAAGMRNTG

SEQ ID NO: 116 - DNA, 4171 bp, synthetic construct
  source 1..4171: mol_type = other DNA; note = Synthetic - I. orientalis FUM gene integration fragment
aattctttgaaggagcttgccaagaaacataattttatgatttttgaagatagaaaatttgctgatattggtaacactgttaaaaatcaatataaatctggtgtcttccgtattgccgaatgggctgacatcactaatgcacatggtgtaacgggtgcaggtattgtttctggcttgaaggaggcagcccaagaaacaaccagtgaacctagaggtttgctaatgcttgctgagttatcatcaaagggttctttagcatatggtgaatatacagaaaaaacagtagaaattgctaaatctgataaagagtttgtcattggttttattgcgcaacacgatatgggcggtagagaagaaggttttgactggatcattatgactccaggggttggtttagatgacaaaggtgatgcacttggtcaacaatatagaactgttgatgaagttgtaaagactggaacggatatcataattgttggtagaggtttgtacggtcaaggaagagatcctatagagcaagctaaaagataccaacaagctggttggaatgcttatttaaacagatttaaatgattcttacacaaagatttgatacatgtacactagtttaaataagcatgaaaagaattacacaagcaaaaaaaaaaaaataaatgaggtactttacgttcacctacaaccaaaaaaactagatagagtaaaatcttaagatttagaaaaagttgtttaacaaaggctttagtatgtgaatttttaatgtagcaaagcgataactaataaacataaacaaaagtatggttttctttatcagtcaaatcattatcgattgattgttccgcgtatctgcagatagcctcatgaaatcagccatttgcttttgttcaacgatcttttgaaattgttgttgttcttggtagttaagttgatccatcttggcttatgttgtgtgtatgttgtagttattcttagtatattcctgtcctgagtttagtgaaacataatatcgccttgaaatgaaaatgctgaaattcgtcgacatacaatttttcaaactttttttttttcttggtgcacggacatgtttttaaaggaagtactctataccagttattcttcacaaatttaattgctggagaatagatcttcaacgcgtttcctcgacatttgctgcaacggcaacatcaatgtccacgtttacacacctacatttatatctatatttatatttatatttatttatttatgctacttagcttctatagttagttaatgcactcacgatattcaaaattgacacccttcaactactccctactattgtctactactgtctactactcctctttactatagctgctcccaataggctccaccaataggctctgtcaatacattttgcgccgccacctttcaggttgtgtcactcctgaaggaccatattgggtaatcgtgcaatttctggaagagagtccgcgagaagtgaggcccccactgtaaatcctcgagggggcatggagtatggggcatggaggatggaggatggggggggggggggggaaaataggtagcgaaaggacccgctatcaccccacccggagaactcgttgccgggaagtcatatttcgacactccggggagtctataaaaggcgggttttgtcttttgccagttgatgttgctgagaggacttgtttgccgtttcttccgatttaacagtatagaatcaaccactgttaattatacacgttatactaacacaacaaaaacaaaaacaacgacaacaacaacaacatctagataaaatgttagctgctagatcattaaaggcaagaatgtcaacaagagctttctcaactacctcaattgcaaaaagaatcgaaaaagatgcatttggtgacattgaagtcccaaatgagaaatattggggtgctcaaactcaaagatctttacaaaatttcaaaattggtggtaagagagaagttatgccagaaccaatcatcaaatcttttggtattttaaagaaggctactgctaagatcaatgctgagtctggtgctttagacccaaagttatctgaagccatccaacaagctgcaaccgaagtttatgaaggtaaactaatggaccatttcccattagttgtctttcaaaccggttctggtactcaatctaacatgaatgccaatgaagtcatctctaatagagcaattgaaatcttgggtggtgaattaggctctaaaactccagtccatcctaatgatcatgttaatatgtcccaatcttctaatgatactttccctactgtcatgcatattgcagcagttacagaagtttcatcccatttattaccagaattaactgcactaagagatgcattgcaaaagaaatccgatgaatttaagaatattatcaaaatcggtagaacccatttacaagatgcaactcctttaactttaggtcaagaattttctggttatgttcaacaatgtactaatggtatcaaaagaatcgaaattgctcttgaacatttgagatacttagctcaaggtggtactgccgttggtactggtcttaacaccaagaaaggttttgctgaaaaggttgcaaatgaagtcactaaattgactggtttacaattctataccgctccaaataaattcgaagcccttgcagctcacgatgctgttgttgaaatgtctggtgctttgaataccgttgcagtctcattattcaaaatcgctcaagatatcagatatttgggttccggcccaagatgtggttatggtgaattggctttaccagaaaatgaaccaggttcttccatcatgccgggtaaagttaacccaactcaaaacgaagctttgactatgctttgtacccaagtctttggtaaccactcttgtattacctttgcaggtgcttcaggtcaattcgaattgaatgtctttaagccagttatgatctccaacttgttatcttctattaggttattaggtgatggttgtaattcttttagaatccactgtgttgaaggtatcattgcaaataccgacaagattgataaattactacatgaatctctcatgttagttactgctttgaacccacacattggttacgataaggcttccaagattgcaaagaatgcacacaagaagggcttgacattgaaacaatctgcattggaattaggttacttgaccgaagaacaattcaatgaatgggttagaccagaaaacatgattggtccaaaggattaagttaattaacatctgaatgtaaaatgaacattaaaatgaattactaaactttacgtctactttacaatctataaactttgtttaatcatataacgaaatacactaatacacaatcctgtacgtatgtaatacttttatccatcaaggattgagaaaaaaaagtaatgattccctgggccattaaaacttagacccccaagcttggataggtcactctctattttcgtttctcccttccctgatagaagggtgatatgtaattaagaataatatataattttataataaaagcggccgcaccaggggtttagtgaagtcaccaattaagattgttggtttgagtgagttgccaaagatctatgaattgatggagcaaggtaagattttaggcagatatgttgttgacacttcgaaatgatgggctgacttgggtgtactggtgtgacgtttttatgtgtatattgatatgcatgggggatgtatagtgatgaggagtagagtatataacgaaatgaaatgaaataatatgatatgataagataagatgagatcaatacgataatataagatgcgacatgaggagttcaatgtagcatactacacgatgctgcagtacaactctgatacgctagactatactatacaaaactgtagtacactatacgttagtgtagtatccagaaacaacactgctttatagtacaatacaactctataatactatagtatactatgccaaaccacgtaataccataatatgctccacgacatggtacaatgtgctatacttcatactattataccatatatactccgatatattattgatatactattttatactataataccataccacacaacactacattacaacgagcaaccttaccataaatgtcagttatggccgc

SEQ ID NO: 117 - protein, 466 aa, Escherichia coli
MPHSYDYDAIVIGSGPGGEGAAMGLVKQGARVAVIERYQNVGGGCTHWGTIPSKALRHAVSRIIEFNQNPLYSDHSRLLRSSFADILNHADNVINQQTRMRQGFYERNHCEILQGNARFVDEHTLALDCPDGSVETLTAEKFVIACGSRPYHPTDVDFTHPRIYDSDSILSMHHEPRHVLIYGAGVIGCEYASIFRGMDVKVDLINTRDRLLAFLDQEMSDSLSYHFWNSGVVIRHNEEYEKIEGCDDGVIMHLKSGKKLKADCLLYANGRTGNTDSLALQNIGLETDSRGQLKVNSMYQTAQPHVYAVGDVIGYPSLASAAYDQGRIAAQALVKGEATAHLIEDIPTGIYTIPEISSVGKTEQQLTAMKVPYEVGRAQFKHLARAQIVGMNVGTLKILFHRETKEILGIHCFGERAAEIIHIGQAIMEQKGGGNTIEYFVNTTFNYPTMAEAYRVAALNGLNRLF

SEQ ID NO: 118 - protein, 466 aa, synthetic construct; identical to SEQ ID NO: 117
  source 1..466: note = Synthetic - Codon optimized E. coli Stha enzyme

SEQ ID NO: 119 - protein, 464 aa, Azotobacter vinelandii
MAVYNYDVVVIGTGPAGEGAAMNAVKAGRKVAVVDDRPQVGGNCTHLGTIPSKALRHSVRQIMQYNNNPLFRQIGEPRWFSFADVLKSAEQVIAKQVSSRTGYYARNRIDTFFGTASFCDEHTIEVVHLNGMVETLVAKQFVIATGSRPYRPADVDFTHPRIYDSDTILSLGHTPRRLIIYGAGVIGCEYASIFSGLGVLVDLIDNRDQLLSFLDDEISDSLSYHLRNNNVLIRHNEEYERVEGLDNGVILHLKSGKKIKADAFLWSNGRTGNTDKLGLENIGLKANGRGQIQVDEHYRTEVSNIYAAGDVIGWPSLASAAYDQGRSAAGSITENDSWRFVDDVPTGIYTIPEISSVGKTERELTQAKVPYEVGKAFFKGMARAQIAVEKAGMLKILFHRETLEILGVHCFGYQASEIVHIGQAIMNQKGEANTLKYFINTTFNYPTMAEAYRVAAYDGLNRLF

SEQ ID NO: 120 - DNA, 20 bp, synthetic construct
  source 1..20: mol_type = other DNA; note = Synthetic - Primer
cacagaggtgcagtaacgag

SEQ ID NO: 121 - protein, 506 aa, Issatchenkia orientalis; identical to SEQ ID NO: 31

SEQ ID NO: 122 - DNA, 27 bp, synthetic construct
  source 1..27: mol_type = other DNA; note = Synthetic - Primer
gctggagaatagatcttcaacgccccg

SEQ ID NO: 123 - DNA, 27 bp, synthetic construct
  source 1..27: mol_type = other DNA; note = Synthetic - Primer
catcactgttaaaggaatgggtaaatc

SEQ ID NO: 124 - DNA, 41 bp, synthetic construct
  source 1..41: mol_type = other DNA; note = Synthetic - Primer
gtaagctggcaaacctgcaggttagccggtattacgcatac

SEQ ID NO: 125 - DNA, 4321 bp, synthetic construct
  source 1..4321: mol_type = other DNA; note = Synthetic - I. orientalis FUM integration fragment
ttgaaggagcttgccaagaaacataattttatgatttttgaagatagaaaatttgctgatattggtaacactgttaaaaatcaatataaatctggtgtcttccgtattgccgaatgggctgacatcactaatgcacatggtgtaacgggtgcaggtattgtttctggcttgaaggaggcagcccaagaaacaaccagtgaacctagaggtttgctaatgcttgctgagttatcatcaaagggttctttagcatatggtgaatatacagaaaaaacagtagaaattgctaaatctgataaagagtttgtcattggttttattgcgcaacacgatatgggcggtagagaagaaggttttgactggatcattatgactccaggggttggtttagatgacaaaggtgatgcacttggtcaacaatatagaactgttgatgaagttgtaaagactggaacggatatcataattgttggtagaggtttgtacggtcaaggaagagatcctatagagcaagctaaaagataccaacaagctggttggaatgcttatttaaacagatttaaatgattcttacacaaagatttgatacatgtacactagtttaaataagcatgaaaagaattacacaagcaaaaaaaaaaaaataaatgaggtactttacgttcacctacaaccaaaaaaactagatagagtaaaatcttaagatttagaaaaagttgtttaacaaaggctttagtatgtgaatttttaatgtagcaaagcgataactaataaacataaacaaaagtatggttttctttatcagtcaaatcattatcgattgattgttccgcgtatctgcagatagcctcatgaaatcagccatttgcttttgttcaacgatcttttgaaattgttgttgttcttggtagttaagttgatccatcttggcttatgttgtgtgtatgttgtagttattcttagtatattcctgtcctgagtttagtgaaacataatatcgccttgaaatgaaaatgctgaaattcgtcgacatacaatttttcaaactttttttttttcttggtgcacggacatgtttttaaaggaagtactctataccagttattcttcacaaatttaattgctggagaatagatcttcaacgcgtttcctcgacatttgctgcaacggcaacatcaatgtccacgtttacacacctacatttatatctatatttatatttatatttatttatttatgctacttagcttctatagttagttaatgcactcacgatattcaaaattgacacccttcaactactccctactattgtctactactgtctactactcctctttactatagctgctcccaataggctccaccaataggctctgtcaatacattttgcgccgccacctttcaggttgtgtcactcctgaaggaccatattgggtaatcgtgcaatttctggaagagagtccgcgagaagtgaggcccccactgtaaatcctcgagggggcatggagtatggggcatggaggatggaggatggggggggggggggggaaaataggtagcgaaaggacccgctatcaccccacccggagaactcgttgccgggaagtcatatttcgacactccggggagtctataaaaggcgggttttgtcttttgccagttgatgttgctgagaggacttgtttgccgtttcttccgatttaacagtatagaatcaaccactgttaattatacacgttatactaacacaacaaaaacaaaaacaacgacaacaacaacaacatctagataaaatgttagctgctagatcattaaaggcaagaatgtcaacaagagctttctcaactacctcaattgcaaaaagaatcgaaaaagatgcatttggtgacattgaagtcccaaatgagaaatattggggtgctcaaactcaaagatctttacaaaatttcaaaattggtggtaagagagaagttatgccagaaccaatcatcaaatcttttggtattttaaagaaggctactgctaagatcaatgctgagtctggtgctttagacccaaagttatctgaagccatccaacaagctgcaaccgaagtttatgaaggtaaactaatggaccatttcccattagttgtctttcaaaccggttctggtactcaatctaacatgaatgccaatgaagtcatctctaatagagcaattgaaatcttgggtggtgaattaggctctaaaactccagtccatcctaatgatcatgttaatatgtcccaatcttctaatgatactttccctactgtcatgcatattgcagcagttacagaagtttcatcccatttattaccagaattaactgcactaagagatgcattgcaaaagaaatccgatgaatttaagaatattatcaaaatcggtagaacccatttacaagatgcaactcctttaactttaggtcaagaattttctggttatgttcaacaatgtactaatggtatcaaaagaatcgaaattgctcttgaacatttgagatacttagctcaaggtggtactgccgttggtactggtcttaacaccaagaaaggttttgctgaaaaggttgcaaatgaagtcactaaattgactggtttacaattctataccgctccaaataaattcgaagcccttgcagctcacgatgctgttgttgaaatgtctggtgctttgaataccgttgcagtctcattattcaaaatcgctcaagatatcagatatttgggttccggcccaagatgtggttatggtgaattggctttaccagaaaatgaaccaggttcttccatcatgccgggtaaagttaacccaactcaaaacgaagctttgactatgctttgtacccaagtctttggtaaccactcttgtattacctttgcaggtgcttcaggtcaattcgaattgaatgtctttaagccagttatgatctccaacttgttatcttctattaggttattaggtgatggttgtaattcttttagaatccactgtgttgaaggtatcattgcaaataccgacaagattgataaattactacatgaatctctcatgttagttactgctttgaacccacacattggttacgataaggcttccaagattgcaaagaatgcacacaagaagggcttgacattgaaacaatctgcattggaattaggttacttgaccgaagaacaattcaatgaatgggttagaccagaaaacatgattggtccaaaggattaagttaattaacatctgaatgtaaaatgaacattaaaatgaattactaaactttacgtctactttacaatctataaactttgtttaatcatataacgaaatacactaatacacaatcctgtacgtatgtaatacttttatccatcaaggattgagaaaaaaaagtaatgattccctgggccattaaaacttagacccccaagcttggataggtcactctctattttcgtttctcccttccctgatagaagggtgatatgtaattaagaataatatataattttataataaaagcggccgcctcccttctctaaatggactgcttggataacttggacccccttcccattttatagtcattctcttccccctcattttcccactattcccaacaatgaccatctctccaccttgtttccccattcttcctgctctacctggtgggggtgtttcaccccattaacggtcggattccgctgtggagatggctctggcctttttcccattccttccccccctcaatcttctccatgcggggaaaaaaaaattttatccataaacaaccaaaccggcggctcaacggggggtttatactgacagaaatggggtcaatacacccactgactgtacccgctctaatcttaagctttcccccccccctcctgtattaacggcgcggagtgcccgcagcgcccaatggagaaggcgcgcagtgggggatgcccagggaggggacaggtacacgcacaggccatgccaacaccgcatagacgtgcgacctcctctcccccactgcagagctgcccttttcggacacactccgtgcaagaggactcggccggctcggcttttctgccgaattcggcagccctgatattgtcttacgtaatacgaatggagggggtgtctcattttccctgcgatttcccaattgggtagcaatgtgcacacactgaaacagtgcagagtattggtgatttgctaatgtatggtaatgtataatacttttttagagtgtagtgcattgcagacagtatagtatccacttctgggaatcccatcgaaacggc

SEQ ID NO: 126 - DNA, 34 bp, synthetic construct
  source 1..34: mol_type = other DNA; note = Synthetic - Primer
gatcgagctccaccttatttatgggagttatttc

SEQ ID NO: 127 - DNA, 47 bp, synthetic construct; identical to SEQ ID NO: 54
  source 1..47: mol_type = other DNA; note = Synthetic - Primer

SEQ ID NO: 128 - protein, 328 aa, Rhizopus delemar
MVKVTVCGAAGGIGQPLSLLLKQSSHITHLSLYDIVNTPGVAADLSHIDTKSKVTGHVGAAQLEEAIKDSDVVVIPAGVPRKPGMTRDDLFKINAGIVRDLATAAAKYAPKAFMCIISNPVNSTVPIVTEVFKQHNVYDPKRIFGVTTLDIVRASTFVSELIGGEPNSLRVPVIGGHSGVTILPLLSQVPGIEKLNQEQIEKVTHRIQFGGDEVVKAKDGAGSATLSMAYAGARFATNIIEAAFAGKKGIVECTYVQLDADKSGAQSVKDLVGSELEYFSVPVELGPSGVEKILPIGNVNEYEKKLLNEASPELKTNIDKGCTFVTEG

SEQ ID NO: 129 - DNA, 19 bp, synthetic construct
  source 1..19: mol_type = other DNA; note = Synthetic - Primer
tggcccagggaatcattac

SEQ ID NO: 130 - DNA, 28 bp, synthetic construct
  source 1..28: mol_type = other DNA; note = Synthetic - Primer
tcaccacctgtcagtgacgagccacttc

SEQ ID NO: 131 - DNA, 20 bp, synthetic construct
  source 1..20: mol_type = other DNA; note = Synthetic - Primer
ggacccaatgcctcccaatc

SEQ ID NO: 132 - DNA, 21 bp, synthetic construct
  source 1..21: mol_type = other DNA; note = Synthetic - Primer
cgtttctcccttccctgatag

SEQ ID NO: 133 - DNA, 6890 bp, synthetic construct
  source 1..6890: mol_type = other DNA; note = Synthetic - E. coli SthA MEL integration fragment
cggatttggttggttcatagctcttctttagcgttgattgtagcctctgttgcagaaaagaccttgttttcaagaaactggttggtctgagtgttctgacaccaatggttatatttctagttgcaaacatgttggaataatggtagtaatgttgatgctggcagtgacagtagtgctagttcttgttcttgttctcgttctcgttctcgttctcttttttgtgctgtagctgttactgtattggctactctatataatatgcttgcaaaggaaaggaaatctatgcaaaccactctctcctgcacaaacgctagttcctttgtcagggttgaatgtagccactctacgaatgcgattcctcttcccctctctttcgcgttgagcatattcaaaattgtgagaggtggcaaggaaaaccatacgattttcggggtgcacggatgcacagtggacgtaagatctgctctatttaagatactaaagaaagtggcagcgggaagaccatcatggaggacaacgtacatggacgattccctgctagaaacaatgacatgcaaacccgtgcagtaggtagaagcagatttgctgagacagccatgccaagtggaattgttgtttaactctagtagatattattgttatagaaaagatttatatataagatccatggaggggggagggaggtaagagaaatacgaaaaagaatgtgtaatgaatcttaatgtagacaagtggaaatgcagctaaagggggtcaaagggaatgtgataatgcaaggttaggtttaacaagaatgggttggcggactcgtcaatggagagtacaatgccaaagttctccctgaggttattgcggccgcggatccctcgagattggtagttctttccccctctcaagctggcgtgaaatgcaaccttacggcgtctacgttactacaaggtccagaaagtgtaggtattgctactatttttattttttattggttctggagaaatgcagacagtcaatgaacacaactgtctcaatatgcatctatgcacatgcacacacacacacatcacaggtacccctacaaagagaggtctcttgataatgtttcattaccacgtggcatccccccccccccccccaataaacaagtggccgagttcccctgttgcagaggaggacaaaaaaaccgctggtgttggtaccattatgcagcaactagcacaacaaacaaccgacccagacatacaaatcaacaacacttcgccaaagacaccctttccagggaggatccactcccaacgtctctccataatgtctctgttggcccatgtctctgtcgttgacaccgtaaccacaccaaccaacccgtccattgtactgggatggtcgtccatagacacctctccaacggggaacacctcattcgtaaaccgccaaggttaccgttcctcctgactcgccccgttgttgatgctgcgcacctgtggttgcccaacatggttgtatatcgtgtaaccacaccaacacatgtgcagcacatgtgtttaaaagagtgtcatggaggtggatcatgatggaagtggactttaccacttgggaactgtctccactcccgggaagaaaagacccggcgtatcacgcggttgcctcaatggggcaatttggaaggagaaatatagggaaaatcacgtcgctctcggacggggaagagttccagactatgaggggggggggtggtatataaagacaggagatgtccacccccagagagaggaagaagttggaactttagaagagagagataactttccccagtgtccatcaatacacaaccaaacacaaactctatatttacacatataaccccctctctagaatgccacattcctatgactacgatgccattgtcattggttccggtccaggtggtgaaggtgctgcaatgggcttagttaagcagggtgctagagttgctgtcatcgaaagatatcaaaatgttggtggtggttgtactcactggggtacaattccatctaaggcattgagacatgcagtttccagaattattgagtttaaccaaaaccctttatactctgatcattcaagattgttgagatcatcttttgctgatattttgaaccatgctgacaacgtcatcaaccaacaaactcgtatgcgtcaaggcttctatgagagaaatcattgtgagattttacaaggtaacgctagatttgtcgatgagcatactcttgcattagactgtccagacggttccgttgagactcttaccgctgaaaaattcgttattgcttgtggttccagaccataccacccaaccgatgtcgatttcactcaccctcgtatctacgattccgattctattttgtctatgcatcatgaaccaagacatgttttgatttatggtgctggtgttatcggttgtgaatatgcttctattttcagaggtatggatgttaaggttgacttgattaatacaagagacagattattagctttccttgatcaggaaatgtctgattccctttcctaccatttttggaactccggtgtcgtcatcagacacaacgaggaatatgaaaagattgaaggttgtgatgacggcgttattatgcaccttaagtctggtaaaaagttaaaagcagattgcttgttatatgcaaatggtagaaccggtaacacagactccttggctttacaaaacattggtttagaaaccgattcaagaggtcaattaaaggtcaattcaatgtatcaaactgcacaaccacacgtttacgcagttggtgacgttattggttacccttcattggcatctgccgcttacgatcaaggtagaatcgccgctcaagcacttgttaagggtgaagcaactgcacacttaatcgaagatatccctaccggtatctacactatcccagaaatctcttctgttggcaagactgaacaacaattaaccgcaatgaaggttccatacgaagtcggtcgtgcccagttcaagcatttggctagagcacaaattgttggtatgaatgttggtactttgaaaatcttgtttcacagagaaacaaaggaaatcttgggcattcactgtttcggcgaaagagctgcagagattattcacatcggtcaagccattatggaacaaaaaggcggtggtaataccattgaatatttcgttaataccaccttcaactacccaacaatggccgaagcatatagagtcgctgctttaaacggtttaaacagattgttttaattaacatctgaatgtaaaatgaacattaaaatgaattactaaactttacgtctactttacaatctataaactttgtttaatcatataacgaaatacactaatacacaatcctgtacgtatgtaatacttttatccatcaaggattgagaaaaaaaagtaatgattccctgggccattaaaacttagacccccaagcttggataggtcactctctattttcgtttctcccttccctgatagaagggtgatatgtaattaagaataatatataattttataataaaagaattcgcccttacctgcagggataacttcgtataatgtatgctatacgaagttatgctgcaacggcaacatcaatgtccacgtttacacacctacatttatatctatatttatatttatatttatttatttatgctacttagcttctatagttagttaatgcactcacgatattcaaaattgacacccttcaactactccctactattgtctactactgtctactactcctctttactatagctgctcccaataggctccaccaataggctctgtcaatacattttgcgccgccacctttcaggttgtgtcactcctgaaggaccatattgggtaatcgtgcaatttctggaagagagtgccgcgagaagtgaggcccccactgtaaatcctcgagggggcatggagtatggggcatgnaggatggaggatggggggggggggggaaaataggtagcgaaaggacccgctatcaccccacccggagaactcgttgccgggaagtcatatttcgacactccggggagtctataaaaggcgggttttgtcttttgccagttgatgttgctgagaggacttgtttgccgtttcttccgatttaacagtatagaatcaaccactgttaattatacacgttatactaacacaacaaaaacaaaaacaacgacaacaacaacaacaatgtttgctttctactttctcaccgcatgcaccactttgaagggtgttttcggagtttctccgagttacaatggtcttggtctcaccccacagatgggttgggacagctggaatacgtttgcctgcgatgtcagtgaacagctacttctagacactgctgatagaatttctgacttggggctaaaggatatgggttacaagtatgtcatcctagatgactgttggtctagcggcagggattccgacggtttcctcgttgcagacaagcacaaatttcccaacggtatgggccatgttgcagaccacctgcataataacagctttcttttcggtatgtattcgtctgctggtgagtacacctgtgctgggtaccctgggtctctggggcgtgaggaagaagatgctcaattctttgcaaataaccgcgttgactacttgaagtatgataattgttacaataaaggtcaatttggtacaccagacgtttcttaccaccgttacaaggccatgtcagatgctttgaataaaactggtaggcctattttctattctctatgtaactggggtcaggatttgacattttactggggctctggtatcgccaattcttggagaatgagcggagatattactgctgagttcacccgtccagatagcagatgtccctgtgacggtgacgaatatgattgcaagtacgccggtttccattgttctattatgaatattcttaacaaggcagctccaatggggcaaaatgcaggtgttggtggttggaacgatctggacaatctagaggtcggagtcggtaatttgactgacgatgaggaaaaggcccatttctctatgtgggcaatggtaaagtccccacttatcattggtgccgacgtgaatcacttaaaggcatcttcgtactcgatctacagtcaagcctctgtcatcgcaattaatcaagatccaaagggtattccagccacaagagtctggagatattatgtttcagacaccgatgaatatggacaaggtgaaattcaaatgtggagtggtccgcttgacaatggtgaccaagtggttgctttattgaatggaggaagcgtagcaagaccaatgaacacgaccttggaagagattttctttgacagcaatttgggttcaaaggaactgacatcgacttgggatatttacgacttatgggccaacagagttgacaactctacggcgtctgctatccttgaacagaataaggcagccaccggtattctctacaatgctacagagcagtcttataaagacggtttgtctaagaatgatacaagactgtttggccagaaaattggtagtctttctccaaatgctatacttaacacaactgttccagctcatggtatcgccttctataggttgagaccctcggcttaagctcaatgttgagcaaagcaggacgagaaaaaaaaaaataatgattgttaagaagttcatgaaaaaaaaaaggaaaaatactcaaatacttataacagagtgattaaataataaacggcagtataccctatcaggtattgagatagttttatttttgtaggtatataatctgaagcctttgaactattttctcgtatatatcatggagtatacattgcattagcaacattgcatactagttcataacttcgtataatgtatgctatacgaagttattaattaacaagggcgaattccttgatttatatacacctttgccaaccgcttgttacttgataaggaaaagatagatttctaaagtgcaggaaaagaaacgccactacgtcatgaaacaaaagaaatgaaacactctgcaaaagggaaaaccaatgacgccttcaaaacgtactgactttccgcctccttttctgccttttttttttctccctcaatttgccaattcccctttccgctaattttacatcacctttttgtttgtttcccttttcggccaagttttccatttcttttttcggctgagcccttctttggcgtcgacgtaatttctcggcatgtggccaatgtatattgacagtagatgaagtagacgttcttagtaactgttagggtgagattgccacccccccttccttcttttactatctgtaataccatcaccatagcaatagtttaaccatgttggagctggaaatacaacgtctatagagggaagtcatcatattacgccattttacggaccagggacaccctgtagtgtgtttcctctcttgtagaggtaggttttcaaatggactctggcgtcgatttccagcaagtcattcccgtggttcaccatttctactttttgcgctacctctcttgacacagaaatgaatgatgacgtgtaaattacccgtccgagacctggactccggagaaactgtattaattacgcgccaaacaagacaggtgtcggataaacgtgcatgtacagactgcgagccgaaaacggaaggggggaaagaaaacagtggagtcccattgttgttccggaaatggaaaacgggaactggcggaaaagaaacgaaacaaaacaaaagaaaaagaggaaaaaaaagaaaaaaaaaagaaaaagacactgcacgtgattgctggtgtgtgctgcgtaaccgcggcactttatttcgtaaatgaaggggcc

SEQ ID NO: 134 - DNA, 1656 bp, Saccharomyces cerevisiae
  source 1..1656: mol_type = other DNA
atgtttgctttctactttctcaccgcatgcaccactttgaagggtgttttcggagtttctccgagttacaatggtcttggtctcaccccacagatgggttgggacagctggaatacgtttgcctgcgatgtcagtgaacagctacttctagacactgctgatagaatttctgacttggggctaaaggatatgggttacaagtatgtcatcctagatgactgttggtctagcggcagggattccgacggtttcctcgttgcagacaagcacaaatttcccaacggtatgggccatgttgcagaccacctgcataataacagctttcttttcggtatgtattcgtctgctggtgagtacacctgtgctgggtaccctgggtctctggggcgtgaggaagaagatgctcaattctttgcaaataaccgcgttgactacttgaagtatgataattgttacaataaaggtcaatttggtacaccagacgtttcttaccaccgttacaaggccatgtcagatgctttgaataaaactggtaggcctattttctattctctatgtaactggggtcaggatttgacattttactggggctctggtatcgccaattcttggagaatgagcggagatattactgctgagttcacccgtccagatagcagatgtccctgtgacggtgacgaatatgattgcaagtacgccggtttccattgttctattatgaatattcttaacaaggcagctccaatggggcaaaatgcaggtgttggtggttggaacgatctggacaatctagaggtcggagtcggtaatttgactgacgatgaggaaaaggcccatttctctatgtgggcaatggtaaagtccccacttatcattggtgccgacgtgaatcacttaaaggcatcttcgtactcgatctacagtcaagcctctgtcatcgcaattaatcaagatccaaagggtattccagccacaagagtctggagatattatgtttcagacaccgatgaatatggacaaggtgaaattcaaatgtggagtggtccgcttgacaatggtgaccaagtggttgctttattgaatggaggaagcgtagcaagaccaatgaacacgaccttggaagagattttctttgacagcaatttgggttcaaaggaactgacatcgacttgggatatttacgacttatgggccaacagagttgacaactctacggcgtctgctatccttgaacagaataaggcagccaccggtattctctacaatgctacagagcagtcttataaagacggtttgtctaagaatgatacaagactgtttggccagaaaattggtagtctttctccaaatgctatacttaacacaactgttccagctcatggtatcgccttctataggttgagaccctcggcttaagctcaatgttgagcaaagcaggacgagaaaaaaaaaaataatgattgttaagaagttcatgaaaaaaaaaaggaaaaatactcaaatacttataacagagtgattaaataataaacggcagtataccctatcaggtattgagatagttttatttttgtaggtatataatctgaagcctttgaactattttctcgtatatatcatggagtatacattgcattagcaacattgcatactagt

SEQ ID NO: 135 - DNA, 6279 bp, synthetic construct
  source 1..6279: mol_type = other DNA; note = E. coli SthA URA integration fragment
ggccccttcatttacgaaataaagtgccgcggttacgcagcacacaccagcaatcacgtgcagtgtctttttcttttttttttcttttttttcctctttttcttttgttttgtttcgtttcttttccgccagttcccgttttccatttccggaacaacaatgggactccactgttttctttccccccttccgttttcggctcgcagtctgtacatgcacgtttatccgacacctgtcttgtttggcgcgtaattaatacagtttctccggagtccaggtctcggacgggtaatttacacgtcatcattcatttctgtgtcaagagaggtagcgcaaaaagtagaaatggtgaaccacgggaatgacttgctggaaatcgacgccagagtccatttgaaaacctacctctacaagagaggaaacacactacagggtgtccctggtccgtaaaatggcgtaatatgatgacttccctctatagacgttgtatttccagctccaacatggttaaactattgctatggtgatggtattacagatagtaaaagaaggaagggggggtggcaatctcaccctaacagttactaagaacgtctacttcatctactgtcaatatacattggccacatgccgagaaattacgtcgacgccaaagaagggctcagccgaaaaaagaaatggaaaacttggccgaaaagggaaacaaacaaaaaggtgatgtaaaattagcggaaaggggaattggcaaattgagggagaaaaaaaaaaggcagaaaaggaggcggaaagtcagtacgttttgaaggcgtcattggttttcccttttgcagagtgtttcatttcttttgtttcatgacgtagtggcgtttcttttcctgcactttagaaatctatcttttccttatcaagtaacaagcggttggcaaaggtgtatataaatcaaggaattcccactttgaaccctttgaattttgatatcgtttattttaaatttatttgcggccgcggatccctcgagattggtagttctttccccctctcaagctggcgtgaaatgcaaccttacggcgtctacgttactacaaggtccagaaagtgtaggtattgctactatttttattttttattggttctggagaaatgcagacagtcaatgaacacaactgtctcaatatgcatctatgcacatgcacacacacacacatcacaggtacccctacaaagagaggtctcttgataatgtttcattaccacgtggcatccccccccccccccccaataaacaagtggccgagttcccctgttgcagaggaggacaaaaaaaccgctggtgttggtaccattatgcagcaactagcacaacaaacaaccgacccagacatacaaatcaacaacacttcgccaaagacaccctttccagggaggatccactcccaacgtctctccataatgtctctgttggcccatgtctctgtcgttgacaccgtaaccacaccaaccaacccgtccattgtactgggatggtcgtccatagacacctctccaacggggaacacctcattcgtaaaccgccaaggttaccgttcctcctgactcgccccgttgttgatgctgcgcacctgtggttgcccaacatggttgtatatcgtgtaaccacaccaacacatgtgcagcacatgtgtttaaaagagtgtcatggaggtggatcatgatggaagtggactttaccacttgggaactgtctccactcccgggaagaaaagacccggcgtatcacgcggttgcctcaatggggcaatttggaaggagaaatatagggaaaatcacgtcgctctcggacggggaagagttccagactatgaggggggggggtggtatataaagacaggagatgtccacccccagagagaggaagaagttggaactttagaagagagagataactttccccagtgtccatcaatacacaaccaaacacaaactctatatttacacatataaccccctctctagaatgccacattcctatgactacgatgccattgtcattggttccggtccaggtggtgaaggtgctgcaatgggcttagttaagcagggtgctagagttgctgtcatcgaaagatatcaaaatgttggtggtggttgtactcactggggtacaattccatctaaggcattgagacatgcagtttccagaattattgagtttaaccaaaaccctttatactctgatcattcaagattgttgagatcatcttttgctgatattttgaaccatgctgacaacgtcatcaaccaacaaactcgtatgcgtcaaggcttctatgagagaaatcattgtgagattttacaaggtaacgctagatttgtcgatgagcatactcttgcattagactgtccagacggttccgttgagactcttaccgctgaaaaattcgttattgcttgtggttccagaccataccacccaaccgatgtcgatttcactcaccctcgtatctacgattccgattctattttgtctatgcatcatgaaccaagacatgttttgatttatggtgctggtgttatcggttgtgaatatgcttctattttcagaggtatggatgttaaggttgacttgattaatacaagagacagattattagctttccttgatcaggaaatgtctgattccctttcctaccatttttggaactccggtgtcgtcatcagacacaacgaggaatatgaaaagattgaaggttgtgatgacggcgttattatgcaccttaagtctggtaaaaagttaaaagcagattgcttgttatatgcaaatggtagaaccggtaacacagactccttggctttacaaaacattggtttagaaaccgattcaagaggtcaattaaaggtcaattcaatgtatcaaactgcacaaccacacgtttacgcagttggtgacgttattggttacccttcattggcatctgccgcttacgatcaaggtagaatcgccgctcaagcacttgttaagggtgaagcaactgcacacttaatcgaagatatccctaccggtatctacactatcccagaaatctcttctgttggcaagactgaacaacaattaaccgcaatgaaggttccatacgaagtcggtcgtgcccagttcaagcatttggctagagcacaaattgttggtatgaatgttggtactttgaaaatcttgtttcacagagaaacaaaggaaatcttgggcattcactgtttcggcgaaagagctgcagagattattcacatcggtcaagccattatggaacaaaaaggcggtggtaataccattgaatatttcgttaataccaccttcaactacccaacaatggccgaagcatatagagtcgctgctttaaacggtttaaacagattgttttaattaacatctgaatgtaaaatgaacattaaaatgaattactaaactttacgtctactttacaatctataaactttgtttaatcatataacgaaatacactaatacacaatcctgtacgtatgtaatacttttatccatcaaggattgagaaaaaaaagtaatgattccctgggccattaaaacttagacccccaagcttggataggtcactctctattttcgtttctcccttccctgatagaagggtgatatgtaattaagaataatatataattttataataaaagaattcatagcctcatgaaatcagccatttgcttttgttcaacgatcttttgaaattgttgttgttcttggtagttaagttgatccatcttggcttatgttgtgtgtatgttgtagttattcttagtatattcctgtcctgagtttagtgaaacataatatcgccttgaaatgaaaatgctgaaattcgtcgacatacaatttttcaaactttttttttttcttggtgcacggacatgtttttaaaggaagtactctataccagttattcttcacaaatttaattgctggagaatagatcttcaacgctttaataaagtagtttgtttgtcaaggatggcgtcatacaaagaaagatcagaatcacacacttcccctgttgctaggagacttttctccatcatggaggaaaagaagtctaacctttgtgcatcattggatattactgaaactgaaaagcttctctctattttggacactattggtccttacatctgtctagttaaaacacacatcgatattgtttctgattttacgtatgaaggaactgtgttgcctttgaaggagcttgccaagaaacataattttatgatttttgaagatagaaaatttgctgatattggtaacactgttaaaaatcaatataaatctggtgtcttccgtattgccgaatgggctgacatcactaatgcacatggtgtaacgggtgcaggtattgtttctggcttgaaggaggcagcccaagaaacaaccagtgaacctagaggtttgctaatgcttgctgagttatcatcaaagggttctttagcatatggtgaatatacagaaaaaacagtagaaattgctaaatctgataaagagtttgtcattggttttattgcgcaacacgatatgggcggtagagaagaaggttttgactggatcattatgactccaggggttggtttagatgacaaaggtgatgcacttggtcaacaatatagaactgttgatgaagttgtaaagactggaacggatatcataattgttggtagaggtttgtacggtcaaggaagagatcctatagagcaagctaaaagataccaacaagctggttggaatgcttatttaaacagatttaaatgattcttacacaaagatttgatacatgtacactagtttaaataagcatgaaaagaattacacaagcaaaaaaaaaaaaataaatgaggtactttacgttcacctacaaccaaaaaaactagatagagtaaaatcttaagatttagaaaaagttgtttaacaaaggctttagtatgtgaatttttaatgtagcaaagcgataactaataaacataaacaaaagtatggttttctttatcagtcaaatcattatcgattgattgttccgcgtatctgcagatagcctcatgaaatcagccatttgcttttgttcaacgatcttttgaaattgttgttgttcttggtagttaagttgatccatcttggcttatgttgtgtgtatgttgtagttattcttagtatattcctgtcctgagtttagtgaaacataatatcgccttgaaatgaaaatgctgaaattcgtcgacatacaatttttcaaactttttttttttcttggtgcacggacatgtttttaaaggaagtactctataccagttattcttcacaaatttaattgctggagaatagatcttcaacgccccgggggatctggatccgcggccgcaataacctcagggagaactttggcattgtactctccattgacgagtccgccaacccattcttgttaaacctaaccttgcattatcacattccctttgaccccctttagctgcatttccacttgtctacattaagattcattacacattctttttcgtatttctcttacctccctcccccctccatggatcttatatataaatcttttctataacaataatatctactagagttaaacaacaattccacttggcatggctgtctcagcaaatctgcttctacctactgcacgggtttgcatgtcattgtttctagcagggaatcgtccatgtacgttgtcctccatgatggtcttcccgctgccactttctttagtatcttaaatagagcagatcttacgtccactgtgcatccgtgcaccccgaaaatcgtatggttttccttgccacctctcacaattttgaatatgctcaacgcgaaagagaggggaagaggaatcgcattcgtagagtggctacattcaaccctgacaaaggaactagcgtttgtgcaggagagagtggtttgcatagatttcctttcctttgcaagcatattatatagagtagccaatacagtaacagctacagcacaaaaaagagaacgagaacgagaacgagaacaagaacaagaactagcactactgtcactgccagcatcaacattactaccattattccaacatgtttgcaactagaaatataaccattggtgtcagaacactcagaccaaccagtttcttgaaaacaaggtcttttctgcaacagaggctacaatcaacgctaaagaagagctatgaaccaaccaaatccg

SEQ ID NO: 136 - DNA, 6888 bp, synthetic construct
  source 1..6888: mol_type = other DNA; note = Synthetic - A. vinelandii MEL integration fragment
ccttcatttacgaaataaagtgccgcggttacgcagcacacaccagcaatcacgtgcagtgtctttttcttttttttttcttttttttcctctttttcttttgttttgtttcgtttcttttccgccagttcccgttttccatttccggaacaacaatgggactccactgttttctttccccccttccgttttcggctcgcagtctgtacatgcacgtttatccgacacctgtcttgtttggcgcgtaattaatacagtttctccggagtccaggtctcggacgggtaatttacacgtcatcattcatttctgtgtcaagagaggtagcgcaaaaagtagaaatggtgaaccacgggaatgacttgctggaaatcgacgccagagtccatttgaaaacctacctctacaagagaggaaacacactacagggtgtccctggtccgtaaaatggcgtaatatgatgacttccctctatagacgttgtatttccagctccaacatggttaaactattgctatggtgatggtattacagatagtaaaagaaggaagggggggtggcaatctcaccctaacagttactaagaacgtctacttcatctactgtcaatatacattggccacatgccgagaaattacgtcgacgccaaagaagggctcagccgaaaaaagaaatggaaaacttggccgaaaagggaaacaaacaaaaaggtgatgtaaaattagcggaaaggggaattggcaaattgagggagaaaaaaaaaaggcagaaaaggaggcggaaagtcagtacgttttgaaggcgtcattggttttcccttttgcagagtgtttcatttcttttgtttcatgacgtagtggcgtttcttttcctgcactttagaaatctatcttttccttatcaagtaacaagcggttggcaaaggtgtatataaatcaaggaattcgcccttgttaattaataacttcgtatagcatacattatacgaagttatgaactagtatgcaatgttgctaatgcaatgtatactccatgatatatacgagaaaatagttcaaaggcttcagattatatacctacaaaaataaaactatctcaatacctgatagggtatactgccgtttattatttaatcactctgttataagtatttgagtatttttccttttttttttcatgaacttcttaacaatcattatttttttttttctcgtcctgctttgctcaacattgagcttaagccgagggtctcaacctatagaaggcgataccatgagctggaacagttgtgttaagtatagcatttggagaaagactaccaattttctggccaaacagtcttgtatcattcttagacaaaccgtctttataagactgctctgtagcattgtagagaataccggtggctgccttattctgttcaaggatagcagacgccgtagagttgtcaactctgttggcccataagtcgtaaatatcccaagtcgatgtcagttcctttgaacccaaattgctgtcaaagaaaatctcttccaaggtcgtgttcattggtcttgctacgcttcctccattcaataaagcaaccacttggtcaccattgtcaagcggaccactccacatttgaatttcaccttgtccatattcatcggtgtctgaaacataatatctccagactcttgtggctggaataccctttggatcttgattaattgcgatgacagaggcttgactgtagatcgagtacgaagatgcctttaagtgattcacgtcggcaccaatgataagtggggactttaccattgcccacatagagaaatgggccttttcctcatcgtcagtcaaattaccgactccgacctctagattgtccagatcgttccaaccaccaacacctgcattttgccccattggagctgccttgttaagaatattcataatagaacaatggaaaccggcgtacttgcaatcatattcgtcaccgtcacagggacatctgctatctggacgggtgaactcagcagtaatatctccgctcattctccaagaattggcgataccagagccccagtaaaatgtcaaatcctgaccccagttacatagagaatagaaaataggcctaccagttttattcaaagcatctgacatggccttgtaacggtggtaagaaacgtctggtgtaccaaattgacctttattgtaacaattatcatacttcaagtagtcaacgcggttatttgcaaagaattgagcatcttcttcctcacgccccagagacccagggtacccagcacaggtgtactcaccagcagacgaatacataccgaaaagaaagctgttattatgcaggtggtctgcaacatggcccataccgttgggaaatttgtgcttgtctgcaacgaggaaaccgtcggaatccctgccgctagaccaacagtcatctaggatgacatacttgtaacccatatcctttagccccaagtcagaaattctatcagcagtgtctagaagtagctgttcactgacatcgcaggcaaacgtattccagctgtcccaacccatctgtggggtgagaccaagaccattgtaactcggagaaactccgaaaacacccttcaaagtggtgcatgcggtgagaaagtagaaagcaaacattgttgttgttgttgtcgttgtttttgtttttgttgtgttagtataacgtgtataattaacagtggttgattctatactgttaaatcggaagaaacggcaaacaagtcctctcagcaacatcaactggcaaaagacaaaacccgccttttatagactccccggagtgtcgaaatatgacttcccggcaacgagttctccgggtggggtgatagcgggtcctttcgctacctattttccccccccccccccatcctccatcctncatgccccatactccatgccccctcgaggatttacagtgggggcctcacttctcgcggcactctcttccagaaattgcacgattacccaatatggtccttcaggagtgacacaacctgaaaggtggcggcgcaaaatgtattgacagagcctattggtggagcctattgggagcagctatagtaaagaggagtagtagacagtagtagacaatagtagggagtagttgaagggtgtcaattttgaatatcgtgagtgcattaactaactatagaagctaagtagcataaataaataaatataaatataaatatagatataaatgtaggtgtgtaaacgtggacattgatgttgccgttgcagcataacttcgtatagcatacattatacgaagttatccctgcaggtaagggcgaattcttttattataaaattatatattattcttaattacatatcacccttctatcagggaagggagaaacgaaaatagagagtgacctatccaagcttgggggtctaagttttaatggcccagggaatcattacttttttttctcaatccttgatggataaaagtattacatacgtacaggattgtgtattagtgtatttcgttatatgattaaacaaagtttatagattgtaaagtagacgtaaagtttagtaattcattttaatgttcattttacattcagatgttaattaattagaacaatctgttcaaaccatcgtacgcagccactctatatgcttctgccattgtaggataattgaatgtagtattaatgaaatacttcaaagtgttggcttcacccttttggttcataattgcttgaccgatgtgcacaatttcagatgcttgataaccaaaacaatggacgcctaagatttcgagtgtttctctgtgaaacaaaatctttaacatgcctgccttttcaacagcaatctgggctcttgccatacctttgaaaaaggccttaccaacctcgtatggaacttttgcctgagtaagttctctttctgtcttaccaacactggaaatttctggaatggtgtagataccggtagggacatcgtcaacaaatctccaggagtcgttttcagtgatggagccagcagcggatctaccttgatcgtaagcagcagatgccaaagatggccaaccaataacatcaccagcagcgtagatgttggaaacttcggttctataatgctcatcgacttgaatttgacctctaccattagcctttaaaccaatgttttcaagacccaacttatcagtgttaccggttctaccgttggaccaaaggaatgcatctgccttgatctttttaccagacttcaaatgtaaaatgacaccgttgtcgaggccttcaactctttcgtactcctcgttatgtctgattaagacattgttattccttaagtgataagacaatgagtcggaaatttcgtcatcgagaaaagatagtaattgatcacgattgtcaatcaaatcgactaaaacacctagtcctgagaaaatacttgcgtattcgcaaccgataacaccagctccgtagataatcaaacgtcttggagtatgacccaaagataagatagtatcagaatcgtagatcctagggtgagtaaagtccacatcggctggtctataaggtcttgaaccggtagcaataacaaattgctttgcgaccaaggtttcgaccataccattcaaatgaacgacttcaatagtatgctcgtcacagaatgatgcggtaccgaaaaaggtatcaattctgtttctagcataatatccggttctggaagagacttgcttcgcaataacttgctctgctgattttagaacgtcagcgaaagagaaccatcttggttcaccaatttgacggaataatgggttgttgttgtactgcataatttgtctaacagagtgccttaaagccttacttgggatagtgcctaagtgcgtacaattaccgccaacttgtggtctatcatcaacaactgcaacttttctaccagctttgactgcattcatagcggcaccttcacccgctggacctgtaccgataacaacaacatcataattgtaaacagccattctagagagggggttatatgtgtaaatatagagtttgtgtttggttgtgtattgatggacactggggaaagttatctctctcttctaaagttccaacttcttcctctctctgggggtggacatctcctgtctttatataccacccccccccctcatagtctggaactcttccccgtccgagagcgacgtgattttccctatatttctccttccaaattgccccattgaggcaaccgcgtgatacgccgggtcttttcttcccgggagtggagacagttcccaagtggtaaagtccacttccatcatgatccacctccatgacactcttttaaacacatgtgctgcacatgtgttggtgtggttacacgatatacaaccatgttgggcaaccacaggtgcgcagcatcaacaacggggcgagtcaggaggaacggtaaccttggcggtttacgaatgaggtgttccccgttggagaggtgtctatggacgaccatcccagtacaatggacgggttggttggtgtggttacggtgtcaacgacagagacatgggccaacagagacattatggagagacgttgggagtggatcctccctggaaagggtgtctttggcgaagtgttgttgatttgtatgtctgggtcggttgtttgttgtgctagttgctgcataatggtaccaacaccagcggtttttttgtcctcctctgcaacaggggaactcggccacttgtttattggggggggggggggggatgccacgtggtaatgaaacattatcaagagacctctctttgtaggggtacctgtgatgtgtgtgtgtgtgcatgtgcatagatgcatattgagacagttgtgttcattgactgtctgcatttctccagaaccaataaaaaataaaaatagtagcaatacctacactttctggaccttgtagtaacgtagacgccgtaaggttgcatttcacgccagcttgagagggggaaagaactaccaatctcgagggatccgcggccgcaataacctcagggagaactttggcattgtactctccattgacgagtccgccaacccattcttgttaaacctaaccttgcattatcacattccctttgaccccctttagctgcatttccacttgtctacattaagattcattacacattctttttcgtatttctcttacctccctcccccctccatggatcttatatataaatcttttctataacaataatatctactagagttaaacaacaattccacttggcatggctgtctcagcaaatctgcttctacctactgcacgggtttgcatgtcattgtttctagcagggaatcgtccatgtacgttgtcctccatgatggtcttcccgctgccactttctttagtatcttaaatagagcagatcttacgtccactgtgcatccgtgcaccccgaaaatcgtatggttttccttgccacctctcacaattttgaatatgctcaacgcgaaagagaggggaagaggaatcgcattcgtagagtggctacattcaaccctgacaaaggaactagcgtttgtgcaggagagagtggtttgcatagatttcctttcctttgcaagcatattatatagagtagccaatacagtaacagctacagcacaaaaaagagaacgagaacgagaacgagaacaagaacaagaactagcactactgtcactgccagcatcaacattactaccattattccaacatgtttgcaactagaaatataaccattggtgtcagaacactcagaccaaccagtttcttgaaaacaaggtcttttctgcaacagaggctacaatcaacgctaaagaagagctatgaaccaaccaaatccgagct

SEQ ID NO: 137 - DNA, 6276 bp, synthetic construct
  source 1..6276: mol_type = other DNA; note = Synthetic - A. vinelandii URA integration fragment
ggccccttcatttacgaaataaagtgccgcggttacgcagcacacaccagcaatcacgtgcagtgtctttttcttttttttttcttttttttcctctttttcttttgttttgtttcgtttcttttccgccagttcccgttttccatttccggaacaacaatgggactccactgttttctttccccccttccgttttcggctcgcagtctgtacatgcacgtttatccgacacctgtcttgtttggcgcgtaattaatacagtttctccggagtccaggtctcggacgggtaatttacacgtcatcattcatttctgtgtcaagagaggtagcgcaaaaagtagaaatggtgaaccacgggaatgacttgctggaaatcgacgccagagtccatttgaaaacctacctctacaagagaggaaacacactacagggtgtccctggtccgtaaaatggcgtaatatgatgacttccctctatagacgttgtatttccagctccaacatggttaaactattgctatggtgatggtattacagatagtaaaagaaggaagggggggtggcaatctcaccctaacagttactaagaacgtctacttcatctactgtcaatatacattggccacatgccgagaaattacgtcgacgccaaagaagggctcagccgaaaaaagaaatggaaaacttggccgaaaagggaaacaaacaaaaaggtgatgtaaaattagcggaaaggggaattggcaaattgagggagaaaaaaaaaaggcagaaaaggaggcggaaagtcagtacgttttgaaggcgtcattggttttcccttttgcagagtgtttcatttcttttgtttcatgacgtagtggcgtttcttttcctgcactttagaaatctatcttttccttatcaagtaacaagcggttggcaaaggtgtatataaatcaaggaattcccactttgaaccctttgaattttgatatcgtttattttaaatttatttgcggccgcggatccctcgagattggtagttctttccccctctcaagctggcgtgaaatgcaaccttacggcgtctacgttactacaaggtccagaaagtgtaggtattgctactatttttattttttattggttctggagaaatgcagacagtcaatgaacacaactgtctcaatatgcatctatgcacatgcacacacacacacatcacaggtacccctacaaagagaggtctcttgataatgtttcattaccacgtggcatccccccccccccccccaataaacaagtggccgagttcccctgttgcagaggaggacaaaaaaaccgctggtgttggtaccattatgcagcaactagcacaacaaacaaccgacccagacatacaaatcaacaacacttcgccaaagacaccctttccagggaggatccactcccaacgtctctccataatgtctctgttggcccatgtctctgtcgttgacaccgtaaccacaccaaccaacccgtccattgtactgggatggtcgtccatagacacctctccaacggggaacacctcattcgtaaaccgccaaggttaccgttcctcctgactcgccccgttgttgatgctgcgcacctgtggttgcccaacatggttgtatatcgtgtaaccacaccaacacatgtgcagcacatgtgtttaaaagagtgtcatggaggtggatcatgatggaagtggactttaccacttgggaactgtctccactcccgggaagaaaagacccggcgtatcacgcggttgcctcaatggggcaatttggaaggagaaatatagggaaaatcacgtcgctctcggacggggaagagttccagactatgaggggggggggtggtatataaagacaggagatgtccacccccagagagaggaagaagttggaactttagaagagagagataactttccccagtgtccatcaatacacaaccaaacacaaactctatatttacacatataaccccctctctagaatggctgtttacaattatgatgttgttgttatcggtacaggtccagcgggtgaaggtgccgctatgaatgcagtcaaagctggtagaaaagttgcagttgttgatgatagaccacaagttggcggtaattgtacgcacttaggcactatcccaagtaaggctttaaggcactctgttagacaaattatgcagtacaacaacaacccattattccgtcaaattggtgaaccaagatggttctctttcgctgacgttctaaaatcagcagagcaagttattgcgaagcaagtctcttccagaaccggatattatgctagaaacagaattgatacctttttcggtaccgcatcattctgtgacgagcatactattgaagtcgttcatttgaatggtatggtcgaaaccttggtcgcaaagcaatttgttattgctaccggttcaagaccttatagaccagccgatgtggactttactcaccctaggatctacgattctgatactatcttatctttgggtcatactccaagacgtttgattatctacggagctggtgttatcggttgcgaatacgcaagtattttctcaggactaggtgttttagtcgatttgattgacaatcgtgatcaattactatcttttctcgatgacgaaatttccgactcattgtcttatcacttaaggaataacaatgtcttaatcagacataacgaggagtacgaaagagttgaaggcctcgacaacggtgtcattttacatttgaagtctggtaaaaagatcaaggcagatgcattcctttggtccaacggtagaaccggtaacactgataagttgggtcttgaaaacattggtttaaaggctaatggtagaggtcaaattcaagtcgatgagcattatagaaccgaagtttccaacatctacgctgctggtgatgttattggttggccatctttggcatctgctgcttacgatcaaggtagatccgctgctggctccatcactgaaaacgactcctggagatttgttgacgatgtccctaccggtatctacaccattccagaaatttccagtgttggtaagacagaaagagaacttactcaggcaaaagttccatacgaggttggtaaggcctttttcaaaggtatggcaagagcccagattgctgttgaaaaggcaggcatgttaaagattttgtttcacagagaaacactcgaaatcttaggcgtccattgttttggttatcaagcatctgaaattgtgcacatcggtcaagcaattatgaaccaaaagggtgaagccaacactttgaagtatttcattaatactacattcaattatcctacaatggcagaagcatatagagtggctgcgtacgatggtttgaacagattgttctaattaattaacatctgaatgtaaaatgaacattaaaatgaattactaaactttacgtctactttacaatctataaactttgtttaatcatataacgaaatacactaatacacaatcctgtacgtatgtaatacttttatccatcaaggattgagaaaaaaaagtaatgattccctgggccattaaaacttagacccccaagcttggataggtcactctctattttcgtttctcccttccctgatagaagggtgatatgtaattaagaataatatataattttataataaaagaattcatagcctcatgaaatcagccatttgcttttgttcaacgatcttttgaaattgttgttgttcttggtagttaagttgatccatcttggcttatgttgtgtgtatgttgtagttattcttagtatattcctgtcctgagtttagtgaaacataatatcgccttgaaatgaaaatgctgaaattcgtcgacatacaatttttcaaactttttttttttcttggtgcacggacatgtttttaaaggaagtactctataccagttattcttcacaaatttaattgctggagaatagatcttcaacgctttaataaagtagtttgtttgtcaaggatggcgtcatacaaagaaagatcagaatcacacacttcccctgttgctaggagacttttctccatcatggaggaaaagaagtctaacctttgtgcatcattggatattactgaaactgaaaagcttctctctattttggacactattggtccttacatctgtctagttaaaacacacatcgatattgtttctgattttacgtatgaaggaactgtgttgcctttgaaggagcttgccaagaaacataattttatgatttttgaagatagaaaatttgctgatattggtaacactgttaaaaatcaatataaatctggtgtcttccgtattgccgaatgggctgacatcactaatgcacatggtgtaacgggtgcaggtattgtttctggcttgaaggaggcagcccaagaaacaaccagtgaacctagaggtttgctaatgcttgctgagttatcatcaaagggttctttagcatatggtgaatatacagaaaaaacagtagaaattgctaaatctgataaagagtttgtcattggttttattgcgcaacacgatatgggcggtagagaagaaggttttgactggatcattatgactccaggggttggtttagatgacaaaggtgatgcacttggtcaacaatatagaactgttgatgaagttgtaaagactggaacggatatcataattgttggtagaggtttgtacggtcaaggaagagatcctatagagcaagctaaaagataccaacaagctggttggaatgcttatttaaacagatttaaatgattcttacacaaagatttgatacatgtacactagtttaaataagcatgaaaagaattacacaagcaaaaaaaaaaaaataaatgaggtactttacgttcacctacaaccaaaaaaactagatagagtaaaatcttaagatttagaaaaagttgtttaacaaaggctttagtatgtgaatttttaatgtagcaaagcgataactaataaacataaacaaaagtatggttttctttatcagtcaaatcattatcgattgattgttccgcgtatctgcagatagcctcatgaaatcagccatttgcttttgttcaacgatcttttgaaattgttgttgttcttggtagttaagttgatccatcttggcttatgttgtgtgtatgttgtagttattcttagtatattcctgtcctgagtttagtgaaacataatatcgccttgaaatgaaaatgctgaaattcgtcgacatacaatttttcaaactttttttttttcttggtgcacggacatgtttttaaaggaagtactctataccagttattcttcacaaatttaattgctggagaatagatcttcaacgccccgggggatctggatccgcggccgcaataacctcagggagaactttggcattgtactctccattgacgagtccgccaacccattcttgttaaacctaaccttgcattatcacattccctttgaccccctttagctgcatttccacttgtctacattaagattcattacacattctttttcgtatttctcttacctccctcccccctccatggatcttatatataaatcttttctataacaataatatctactagagttaaacaacaattccacttggcatggctgtctcagcaaatctgcttctacctactgcacgggtttgcatgtcattgtttctagcagggaatcgtccatgtacgttgtcctccatgatggtcttcccgctgccactttctttagtatcttaaatagagcagatcttacgtccactgtgcatccgtgcaccccgaaaatcgtatggttttccttgccacctctcacaattttgaatatgctcaacgcgaaagagaggggaagaggaatcgcattcgtagagtggctacattcaaccctgacaaaggaactagcgtttgtgcaggagagagtggtttgcatagatttcctttcctttgcaagcatattatatagagtagccaatacagtaacagctacagcacaaaaaagagaacgagaacgagaacgagaacaagaacaagaactagcactactgtcactgccagcatcaacattactaccattattccaacatgtttgcaactagaaatataaccattggtgtcagaacactcagaccaaccagtttcttgaaaacaaggtcttttctgcaacagaggctacaatcaacgctaaagaagagctatgaaccaaccaaatcc

SEQ ID NO: 138 - DNA, 6892 bp, synthetic construct
  source 1..6892: mol_type = other DNA; note = Synthetic - Pseudomonas fluoresens SthA MEL integration fragment
cggatttggttggttcatagctcttctttagcgttgattgtagcctctgttgcagaaaagaccttgttttcaagaaactggttggtctgagtgttctgacaccaatggttatatttctagttgcaaacatgttggaataatggtagtaatgttgatgctggcagtgacagtagtgctagttcttgttcttgttctcgttctcgttctcgttctcttttttgtgctgtagctgttactgtattggctactctatataatatgcttgcaaaggaaaggaaatctatgcaaaccactctctcctgcacaaacgctagttcctttgtcagggttgaatgtagccactctacgaatgcgattcctcttcccctctctttcgcgttgagcatattcaaaattgtgagaggtggcaaggaaaaccatacgattttcggggtgcacggatgcacagtggacgtaagatctgctctatttaagatactaaagaaagtggcagcgggaagaccatcatggaggacaacgtacatggacgattccctgctagaaacaatgacatgcaaacccgtgcagtaggtagaagcagatttgctgagacagccatgccaagtggaattgttgtttaactctagtagatattattgttatagaaaagatttatatataagatccatggaggggggagggaggtaagagaaatacgaaaaagaatgtgtaatgaatcttaatgtagacaagtggaaatgcagctaaagggggtcaaagggaatgtgataatgcaaggttaggtttaacaagaatgggttggcggactcgtcaatggagagtacaatgccaaagttctccctgaggttattgcggccgcggatccctcgagattggtagttctttccccctctcaagctggcgtgaaatgcaaccttacggcgtctacgttactacaaggtccagaaagtgtaggtattgctactatttttattttttattggttctggagaaatgcagacagtcaatgaacacaactgtctcaatatgcatctatgcacatgcacacacacacacatcacaggtacccctacaaagagaggtctcttgataatgtttcattaccacgtggcatccccccccccccccccaataaacaagtggccgagttcccctgttgcagaggaggacaaaaaaaccgctggtgttggtaccattatgcagcaactagcacaacaaacaaccgacccagacatacaaatcaacaacacttcgccaaagacaccctttccagggaggatccactcccaacgtctctccataatgtctctgttggcccatgtctctgtcgttgacaccgtaaccacaccaaccaacccgtccattgtactgggatggtcgtccatagacacctctccaacggggaacacctcattcgtaaaccgccaaggttaccgttcctcctgactcgccccgttgttgatgctgcgcacctgtggttgcccaacatggttgtatatcgtgtaaccacaccaacacatgtgcagcacatgtgtttaaaagagtgtcatggaggtggatcatgatggaagtggactttaccacttgggaactgtctccactcccgggaagaaaagacccggcgtatcacgcggttgcctcaatggggcaatttggaaggagaaatatagggaaaatcacgtcgctctcggacggggaagagttccagactatgaggggggggggtggtatataaagacaggagatgtccacccccagagagaggaagaagttggaactttagaagagagagataactttccccagtgtccatcaatacacaaccaaacacaaactctatatttacacatataaccccctctctagataaaatggctgtttataactacgacgttgttgttttgggttctggtccagcaggcgaaggtgctgctatgaatgcagctaaagcaggcagaaaagttgctatggttgattcacgtagacaagtcggtggtaactgtacccacttaggtactattccttctaaggctttgagacactctgttcgtcaaatcatgcaattcaacactaatccaatgttcagagccattggcgaaccaagatggttctcctttccagatgttttaaagtctgcagaaaaggttatttccaagcaagtcgcttctcgtaccggctattacgctagaaacagagttgatttgtttttcggtactggttccttcgcagatgaacagactgttgaagtcgtttgtgcaaatggtgttgtcgagaagttagttgctaagcatattatcatcgccacaggttccagaccttacagaccagcagacatcgatttccatcatccacgtatctacgactctgataccatcttatctttaggccacacccctagaaagttgattatctacggtgccggtgttatcggttgcgagtatgcttctatcttttcaggtttgggtgtcttagtcgagttggtcgataacagagatcaacttttgtcctttttagactctgaaatttctcaagctctttcctatcacttttctaataacaacattacagttagacataatgaggaatacgacagagtcgaaggtttagataacggtgttattttgcatttgaagtccggtaaaaagattaaggccgatgcattgttatggtgtaacggtagaactggtaatactgacaagttaggtatggaaaacattggtgttaaggtcaactccagaggtcaaattgaagttgacgagaattacagaacctgtgtcacaaacatttatggtgctggtgatgttattggttggccatcacttgcctcagcagctcacgaccaaggtagatcagcagctggctctatcgttgataatggttcctggagatatgtcaacgatgttccaaccggtatctacactattccagaaatttcctcaattggtaaaaatgaacacgaattgactaaagctaaggttccttatgaggtcggtaaagcctttttcaagtctatggcaagagcacaaattgctggtgaaccacagggtatgcttaaaatcttattccatagagaaactttagaagtcttaggtgttcactgttttggttatcaagcatccgaaattgttcatattggccaggcaattatgaaccaaccaggtgaacaaaatactcttaagtacttcgtcaataccaccttcaactacccaacaatggctgaagcatatagagttgcagcttacgatggtttgaacagattgttctaattaattaacatctgaatgtaaaatgaacattaaaatgaattactaaactttacgtctactttacaatctataaactttgtttaatcatataacgaaatacactaatacacaatcctgtacgtatgtaatacttttatccatcaaggattgagaaaaaaaagtaatgattccctgggccattaaaacttagacccccaagcttggataggtcactctctattttcgtttctcccttccctgatagaagggtgatatgtaattaagaataatatataattttataataaaagaattcgcccttacctgcagggataacttcgtataatgtatgctatacgaagttatgctgcaacggcaacatcaatgtccacgtttacacacctacatttatatctatatttatatttatatttatttatttatgctacttagcttctatagttagttaatgcactcacgatattcaaaattgacacccttcaactactccctactattgtctactactgtctactactcctctttactatagctgctcccaataggctccaccaataggctctgtcaatacattttgcgccgccacctttcaggttgtgtcactcctgaaggaccatattgggtaatcgtgcaatttctggaagagagtgccgcgagaagtgaggcccccactgtaaatcctcgagggggcatggagtatggggcatgnaggatggaggatggggggggggggggaaaataggtagcgaaaggacccgctatcaccccacccggagaactcgttgccgggaagtcatatttcgacactccggggagtctataaaaggcgggttttgtcttttgccagttgatgttgctgagaggacttgtttgccgtttcttccgatttaacagtatagaatcaaccactgttaattatacacgttatactaacacaacaaaaacaaaaacaacgacaacaacaacaacaatgtttgctttctactttctcaccgcatgcaccactttgaagggtgttttcggagtttctccgagttacaatggtcttggtctcaccccacagatgggttgggacagctggaatacgtttgcctgcgatgtcagtgaacagctacttctagacactgctgatagaatttctgacttggggctaaaggatatgggttacaagtatgtcatcctagatgactgttggtctagcggcagggattccgacggtttcctcgttgcagacaagcacaaatttcccaacggtatgggccatgttgcagaccacctgcataataacagctttcttttcggtatgtattcgtctgctggtgagtacacctgtgctgggtaccctgggtctctggggcgtgaggaagaagatgctcaattctttgcaaataaccgcgttgactacttgaagtatgataattgttacaataaaggtcaatttggtacaccagacgtttcttaccaccgttacaaggccatgtcagatgctttgaataaaactggtaggcctattttctattctctatgtaactggggtcaggatttgacattttactggggctctggtatcgccaattcttggagaatgagcggagatattactgctgagttcacccgtccagatagcagatgtccctgtgacggtgacgaatatgattgcaagtacgccggtttccattgttctattatgaatattcttaacaaggcagctccaatggggcaaaatgcaggtgttggtggttggaacgatctggacaatctagaggtcggagtcggtaatttgactgacgatgaggaaaaggcccatttctctatgtgggcaatggtaaagtccccacttatcattggtgccgacgtgaatcacttaaaggcatcttcgtactcgatctacagtcaagcctctgtcatcgcaattaatcaagatccaaagggtattccagccacaagagtctggagatattatgtttcagacaccgatgaatatggacaaggtgaaattcaaatgtggagtggtccgcttgacaatggtgaccaagtggttgctttattgaatggaggaagcgtagcaagaccaatgaacacgaccttggaagagattttctttgacagcaatttgggttcaaaggaactgacatcgacttgggatatttacgacttatgggccaacagagttgacaactctacggcgtctgctatccttgaacagaataaggcagccaccggtattctctacaatgctacagagcagtcttataaagacggtttgtctaagaatgatacaagactgtttggccagaaaattggtagtctttctccaaatgctatacttaacacaactgttccagctcatggtatcgccttctataggttgagaccctcggcttaagctcaatgttgagcaaagcaggacgagaaaaaaaaaaataatgattgttaagaagttcatgaaaaaaaaaaggaaaaatactcaaatacttataacagagtgattaaataataaacggcagtataccctatcaggtattgagatagttttatttttgtaggtatataatctgaagcctttgaactattttctcgtatatatcatggagtatacattgcattagcaacattgcatactagttcataacttcgtataatgtatgctatacgaagttattaattaacaagggcgaattccttgatttatatacacctttgccaaccgcttgttacttgataaggaaaagatagatttctaaagtgcaggaaaagaaacgccactacgtcatgaaacaaaagaaatgaaacactctgcaaaagggaaaaccaatgacgccttcaaaacgtactgactttccgcctccttttctgccttttttttttctccctcaatttgccaattcccctttccgctaattttacatcacctttttgtttgtttcccttttcggccaagttttccatttcttttttcggctgagcccttctttggcgtcgacgtaatttctcggcatgtggccaatgtatattgacagtagatgaagtagacgttcttagtaactgttagggtgagattgccacccccccttccttcttttactatctgtaataccatcaccatagcaatagtttaaccatgttggagctggaaatacaacgtctatagagggaagtcatcatattacgccattttacggaccagggacaccctgtagtgtgtttcctctcttgtagaggtaggttttcaaatggactctggcgtcgatttccagcaagtcattcccgtggttcaccatttctactttttgcgctacctctcttgacacagaaatgaatgatgacgtgtaaattacccgtccgagacctggactccggagaaactgtattaattacgcgccaaacaagacaggtgtcggataaacgtgcatgtacagactgcgagccgaaaacggaaggggggaaagaaaacagtggagtcccattgttgttccggaaatggaaaacgggaactggcggaaaagaaacgaaacaaaacaaaagaaaaagaggaaaaaaaagaaaaaaaaaagaaaaagacactgcacgtgattgctggtgtgtgctgcgtaaccgcggcactttatttcgtaaatgaaggggcc

SEQ ID NO: 139 - DNA, 1395 bp, Pseudomonas fluorescens
  source 1..1395: mol_type = other DNA
ttagaacaatctgttcaaaccatcgtaagctgcaactctatatgcttcagccattgttgggtagttgaaggtggtattgacgaagtacttaagagtattttgttcacctggttggttcataattgcctggccaatatgaacaatttcggatgcttgataaccaaaacagtgaacacctaagacttctaaagtttctctatggaataagattttaagcataccctgtggttcaccagcaatttgtgctcttgccatagacttgaaaaaggctttaccgacctcataaggaaccttagctttagtcaattcgtgttcatttttaccaattgaggaaatttctggaatagtgtagataccggttggaacatcgttgacatatctccaggaaccattatcaacgatagagccagctgctgatctaccttggtcgtgagctgctgaggcaagtgatggccaaccaataacatcaccagcaccataaatgtttgtgacacaggttctgtaattctcgtcaacttcaatttgacctctggagttgaccttaacaccaatgttttccatacctaacttgtcagtattaccagttctaccgttacaccataacaatgcatcggccttaatctttttaccggacttcaaatgcaaaataacaccgttatctaaaccttcgactctgtcgtattcctcattatgtctaactgtaatgttgttattagaaaagtgataggaaagagcttgagaaatttcagagtctaaaaaggacaaaagttgatctctgttatcgaccaactcgactaagacacccaaacctgaaaagatagaagcatactcgcaaccgataacaccggcaccgtagataatcaactttctaggggtgtggcctaaagataagatggtatcagagtcgtagatacgtggatgatggaaatcgatgtctgctggtctgtaaggtctggaacctgtggcgatgataatatgcttagcaactaacttctcgacaacaccatttgcacaaacgacttcaacagtctgttcatctgcgaaggaaccagtaccgaaaaacaaatcaactctgtttctagcgtaatagccggtacgagaagcgacttgcttggaaataaccttttctgcagactttaaaacatctggaaaggagaaccatcttggttcgccaatggctctgaacattggattagtgttgaattgcatgatttgacgaacagagtgtctcaaagccttagaaggaatagtacctaagtgggtacagttaccaccgacttgtctacgtgaatcaaccatagcaacttttctgcctgctttagctgcattcatagcagcaccttcgcctgctggaccagaacccaaaacaacaacgtcgtagttataaacagccat

SEQ ID NO: 140 - DNA, 6281 bp, synthetic construct
  source 1..6281: mol_type = other DNA; note = Synthetic - Pseudomonas fluorescens URA integration fragment
cggatttggttggttcatagctcttctttagcgttgattgtagcctctgttgcagaaaagaccttgttttcaagaaactggttggtctgagtgttctgacaccaatggttatatttctagttgcaaacatgttggaataatggtagtaatgttgatgctggcagtgacagtagtgctagttcttgttcttgttctcgttctcgttctcgttctcttttttgtgctgtagctgttactgtattggctactctatataatatgcttgcaaaggaaaggaaatctatgcaaaccactctctcctgcacaaacgctagttcctttgtcagggttgaatgtagccactctacgaatgcgattcctcttcccctctctttcgcgttgagcatattcaaaattgtgagaggtggcaaggaaaaccatacgattttcggggtgcacggatgcacagtggacgtaagatctgctctatttaagatactaaagaaagtggcagcgggaagaccatcatggaggacaacgtacatggacgattccctgctagaaacaatgacatgcaaacccgtgcagtaggtagaagcagatttgctgagacagccatgccaagtggaattgttgtttaactctagtagatattattgttatagaaaagatttatatataagatccatggaggggggagggaggtaagagaaatacgaaaaagaatgtgtaatgaatcttaatgtagacaagtggaaatgcagctaaagggggtcaaagggaatgtgataatgcaaggttaggtttaacaagaatgggttggcggactcgtcaatggagagtacaatgccaaagttctccctgaggttattgcggccgcggatccagatcccccggggcgttgaagatctattctccagcaattaaatttgtgaagaataactggtatagagtacttcctttaaaaacatgtccgtgcaccaagaaaaaaaaaaagtttgaaaaattgtatgtcgacgaatttcagcattttcatttcaaggcgatattatgtttcactaaactcaggacaggaatatactaagaataactacaacatacacacaacataagccaagatggatcaacttaactaccaagaacaacaacaatttcaaaagatcgttgaacaaaagcaaatggctgatttcatgaggctatctgcagatacgcggaacaatcaatcgataatgatttgactgataaagaaaaccatacttttgtttatgtttattagttatcgctttgctacattaaaaattcacatactaaagcctttgttaaacaactttttctaaatcttaagattttactctatctagtttttttggttgtaggtgaacgtaaagtacctcatttatttttttttttttgcttgtgtaattcttttcatgcttatttaaactagtgtacatgtatcaaatctttgtgtaagaatcatttaaatctgtttaaataagcattccaaccagcttgttggtatcttttagcttgctctataggatctcttccttgaccgtacaaacctctaccaacaattatgatatccgttccagtctttacaacttcatcaacagttctatattgttgaccaagtgcatcacctttgtcatctaaaccaacccctggagtcataatgatccagtcaaaaccttcttctctaccgcccatatcgtgttgcgcaataaaaccaatgacaaactctttatcagatttagcaatttctactgttttttctgtatattcaccatatgctaaagaaccctttgatgataactcagcaagcattagcaaacctctaggttcactggttgtttcttgggctgcctccttcaagccagaaacaatacctgcacccgttacaccatgtgcattagtgatgtcagcccattcggcaatacggaagacaccagatttatattgatttttaacagtgttaccaatatcagcaaattttctatcttcaaaaatcataaaattatgtttcttggcaagctccttcaaaggcaacacagttccttcatacgtaaaatcagaaacaatatcgatgtgtgttttaactagacagatgtaaggaccaatagtgtccaaaatagagagaagcttttcagtttcagtaatatccaatgatgcacaaaggttagacttcttttcctccatgatggagaaaagtctcctagcaacaggggaagtgtgtgattctgatctttctttgtatgacgccatccttgacaaacaaactactttattaaagcgttgaagatctattctccagcaattaaatttgtgaagaataactggtatagagtacttcctttaaaaacatgtccgtgcaccaagaaaaaaaaaaagtttgaaaaattgtatgtcgacgaatttcagcattttcatttcaaggcgatattatgtttcactaaactcaggacaggaatatactaagaataactacaacatacacacaacataagccaagatggatcaacttaactaccaagaacaacaacaatttcaaaagatcgttgaacaaaagcaaatggctgatttcatgaggctatgaattcttttattataaaattatatattattcttaattacatatcacccttctatcagggaagggagaaacgaaaatagagagtgacctatccaagcttgggggtctaagttttaatggcccagggaatcattacttttttttctcaatccttgatggataaaagtattacatacgtacaggattgtgtattagtgtatttcgttatatgattaaacaaagtttatagattgtaaagtagacgtaaagtttagtaattcattttaatgttcattttacattcagatgttaattaattagaacaatctgttcaaaccatcgtaagctgcaactctatatgcttcagccattgttgggtagttgaaggtggtattgacgaagtacttaagagtattttgttcacctggttggttcataattgcctggccaatatgaacaatttcggatgcttgataaccaaaacagtgaacacctaagacttctaaagtttctctatggaataagattttaagcataccctgtggttcaccagcaatttgtgctcttgccatagacttgaaaaaggctttaccgacctcataaggaaccttagctttagtcaattcgtgttcatttttaccaattgaggaaatttctggaatagtgtagataccggttggaacatcgttgacatatctccaggaaccattatcaacgatagagccagctgctgatctaccttggtcgtgagctgctgaggcaagtgatggccaaccaataacatcaccagcaccataaatgtttgtgacacaggttctgtaattctcgtcaacttcaatttgacctctggagttgaccttaacaccaatgttttccatacctaacttgtcagtattaccagttctaccgttacaccataacaatgcatcggccttaatctttttaccggacttcaaatgcaaaataacaccgttatctaaaccttcgactctgtcgtattcctcattatgtctaactgtaatgttgttattagaaaagtgataggaaagagcttgagaaatttcagagtctaaaaaggacaaaagttgatctctgttatcgaccaactcgactaagacacccaaacctgaaaagatagaagcatactcgcaaccgataacaccggcaccgtagataatcaactttctaggggtgtggcctaaagataagatggtatcagagtcgtagatacgtggatgatggaaatcgatgtctgctggtctgtaaggtctggaacctgtggcgatgataatatgcttagcaactaacttctcgacaacaccatttgcacaaacgacttcaacagtctgttcatctgcgaaggaaccagtaccgaaaaacaaatcaactctgtttctagcgtaatagccggtacgagaagcgacttgcttggaaataaccttttctgcagactttaaaacatctggaaaggagaaccatcttggttcgccaatggctctgaacattggattagtgttgaattgcatgatttgacgaacagagtgtctcaaagccttagaaggaatagtacctaagtgggtacagttaccaccgacttgtctacgtgaatcaaccatagcaacttttctgcctgctttagctgcattcatagcagcaccttcgcctgctggaccagaacccaaaacaacaacgtcgtagttataaacagccattttatctagagagggggttatatgtgtaaatatagagtttgtgtttggttgtgtattgatggacactggggaaagttatctctctcttctaaagttccaacttcttcctctctctgggggtggacatctcctgtctttatataccacccccccccctcatagtctggaactcttccccgtccgagagcgacgtgattttccctatatttctccttccaaattgccccattgaggcaaccgcgtgatacgccgggtcttttcttcccgggagtggagacagttcccaagtggtaaagtccacttccatcatgatccacctccatgacactcttttaaacacatgtgctgcacatgtgttggtgtggttacacgatatacaaccatgttgggcaaccacaggtgcgcagcatcaacaacggggcgagtcaggaggaacggtaaccttggcggtttacgaatgaggtgttccccgttggagaggtgtctatggacgaccatcccagtacaatggacgggttggttggtgtggttacggtgtcaacgacagagacatgggccaacagagacattatggagagacgttgggagtggatcctccctggaaagggtgtctttggcgaagtgttgttgatttgtatgtctgggtcggttgtttgttgtgctagttgctgcataatggtaccaacaccagcggtttttttgtcctcctctgcaacaggggaactcggccacttgtttattggggggggggggggggatgccacgtggtaatgaaacattatcaagagacctctctttgtaggggtacctgtgatgtgtgtgtgtgtgcatgtgcatagatgcatattgagacagttgtgttcattgactgtctgcatttctccagaaccaataaaaaataaaaatagtagcaatacctacactttctggaccttgtagtaacgtagacgccgtaaggttgcatttcacgccagcttgagagggggaaagaactaccaatctcgagggatccgcggccgcaaataaatttaaaataaacgatatcaaaattcaaagggttcaaagtgggaattccttgatttatatacacctttgccaaccgcttgttacttgataaggaaaagatagatttctaaagtgcaggaaaagaaacgccactacgtcatgaaacaaaagaaatgaaacactctgcaaaagggaaaaccaatgacgccttcaaaacgtactgactttccgcctccttttctgccttttttttttctccctcaatttgccaattcccctttccgctaattttacatcacctttttgtttgtttcccttttcggccaagttttccatttcttttttcggctgagcccttctttggcgtcgacgtaatttctcggcatgtggccaatgtatattgacagtagatgaagtagacgttcttagtaactgttagggtgagattgccacccccccttccttcttttactatctgtaataccatcaccatagcaatagtttaaccatgttggagctggaaatacaacgtctatagagggaagtcatcatattacgccattttacggaccagggacaccctgtagtgtgtttcctctcttgtagaggtaggttttcaaatggactctggcgtcgatttccagcaagtcattcccgtggttcaccatttctactttttgcgctacctctcttgacacagaaatgaatgatgacgtgtaaattacccgtccgagacctggactccggagaaactgtattaattacgcgccaaacaagacaggtgtcggataaacgtgcatgtacagactgcgagccgaaaacggaaggggggaaagaaaacagtggagtcccattgttgttccggaaatggaaaacgggaactggcggaaaagaaacgaaacaaaacaaaagaaaaagaggaaaaaaaagaaaaaaaaaagaaaaagacactgcacgtgattgctggtgtgtgctgcgtaaccgcggcactttatttcgtaaatgaaggggcc

SEQ ID NO: 141 - DNA, 34 bp, synthetic construct
  source 1..34: mol_type = other DNA; note = Synthetic - Primer
aaagggttaattaattagaacaatctgttcaaac

SEQ ID NO: 142 - DNA, 20 bp, synthetic construct
  source 1..20: mol_type = other DNA; note = Synthetic - Primer
ctgttcaaaccatcgtaagc

SEQ ID NO: 143 - DNA, 30 bp, synthetic construct
  source 1..30: mol_type = other DNA; note = Synthetic - Primer
gtagttgaaggtggtattaacgaaatattc

SEQ ID NO: 144 - DNA, 18 bp, synthetic construct
  source 1..18: mol_type = other DNA; note = Synthetic - Primer
ggaactgtgttgcctttg

SEQ ID NO: 145 - DNA, 32 bp, synthetic construct
  source 1..32: mol_type = other DNA; note = Synthetic - Primer
gaataaaactggtaggcctattttctattctc

SEQ ID NO: 146 - protein, 464 aa, Pseudomonas fluorescens
MAVYNYDVVVLGSGPAGEGAAMNAAKAGRKVAMVDSRRQVGGNCTHLGTIPSKALRHSVRQIMQFNTNPMFRAIGEPRWFSFPDVLKSAEKVISKQVASRTGYYARNRVDLFFGTGSFADEQTVEVVCANGVVEKLVAKHIIIATGSRPYRPADIDFHHPRIYDSDTILSLGHTPRKLIIYGAGVIGCEYASIFSGLGVLVELVDNRDQLLSFLDSEISQALSYHFSNNNITVRHNEEYDRVEGLDNGVILHLKSGKKIKADALLWCNGRTGNTDKLGMENIGVKVNSRGQIEVDENYRTCVTNIYGAGDVIGWPSLASAAHDQGRSAAGSIVDNGSWRYVNDVPTGIYTIPEISSIGKNEHELTKAKVPYEVGKAFFKSMARAQIAGEPQGMLKILFHRETLEVLGVHCFGYQASEIVHIGQAIMNQPGEQNTLKYFVNTTFNYPTMAEAYRVAAYDGLNRLF

(19) United States
(12) Patent Application Publication
(10) Pub. No.: US 2024/0052383 A1
(43) Pub. Date: Feb. 15, 2024

(54) YEAST CELLS HAVING REDUCTIVE TCA PATHWAY FROM PYRUVATE TO SUCCINATE AND OVEREXPRESSING AN EXOGENOUS NAD(P+) TRANSHYDROGENASE ENZYME

(71) Applicant: CARGILL, INCORPORATED, Wayzata, MN (US)

(72) Inventors: Arlene M. FOSMER, Eden Prairie, MN (US); Vernon L. MCINTOSH, Jr., Minneapolis, MN (US); Thomas W. MCMULLIN, Minnetonka, MN (US); Gregory M. POYNTER, St. Paul, MN (US); Brian J. RUSH, Minneapolis, MN (US); Kevin T. WATTS, Minneapolis, MN (US)

(73) Assignee: CARGILL, INCORPORATED, Wayzata, MN (US)

(21) Appl. No.: 18/497,253

(22) Filed: Oct. 30, 2023

Related U.S. Application Data
(63) Continuation of application No. 17/348,363, filed on Jun. 15, 2021, now Pat. No. 11,821,021.
Application No. 17/348,363 is a continuation of application No. 15/816,779, filed on Nov. 17, 2017, now Pat. No. 11,041,176.
Application No. 15/816,779 is a division of application No. 14/416,633, filed on Jan. 22, 2015, now Pat. No. 9,850,507.
(60) Provisional application No. 61/675,788, filed on Jul. 25, 2012.

(51) Int. Cl. C12P 7/52; C12P 5/02; C12P 7/42; C12N 15/52; C12P 7/18; C12P 7/46; C12N 9/02; C12N 9/04; C12N 9/88; C12N 9/00

(52) U.S. Cl. CPC C12P 7/52; C12P 5/026; C12P 7/42; C12N 15/52; C12P 7/18; C12P 7/46; C12Y 106/01001; C12N 9/0036; C12N 9/0006; C12N 9/001; C12N 9/88; C12N 9/93; Y02E 50/30; C12Y 101/01; C12Y 106/01; C12Y 402/01002; C12Y 604/01001

(57) ABSTRACT

Yeast cells having a reductive TCA pathway from pyruvate or phosphoenolpyruvate to succinate, and which include at least one exogenous gene overexpressing an enzyme in that pathway, further contain an exogenous transhydrogenase gene.

### CROSS-REFERENCE TO RELATED APPLICATIONS

This application is a Continuation of U.S. application Ser. No. 17/348,363, filed Jun. 15, 2021, which is a Continuation of U.S. Pat. No. 11,041,176, issued Jun. 22, 2021, which is a Divisional of U.S. application Ser. No. 9,580,507, issued Dec. 26, 2017, which is a national phase application of PCT Application No. PCT/US2013/052069, filed Jul. 25, 2013, which claims the benefit of U.S. Provisional Application No. 61/675,788, filed Jul. 25, 2012, each of which are hereby incorporated by reference in their entirety.

### INCORPORATION BY REFERENCE OF SEQUENCE LISTING

A Sequence Listing is provided herewith as a text file, “N00188-US-CNT2” created on Oct. 27, 2023 and having a size of 419,288 bytes. The contents of the text file are incorporated by reference herein in their entirety.

This invention relates to recombinant yeast having an active reductive TCA pathway from pyruvate to succinate. The inventions disclosed and claimed herein were made pursuant to a joint research agreement between Cargill Incorporated, Wayzta, Minnesota, US, and BioAmber S.A.S, Bazancourt, France.

Succinic acid is a chemical intermediate useful as a precursor for making compounds such as 1,4-butanediol, tetrahydrofuran and gamma-butyrolactone. It is also a useful diacid that can be polymerized with a polyol to make polyester resins. Succinic acid can be produced industrially from butane. However, butane is a petrochemical, and there is a strong desire to develop processes for making many chemical compounds from annually renewable resources such as plant or animal feedstocks.

Some microorganisms have evolved the ability to produce succinate from carbohydrate feedstocks. In some cases, these strains have been engineered to improve yield and/or productivity. WO 2007/061590 describes recombinant yeast cells that produce succinate. Some yeast species are of interest as candidates for succinic acid-producing fermentations because they are resistant to low pH conditions, and so can produce acidic fermentation products at a low pH at which the product acid exists mainly in the acid form rather than in the salt form. Producing the acid directly in the acid form simplifies recovery and purification, as salt splitting, with its attendant requirements for raw materials, capital, operating and disposal costs, can be reduced if not eliminated.

There are three primary fermentation pathways for by which a microorganism can produce succinate: oxidative tricarboxylic acid (TCA), glyoxylate shunt, and reductive TCA. The oxidative TCA pathway begins with the conversion of oxaloacetate (OAA) and acetyl-CoA to citrate. OAA can be generated from carboxylation of phosphoenolpyruvate (PEP) or pyruvate, while acetyl-CoA is generated from the decarboxylation of pyruvate by pyruvate dehydrogenase (PDH) or pyruvate formate lyase (PFL). Citrate is converted to isocitrate, isocitrate is converted to a-ketoglutarate, α-ketoglutarate is converted to succinyl-CoA, and succinyl-CoA is converted to succinate.

Like the oxidative TCA pathway, the glyoxylate shunt pathway begins with the generation of citrate from OAA and acetyl-CoA and the conversion of citrate to isocitrate. Isocitrate is converted to glyoxylate and succinate. Glyoxylate is condensed with acetyl-CoA to form malate, and the resultant malate is converted to succinate via a fumarate intermediate.

The reductive TCA pathway begins with carboxylation of phosphoenolpyruvate (PEP) or pyruvate to oxaloacetate (OAA) (by PEP carboxylase (PPC) and pyruvate carboxylase (PYC), respectively). OAA is converted to malate by malate dehydrogenase (MDH), malate is converted to fumarate by fumarase (FUM, also known as fumarate hydratase), and fumarate is converted to succinate by fumarate reductase (FRD). The reductive TCA pathway provides the highest succinate yield of the three succinate fermentation pathways, per mole of glucose consumed, and for that reason offers the best economic potential.

A problem with the reductive TCA pathway is that the MDH enzyme consumes NADH as a cofactor. In addition, certain efficient FRD enzymes also consume NADH. Examples of such NADH-dependent FRD enzymes are described, for example, in WO 2009/065778 and PCT/US2011/022612. Thus, certain efficient metabolic pathways from pyruvate to succinate consume two molecules of NADH. One molecule of NADH is produced when sugars such as glucose are metabolized to pyruvate via the glycolytic pathway, but this still leaves a net deficit of one NADH, which results in a redox imbalance. A living cell must correct this redox balance if it is to remain healthy and continue to metabolize through the reductive TCA pathway. This typically means that the cell must balance the net NADH consumption by replacing the consumed NADH from other metabolic processes that produce NADH. For example, the reductive TCA pathway can be combined with one or both of the oxidative TCA or glyoxylate shunt pathways to help with the redox balance, but the oxidative TCA and glyoxylate shunt pathways produce less succinic acid per mole of starting sugar, and taking this approach therefore results in a loss of yield. It is possible for the cell to use one or more unrelated pathways to produce the needed NADH, but this can have adverse consequences for cell health and productivity, and may create other imbalances within the cell.

Therefore, there remains a desire to provide a yeast strain that efficiently produces succinic acid (or its salts).

In one aspect, this invention is a recombinant yeast cell having an active reductive TCA metabolic pathway from pyruvate to succinate and which further overexpresses a NAD(P)⁺ transhydrogenase enzyme.

In particular embodiments, the yeast cell of the invention has integrated into its genome at least one exogenous NAD(P)+ transhydrogenase gene that encodes for the NAD(P)+ transhydrogenase enzyme.

In other particular embodiments, the recombinant yeast cell of the invention (a) expresses an NADPH-dependent malate dehydrogenase enzyme, (b) has at least one exogenous NADPH-dependent malate dehydrogenase gene integrated into its genome, (c) expresses an NADPH-dependent fumarate reductase enzyme, (d) has at least one exogenous NADPH-dependent fumarate reductase gene integrated into its genome or (e) has a combination of any two or more of (a), (b), (c) and (d).

The recombinant yeast cell of the invention in some embodiments has integrated into its genome one or more of (i) an exogenous pyruvate carboxylase gene that encodes for an enzyme which catalyzes the conversion of pyruvate to oxaloacetate, (ii) an exogenous malate dehydrogenase gene which encodes for an enzyme that catalyzes the conversion of oxaloacetate to malate, (iii) an exogenous fumarase gene that encodes for an enzyme which catalyzes the conversion of malate to fumarate and (iv) an exogenous fumarate reductase gene that encodes an enzyme which catalyzes the conversion of fumarate to succinate. In some embodiments, the recombinant cell of the invention has integrated into its genome one or more of (i) a non-native pyruvate carboxylase gene that encodes for an enzyme which catalyzes the conversion of pyruvate to oxaloacetate, (ii) a non-native malate dehydrogenase gene which encodes for an enzyme that catalyzes the conversion of oxaloacetate to malate, (iii) a non-native exogenous fumarase gene that encodes for an enzyme which catalyzes the conversion of malate to fumarate and (iv) a non-native exogenous fumarate reductase gene which encodes an enzyme which catalyzes the conversion of fumarate to succinate.

In preferred embodiments, the recombinant cell of the invention has integrated into its genome at least one exogenous malate dehydrogenase gene which encodes for an NADH-dependent enzyme that catalyzes the conversion of oxaloacetate to malate. In other preferred embodiments, the recombinant cell of the invention has integrated into its genome at least one exogenous fumarate reductase gene which encodes for an NADH-dependent enzyme that catalyzes the conversion of fumarate to succinate. In especially preferred embodiments, the recombinant cell of the invention has both of these features.

In other specific embodiments, the recombinant cell of the invention overexpresses at least one enzyme which catalyzes a reaction that includes the reduction of NADP+ to NADPH. This reaction may be a reaction in the pentose phosphate pathway. The enzyme catalyzing that reaction may be, for example, a 6-phosphogluconate dehydrogenase (6PDGH) enzyme and/or a glucose 6-phosphate dehydrogenase (G6PDH) enzyme.

In still other specific embodiments, the recombinant cell of the invention overexpresses at least one Stb5p protein, and/or has at least one exogenous Stb5p gene (i.e., a gene that encodes for the Stb5p protein) integrated into its genome.

In still other specific embodiments, the recombinant cell of the invention has a deletion or disruption of a native phosphoglucose isomerase gene.

In the cells of any of the foregoing aspects of the invention, the NADH/NAD+ redox imbalance that is produced in the reductive TCA pathway to succinate is compensated for, at least in part, by converting NADPH formed in other cellular metabolic processes to NADH, which can be consumed in the succinate-producing pathway. This is a beneficial approach to solving the NADH/NAD+ redox imbalance, because yeast cells typically have, or can be easily engineered to have, active metabolic pathways that produce NADPH. A yeast cell's native pentose phosphate pathway is an example of a metabolic pathway that produces NADPH. Thus, NADPH can be produced in the cell by directing carbon flux through a pentose phosphate pathway, and all or a portion of the NADPH so produced can be converted to NADH by action of the overexpressed NAD(P)⁺ transhydrogenase enzyme. Some or all of the NADH so produced can alleviate or even eliminate the NADH/NAD+ redox imbalance that results from succinate production through the reductive TCA pathway.

NADPH production can be increased (relative to the wild-type host cell), for example, by increasing carbon flux through the pentose phosphate pathway and/or by overexpressing at least one enzyme (including an enzyme in the pentose phosphate pathway) which catalyzes a reaction that includes the reduction of NADP+ to NADPH. Again, the increased NADPH so produced can be converted to NADH by action of the NAD(P)⁺ transhydrogenase enzyme. As before, some or all of the NADH so produced can alleviate or even eliminate the NADH/NAD+ redox imbalance that results from succinate production through the reductive TCA pathway.

This, in some embodiments, the recombinant cell of the invention includes one or more genetic modifications that (1) increase flux through the pentose phosphate pathway and/or (2) overexpress one or more enzymes in the pentose phosphate pathway that catalyze a reaction that includes the reduction of NADP+ to NADPH. In certain embodiments, therefore, the recombinant cell of the invention also (a) overexpresses at least one Stb5p protein (b) has at least one exogenous Stb5p gene integrated into its genome, (c) produces a severely reduced quantity of an active phosphoglucose isomerase (PGI) enzyme, (d) produces a PGI enzyme that has a severely reduced activity, (e) has a deletion or disruption of a native PGI gene, (f) overexpresses at least one 6-phosphogluconate dehydrogenase (6PGDH) enzyme, (g) has at least one exogenous 6PGDH gene integrated into its genome, (h) overexpresses at least one glucose-6-phosphate dehydrogenase (G6PDH) enzyme, (i) has at least one exogenous G6PDH gene integrated into its genome, or (j) an combination of any two or more of (a)-(i).

The cell of the invention may produce succinate and transport it from the cell. In some embodiments, the cell may further metabolize some or all of the succinate into one or more other succinate metabolization products, and transport one or more of such succinate metabolization products from the cell. In such embodiments, the cell contains native or non-native metabolic pathways which perform the further metabolization of succinate into such succinate metabolization product(s).

In yet other aspects, the invention is a method of producing succinate or a metabolization product of succinate, comprising culturing a cell of any of the foregoing aspects in a fermentation medium that includes at least one carbon source. The cells of the invention are capable of producing succinate or metabolization products of succinate in high yields at commercially reasonable production rates.

The term “NADH-dependent” as used herein refers to the property of an enzyme to preferentially use NADH as the redox cofactor. An NADH-dependent enzyme has a higher specificity constant (k_{cat}/K_{M}) with the cofactor NADH than with other cofactors, including the cofactor NADPH, as determined by in vitro enzyme activity assays.

For purposes of this application, “native” as used herein with regard to a metabolic pathway refers to a metabolic pathway that exists and is active in the wild-type host strain. Genetic material such as genes, promoters and terminators is “native” for purposes of this application if the genetic material has a sequence identical to (apart from individual-to-individual mutations which do not affect function) a genetic component that is present in the genome of the wild-type host cell (i.e., the exogenous genetic component is identical to an endogenous genetic component).

For purposes of this application, genetic material such as a gene, a promoter and a terminator is “endogenous” to a cell if it is (i) native to the cell, (ii) present at the same location as that genetic material is present in the wild-type cell and (iii) under the regulatory control of its native promoter and its native terminator.

For purposes of this application, genetic material such as genes, promoters and terminators is “exogenous” to a cell if it is (i) non-native to the cell and/or (ii) is native to the cell, but is present at a location different than where that genetic material is present in the wild-type cell and/or (iii) is under the regulatory control of a non-native promoter and/or non-native terminator. Extra copies of native genetic material are considered as “exogenous” for purposes of this invention, even if such extra copies are present at the same locus as that genetic material is present in the wild-type host strain.

As used herein, the term “promoter” refers to an untranslated sequence located upstream (i.e., 5′) to the translation start codon of a gene (generally a sequence of about 1 to 1500 base pairs (bp), preferably about 100 to 1000 bp and especially of about 200 to 1000 bp) which controls the start of transcription of the gene. The term “terminator” as used herein refers to an untranslated sequence located downstream (i.e., 3′) to the translation finish codon of a gene (generally a sequence of about 1 to 1500 bp, preferably of about 100 to 1000 bp, and especially of about 200 to 500 bp) which controls the end of transcription of the gene. A promoter or terminator is “operatively linked” to a gene if its position in the genome relative to that of the gene is such that the promoter or terminator, as the case may be, performs its transcriptional control function.

“Identity” for nucleotide or amino acid sequences are for purposes of this invention calculated using BLAST (National Center for Biological Information (NCBI) Basic Local Alignment Search Tool) version 2.2.13 software with default parameters. A sequence having an identity score of XX% with regard to a reference sequence using the BLAST version 2.2.13 algorithm with default parameters is considered to be at least XX% identical or, equivalently, have XX% sequence identity to the reference sequence.

“Deletion or disruption” with regard to a gene means that either the entire coding region of the gene is eliminated (deletion) or the coding region of the gene, its promoter, and/or its terminator region is modified (such as by deletion, insertion, or mutation) such that the gene no longer produces an active enzyme, produces a severely reduced quantity (at least 75% reduction, preferably at least 85% reduction, more preferably at least 95% reduction) of the enzyme, or produces an enzyme with severely reduced (at least 75% reduced, preferably at least 85% reduced, more preferably at least 95% reduced) activity. A deletion or disruption of a gene can be accomplished by, for example, forced evolution, mutagenesis or genetic engineering methods, followed by appropriate selection or screening to identify the desired mutants.

“Overexpress” means the artificial expression of an enzyme in increased quantity by a gene. Overexpression of an enzyme may result from the presence of one or more exogeneous gene(s), or from other conditions. For purposes of this invention, a yeast cell containing at least one exogenous gene is considered to overexpress the enzyme(s) encoded by such exogenous gene(s).

The recombinant yeast of the invention is made by performing certain genetic modifications to a host yeast cell. The host yeast cell is one which as a wild-type strain is natively capable of metabolizing at least one sugar to pyruvate. Suitable host yeast cells include (but are not limited to) yeast cells classified under the genera Candida, Pichia, Saccharomyces, Schizosaccharomyces, Zygosaccharomyces, Kluyveromyces, Debaryomyces, Pichia, Issatchenkia, Yarrowia and Hansenula. Examples of specific host yeast cells include C. sonorensis, K. marxianus, K. thermotolerans, C. methanesorbosa, Saccharomyces bulderi (S. bulderi), I. orientalis, C. lambica, C. sorboxylosa, C. zemplinina, C. geochares, P. membranifaciens, Z. kombuchaensis, C. sorbosivorans, C. vanderwaltii, C. sorbophila, Z. bisporus, Z. lentus, Saccharomyces bayanus (S. bayanus), D. castellii, C. boidinii, C. etchellsii, K. lactis, P. jadinii, P. anomala, Saccharomyces cerevisiae (S. cerevisia) Pichia galeiformis, Pichia sp. YB-4149 (NRRL designation), Candida ethanolica, P. deserticola, P. membranifaciens, P. fermentans and Saccharomycopsis crataegensis (S. crataegensis). Suitable strains of K. marxianus and C. sonorensis include those described in WO 00/71738 A1, WO 02/42471 A2, WO 03/049525 A2, WO 03/102152 A2 and WO 03/102201A2. Suitable strains of I. orientalis are ATCC strain 32196 and ATCC strain PTA-6648.

In some embodiments of the invention the host cell is Crabtree negative as a wild-type strain. The Crabtree effect is defined as the occurrence of fermentative metabolism under aerobic conditions due to the inhibition of oxygen consumption by a microorganism when cultured at high specific growth rates (long-term effect) or in the presence of high concentrations of glucose (short-term effect). Crabtree negative phenotypes do not exhibit this effect, and are thus able to consume oxygen even in the presence of high concentrations of glucose or at high growth rates.

In some embodiments, the host cell is succinate-resistant as a wild-type strain. A cell is considered to be “succinate-resistant” if the cell exhibits a growth rate in media containing 75 g/L or greater succinate at pH 2.8 that is at least 50% as high as its growth rate in the same media containing 0 g/L succinate, according to the test method described in Example 1A of WO 2012/103261.

In some embodiments, the host cell exhibits a volumetric glucose consumption rate of at least 3, at least 5 or at least 8 grams of glucose per liter of broth per hour, as a wild-type strain.

In some embodiments, the host cell exhibits a specific glucose consumption rate of at least 0.5, at least 1.0 or at least 1.5 gram of glucose per gram dry weight of cells per hour, as a wild-type strain.

Volumetric and specific glucose consumption can be measured by cultivating the cells in shake flasks yeast in extract peptone dextrose (YPD) media containing 0 g/l 75 g/L succinate at pH 3.0 a described in Example 1 of WO 2012/103261. The flasks are inoculated with biomass harvested from seed flasks grown overnight to an OD₆₀₀ of 6 to 10. 250 mL baffled glycolytic assay flasks (50 mL working volume) are inoculated to an OD600 of 0.1 and grown at 250 RPM and 30° C. Samples are taken throughout the time course for the assay and analyzed for glucose consumption by electrophoretic methods (such as by using a 2700 Biochemistry Analyzer from Yellow Springs Instruments or equivalent device). The data is plotted and volumetric glucose consumption rate calculated. Specific glucose consumption rate is calculated by dividing the glucose consumption by the cell dry weight at the end of fermentation.

The genetically modified yeast cells provided herein have an active reductive TCA active pathway from pyruvate to succinate. Such an active reductive TCA pathway includes a step of converting pyruvate or phosphoenolpyruvate (PEP) (or each) to oxaloacetate (OAA), a step of converting oxaloacetate to malate, a step of converting malate to fumarate, and a step of converting fumarate to succinate.

The step of converting pyruvate to OAA is catalyzed by a PYC (pyruvate carboxylase) enzyme, i.e., an enzyme having the ability to catalyze the conversion of pyruvate to OAA. A PYC enzyme is encoded by a PYC (pyruvate carboxylase) gene integrated into the genome of the recombinant yeast cell. The PYC gene may be native or non-native to the host cell, and may be endogenous (if native) or exogenous (if non-native or if additional copies of a native gene are present). In certain embodiments, a PYC gene may be a yeast gene. For example, the PYC gene may be an I. orientalis PYC gene encoding for an enzyme having amino acid sequence SEQ ID NO: 94, an S. cerevisiae PYC1 gene encoding for an enzyme having amino acid sequence SEQ ID NO: 95, or a K. marxianus PYC1 gene encoding for an enzyme having amino acid SEQ ID NO: 96. In other embodiments, the gene may encode for an enzyme having an amino acid sequence with at least 50%, at least 60%, at least 70%, at least 80%, at least 85%, at least 90%, at least 95%, at least 97%, or at least 99% sequence identity to any of SEQ ID NOs: 94, 95 or 96. In certain embodiments, the gene may have the nucleotide sequence set forth in SEQ ID NOs: 4, 45 or 46, or a nucleotide sequence with at least 50%, at least 60%, at least 70%, at least 80%, at least 85%, at least 90%, at least 95%, at least 97%, or at least 99% sequence identity to any of SEQ ID NOs: 4, 45 or 46. In other embodiments, the PYC gene may be fungal.

The step of converting PEP to OAA is catalyzed by a PPC (phosphoenolpyruvate carboxylase) enzyme, i.e., an enzyme having the ability to catalyze the conversion of PEP to OAA. A PPC enzyme is encoded by a PPC (phosphoenolpyruvate carboxylase) gene integrated into the genome of the recombinant yeast cell. The PPC gene may be native or non-native to the host cell, and may be endogenous (if native) or exogenous (if non-native or if additional copies of a native gene are present). The PPC gene may encode for an enzyme having either of amino acid sequences SEQ ID NO: 97 or 115, or for an enzyme having an amino acid sequence with at least 50%, at least 60%, at least 70%, at least 80%, at least 85%, at least 90%, at least 95%, at least 97%, or at least 99% sequence identity to either of SEQ ID NOs: 97 or 115. In certain embodiments, the PPC gene may have the nucleotide sequence set forth in either of SEQ ID NOs: 49 or 50, or a nucleotide sequence with at least 50%, at least 60%, at least 70%, at least 80%, at least 85%, at least 90%, at least 95%, at least 97%, or at least 99% sequence identity to either of SEQ ID NOs: 49 or 50.

The step of converting OAA to malate is catalyzed by a MDH (malate dehydrogenase) enzyme, i.e., an enzyme having the ability to catalyze the conversion of OAA to malate. A MDH enzyme is encoded by a MDH (malate dehydrogenase) gene present in the genome of the recombinant yeast cell. The MDH gene may be native or non-native to the host cell, and may be endogenous (if native) or exogenous (if non-native or if additional copies of a native gene are present). The MDH enzyme preferably is NADH-dependent, i.e., one which uses NADH preferentially as a cofactor, and in converting OAA to malate also oxidizes NADH to NAD+. In the cells of this invention, the MDH enzyme preferably is overexpressed, by integrating one or more copies of an exogenous MDH gene (preferably at least two copies) into the genome of the cell. Preferred MDH genes encode for NADH-dependent MDH enzymes.

In certain embodiments, the MDH gene is a yeast MDH gene that encodes for an NADH-dependent MDH enzyme. For example, the MDH gene may be an I. orientalis MDH1, MDH2, or MDH3 gene encoding for an enzyme having any of the amino acid sequences SEQ ID NOs: 98, 99 or 100, respectively, a Z. rouxii MDH gene encoding for an enzyme having amino acid sequence SEQ ID NO: 101, a K. marxianus MDH1, MDH2, or MDH3 gene encoding for an enzyme having any of amino acid sequences SEQ ID NOs: 102, 103 or 104, respectively, or a gene encoding for an enzyme having an amino acid sequence with at least 50%, at least 60%, at least 70%, at least 80%, at least 85%, at least 90%, at least 95%, at least 97%, or at least 99% sequence identity to any thereof. In certain embodiments, the yeast MDH gene has the nucleotide sequence set forth in any of SEQ ID NOs: 58, 59, 60, 61, 62, 63 or 64 or a nucleotide sequence with at least 50%, at least 60%, at least 70%, at least 80%, at least 85%, at least 90%, at least 95%, at least 97%, or at least 99% sequence identity to any thereof.

In certain embodiments, the MDH gene is a bacterial MDH gene that encodes for an NADH-dependent MDH enzyme. For example, the MDH gene is in some embodiments an Escherichia coli (E. coli) MDH gene encoding for an enzyme having amino acid sequence SEQ ID NO: 105 or a gene that encodes for an enzyme having an amino acid sequence with at least 50%, at least 60%, at least 70%, at least 80%, at least 85%, at least 90%, at least 95%, at least 97%, or at least 99% sequence identity thereto. In certain embodiments, the bacterial MDH gene has the nucleotide sequence SEQ ID NO: 66 or a nucleotide sequence with at least 50%, at least 60%, at least 70%, at least 80%, at least 85%, at least 90%, at least 95%, at least 97%, or at least 99% sequence identity to either of those.

In certain embodiments, an MDH gene is a fungal MDH gene that encodes for an NADH-dependent MDH enzyme. For example, the MDH gene in some embodiments is a Rhizopus. oryzae (R. oryzae) MDH gene or a Rhizopus delemar (R. delemar) MDH gene encoding for an enzyme having amino acid sequence SEQ ID NO: 106 or 128 or a gene which encodes for an enzyme having amino acid sequence with at least 50%, at least 60%, at least 70%, at least 80%, at least 85%, at least 90%, at least 95%, at least 97%, or at least 99% sequence identity to either thereof. In certain embodiments, the fungal MDH gene has nucleotide sequence SEQ ID NO: 68 or 13 or a nucleotide sequence with at least 50%, at least 60%, at least 70%, at least 80%, at least 85%, at least 90%, at least 95%, at least 97%, or at least 99% sequence identity thereto.

The step of converting malate to fumarate is catalyzed by a FUM (fumarase) enzyme, i.e., an enzyme having the ability to catalyze the conversion of malate to fumarate. A FUM (fumarase) enzyme is encoded by a FUM (fumarase) gene integrated into the genome of the recombinant yeast cell. The FUM gene may be native or non-native to the host cell, and may be endogenous (if native) or exogenous (if non-native or if additional copies of a native gene are present). In certain embodiments, a FUM gene is a yeast gene. The FUM gene is in some embodiments an I. orientalis FUM gene encoding an enzyme having amino acid sequence SEQ ID NO: 107, or for an enzyme having an amino acid sequence with at least 50%, at least 60%, at least 70%, at least 80%, at least 85%, at least 90%, at least 95%, at least 97%, or at least 99% sequence identity to SEQ ID NO: 107. In certain embodiments, the FUM gene may have nucleotide sequence SEQ ID NO: 70 or a nucleotide sequence with at least 50%, at least 60%, at least 70%, at least 80%, at least 85%, at least 90%, at least 95%, at least 97%, or at least 99% sequence identity to SEQ ID NO: 70. In other embodiments, a FUM gene may be a bacterial gene.

The step of converting fumarate to succinate is catalyzed by a FRD (fumarate reductase) enzyme, i.e., an enzyme having the ability to catalyze the conversion of fumarate to succinate. A FRD (fumarate reductase) enzyme is encoded by a FRD (fumarate reductase) gene present in the genome of the recombinant yeast cell. The FRD gene may be native or non-native to the host cell, and may be endogenous (if native) or exogenous (if non-native or if additional copies of a native gene are present). The FRD enzyme preferably is NADH-dependent, i.e., one which uses NADH preferentially as a cofactor, and in converting fumarate to succinate also oxidizes NADH to NAD+. In the cells of this invention, the FRD enzyme preferably is overexpressed, by integrating one or more copies of an exogenous FRD gene (preferably at least two copies) into the genome of the cell. The FRD gene preferably encodes for an NADH-dependent FRD enzyme.

In certain embodiments, the FRD gene is a yeast FRD gene that encodes for an NADH-dependent FRD enzyme. For example, the FRD gene may be an S. cerevisiae FRD1 gene encoding for an enzyme having amino acid sequence SEQ ID NO: 108, a Saccharomyces mikatae (S. mikatae) FRD1 gene encoding for an enzyme having amino acid sequence SEQ ID NO: 109, a K. polyspora FRD1 gene encoding for an enzyme having amino acid sequence SEQ ID NO: 110, a K. marxianus FRD1 gene encoding for an enzyme having amino acid sequence SEQ ID NO: 111, or a gene encoding for an enzyme having an amino acid sequence with at least 50%, at least 60%, at least 70%, at least 80%, at least 85%, at least 90%, at least 95%, at least 97%, or at least 99% sequence identity to any thereof. In certain embodiments, the yeast FRD gene may have any of nucleotide sequences SEQ ID NOs: 75, 76, 77 or 78, or have a nucleotide sequence with at least 50%, at least 60%, at least 70%, at least 80%, at least 85%, at least 90%, at least 95%, at least 97%, or at least 99% sequence identity to any thereof.

In certain embodiments, the FRD gene may be a protozoan gene that encodes for an NADH-dependent FRD enzyme. For example, the FRD gene may be a Trypanosoma brucei (T. brucei) FRD gene encoding for an enzyme having amino acid sequence SEQ ID NO: 112, a Trypanosoma cruzi (T. cruzi) FRD gene encoding for an enzyme having amino acid sequence SEQ ID NO: 113, a Leishmania braziliensis (L. braziliensis) FRD gene encoding for an enzyme having amino acid sequence SEQ ID NO: 114, a Leishmania mexicana (L. mexicana) FRD gene encoding for an enzyme having amino acid sequence SEQ ID NO: 82, or a gene encoding for an enzyme having an amino acid sequence having at least 50%, at least 60%, at least 70%, at least 80%, at least 85%, at least 90%, at least 95%, at least 97%, or at least 99% sequence identity to any thereof. In certain embodiments, the FRD gene may have a nucleotide sequence as set forth in any of SEQ ID NOs: 42, 43, 44 or 10, or a nucleotide sequence with at least 50%, at least 60%, at least 70%, at least 80%, at least 85%, at least 90%, at least 95%, at least 97%, or at least 99% sequence identity to any thereof.

In this invention, it is preferred that the reaction of OAA to malate and the reaction of fumarate to succinate each oxidizes NADH to NAD+. The oxidation of NADH to NAD+ typically occurs in cases in which the reaction in any one or more of these steps is catalyzed by an NADH-dependent enzyme as described before.

The recombinant cell of the invention overxpresses an active NAD(P)+ transhydrogenase enzyme and/or includes one or more exogenous NAD(P)+ transhydrogenase genes, which may be native or non-native to the host cell. A “NAD(P)+ transhydrogenase” (SthA) gene refers to any gene that encodes a polypeptide that catalyzes the reaction of NADP(H) to form NAD(H). The NAD(P)+ transhydrogenase (SthA) enzyme preferably is soluble in the cytosol of the recombinant cell. The exogenous SthA gene may be of bacterial, fungal, yeast or other origin. The exogenous SthA gene in some embodiments is an E. coli, Azotobacter vinelandii (A. vinelandii) or Pseudomona flourescens SthA gene. The exogenous SthA gene in some embodiments encodes for an enzyme having any of amino acid sequences SEQ ID NOs: 117, 118, 119, or 146, or which is at least 50%, at least 60%, at least 70%, at least 80%, at least 85%, at least 90%, at least 95%, at least 97%, or at least 99% identical to any thereof. In certain embodiments, the exogenous SthA gene has any of nucleotide sequences SEQ ID NOs: 21, 24, 27, or 139, or a nucleotide sequence with at least 50%, at least 60%, at least 70%, at least 80%, at least 85%, at least 90%, at least 95%, at least 97%, or at least 99% sequence identity to any thereof.

In some embodiments, the recombinant cell exhibits increased flux (relative to the wild-type host strain) through the pentose phosphate pathway and/or overexpresses at least one enzyme which catalyzes a reaction that includes the reduction of NADP+ to NADPH.

The overexpressed enzyme may be an enzyme that catalyzes a reaction in the pentose phosphate pathway. The pentose phosphate pathway metabolizes glucose-6-phosphate to glyceraldehyde-3-phosphate through 6-phosphogluconolactone, 6-phosphogluconate and ribulose 5-phosphate intermediates. The conversion of glucose-6-phosphate to 6-phosphogluconolactone is catalyzed by a glucose-6-phosphate dehydrogenase (G6PDH) enzyme that uses NADP+ as a cofactor, thereby reducing NADP+ to NADPH. Similarly, the conversion of 6-phosphogluconate to ribulose-5-phosphate is catalyzed by a 6-phosphogluconate dehydrogenase (6PGDH) enzyme that uses NADP+ as a cofactor, thereby reducing NADP+ to NADPH. Overexpressing one or both of these enzymes, or increasing flux through the pentose phosphate pathway, produces NADPH, which can be converted to NADH by action of the NAD(P)+ transhydrogenase enzyme, helping to maintain cofactor balance in the cell.

One way of increasing flux through the pentose phosphate pathway is to disrupt the glycolytic pathway from glucose to pyruvate. This can be done, for example, by disrupting or removing the step of isomerising glucose-6-phosphate to fructose-6-phosphate, which is catalyzed by a phosphoglucose (PGI) enzyme. Therefore, in certain embodiments, the recombinant cell of the invention produces a severely reduced quantity (at least 75% reduction, preferably at least 85% reduction, more preferably at least 95% reduction) of an active phosphoglucose isomerase (PGI) enzyme, or produces a PGI enzyme with severely reduced (at least 75% reduced, preferably at least 85% reduced, more preferably at least 95% reduced) activity. In some embodiments, the recombinant cell includes a deletion or disruption of at least one native phosphoglucose isomerase (PGI) gene. If the host cell contains multiple alleles of the PGI gene, all such alleles may be deleted or disrupted.

The overexpressed enzyme which catalyzes a reaction that includes the reduction of NADP+ to NADPH may be an enzyme that catalyzes a reaction in the pentose phosphate pathway. The pentose phosphate pathway metabolizes glucose-6-phosphate to glyceraldehyde-3-phosphate through 6-phosphogluconolactone, 6-phosphogluconate and ribulose 5-phosphate intermediates. The conversion of glucose-6-phosphate to 6-phosphogluconolactone is catalyzed by a glucose-6-phosphate dehydrogenase (G6PDH) enzyme that uses NADP+as a cofactor, thereby reducing NADP+ to NADPH. Similarly, the conversion of 6-phosphogluconate to ribulose-5-phosphate is catalyzed by a 6-phosphogluconate dehydrogenase (6PGDH) enzyme that uses NADP+ as a cofactor, thereby reducing NADP+ to NADPH.

Therefore, in certain embodiments, the yeast cell of the invention overexpresses a G6PDH enzyme. Such a yeast cell in some embodiments includes one or more exogenous G6PDH genes, which may be native or non-native to the strain, integrated into its genome. In certain of these embodiments, the exogenous G6PDH gene may be an I. orientalis G6PDH gene (ZWF1) that encodes for an enzyme having amino acid sequence SEQ ID NO: 121 or which encodes for an enzyme having with at least 50%, at least 60%, at least 70%, at least 80%, at least 85%, at least 90%, at least 95%, at least 97%, or at least 99% sequence identity SEQ ID NO: 121. In certain embodiments, the G6PDH gene may have nucleotide sequence SEQ ID NO: 87 or a nucleotide sequence with at least 50%, at least 60%, at least 70%, at least 80%, at least 85%, at least 90%, at least 95%, at least 97%, or at least 99% sequence identity to nucleotide sequence SEQ ID NO: 87.

Similarly, in other embodiments, the recombinant yeast cells provided herein contains one or more exogenous 6PGDH genes, which may be native or non-native to the host strain, integrated into its genome. In certain embodiments, a 6PGDH gene may be a yeast 6PGDH gene such as an I. orientalis 6PGDH gene. In certain embodiments, the exogenous 6PGDH gene encodes for an enzyme having amino acid sequence SEQ ID NO: 88, or an amino acid sequence with at least 50%, at least 60%, at least 70%, at least 80%, at least 85%, at least 90%, at least 95%, at least 97%, or at least 99% sequence identity to SEQ ID NO: 88. In certain embodiments, the exogenous 6PGDH gene has the nucleotide sequence of SEQ ID NO: 89, or a nucleotide sequence with at least 50%, at least 60%, at least 70%, at least 80%, at least 85%, at least 90%, at least 95%, at least 97%, or at least 99% sequence identity to the nucleotide sequence of SEQ ID NO: 89.

In certain embodiments, the recombinant cell of the invention overexpresses an oxidative stress-activated zinc cluster protein Stb5p. This zinc cluster protein regulates genes involved in certain NADPH-producing reactions, including the G6PDH and 6PGDH genes. In certain embodiments, the recombinant cell includes one or more exogenous Stb5p genes, which may be native or non-native to the host cell, integrated into its genome. In certain embodiments, the exogenous Stb5p gene encodes for an enzyme having amino acid sequence SEQ ID NO: 83, or an amino acid sequence with at least 50%, at least 60%, at least 70%, at least 80%, at least 85%, at least 90%, at least 95%, at least 97%, or at least 99% sequence identity to SEQ ID NO: 83. In certain embodiments, the exogenous Stb5p gene has the nucleotide sequence of SEQ ID NO: 30, or a nucleotide sequence with at least 50%, at least 60%, at least 70%, at least 80%, at least 85%, at least 90%, at least 95%, at least 97%, or at least 99% sequence identity to the nucleotide sequence of SEQ ID NO: 30.

The recombinant cell of the invention may further include one or more exogenous succinate exporter genes, which may be native or non-native to the host cell. A “succinate exporter gene” as used herein refers to any gene that encodes a polypeptide with succinate export activity, meaning the ability to transport succinate out of a cell and into the extracellular environment. The exogenous succinate exporter gene may be a fungal succinate exporter gene such as a Schizosaccharomyces pombe (S. pombe) succinate exporter gene or Aspergillus oryzae (A. oryzae) source succinate exporter gene. The exogenous succinate exporter gene in some embodiments encodes for an enzyme having amino acid sequence SEQ ID NOs: 90 or 91, or at least 50%, at least 60%, at least 70%, at least 80%, at least 85%, at least 90%, at least 95%, at least 97%, or at least 99% sequence identity to either of SEQ ID NOs: 90 or 91. In certain embodiments, the exogenous succinate exporter gene has either of nucleotide sequence SEQ ID NOs: 92 or 93, or a nucleotide sequence with at least 50%, at least 60%, at least 70%, at least 80%, at least 85%, at least 90%, at least 95%, at least 97%, or at least 99% sequence identity to either SEQ ID NOs: 92 or 93.

In certain embodiments, the recombinant yeast cells provided herein may have a deletion or disruption of one or more other endogenous genes. The other deleted or disrupted genes may include genes which produce enzymes that catalyze the reaction of pyruvate or phosphoenolpyruvate (or their metabolizes) to downstream products other than succinate. Among such genes are, for example, native pyruvate decarboxylase, alcohol dehydrogenase 1 (ADH1, catalyzes the conversion of acetaldehyde to ethanol), alcohol dehydrogenase 2 (ADH2, catalyzes the conversion of ethanol to acetaldehyde), glycerol-3-phosphate dehydrogenase (GPD, systematic name sn-glycerol-3-phosphate:NAD+ 2-oxidoreductase, EC 1.1.1.8), and glycerol-3-phosphatase enzyme (GPP, systematic name glycerol-1-phosphate phosphohydrolase, EC 3.1.3.21) and NADH⁺-dependent glycerol dehydrogenase (systematic name glycerol:NAD+ 2-oxidoreductase, EC 1.1.1.6) genes.

Other endogenous genes that may be deleted in certain embodiments of the invention include genes which encode for enzymes that catalyze a reaction that consumes PEP, pyruvate, succinate or any intermediates produced in the reductive TCA pathway (other than the TCA pathway reactions leading to succinate). Examples of such genes include a native pyruvate carboxylase gene (which encodes an enzyme that converts OAA to pyruvate), a native PEP carboxykinase (PCK) gene (which encodes an enzyme that converts OAA to PEP), a native malic enzyme (MAE) gene (which encodes an enzyme that converts malate to pyruvate) and a native succinate dehydrogenase (SDH) gene (which encodes an enzyme that catalyzes the back-reaction of succinate to fumarate).

In some embodiments, the modified yeast cells provided herein have a deletion or disruption of a native succinate importer gene, which as used herein refers to any gene that encodes a polypeptide that allows for growth on and consumption of succinate.

In certain embodiments, the cells may contain all or part of an active oxidative TCA or glyoxylate shunt succinate fermentation pathway. In these embodiments, the cells comprise one or more genes encoding enzymes selected from the group consisting of citrate synthase, PDH (pyruvate dehydrogenase), PFL (pyruvate formate lyase), aconitase, IDH (isocitrate dehydrogenase), α-KGDH (α-ketoglutarate dehydrogenase), succinate thiokinase, isocitrate lyase, and malate synthase.

The recombinant cell of the invention may further include one or more modifications which individually or collectively confers to the cell the ability to ferment pentose sugars to xylulose 5-phosphate. Among such modifications are (1) insertion of a functional xylose isomerase gene, (2) a deletion or disruption of a native gene that produces an enzyme that catalyzes the conversion of xylose to xylitol, (3) a deletion or disruption of a functional xylitol dehydrogenase gene and/or (4) modifications that cause the cell to overexpress a functional xylulokinase. Methods for introducing those modifications into yeast cells are described, for example, in WO 04/099381, incorporated herein by reference. Suitable methods for inserting a functional xylose isomerase gene, deleting or disrupting a native gene that produces an enzyme that catalyzes the conversion of xylose to xylitol, deleting or disrupting a functional xylitol dehydrogenase gene, and modifying the cell to overexpress a functional xylulokinase are described, for example, in WO 04/099381, incorporated herein by reference.

In this invention, any exogenous gene, including without limitation any of the exogeneous genes in the reductive TCA pathway from pyruvate to succinate, any succinate exporter gene, any G6PDH gene, any 6PGDH gene, any SthA gene, or any other exogenous gene introduced into the host cell, is operatively linked to one or more regulatory elements, and in particular to a promoter sequence and a terminator sequence that each are functional in the host cell. Such regulatory elements may be native or non-native to the host cell.

Examples of promoters that may be linked to one or more exogenous genes in the yeast cells provided herein include, but are not limited to, promoters for pyruvate decarboxylase (PDC1), phosphoglycerate kinase (PGK), xylose reductase (XR), xylitol dehydrogenase (XDH), L-(+)-lactate-cytochrome c oxidoreductase (CYB2), translation elongation factor-1 or -2 (TEF1, TEF2), enolase (ENO1), glyceraldehyde-3-phosphate dehydrogenase (GAPDH), orotidine 5′-phosphate decarboxylase (URA3) genes, as well as any of those described in the various Examples that follow. Where the promoters are non-native, they may be identical to or share a high degree of sequence identity (i.e., at least about 80%, at least about 85%, at least about 90%, at least about 95%, or at least about 99%) with one or more native promoters. Other suitable promoters and terminators include those described, for example, in WO99/14335, WO00/71738, WO02/42471, WO03/102201, WO03/102152 and WO03/049525.

Examples of terminators that may be linked to one or more exogenous genes in the yeast cells provided herein include, but are not limited to, terminators for PDC1, XR, XDH, transaldolase (TAL), transketolase (TKL), ribose 5-phosphate ketol-isomerase (RKI), CYB2, or iso-2-cytochrome c (CYC) genes or the galactose family of genes (especially the GAL10 terminator), as well as any of those described in the various Examples that follow. Where the terminators are non-native, they may be identical to or share a high degree of sequence identity (i.e., at least about 80%, at least about 85%, at least about 90%, at least about 95%, or at least about 99%) with one or more native terminators.

Modifications (insertion, deletions and/or disruptions) to the genome of the host cell described herein can be performed using methods known in the art. Exogeneous genes may be integrated into the genome in a targeted or a random manner using, for example, well known electroporosis and chemical methods (including calcium chloride and/or lithium acetate methods). In those embodiments where an exogenous gene is integrated in a targeted manner, it may be integrated into the locus for a particular native gene, such that integration of the exogenous gene is coupled with deletion or disruption of a native gene. Alternatively, the exogenous gene may be integrated into a portion of the native genome that does not correspond to a gene. Methods for transforming a yeast cell with an exogenous construct are described in, for example, WO99/14335, WO00/71738, WO02/42471, WO03/102201, WO03/102152, WO03/049525, WO2007/061590, WO 2009/065778 and PCT/US2011/022612.

Insertion of exogenous genes is generally performed by transforming the cell with one or more integration constructs or fragments. The terms “construct” and “fragment” are used interchangeably herein to refer to a DNA sequence that is used to transform a cell. The construct or fragment may be, for example, a circular plasmid or vector, a portion of a circular plasmid or vector (such as a restriction enzyme digestion product), a linearized plasmid or vector, or a PCR product prepared using a plasmid or genomic DNA as a template. An integration construct can be assembled using two cloned target DNA sequences from an insertion site target. The two target DNA sequences may be contiguous or non-contiguous in the native host genome. In this context, “non-contiguous” means that the DNA sequences are not immediately adjacent to one another in the native genome, but are instead are separated by a region that is to be deleted. “Contiguous” sequences as used herein are directly adjacent to one another in the native genome. Where targeted integration is to be coupled with deletion or disruption of a target gene, the integration construct also functions as a deletion construct. In such an integration/deletion construct, one of the target sequences may include a region 5′ to the promoter of the target gene, all or a portion of the promoter region, all or a portion of the target gene coding sequence, or some combination thereof. The other target sequence may include a region 3′ to the terminator of the target gene, all or a portion of the terminator region, and/or all or a portion of the target gene coding sequence. Where targeted integration is not to be coupled to deletion or disruption of a native gene, the target sequences are selected such that insertion of an intervening sequence will not disrupt native gene expression. An integration or deletion construct is prepared such that the two target sequences are oriented in the same direction in relation to one another as they natively appear in the genome of the host cell. The gene expression cassette is cloned into the construct between the two target gene sequences to allow for expression of the exogenous gene. The gene expression cassette contains the exogenous gene, and may further include one or more regulatory sequences such as promoters or terminators operatively linked to the exogenous gene.

It is usually desirable that the deletion construct may also include a functional selection marker cassette. When a single deletion construct is used, the marker cassette resides on the vector downstream (i.e., in the 3′ direction) of the 5′ sequence from the target locus and upstream (i.e., in the 5′ direction) of the 3′ sequence from the target locus. Successful transformants will contain the selection marker cassette, which imparts to the successfully transformed cell some characteristic that provides a basis for selection.

A “selection marker gene” may encode for a protein needed for the survival and/or growth of the transformed cell in a selective culture medium. Typical selection marker genes encode proteins that (a) confer resistance to antibiotics or other toxins, (such as, for example, zeocin (Streptoalloteichus hindustanus ble bleomycin resistance gene), G418 (kanamycin-resistance gene of Tn903) or hygromycin (aminoglycoside antibiotic resistance gene from E. coli)), (b) complement auxotrophic deficiencies of the cell (such as, for example, amino acid leucine deficiency (K. marxianus LEU2 gene) or uracil deficiency (e.g., K. marxianus or S. cerevisiae URA3 gene)); (c) enable the cell to synthesize critical nutrients not available from simple media, or (d) confer ability for the cell to grow on a particular carbon source, (such as a MEL5 gene from S. cerevisiae, which encodes the alpha-galactosidase (melibiase) enzyme and confers the ability to grow on melibiose as the sole carbon source). Preferred selection markers include the zeocin resistance gene, G418 resistance gene, a MEL5 gene, a URA3 gene and hygromycin resistance gene. Another preferred selection marker is an L-lactate:ferricytochrome c oxidoreductase (CYB2) gene cassette, provided that the host cell either natively lacks such a gene or that its native CYB2 gene(s) are first deleted or disrupted.

The construct may be designed so that the selection marker cassette can become spontaneously deleted as a result of a subsequent homologous recombination event. A convenient way of accomplishing this is to design the vector such that the selection marker gene cassette is flanked by direct repeat sequences. Direct repeat sequences are identical DNA sequences, native or not native to the host cell, and oriented on the construct in the same direction with respect to each other. The direct repeat sequences are advantageously about 50-1500 bp in length. It is not necessary that the direct repeat sequences encode for anything. This construct permits a homologous recombination event to occur. This event occurs with some low frequency, resulting in cells containing a deletion of the selection marker gene and one of the direct repeat sequences. It may be necessary to grow transformants for several rounds on nonselective or selective media to allow for the spontaneous homologous recombination to occur in some of the cells. Cells in which the selection marker gene has become spontaneously deleted can be selected or screened on the basis of their loss of the selection characteristic imparted by the selection marker gene, or by using PCR or Southern Analysis methods to confirm the loss of the selection marker.

In some embodiments, an exogenous gene may be inserted using DNA from two or more integration fragments, rather than a single fragment. In these embodiments, the 3′ end of one integration fragment contains a region of homology with the 5′ end of another integration fragment. One of the fragments will contain a first region of homology to the target locus and the other fragment will contain a second region of homology to the target locus. The gene cassette to be inserted can reside on either fragment, or be divided among the fragments, with a region of homology at the 3′ and 5′ ends of the respective fragments, so the entire, functional gene cassette is produced upon a crossover event. The cell is transformed with these fragments simultaneously. A selection marker may reside on any one of the fragments or may be divided between the fragments with a region of homology as described. In other embodiments, transformation from three or more constructs can be used in an analogous way to integrate exogenous genetic material.

Deletions and/or disruptions of native genes can be performed by transformation methods, by mutagenesis and/or by forced evolution methods. In mutagenesis methods cells are exposed to ultraviolet radiation or a mutagenic substance, under conditions sufficient to achieve a high kill rate (60-99.9%, preferably 90-99.9%) of the cells. Surviving cells are then plated and selected or screened for cells having the deleted or disrupted metabolic activity. Disruption or deletion of the desired native gene(s) can be confirmed through PCR or Southern analysis methods.

Cells of the invention can be cultivated to produce succinic acid, either in the free acid form or in salt form (or both), or a metabolization product of succinate. The recombinant cell is cultured in a medium that includes at least one carbon source that can be fermented by the cell. Examples include, but are not limited to, twelve carbon sugars such as sucrose, hexose sugars such as glucose or fructose, glycan, starch, or other polymer of glucose, glucose oligomers such as maltose, maltotriose and isomaltotriose, panose, and fructose oligomers, and pentose sugars such as xylose, xylan, other oligomers of xylose, or arabinose.

The medium will typically contain, in addition to the carbon source, nutrients as required by the particular cell, including a source of nitrogen (such as amino acids, proteins, inorganic nitrogen sources such as ammonia or ammonium salts, and the like), and various vitamins, minerals and the like. In some embodiments, the cells of the invention can be cultured in a chemically defined medium.

Other cultivation conditions, such as temperature, cell density, selection of substrate(s), selection of nutrients, and the like are not considered to be critical to the invention and are generally selected to provide an economical process. Temperatures during each of the growth phase and the production phase may range from above the freezing temperature of the medium to about 50° C., although this depends to some extent on the ability of the strain to tolerate elevated temperatures. A preferred temperature, particularly during the production phase, is about 30 to 45° C.

During cultivation, aeration and agitation conditions may be selected to produce a desired oxygen uptake rate. The cultivation may be conducted aerobically, microaerobically, or anaerobically, depending on pathway requirements. In some embodiments, the cultivation conditions are selected to produce an oxygen uptake rate of around 2-25 mmol/L/hr, preferably from around 5-20 mmol/L/hr, and more preferably from around 8-15 mmol/L/hr. “Oxygen uptake rate” or “OUR” as used herein refers to the volumetric rate at which oxygen is consumed during the fermentation. Inlet and outlet oxygen concentrations can be measured with exhaust gas analysis, for example by mass spectrometers. OUR can be calculated using the Direct Method described in Bioreaction Engineering Principles 2nd Edition, 2003, Kluwer Academic/Plenum Publishers, p. 449, equation 1.

The culturing process may be divided up into phases. For example, the cell culture process may be divided into a cultivation phase, a production phase, and a recovery phase.

The pH may be allowed to range freely during cultivation, or may be buffered if necessary to prevent the pH from falling below or rising above predetermined levels. For example, the medium may be buffered to prevent the pH of the solution from falling below around 2.0 or above about 8.0 during cultivation. In certain of these embodiments, the medium may be buffered to prevent the pH of the solution from falling below around 3.0 or rising above around 7.0, and in certain of these embodiments the medium may be buffered to prevent the pH of the solution from falling below around 3.0 or rising above around 4.5. Suitable buffering agents include basic materials that neutralize the acid as it is formed, and include, for example, calcium hydroxide, calcium carbonate, sodium hydroxide, potassium hydroxide, potassium carbonate, sodium carbonate, ammonium carbonate, ammonia, ammonium hydroxide and the like.

In a buffered fermentation, acidic fermentation products are neutralized to the corresponding salt as they are formed. Recovery of the acid therefore involves regenerating the free acid. This is typically done by removing the cells and acidulating the fermentation broth with a strong acid such as sulfuric acid. A salt by-product is formed (gypsum in the case where a calcium salt is the neutralizing agent and sulfuric acid is the acidulating agent), which is separated from the broth.

In other embodiments, the pH of the fermentation medium may be permitted to drop during cultivation from a starting pH that is at or above the lower pKa (4.207) of succinate, typically 8 or higher, to at or below the lower pKa of the acid fermentation product, such as in the range of about 2.0 to about 4.2, in the range of from about 3.0 to about 4.2, or in the range from about 3.8 to about 4.2.

In still other embodiments, fermentation may be carried out to produce a product acid by adjusting the pH of the fermentation broth to at or below the lower pKa of the product acid prior to or at the start of the fermentation process. The pH may thereafter be maintained at or below the lower pKa of the product acid throughout the cultivation. In certain embodiments, the pH may be maintained at a range of about 2.0 to about 4.2, in the range of from about 3.0 to about 4.2, or in the range from about 3.8 to about 4.2.

When the pH of the fermentation broth is low enough that the succinate is present in acid form, the acid can be recovered from the broth through techniques such as liquid-liquid extraction, distillation, absorption, etc., such as are described in T. B. Vickroy, Vol. 3, Chapter 38 of Comprehensive Biotechnology, (ed. M. Moo-Young), Pergamon, Oxford, 1985; R. Datta, et al., FEMS Microbiol. Rev., 1995, 16:221-231; U.S. Pat. Nos. 4,275,234, 4,771,001, 5,132,456, 5,420,304, 5,510,526, 5,641,406, and 5,831,122, and WO 93/00440.

The cultivation may be continued until a yield of succinate on the carbon source is, for example, at least 10%, at least 20%, at least 30%, at least 40%, at least 50%, or greater than 50% of the theoretical yield. The yield to succinate may at least 80% or at least 90% of the theoretical yield. The concentration, or titer, of succinate produced in the cultivation will be a function of the yield as well as the starting concentration of the carbon source. In certain embodiments, the titer may reach at least 1, at least 3, at least 5, at least 10, at least 20, at least 30, at least 40, at least 50, or greater than 50 g/L at some point during the fermentation, and preferably at the end of the fermentation.

In certain embodiments, the genetically modified yeast cells produce ethanol in a yield of 10% or less, preferably in a yield of 2% or less of the theoretical yield. In certain of these embodiments, ethanol is not detectably produced. In other embodiments, however, succinate and ethanol may be co-produced. In these embodiments, ethanol may be produced at a yield of greater than 10%, greater than 25%, or greater than 50% of the theoretical yield.

The recombinant cell of the invention may exhibit a volumetric glucose consumption rate of at least 0.5 gram, at least 0.75 gram, or at least 0.9 gram of glucose per liter of broth per hour, when cultivated under the conditions described in Examples 253-255.

The cell of the invention may produce succinate as an end-product of the fermentation process. In such a case, the cell preferably transports succinate out of the cell and into the surrounding culture medium.

In some embodiments, the cell may further metabolize some or all of the succinate into one or more succinate metabolization products, i.e., a compound formed in the further metabolization of succinate by the cell. Examples of such downstream succinate metabolization products include, for example, 1,4-butanediol, 1,3-butadiene, propionic acid, and 3-hydroxyisobutryic acid. In such embodiments, the cell contains native or non-native metabolic pathways which perform the such a further metabolization of succinate into such downstream succinate metabolization product(s). The cell may then transport such downstream succinate metabolization products out of the cell and into the surrounding medium. In some embodiments, the cell may transport one or more succinate metabolization products, but not succinate, out of the cell. In other embodiments, the cell may transport both succinate itself and one or more succinate metabolization products out of the cell. For example, the cell may transport less than 10% by weight of succinate from the cell, based on the combined weight of succinate and succinate metabolization products exported from the cell.

The following examples are provided to illustrate the invention, but are not intended to limit the scope thereof. All parts and percentages are by weight unless otherwise indicated.

### EXAMPLES

### Construction of Preparatory Examples

P-1. An I. orientalis strain host strain is generated by evolving I. orientalis strain ATCC PTA-6658 for 91 days in a glucose-limited chemostat. The system is fed 15 g/L glucose in a defined medium and operated at a dilution rate of 0.06 h⁻¹ at pH=3 with added lactic acid in the feed medium. The conditions are maintained with an oxygen transfer rate of approximately 2 mmol L⁻¹h⁻¹, and dissolved oxygen concentration remains constant at 0% of air saturation. Single colony isolates from the final time point are characterized in two shake flask assays. In the first assay, the isolates are characterized for their ability to ferment glucose to ethanol in the presence of 25 g/L total lactic acid with no pH adjustment in the defined medium. In the second assay, the growth rate of the isolates is measured in the presence of 45 g/L of total lactic acid, with no pH adjustment in the defined medium. Strain P-1 is a single isolate exhibiting the highest glucose consumption rate in the first assay and the highest growth rate in the second assay.

P-2. Strain P-1 is transformed with linearized integration fragment P2 (having nucleotide sequence SEQ ID NO: 1) designed to disrupt the URA3 gene, using a LiOAc transformation method as described by Gietz et al., in Met. Enzymol. 350:87 (2002). Integration fragment P2 includes a MEL5 selection marker gene. Transformants are selected on YNB-melibiose plates and screened by PCR to confirm the integration of the integration piece and deletion of a copy of the URA3 gene. A URA3-deletant strain is grown for several rounds until PCR screening identifies an isolate in which the MEL5 selection marker gene has looped out. The PCR screening is performed using primers having nucleotide sequences SEQ ID NOs: 47 and 48 to confirm the 5′-crossover and primers having nucleotide sequences SEQ ID NOs: 51 and 52 to confirm the 3′ crossover. That isolate is again grown for several rounds on 5-fluoroorotic acid (FOA) plates to identify a strain in which the URA3 marker has looped out. PCR screening is performed on this strain using primers having nucleotide sequences SEQ ID NOs: 56 and 124, identifies an isolate in which both URA3 alleles have been deleted. This isolate is named strain P-2.

P-3. Strain P-2 is transformed with integration fragment P3 (having the nucleotide sequence SEQ ID NO: 2), which is designed to disrupt the PDC gene. Integration fragment P3 contains the following elements, 5′ to 3′: a DNA fragment with homology for integration corresponding to the region immediately upstream of the I. orientalis PDC open reading frame, a PDC transcriptional terminator, the URA3 promoter, the I. orientalis URA3 gene, an additional URA3 promoter direct repeat for marker recycling and a DNA fragment with homology for integration corresponding to the region directly downstream of the I. orientalis PDC open reading frame. A successful integrant (and single-copy PDC deletant) is identified on selection plates lacking uracil and confirmed by PCR using primers having nucleotide sequences SEQ ID NOS: 53 and 54 to confirm the 5′-crossover and primers having nucleotide sequences SEQ ID NOs: 55 and 122 to confirm the 3′-crossover. That integrant is grown for several rounds and plated on 5-fluoroorotic acid (FOA) plates to identify a strain in which the URA3 marker has looped out. Loopout of the URA3 marker is confirmed by PCR. That strain is again transformed with integration fragment P3 (SEQ ID NO: 2) to delete the second copy of the native PDC gene. A successful transformant is again identified by selection on selection plates lacking uracil, and further confirmed by culturing the strain over two days and measuring ethanol production. Lack of ethanol production further demonstrates a successful deletion of both copies of the PDC gene in a transformant. That transformant is grown for several rounds and plated on FOA plates until PCR identifies a strain in which the URA3 marker has looped out. The PCR screening is performed using primers having nucleotide sequences SEQ ID NOs: 53 and 54 to confirm the 5′-crossover and SEQ ID NOs: 55 and 122 to confirm the 3′-crossover. That strain is plated on selection plates lacking uracil to confirm the loss of the URA3 marker, and is designated strain P-3.

P-4. Integration fragment P4-1, having nucleotide sequence SEQ ID NO: 3, contains the following elements, 5′ to 3′: a DNA fragment with homology for integration corresponding to the region immediately upstream of the I. orientalis ADH9091 open reading frame, an I. orientalis PDC1 promoter, the I. orientalis PYC gene (having the nucleotide sequence SEQ ID NO: 4), the I. orientalis TAL terminator, the I. orientalis URA3 promoter, and the first 530 bp of the I. orientalis URA3 open reading frame.

Integration fragment P4-2, having nucleotide sequence SEQ ID NO: 5, contains the following elements, 5′ to 3′: a DNA fragment corresponding to the last 568 bp of the I. orientalis URA3 open reading frame, the I. orientalis URA3 terminator, the I. orientalis URA3 promoter, the I. orientalis TDH3 promoter, the S. pombe MAE gene (having the nucleotide sequence SEQ ID NO: 6), the I. orientalis TKL terminator, and a DNA fragment with homology for integration corresponding to the region immediately downstream of the I. orientalis ADH9091 open reading frame.

Strain P-3 is transformed simultaneously with integration fragments P4-1 and P4-2, using lithium acetate methods, to insert the I. orientalis PYC gene and the S. pombe MAE gene at the ADH9091 locus. Integration occurs via three cross-over events: in the regions of the ADH9091 upstream homology, in the regions of the ADH9091 downstream homology and in the region of URA3 homology between SEQ ID NO: 3 and SEQ ID NO: 5. Transformants are streaked to isolates and the correct integration of the cassette at the AHD9091 locus is confirmed in a strain by PCR. The PCR screening is performed using primers having nucleotide sequences SEQ ID NOs: 65 and 69 to confirm the 5′-crossover and SEQ ID NOs: 67 and 71 to confirm the 3′-crossover. That strain is grown and plated on FOA as before until the loopout of the URA3 marker from an isolate is confirmed by PCR.

That isolate is then transformed simultaneously with integration fragments P4-3 and P4-4 using LiOAc transformation methods, to insert a second copy of each of the I. orientalis PYC gene and the S. pombe MAE gene at the ADH9091 locus.

Integration fragment P4-3, having the nucleotide sequence SEQ ID NO: 7, contains the following elements, 5′ to 3′: a DNA fragment with homology for integration corresponding to the region immediately downstream of the I. orientalis ADH9091 open reading frame, an I. orientalis PDC1 promoter, the I. orientalis PYC gene as found in SEQ ID NO: 4, the I. orientalis TAL terminator, the I. orientalis URA3 promoter, and the first 530 bp of the I. orientalis URA3 open reading frame.

Integration fragment P4-4, having the nucleotide sequence SEQ ID NO: 8, contains the following elements, 5′ to 3′: a DNA fragment corresponding to the last 568 bp of the I. orientalis URA3 open reading frame, the I. orientalis URA3 terminator, the I. orientalis URA3 promoter, the I. orientalis TDH3 promoter, the S. pombe MAE gene (having a nucleotide sequences SEQ ID NO: 6), the I. orientalis TKL terminator, and a DNA fragment with homology for integration corresponding to the region immediately upstream of the I. orientalis ADH9091 open reading frame.

Integration again occurs via three crossover events. Transformants are streaked to isolates and screened by PCR to identify a strain containing both copies of the I. orientalis PYCI and S. pombe MAE genes at the ADH9091 locus by PCR. The PCR screening to confirm the first copy is performed using primers having nucleotide sequences SEQ ID NOs: 65 and 69 to confirm the 5′-crossover and SEQ ID NOs: 67 and 71 to confirm the 3′-crossover. The PCR screening to confirm the second copy is performed using primers having nucleotide sequences SEQ ID NOs: 65 and 67 to confirm the 5′-crossover and SEQ ID NOs: 69 and 71 to confirm the 3′-crossover. That strain is grown and replated on FOA until a strain in which the URA3 marker has looped out is identified. That strain is designated strain P-4.

P-5. Strain P-4 is transformed with integration fragment P5-1 (having the nucleotide sequence SEQ ID NO: 9) using LiOAc transformation methods as described in previous examples, to integrate the L. mexicana FRD gene at the locus of the native CYB2b open reading frame. The integration fragment P5-1 contains the following elements, 5′ to 3′: a DNA fragment with homology for integration corresponding to the region immediately downstream of the I. orientalis CYB2b open reading frame, an I. orientalis PDC1 promoter, the L. mexicana FRD gene (having nucleotide sequence SEQ ID NO: 10, and encoding for an enzyme having amino acid sequence SEQ ID NO: 82), the I. orientalis PDC1 Terminator, the I. orientalis URA3 promoter, gene, and terminator in succession, followed by an additional URA3 promoter which serves as a direct repeat for marker recycling, and a region immediately upstream of the I. orientalis CYB2b open reading frame.

Successful integration of a single copy of the L. mexicana FRD gene in one isolate is identified by selection on a selection plates lacking uracil and confirmed by PCR. The PCR screening is performed using primers having nucleotide sequences SEQ ID NOs: 72 and 73 to confirm the 5′-crossover and SEQ ID NOs: 69 and 79 to confirm the 3′-crossover. That isolate is grown and plated on FOA as before until a strain in which the URA3 promoter has looped out is identified by PCR. That isolate is transformed with integration fragment P5-2 in the same manner as before, to integrate a second copy of the L. mexicana FRD gene at the native locus of the CYB2b open reading frame.

Integration fragment P5-2 (having nucleotide sequence SEQ ID NO: 11), contains the following elements, 5′ to 3′: a DNA fragment with homology for integration corresponding to the region immediately upstream of the I. orientalis CYB2b open reading frame, an I. orientalis PDC1 promoter, the L. mexicana FRD gene (having the nucleotide sequence SEQ ID NO: 10), the I. orientalis PDC1 terminator, the I. orientalis URA3 promoter, gene, and terminator in succession, followed by an additional URA3 promoter which serves as a direct repeat for marker recycling, and a region immediately downstream of the I. orientalis CYB2b open reading frame.

Correct integration of the second copy of the L. mexicana FRD gene in one isolate is confirmed by PCR using primers having nucleotide sequences SEQ ID NOs: 69 and 73 to confirm the 5′-crossover and SEQ ID NOs: 72 and 79 to confirm the 3′-crossover. Retention of the first integration is reconfirmed by repeating the PCR reactions used to verify proper integration of fragment P5-1 above. The confirmed isolate is grown and plated on FOA as before until the loop out of the URA3 marker is confirmed by PCR in one isolate. That isolate is designated strain P-5.

P-6. Integration fragment P6-1 (having nucleotide sequence SEQ ID NO: 12) contains the Rhizopus delemar MDH (RdMDH) gene (having the nucleotide sequence SEQ ID NO: 13), an ADHb upstream integration arm, ENO promoter, RKI terminator, URA3 promoter and the first 583 base pairs of the URA3 marker.

Integration fragment P6-2 (having nucleotide sequence SEQ ID NO: 14) contains the Actinobacillus succinogenes FUM (AsFUM) gene (nucleotide sequence SEQ ID NO: 15), the last 568 base pairs of the URA3 marker, URA3 promoter, PGK promoter, PDC terminator and ADHb downstream integration arm.

Strain P-5 is simultaneously transformed with each of integration fragments P6-1 and P6-2 using the standard lithium acetate process described before. Successful transformants are identified by PCR, and grown and plated until a strain in which the URA3 marker has looped out is identified as before. This strain is designated as strain P-6.

Second Rhizopus delemar MDH integration fragment P6-3 (having the nucleotide sequence SEQ ID NO: 16) contains the Rhizopus delemar MDH gene (having nucleotide sequence SEQ ID NO: 13), ADHb downstream integration arm, ENO promoter, RKI terminator, URA3 promoter and the first 583 base pairs of the URA3 marker.

Second A. succinogenes FUM (AsFUM) integration fragment P6-4 (having nucleotide sequence SEQ ID NO: 17) contains the truncated AsFUM gene (nucleotide sequence SEQ ID NO: 15) the last 568 base pairs of the URA3 marker, URA3 promoter, PGK promoter, PDC terminator and ADHb upstream integration arm.

Strain P-6 is simultaneously transformed with integration fragments P6-3 and P6-4, using the standard lithium acetate process described before. Successful transformants are identified by PCR, and grown and plated on FOA as before until a strain in which the URA3 marker has looped out is identified. This strain is designated as strain P-7.

TABLE 1
Preparatory Strains P-1 through P-7

| Strain name | Description | Parent strain |
|---|---|---|
| P-1 | Organic acid tolerant I. orientalis isolate | Wild-type |
| P-2 | URA3 deletion (2) | P-1 |
| P-3 | URA3 deletion (2) | P-2 |
| | PDC deletion (2) | |
| P-4 | URA deletion (2) | P-3 |
| | PDC deletion (2) | |
| | I. orientalis PYC1 insertion at ADHa (2) | |
| | S. pombe MAE insertion at ADHa (2) | |
| P-5 | URA deletion (2) | P-4 |
| | PDC deletion (2) | |
| | I. orientalis PYC1 insertion at ADHa (2) | |
| | S. pombe MAE insertion at ADHa (2) | |
| | L. mexicana FRD insertion at CYB2b (2) | |
| P-6 | URA deletion (2) | P-5 |
| | PDC deletion (2) | |
| | I. orientalis PYC1 insertion at ADHa (2) | |
| | S. pombe MAE insertion at ADHa (2) | |
| | L. mexicana FRD insertion at CYB2b (2) | |
| | R. delemar MDH insertion at ADHb (1) | |
| | A. succinogenes FUM insertion at ADHb (1) | |
| P-7 | URA deletion (2) | P-6 |
| | PDC deletion (2) | |
| | I. orientalis PYC1 insertion at ADHa (2) | |
| | S. pombe MAE insertion at ADHa (2) | |
| | L. mexicana FRD insertion at CYB2b (2) | |
| | R. delemar MDH insertion at ADHb (2) | |
| | A. succinogenes FUM insertion at ADHb (2) | |

### Examples 1-9: Integration of Soluble Transhydrogenase

General procedure for producing Examples 1-9: The host strain (as indicated in Table 2 below) is simultaneously transformed with each of two integration fragments, as indicated in Table 2 below, using the standard lithium acetate process described before. The integration fragments are designed for targeted insertion at the native MAE1 gene of the host strain. Integration occurs via three cross-over events: the MAE1 upstream homology, the MAE1 downstream homology and homology between portions of the URA3 gene that are present in each of the integration fragments. Transformants are streaked to isolates and the correct integration of the cassette at the MAE1 locus is confirmed by PCR using primers having nucleotide sequences SEQ ID NOs: 80 and 81 to confirm the 5′-crossover and SEQ ID NOs: 85 and 126 to confirm the 3′-crossover. That strain is grown and plated on FOA as before until the loopout of the URA3 marker from an isolate is confirmed by PCR.

The integration fragments used to produce strain Examples 1-9 are as follows:

### Integration Fragment 1A: Left Hand Integration Fragment—Marker Only

Integration fragment 1A, having the nucleotide sequence SEQ ID NO: 18, contains the following elements, 5′ to 3′: a DNA fragment with homology for integration corresponding to the region immediately upstream of the I. orientalis MAE1 open reading frame, an I. orientalis PDC1 promoter, the I. orientalis TAL terminator, the I. orientalis ENO promoter, I. orientalis RKI terminator, URA3 promoter, and the first 582 bp of the I. orientalis URA3 open reading frame.

### Integration Fragment 1B: Right Hand Integration Fragment—Marker Only

Integration fragment 1B having the nucleotide sequence SEQ ID NO: 19, contains the following elements, 5′ to 3′: a DNA fragment corresponding to the last 567 bp of the I. orientalis URA3 open reading frame, the I. orientalis URA3 terminator, the I. orientalis URA3 promoter, the I. orientalis TDH3 promoter, the I. orientalis TKL terminator, the I. orientalis PGK promoter, the I. orientalis PDC terminator and a DNA fragment with homology for integration corresponding to the region immediately downstream of the I. orientalis MAE1 open reading frame.

### Integration Fragment 1C: Left Hand Integration Fragment with the E. coli SthA Gene

Integration fragment 1C, having the nucleotide sequence SEQ ID NO: 20, contains the following elements, 5′ to 3′: a DNA fragment with homology for integration corresponding to the region immediately upstream of the I. orientalis MAE1 open reading frame, an I. orientalis PDC1 promoter, the I. orientalis TAL terminator, the I. orientalis ENO promoter, the E. coli SthA gene (having nucleotide sequence SEQ ID NO: 21), I. orientalis RKI terminator, URA3 promoter, and the first 582 bp of the I. orientalis URA3 open reading frame.

### Integration Fragment 1D: Right Hand Integration Fragment with the E. coli SthA Gene

Integration fragment 1D, having nucleotide sequence SEQ ID NO: 22, contains the following elements, 5′ to 3′: a DNA fragment corresponding to the last 567 bp of the I. orientalis URA3 open reading frame, the I. orientalis URA3 terminator, the I. orientalis URA3 promoter, the I. orientalis TDH3 promoter, the I. orientalis TKL terminator, the I. orientalis PGK promoter, the E. coli SthA gene (having nucleotide sequence SEQ ID NO: 21), the I. orientalis PDC terminator and a DNA fragment with homology for integration corresponding to the region immediately downstream of the I. orientalis MAE1 open reading frame.

### Integration Fragment 1E: Left Hand Integration Fragment with a Codon Optimized E. coli SthA Gene

Integration fragment IE, having the nucleotide sequence SEQ ID NO: 23, contains the following elements, 5′ to 3′: a DNA fragment with homology for integration corresponding to the region immediately upstream of the I. orientalis MAE1 open reading frame, an I. orientalis PDC1 promoter, the I. orientalis TAL terminator, the I. orientalis ENO promoter, the codon optimized E. coli SthA gene (having nucleotide sequence SEQ ID NO: 24), I. orientalis RKI terminator, URA3 promoter, and the first 582 bp of the I. orientalis URA3 open reading frame.

### Integration Fragment 1F: Right Hand Integration Fragment with the Codon Optimized E. coli SthA Gene

Integration fragment 1F, having the nucleotide sequence SEQ ID NO: 25, contains the following elements, 5′ to 3′: a DNA fragment corresponding to the last 567 bp of the I. orientalis URA3 open reading frame, the I. orientalis URA3 terminator, the I. orientalis URA3 promoter, the I. orientalis TDH3 promoter, the I. orientalis TKL terminator, the I. orientalis PGK promoter, the codon-optimized E. coli SthA gene (having nucleotide sequence SEQ ID NO: 24), the I. orientalis PDC terminator and a DNA fragment with homology for integration corresponding to the region immediately downstream of the I. orientalis MAE1 open reading frame.

### Integration Fragment 1G: Left Hand Integration Fragment with the A. vinelandii SthA Gene

Integration fragment 1G, having the nucleotide sequence SEQ ID NO: 26, contains the following elements, 5′ to 3′: a DNA fragment with homology for integration corresponding to the region immediately upstream of the I. orientalis MAE1 open reading frame, an I. orientalis PDC1 promoter, the I. orientalis TAL terminator, the I. orientalis ENO promoter, the A. vinelandii SthA gene (having nucleotide sequence, SEQ ID NO: 27), I. orientalis RKI terminator, URA3 promoter, and the first 582 bp of the I. orientalis URA3 open reading frame.

### Integration Fragment 1H: Right Hand Integration Fragment with the A. vinelandii SthA Gene

Integration fragment 1H, having the nucleotide sequence SEQ ID NO: 28, contains the following elements, 5′ to 3′: a DNA fragment corresponding to the last 567 bp of the I. orientalis URA3 open reading frame, the I. orientalis URA3 terminator, the I. orientalis URA3 promoter, the I. orientalis TDH3 promoter, the I. orientalis TKL terminator, the I. orientalis PGK promoter, the A. vinelandii SthA gene (having nucleotide sequence SEQ ID NO: 27), the I. orientalis PDC terminator and a DNA fragment with homology for integration corresponding to the region immediately downstream of the I. orientalis MAE1 open reading frame.

### Integration Fragment 1I: Left Hand Integration Fragment with the S. cerevisiae Stb5p Gene

Integration fragment 1I, having nucleotide sequence SEQ ID NO: 29, contains the following elements, 5′ to 3′: a DNA fragment with homology for integration corresponding to the region immediately upstream of the I. orientalis MAE1 open reading frame, an I. orientalis PDC1 promoter, the S. cerevisiae Stb5p gene (having nucleotide sequence SEQ ID NO: 30), the I. orientalis TAL terminator, the I. orientalis ENO promoter, the I. orientalis RKI terminator, URA3 promoter, and the first 582 bp of the I. orientalis URA3 open reading frame.

TABLE 2
I. orientalis Insertion Strains

| | Description (in addition | Integration | Parent |
|---|---|---|---|
| Designation | to those in strain P-7) | Fragments | strain |
| Ex. 1 | E. coli SthA insertion at MAE1 (1) | 1C/1B | P-7 |
| Ex. 2 | E. coli SthA insertion at MAE1 (2) | 1D/1A | Ex. 1 |
| Ex. 3 | A. vinelandii SthA insertion at MAE1 (1) | 1G/1B | P-7 |
| Ex. 4 | A. vinelandii SthA insertion at MAE1 (2) | 1H/1A | Ex. 3 |
| Ex. 5 | Codon optimized E. coli SthA insertion at MAE1 (1) | 1E/1B | P-7 |
| Ex. 6 | Codon optimized E. coli SthA insertion at MAE1 (2) | 1F/1A | Ex. 5 |
| P-8 | S. cerevisiae Stb5p insertion at MAE1 (1) | 1I/1B | P-7 |
| Ex. 7 | S. cerevisiae Stb5p insertion at MAE1 (1) | 1I/1D | P-8 |
| | E. coli SthA insertion at MAE1 (1) | | |
| Ex. 8 | S. cerevisiae Stb5p insertion at MAE1 (1) | 1I/1F | P-8 |
| | Codon optimized E. coli SthA insertion at MAE1 (1) | | |
| Ex. 9 | S. cerevisiae Stb5p insertion at MAE1 (1) | 1I/1H | P-8 |
| | A. vinelandii SthA insertion at MAE1 (1) | | |

### Examples 9-63

Strains P-9 through P-13 are prepared in the same manner as strain P-7, except the L. mexicana FRD gene in each case has been mutated to render it NADPH-dependent. In each case, the L. mexicana FRD gene having the nucleotide sequence SEQ ID NO: 10 is used as a template to modify the coding sequence to introduce substitutions of amino acid residues of the putative NADH binding domain of the enzyme.

The FRD gene used to prepare strain P-9 is prepared by performing site-directed substitutions at amino acids 219 (glutamic acid) and 220 (tryptophan) to produce a mutated gene having the nucleotide sequence SEQ ID NO. 32.

The FRD gene used to prepare strain P-10 is prepared by performing a site-directed substitution at amino acid 417 (glutamic acid) to produce a mutated gene having nucleotide sequence SEQ ID NO: 33.

The FRD gene used to prepare strain P-11 is prepared by performing a site-directed substitution at amino acid 641 (aspartic acid) to produce a mutated gene having nucleotide sequence SEQ ID NO: 34.

The FRD gene used to prepare strain P-12 is prepared by performing site-directed substitutions at amino acids 861 (glutamic acid) and 862 (cysteine) to produce a mutated gene having nucleotide sequence SEQ ID NO: 35.

The FRD gene used to prepare strain P-13 is prepared by performing site-directed substitutions at amino acids 1035 (aspartic acid) and 1036 (serine) to produce a mutated gene having nucleotide sequence SEQ ID NO: 36.

The FRD gene used to prepare strain P-14 is prepared by performing site-directed substitutions at amino acid 411 of a T. brucei FRD gene having SEQ ID NO: 42 to produce a mutated gene having nucleotide sequence SEQ ID NO: 37.

Examples 10-18 are made in the same manner as Examples 1-9, respectively, except Examples 10-18 are made starting from strain P-9 instead of strain P-7. Examples 10-18 correspond to Examples 1-9, respectively, except the FRD gene in Examples 10-18 is the mutated L. mexicana FRD gene having the nucleotide sequence SEQ ID NO: 32.

Examples 19-27 are made in the same manner as Examples 1-9, respectively, except Examples 19-27 are made starting from strain P-10 instead of strain P-7. Examples 23-33 correspond to Examples 1-9, respectively, except the FRD gene in Examples 19-27 is the mutated L. mexicana FRD gene having the nucleotide sequence SEQ ID NO: 33.

Examples 28-36 are made in the same manner as Examples 1-9, respectively, except Examples 28-36 are made starting from strain P-11 instead of strain P-7. Examples 28-36 correspond to Examples 1-9, respectively, except the FRD gene in Examples 28-36 is the mutated L. mexicana FRD gene having the nucleotide sequence SEQ ID NO: 34.

Examples 37-45 are made in the same manner as Examples 1-9, respectively, except Examples 37-45 are made starting from strain P-12 instead of strain P-7. Examples 37-45 correspond to Examples 1-9, respectively, except the FRD gene in Examples 37-45 is the mutated L. mexicana FRD gene having the nucleotide sequence SEQ ID NO: 35.

Examples 46-54 are made in the same manner as Examples 1-9, respectively, except Examples 46-54 are made starting from strain P-13 instead of strain P-7. Examples 46-54 correspond to Examples 1-9, respectively, except the FRD gene in Examples 46-54 is the mutated L. mexicana FRD gene having the nucleotide sequence SEQ ID NO: 36.

Examples 55-63 are made in the same manner as Examples 1-9, respectively, except Examples 55-63 are made starting from strain P-14 instead of stain P-7. Examples 55-63 correspond to Examples 1-9, respectively, except the FRD gene in Examples 55-63 is the mutated T. brucei FRD gene having the nucleotide sequence SEQ ID NO: 37.

### Examples 64-126—Deletion of Native GPD Gene

Examples 1-63 each are transformed with an integration fragment having nucleotide sequence SEQ ID NO: 38 using lithium acetate methods as described before. This integration fragment contains the following elements, 5′ to 3′: a DNA fragment with homology for integration corresponding to the region immediately upstream of the I. orientalis GPD1 open reading frame, a PDC transcriptional terminator, the URA3 promoter, the I. orientalis URA3 gene, a URA3 terminator, an additional URA3 promoter direct repeat for marker recycling and a DNA fragment with homology for integration corresponding to the region directly downstream of the I. orientalis GPD1 open reading frame. Successful transformants are selected on selection plates lacking uracil, confirmed by PCR using primers having nucleotide sequences SEQ ID NOs: 129 and 130 to confirm the 5′-crossover and SEQ ID NOs: 131 and 132 to confirm the 3′-crossover), and are then grown and plated on FOA as before until a strain in which the URA3 marker has looped out is identified. This strain is then transformed with an integration fragment having nucleotide sequence SEQ ID NO: 39. This integration fragment contains the following elements, 5′ to 3′: a DNA fragment with homology for integration corresponding to the region immediately upstream of the I. orientalis GPD1 open reading frame, the URA3 promoter, the I. orientalis URA3 gene, a URA3 terminator an additional URA3 promoter direct repeat for marker recycling a PDC transcriptional terminator, and a DNA fragment with homology for integration corresponding to the region directly downstream of the I. orientalis GPD1 open reading frame. Successful transformants are again selected on selection plates lacking uracil and confirmed by PCR, using primers having nucleotide sequences SEQ ID NOs: 130 and 132) to confirm the 5′-crossover and SEQ ID NOs: 129 and 131 to confirm the 3′-crossover). Retention of the first GPD1 deletion construct is also reconfirmed by repeating the PCR reactions used to verify proper integration of SEQ ID NO: 38 above. Confirmed isolates are grown and plated on FOA as before until a strain in which the URA3 marker has looped out is identified. The strains resulting from the transformations of Examples 1-63 are designated Examples 64-126, respectively.

### Example 127-252—Deletion of Native PGI gene

Integration fragment 5-1 (having SEQ ID NO: 40) for the deletion of the first copy of the I. orientalis PGI gene, contains the following elements, 5′ to 3′: a DNA fragment with homology for integration corresponding to the region immediately upstream of the I. orientalis PGI open reading frame, a PDC1 transcriptional terminator, the I. orientalis URA3 promoter, gene, and terminator in succession, followed by an additional URA3 promoter which serves as a direct repeat for marker recycling, and a region immediately downstream of the I. orientalis PGI open reading frame.

Integration fragment 5-2 (having SEQ ID NO: 41) for the deletion of the second copy of the I. orientalis PGI gene, contains the following elements, 5′ to 3′: a DNA fragment with homology for integration corresponding to the region immediately downstream of the I. orientalis PGI open reading frame, a PDC1 transcriptional terminator, the I. orientalis URA3 promoter, gene, and terminator in succession, followed by an additional URA3 promoter which serves as a direct repeat for marker recycling, and a region immediately upstream of the I. orientalis PGI open reading frame.

Examples 1-127 each are transformed with integration fragment 5-1 using the lithium acetate process described before. Successful transformants are selected on PGI deletion selection plates lacking uracil (SC −ura, +20 g/L fructose, +0.5 g/L glucose) incubated 3-5 days, confirmed by PCR using primers having nucleotide sequences SEQ ID NOs: 84 and 85 to confirm the 5′-crossover and SEQ ID NOs: 72 and 86 to confirm the 3′-crossover. Successful transformants are grown and plated on FOA as before until a strain in which the URA3 marker has looped out is identified. In each case, the resulting strain is then transformed with integration fragment 5-2 in the same manner and successful transformants selected on PGI deletion selection plates lacking uracil (SC −ura, +20 g/L fructose, +0.5 g/L glucose) incubated 3-5 days and confirmed by PCR using primers having nucleotide sequences SEQ ID NOs: 72 and 84 to confirm the 5′-crossover and SEQ ID NOs: 85 and 86 to confirm the 3′-crossover. A successful deletant in which the URA3 marker has looped out is again identified as before. The strains resulting from the transformations of Examples 1-126 are designated Examples 127-252, respectively.

### Shake Flask Evaluation for Succinate Production

Example 1-1 is streaked out for single colonies on URA selection plates and incubated at 30° C. until single colonies are visible (1-2 days). Cells from plates are scraped into sterile growth medium and the optical density (OD₆₀₀) is measured. Optical density is measured at wavelength of 600 nm with a 1 cm pathlength using a model Genesys20 spectrophotometer (Thermo Scientific). Dry cell mass is calculated from the measured OD₆₀₀ value using an experimentally derived conversion factor of 1.7 OD₆₀₀ units per 1 g dry cell mass.

A shake flask is inoculated with the cell slurry to reach an initial OD₆₀₀ of 0.1-0.3. Prior to inoculation, the 250 mL baffled shake flasks containing 1.75 g/L dry CaCO₃ are sterilized. Immediately prior to inoculating, 50 mL of shake flask medium is added to the dry calcium carbonate. The shake flask medium is a sterilized, 5.5 pH aqueous solution of urea (2.3 g/L), magnesium sulfate heptahydrate (0.5 g/L), potassium phosphate monobasic (3.0 g/L), trace element solution (1 mL/L) and vitamin solution (1 mL/L), glucose (120.0 g/L), glycerol (0.1 g/L), 2-(N-Morpholino) ethanesulfonic acid (MES) (4.0 g/L). For strains lacking the URA3 gene (URA-) 20 mg/L uracil is added to the media. The trace element solution is an aqueous solution of EDTA (15.0 g/L), zinc sulfate heptahydrate (4.5 g/L), manganese chloride dehydrate (1.0 g/L), cobalt(II) chloride hexahydrate (0.3 g/L), copper(II)sulfate pentahydrate (0.3 g/L), disodium molybdenum dehydrate (0.4 g/L), calcium chloride dehydrate (4.5 g/L), iron sulphate heptahydrate (3 g/L), boric acid (1.0 g/L), and potassium iodide (0.1 g/L). The vitamin solution is an aqueous solution of biotin (D−; 0.05 g/L), calcium pantothenate (D+; 1 g/L), nicotinic acid (5 g/L), myo-inositol (25 g/L), pyridoxine hydrochloride (1 g/L), p-aminobenzoic acid (0.2 g/L).

The inoculated flask is incubated at 30° C. with shaking at 150 rpm for 72 hours and taken to analysis. Succinate concentration in the broth at the end of 72 hours fermentation is determined by gas chromatography with flame ionization detector and glucose by high performance liquid chromatography with refractive index detector.

Examples 2 through 252 are cultured in shake flasks in similar manner and found to produce succinate. The succinate concentration in the broth is measured and yield and titer are calculated.

### Examples 253-255

Integration fragment P6-2a (having nucleotide sequence SEQ ID NO: 116) contains the I. orientalis FUM (IoFUM) gene (nucleotide sequence SEQ ID NO: 70), the last 568 base pairs of the URA3 marker, URA3 promoter, PGK promoter, PDC terminator and ADHb downstream integration arm.

Integration fragment P6-4a (having nucleotide sequence SEQ ID NO: 125) contains the I. orientalis FUM (IoFUM) gene (nucleotide sequence SEQ ID NO: 70) the last 568 base pairs of the URA3 marker, URA3 promoter, PGK promoter, PDC terminator and ADHb upstream integration arm.

Strain P-5 is simultaneously transformed with each of integration fragments P6-1 and P6-2a using the standard lithium acetate process described before. Successful transformants are identified by PCR, the transformants are grown and plated on 5FOA plates until a strain in which the URA3 marker has looped out is identified as before. This strain is designated strain P-6a.

Strain P-6a is simultaneously transformed with each of integration fragments P6-3 and P6-4a and using the standard lithium acetate process described before. Successful transformants are identified by PCR, the transformants are grown and plated on 5FOA plates until a strain in which the URA3 marker has looped out is identified as before. This strain is designated strain P-7a.

Strain P-7a is transformed with an integration fragments having nucleotide sequences SEQ ID NO: 38 and SEQ ID NO: 39, deleting the GPD gene as described with respect to Example 64-126 above. The resulting strain is named P-8a. Strain P-8a is grown and plated on 5FOA plates until a strain in which the URA3 marker has looped out is identified as before. The resulting strain is named P-8b.

### Construction of Strains 253, 254, and 255

Integration fragment 6-1, having nucleotide sequence SEQ ID NO: 133, contains the following elements, 5′ to 3′: a DNA fragment with homology for integration corresponding to the region immediately downstream of the I. orientalis GPD1 open reading frame, an I. orientalis ENO1 promoter, the E. coli SthA gene (having the nucleotide sequence SEQ ID NO: 24), the I. orientalis PDC terminator, a LoxP site, the I. orientalis PGK promoter, the S. cerevisiae MEL5 gene and terminator (having the nucleotide sequence SEQ ID NO: 134), another LoxP site, and a DNA fragment with homology for integration corresponding to the region directly upstream of the I. orientalis GPD1 open reading frame.

Integration fragment 6-2, having nucleotide sequence SEQ ID NO: 135, contains the following elements, 5′ to 3′: a DNA fragment with homology for integration corresponding to the region immediately upstream of the I. orientalis GPD1 open reading frame, an I. orientalis ENO1 promoter, the E. coli SthA gene (having the nucleotide sequence SEQ ID NO: 24), the I. orientalis PDC terminator, the URA3 promoter, the I. orientalis URA3 gene, an additional URA3 promoter direct repeat for marker recycling and a DNA fragment with homology for integration corresponding to the region directly downstream of the I. orientalis GPD1 open reading frame.

Integration fragment 6-3, having nucleotide sequence SEQ ID NO: 136 contains the following elements, 5′ to 3′: a DNA fragment with homology for integration corresponding to the region immediately downstream of the I. orientalis GPD1 open reading frame, an I. orientalis ENO1 promoter, the A. vinelandii SthA gene (having the nucleotide sequence SEQ ID NO: 27), the I. orientalis PDC terminator, a LoxP site, the I. orientalis PGK promoter, the S. cerevisiae MEL5 gene and terminator (having the nucleotide sequence SEQ ID NO: 134), another LoxP site, and a DNA fragment with homology for integration corresponding to the region directly upstream of the I. orientalis GPD1 open reading frame.

Integration fragment 6-4, having nucleotide sequence SEQ ID NO: 137, contains the following elements, 5′ to 3′: a DNA fragment with homology for integration corresponding to the region immediately upstream of the I. orientalis GPD1 open reading frame, an I. orientalis ENO1 promoter, the A. vinelandii SthA gene (having the nucleotide sequence SEQ ID NO: 27), the I. orientalis PDC terminator, the URA3 promoter, the I. orientalis URA3 gene, an additional URA3 promoter direct repeat for marker recycling and a DNA fragment with homology for integration corresponding to the region directly downstream of the I. orientalis GPD1 open reading frame.

Integration fragment 6-5, having nucleotide sequence SEQ ID NO: 138, contains the following elements, 5′ to 3′: a DNA fragment with homology for integration corresponding to the region immediately downstream of the I. orientalis GPD1 open reading frame, an I. orientalis ENO1 promoter, the P. fluorescens SthA gene (having the nucleotide sequence SEQ ID NO: 139), the I. orientalis PDC terminator, a LoxP site, the I. orientalis PGK promoter, the S. cerevisiae MEL5 gene and terminator (having the nucleotide sequence SEQ ID NO: 134), another LoxP site, and a DNA fragment with homology for integration corresponding to the region directly upstream of the I. orientalis GPD1 open reading frame.

Integration fragment 6-6, having nucleotide sequence SEQ ID NO: 140, contains the following elements, 5′ to 3′: a DNA fragment with homology for integration corresponding to the region immediately upstream of the I. orientalis GPD1 open reading frame, an I. orientalis ENO1 promoter, the P. fluorescens SthA gene (having the nucleotide sequence SEQ ID NO: 139), the I. orientalis PDC terminator, the URA3 promoter, the I. orientalis URA3 gene, an additional URA3 promoter direct repeat for marker recycling and a DNA fragment with homology for integration corresponding to the region directly downstream of the I. orientalis GPD1 open reading frame.

Examples 253, 254 and 255 are constructed in the following manner. Strain P-8b is co-transformed with the integration fragments listed in the second column of Table 3. Successful integrants in each case are identified as blue colonies on selection plates with 5-bromo-4-chloro-3-indolyl-alpha-D-galactopyranoside and lacking uracil, and confirmed by PCR. PCR oligos used to test the 3′ and 5′ crossovers of each integration fragment are listed in the third through sixth columns of Table 3. In each case, successful transformants are grown for several rounds and plated on 5-fluoroorotic acid (FOA) plates to identify a strain in which the URA3 marker has looped out. Loopout of the URA3 marker is confirmed by PCR.

TABLE 3

| | | 1^{st} | 1^{st} | 2^{nd} | 2^{nd} |
|---|---|---|---|---|---|
| | | integration | integration | integration | integration |
| | | 3′ | 5′ | 3′ | 5′ |
| | | crossover | crossover | crossover | crossover |
| Strain | Integration | oligos | oligos | oligos | oligos |
| name | Fragments | SEQ ID | SEQ ID | SEQ ID | SEQ ID |
| Example | 6-1 and 6-2 | NO: 130 | NO: 131 | NO: 130 | NO: 131 |
| 253 | | and 145 | and 143 | and 143 | and 144 |
| Example | 6-3 and 6-4 | NO: 130 | NO: 131 | NO: 130 | NO: 131 |
| 254 | | and 145 | and 141 | and 141 | and 144 |
| Example | 6-5 and 6-6 | NO: 130 | NO: 131 | NO130 | NO: 131 |
| 255 | | and 145 | and 142 | and 142 | and 144 |

Table 4 summarizes the genetic modifications to Strains 253, 254 and 255 (relative to the wild-type strain):

TABLE 4
Strains 253, 254 and 255

| Strain name | Description |
|---|---|
| 253 | URA deletion (2) |
| | PDC deletion (2) |
| | I. orientalis PYC1 insertion at ADHa (2) |
| | S. pombe MAE insertion at ADHa (2) |
| | L. mexicana FRD insertion at CYB2b (2) |
| | R. delemar MDH insertion at ADHb (2) |
| | I. orientalis FUM insertion at ADHb (2) |
| | GPD deletion |
| | E. coli SthA insertion at GPD (2) |
| 254 | URA deletion (2) |
| | PDC deletion (2) |
| | I. orientalis PYC1 insertion at ADHa (2) |
| | S. pombe MAE insertion at ADHa (2) |
| | L. mexicana FRD insertion at CYB2b (2) |
| | R. delemar MDH insertion at ADHb (2) |
| | I. orientalis FUM insertion at ADHb (2) |
| | GPD deletion |
| | A. vinelandii SthA insertion at GPD (2) |
| 255 | URA deletion (2) |
| | PDC deletion (2) |
| | I. orientalis PYC1 insertion at ADHa (2) |
| | S. pombe MAE insertion at ADHa (2) |
| | L. mexicana FRD insertion at CYB2b (2) |
| | R. delemar MDH insertion at ADHb (2) |
| | I. orientalis FUM insertion at ADHb (2) |
| | GPD deletion |
| | P. fluorescens SthA insertion at GPD (2) |

### Shake Flask Evaluation for Succinate Production for Strains 253-255

Strains P-8, 253, 254 and 255 are separately evaluated for succinate production. In each case, the strain is streaked out for single colonies on plates lacking uracil and incubated at 30° C. until single colonies are visible (1-2 days). Cells from plates are scraped into sterile growth medium and the optical density (OD₆₀₀) is measured. Optical density is measured at wavelength of 600 nm with a 1 cm pathlength using a model Genesys20 spectrophotometer (Thermo Scientific). Dry cell mass is calculated from the measured OD₆₀₀ value using an experimentally derived conversion factor of 1.7 OD₆₀₀ units per 1 g dry cell mass.

A shake flask is inoculated with the cell slurry to reach an initial OD₆₀₀ of 0.1-0.3. Prior to inoculation, the 250 mL baffled shake flasks containing 1.28 g/L dry CaCO₃ are sterilized. Immediately prior to inoculating, 50 mL of shake flask medium is added to the dry calcium carbonate. The shake flask medium is a sterilized, 4.5 pH aqueous solution of urea (2.3 g/L), magnesium sulfate heptahydrate (0.5 g/L), potassium phosphate monobasic (3.0 g/L), trace element solution (1 mL/L) and vitamin solution (1 mL/L), glucose (120.0 g/L), glycerol (0.1 g/L), 2-(N-Morpholino) ethanesulfonic acid (MES) (4.0 g/L). The trace element solution is an aqueous solution of EDTA (15.0 g/L), zinc sulfate heptahydrate (4.5 g/L), manganese chloride dehydrate (1.0 g/L), cobalt(II) chloride hexahydrate (0.3 g/L), copper(II)sulfate pentahydrate (0.3 g/L), disodium molybdenum dehydrate (0.4 g/L), calcium chloride dehydrate (4.5 g/L), iron sulphate heptahydrate (3 g/L), boric acid (1.0 g/L), and potassium iodide (0.1 g/L). The vitamin solution is an aqueous solution of biotin (D-; 0.05 g/L), calcium pantothenate (D+; 1 g/L), nicotinic acid (5 g/L), myo-inositol (25 g/L), pyridoxine hydrochloride (1 g/L), p-aminobenzoic acid (0.2 g/L).

The inoculated flask is incubated at 30° C. with shaking at 150 rpm for 96 hours and taken to analysis. Succinate and glucose concentrations in the broth at the end of 96 hours fermentation are determined by high performance liquid chromatography with refractive index detector. Results are as indicated in Table 5:

TABLE 5

| | | Average | | Average |
|---|---|---|---|---|
| | Glucose | glucose | Succinate | succinate |
| | after | consumption rate, | after | production rate, |
| Strain | 96 hr, g/L | g/L/hr | 96 hr, g/L | g/L/hr |
| P-8a | 5.46 | 1.190 | 57.6 | 0.60 |
| 253 | 6.20 | 1.185 | 88.4 | 0.92 |
| 255 | 6.10 | 1.186 | 84.1 | 0.88 |
| 257 | 8.58 | 1.161 | 89.0 | 0.93 |

As can be seen from the data in Table 5, all strains produce succinate. However, Examples 253-255 produce more succinate, at a 50% greater rate, than Strain P-8a.

### Example 256

The URA3 gene is deleted from a wild type strain of S. cerevisiae (CEN-PK 113-7D) to create a strain with a uracil auxotrophy. This strain is called S-1.

Ethanol production is eliminated in S-1 by deletion of the three PDC genes (PDC1, PDC5, and PDC6), using conventional methods, to produce a strain (S-2) that does not produce ethanol. A pathway from pyruvate to succinate is introduced into strain S-2 by the integration of the following exogenous genes driven by strong promoters: the I. orientalis PYC gene, the R. delemar MDH gene, the I. orientalis FUM (fumarase), the L. Mexicana FRD gene, and the S. pombe MAE gene. The various promoters include the S. cerevisiae CYC1 promoter, the S. cerevisiae ADH1 promoter and the S. cerevisiae GPD1 promoter.

Strain S-3 is transformed with the E. coli soluble transhydrogenase (SthA) gene (SEQ ID NO: 21) under the control of the S. cerevisiae CYC1 promoter. The resulting strain (which still is prototrophic for uracil) is called S-4. Strain S-4 cannot produce ethanol, has an active metabolic pathway to succinate, overexpresses the soluble transhydrogenase enzyme and is prototrophic for uracil.

After deletion of the PDC genes from S. cerevisiae, it becomes necessary to supplement the growth medium with a C2 carbon source to support growth. Additionally, glucose is known to suppress growth of S. cerevisiae strains lacking adequate PDC activity. Therefore, Strains S-3 and S-4 are grown on a medium containing ethanol as a sole carbon source to a suitable cell density in a shake flask. The cells are collected by centrifugation and the ethanol media discarded. The cells are resuspended in a glucose containing medium in a shake flask and cultivated under aeration at 30° in a stirred shake flask, and succinate formation is monitored until glucose depletion. Strain S-4, which exhibits transhydrogenase activity, shows improved succinate production compared with strain S-3, which lacks transhydrogenase activity.

1. A recombinant yeast cell which has an active reductive TCA pathway from pyruvate or phosphoenolpyruvate to succinate and overexpresses a NAD(P)⁺ transhydrogenase enzyme and which further has integrated into its genome one or more of (i) an exogenous pyruvate carboxylase gene that encodes for an enzyme which catalyzes the conversion of pyruvate to oxaloacetate, (ii) an exogenous malate dehydrogenase gene which encodes for an enzyme that catalyzes the conversion of oxaloacetate to malate (iii) an exogenous fumarase gene that encodes for an enzyme which catalyzes the conversion of malate to fumarate and (iv) an exogenous fumarate reductase gene which encodes for an enzyme which catalyzes the conversion of fumarate to succinate.

2.-38. (canceled)

39. The recombinant cell of claim 1 which has a deletion or disruption of a native pyruvate decarboxylase gene.

40. The recombinant cell of claim 1, wherein the host cell is a yeast cells classified under one or more of the genera Candida, Pichia, Saccharomyces, Schizosaccharomyces, Zygosaccharomyces, Kluyveromyces, Debaryomyces, Pichia, Issatchenkia, and Hansenula.

41. The recombinant cell of any preceding claim 1, wherein the host cell is selected from C. sonorensis, K. marxianus, K. thermotolerans, C. methanesorbosa, S. bulderi, I. orientalis, C. lambica, C. sorboxylosa, C. zemplinina, C. geochares, P. membranifaciens, Z. kombuchaensis, C. sorbosivorans, C. vandenvaltii, C. sorbophila, Z. bisporus, Z. lentus, S. bayanus, D. castellii, C, boidinii, C. etchellsii, K. lactis, P. jadinii, P. anomala, Saccharomyces cerevisae and Saccharomycopsis crataegensis.

42. The recombinant cell of claim 1, wherein the host cell is selected from Issatchenkia orientalis, Pichia galeiformis, Pichia sp. YB-4149 (NRRL designation), Candida ethanolica, P. deserticola, P. membranifaciens and P. fermentans.

43. The recombinant cell of claim 1, wherein the host cell is Crabtree negative as a wild-type strain.

44. The recombinant cell of claim 1, wherein the host cell is succinate-resistant as a wild-type strain.

45. The recombinant cell of claim 1, wherein the host cell exhibits a specific glucose consumption rate of at least 0.5 gram of glucose per gram dry weight of cells per hour, as a wild-type strain.

46. The recombinant cell of claim 45, wherein the host cell exhibits a specific glucose consumption rate of at least 1.0 gram of glucose per gram dry weight of cells per hour, as a wild-type strain.

47. The recombinant cell of claim 46, wherein the host cell exhibits a specific glucose consumption rate of at least 1.5 gram of glucose per minute per gram dry weight of cells, as a wild-type strain.

48. The recombinant cell of claim 1, wherein the host cell exhibits a volumetric glucose consumption rate of at least 3 gram of glucose per liter per hour as a wild-type strain.

49. The recombinant cell of claim 48, wherein the host cell exhibits a volumetric glucose consumption rate of at least 5 gram of glucose per liter per hour as a wild-type strain.

50. The recombinant cell of claim 46, wherein the host cell exhibits a volumetric glucose consumption rate of at least 8 gram of glucose per liter per hour as a wild-type strain.

51. The recombinant cell of claim 1 which exhibits a volumetric glucose consumption rate of at least 0.5 gram of glucose per liter per hour.

52. The recombinant cell of claim 1 which exhibits a volumetric glucose consumption rate of at least 0.75 gram of glucose per liter per hour.

53. The recombinant cell of claim 1 which exhibits a volumetric glucose consumption rate of at least 0.9 gram of glucose per liter per hour.

54. The recombinant cell of claim 1, wherein the host cell is I. orientalis.

55.-63. (canceled)
